(19) United States
(12) Patent Application Publication
(10) Pub. No.: US 2023/0399640 A1
(43) Pub. Date: Dec. 14, 2023

(54) COMPOSITIONS AND METHODS FOR INHIBITING GENE EXPRESSION

(71) Applicant: FLAGSHIP PIONEERING INNOVATIONS V, INC., Cambridge, MA (US)

(72) Inventors: Jodi Michelle KENNEDY, Dedham, MA (US); Jeremiah Dale FARELLI, Marblehead, MA (US)

(21) Appl. No.: 18/246,480

(22) PCT Filed: Sep. 24, 2021
(86) PCT No.: PCT/US2021/051945

§ 371 Date: Mar. 23, 2023

Related U.S. Application Data
(60) Provisional application No. 63/082,555, filed on Sep. 24, 2020.
(60) Provisional application No. 63/242,957, filed on Sep. 10, 2021.

(51) Int. Cl. C12N 15/11; C12N 9/22; C12N 9/10; C07K 14/195

(52) U.S. Cl. CPC C12N 15/11; C12N 9/22; C12N 9/1007; C12Y 201/01043; C07K 14/195; C12N 2310/20; C07K 2319/80

(57) ABSTRACT

The present disclosure relates to expression repressors and expression repression systems for modulating expression of a target gene in a cell.

### CROSS-REFERENCE TO RELATED APPLICATIONS

The present application claims the benefit of U.S. provisional applications 63/082,555 filed Sep. 24, 2020 and 63/242,957 filed Sep. 10, 2021. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Sep. 17, 2021, is named 02057-7017 WO_SL.txt and is 694,831 bytes in size.

### BACKGROUND

Mis regulation of gene expression is the underlying cause of many diseases (e.g., in mammals, e.g., humans). While techniques exist that modulate gene expression for short periods of time, the treatment of many diseases calls for stable, long-term modulation of gene expression. There is a need for novel tools, systems, and methods to stably alter, e.g., decrease, expression of disease associated genes.

### SUMMARY

The disclosure provides, among other things, expression repressors and expression repression systems that may be used to modulate, e.g., decrease, expression of a target gene.

In some aspects, an expression repressor comprises a DNA targeting moiety and a repressor domain capable of modulating (e.g., decreasing) the expression of a target gene. In some embodiments, an expression repressor comprises a DNA targeting moiety, a first repressor domain, and a second repressor domain. In some embodiments, the first repressor domain is different from the second repressor domain. In some embodiments, the first repressor domain is identical to the second repressor domain.

In some aspects, the disclosure features an expression repression system comprising a first expression repressor comprising a first DNA-targeting moiety and a first repressor domain, and a second expression repressor comprising a second DNA-targeting moiety and a second repressor domain. In some embodiments, the first DNA-targeting moiety specifically binds a first DNA sequence, and the second DNA-targeting moiety specifically binds a second DNA sequence different from the first DNA sequence. In some embodiments, the first repressor domain is different from the second repressor domain. In some embodiments, an expression repression system comprises: (i) a first expression repressor comprising a first DNA-targeting moiety, a first repressor domain, and a second repressor domain, and (ii) a second expression repressor comprising a second DNA-targeting moiety and a first repressor domain. In some embodiments, all three of the repressor domains are different. In some embodiments, at least two repressor domains are identical. In some embodiments, an expression repression system comprises: (i) a first expression repressor comprising a first DNA-targeting moiety, a first repressor domain, and a second repressor domain, and (ii) a second expression repressor comprising a second DNA-targeting moiety, a first repressor domain and a second repressor domain. In some embodiments, all four of the repressor domains are different. In some embodiments, at least two repressor domains are identical.

Generally, modulation of expression of a target gene by an expression repression system involves the binding of the first expression repressor and second expression repressor to the first and second DNA sequences, respectively. Binding of the first and second DNA sequences localizes the functionalities of the first and second repressor domains to those sites. Without wishing to be bound by theory, in some embodiments it is thought that employing the functionalities of both the first and second repressor domains stably represses expression of a target gene associated with or comprising the first and/or second DNA sequences, e.g., wherein the first and/or second DNA sequences are or comprise sequences of the target gene or one or more operably linked transcription control elements.

In some aspects, the disclosure provides an expression repressor or an expression repression system comprising: a targeting moiety that binds a genomic locus comprising at least 14, 15, 16, 17, 18, 19, or 20 nucleotides of the sequence of SEQ ID NO: 1-21, wherein the expression repressor or the expression repression system is capable of decreasing the expression of a target gene.

In some aspects, the disclosure provides an expression repressor comprising: a DNA-targeting moiety (wherein optionally the DNA-targeting moiety comprises a CRISPR/Cas molecule, e.g., a catalytically inactive CRISPR/Cas protein), that binds to a transcription regulatory element (e.g., a promoter or transcription start site (TSS)) operably linked to a target gene, or a sequence proximal to said transcription regulatory element; and a repressor domain. In some embodiments, an expression repressor comprises a DNA-targeting moiety (wherein optionally the DNA-targeting moiety comprises a CRISPR/Cas molecule, e.g., a catalytically inactive CRISPR/Cas protein), that binds to a transcription regulatory element (e.g., a promoter or transcription start site (TSS)) operably linked to a target gene, or a sequence proximal to said transcription regulatory element, a first repressor domain, and a second repressor domain. In some embodiments, the first repressor domain is identical to the second repressor domain. In some embodiments, the first repressor domain is not identical to the second repressor domain.

In some aspects, the disclosure provides an expression repression system comprising: (i) a first expression repressor comprising a first DNA-targeting moiety (wherein optionally the DNA-targeting moiety comprises a CRISPR/Cas molecule, e.g., a catalytically inactive CRISPR/Cas protein), that binds to a transcription regulatory element (e.g., a promoter or transcription start site (TSS)) operably linked to a target gene, or a sequence proximal to said transcription regulatory element; and a first repressor domain and a second expression repressor comprising a second DNA-targeting moiety (wherein optionally the DNA-targeting moiety comprises a CRISPR/Cas molecule, e.g., a catalytically inactive CRISPR/Cas protein), that binds to a transcription regulatory element (e.g., a promoter or transcription start site (TSS)) operably linked to a target gene, or a sequence proximal to said transcription regulatory element; and a first repressor domain. In some embodiments, the first repressor domains of the first expression repressor and the second expression repressors are identical. In some embodiments, the first repression domains of the first and the second expression pressors are different. In some embodiments, the first DNA-targeting moiety and the second DNA-targeting moiety are identical. In some embodiments, the first DNA-targeting moiety and the second DNA-targeting moiety are different. In some embodiments, an expression repression system comprises a first expression repressor comprising a first DNA-targeting moiety (wherein optionally the DNA-targeting moiety comprises a CRISPR/Cas molecule, e.g., a catalytically inactive CRISPR/Cas protein), that binds to a transcription regulatory element (e.g., a promoter or transcription start site (TSS)) operably linked to a target gene, or a sequence proximal to said transcription regulatory element, a first repressor domain, and a second repressor domain and a second expression repressor comprising a second DNA-targeting moiety (wherein optionally the DNA-targeting moiety comprises a CRISPR/Cas molecule, e.g., a catalytically inactive CRISPR/Cas protein), that binds to a transcription regulatory element (e.g., a promoter or transcription start site (TSS)) operably linked to a target gene, or a sequence proximal to said transcription regulatory element; a first repressor domain and a second repressor domain. In some embodiments, the first repressor domains of the first expression repressor and the second expression repressors are identical. In some embodiments, the first repression domains of the first and the second expression pressors are different. In some embodiments, the second repressor domains of the first expression repressor and the second expression repressors are identical. In some embodiments, the second repression domains of the first and the second expression pressors are different.

In some aspects, the disclosure features a nucleic acid encoding an expression repressor or a component thereof (e.g., a gRNA). In some aspects, the disclosure is directed to a nucleic acid encoding the first expression repressor, second expression repressor, both, or a component thereof (e.g., a gRNA). In some aspects, the disclosure is directed to a vector comprising a nucleic acid described herein. In some aspects, the disclosure is directed to a cell comprising an expression repressor, an expression repression system, nucleic acid, or vector described herein. In some aspects, the disclosure is directed to a lipid nanoparticle comprising a vector, a nucleic acid, an expression repression system, or an expression repressor described herein. In some aspects, the disclosure is directed to a reaction mixture comprising an expression repressor, an expression repression system, a nucleic acid, a vector, or a lipid nanoparticle described herein. In some aspects, the disclosure is directed to a pharmaceutical composition comprising an expression repression system, nucleic acid, or vector described herein.

In some aspects, the disclosure is directed to a method of decreasing expression of a target gene comprising providing an expression repressor or an expression repression system described herein and contacting the target gene and/or one or more operably linked transcription control elements with the expression repressor or the expression repression system, thereby decreasing expression of the target gene.

In some aspects, the disclosure is directed to a method of treating a condition associated with over-expression of a target gene in a subject, comprising administering an expression repressor or an expression repression system, nucleic acid, or vector described herein to the subject, thereby treating the condition.

In some aspects, the disclosure is directed to a method of treating a condition associated with mis-regulation of a target gene in a subject, comprising administering an expression repressor or an expression repression system, nucleic acid, or vector described herein to the subject, thereby treating the condition.

Additional features of any of the aforesaid methods or compositions include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Sep. 23, 2019. When one gene or protein references a plurality of sequence accession numbers, all of the sequence variants are encompassed.

### ENUMERATED EMBODIMENTS

1. An expression repressor comprising:
   a DNA-targeting moiety and
   a repressor domain,
      wherein the expression repressor is capable of decreasing expression of a target gene.
2. The expression repressor of embodiment 1, wherein the DNA-targeting moiety binds to a transcription regulatory element (e.g., a promoter, an enhancer, a super enhancer, or transcription start site (TSS)) operably linked to the target gene or binds to a sequence proximal to said transcription regulatory element.
3. The expression repressor of embodiment 1 or 2, wherein the repressor domain is covalently linked to the DNA-targeting moiety.
4. The expression repressor of embodiments 1 or 2, wherein the repressor domain is linked to the DNA-targeting moiety via a linker.
5. The expression repressor of any of embodiment 1-4, wherein the repressor domain is C-terminal of the DNA-targeting moiety.
6. The expression repressor of any of embodiments 1-4, wherein the repressor domain is N-terminal of the DNA-targeting moiety.
7. The expression repressor of any of embodiments 1-6, wherein the repressor domain is encoded by a nucleotide sequence chosen from any of SEQ ID NOs: 47-56, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
8. The expression repressor of any of embodiments 1-7, wherein the repressor domain comprises an amino acid sequence according to any of SEQ ID NOs: 57-66, 90 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
9. The expression repressor of any of the preceding embodiments, wherein the repressor domain is MQ1 or a functional variant or fragment thereof, e.g., wherein the repressor domain comprises an amino acid sequence of SEQ ID NO: 57 or 90 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is C-terminal of the DNA-targeting moiety.
10. The expression repressor of any of the preceding embodiments, wherein the repressor domain is DNMT1 or a functional variant or fragment thereof, e.g., wherein the repressor domain comprises an amino acid sequence of SEQ ID NO: 58 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is C-terminal of the DNA-targeting moiety.
11. The expression repressor of any of the preceding embodiments, wherein the repressor domain is DNMT3a/3L, or a functional variant or fragment thereof, e.g., wherein the repressor domain comprises an amino acid sequence of SEQ ID NO: 59 or 60 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is C-terminal of the DNA-targeting moiety.
12. The expression repressor of any of the preceding embodiments, wherein the repressor domain is KRAB, or a functional variant or fragment thereof, e.g., wherein the repressor domain comprises an amino acid sequence of SEQ ID NO: 61 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is C-terminal of the DNA-targeting moiety.
13. The expression repressor of any of the preceding embodiments, wherein the repressor domain is G9A, or a functional variant or fragment thereof, e.g., wherein the repressor domain comprises an amino acid sequence of SEQ ID NO: 62 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is N-terminal of the DNA-targeting moiety.
14. The expression repressor of any of the preceding embodiments, wherein the repressor domain is HDAC8, or a functional variant or fragment thereof, e.g., wherein the repressor domain comprises an amino acid sequence of SEQ ID NO: 63 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is C-terminal of the DNA-targeting moiety.
15. The expression repressor of any of the preceding embodiments, wherein the repressor domain is LSD1, or a functional variant or fragment thereof, e.g., wherein the repressor domain comprises an amino acid sequence of SEQ ID NO: 64 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is C-terminal of the DNA-targeting moiety.
16. The expression repressor of any of the preceding embodiments, wherein the repressor domain is EZH2, or a functional variant or fragment thereof, e.g., wherein the repressor domain comprises an amino acid sequence of SEQ ID NO: 64 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the repressor domain is N-terminal of the DNA-targeting moiety.
17. The expression repressor of any of the preceding embodiments, wherein the repressor domain is FOG1, or a functional variant or fragment thereof, e.g., wherein the repressor domain comprises an amino acid sequence of SEQ ID NO: 66 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
18. The expression repressor of any of the preceding embodiments, which further comprises a second repressor domain.
19. The expression repressor of embodiment 18, wherein the DNA-targeting moiety is situated between the repressor domain and the second repressor domain.
20. An expression repressor comprising:
   a DNA-targeting moiety,
   a first repressor domain and
   a second repressor domain,
   wherein the expression repressor is capable of decreasing expression of a target gene.
21. The expression repressor of any preceding embodiment, wherein the DNA-targeting moiety binds to a transcription regulatory element (e.g., a promoter, an enhancer, a super enhancer, or transcription start site (TSS)) operably linked to the target gene or binds to a sequence proximal to said transcription regulatory element.
22. The expression repressor of any of embodiments 18-21, wherein the first repressor domain is identical to the second repressor domain.
23. The expression repressor of any of embodiments 18-21, wherein the first repressor domain is not identical to the second repressor domain.
24. The expression repressor of any of embodiments 18-23, wherein the first repressor domain and the second repressor domain are covalently linked to the DNA-targeting moiety.
25. The expression repressor of any of embodiments 18-24, wherein the first repressor domain is linked to the DNA-targeting moiety via a first linker and the second repressor domain is linked to the DNA-targeting moiety via a second linker.
26. The expression repressor of embodiment 25, wherein the first linker is identical to the second linker.
27. The expression repressor of embodiment 25, wherein the first linker is not identical to the second linker.
28. The expression repressor of any of embodiments 18-27, wherein the first repressor domain is N-terminal of the DNA-targeting moiety.
29. The expression repressor of any of embodiments 18-28, wherein the second repressor domain is C-terminal of the DNA-targeting moiety.
30. The expression repressor of any of embodiments 25-29, wherein the C-terminal end of the first repressor domain is linked to the N-terminal end of the DNA-targeting moiety via the first linker and the N-terminal end of the second repressor domain is linked to the C-terminal end of the DNA-targeting moiety via the second linker.
31. The expression repressor of any of embodiments 18-30, wherein:
   the first repressor domain is EZH2, or a functional variant or fragment thereof, e.g., wherein the first repressor domain comprises an amino acid sequence of SEQ ID NO: 64 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first repressor domain is N-terminal of the DNA-targeting moiety; and
   the second repressor domain is KRAB, or a functional variant or fragment thereof, e.g., wherein the second repressor domain comprises an amino acid sequence of SEQ ID NO: 61 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second repressor domain is C-terminal of the DNA-targeting moiety.
32. The expression repressor of any of embodiments 18-30, wherein:
   the first repressor domain is G9A, or a functional variant or fragment thereof, e.g., wherein the first repressor domain comprises an amino acid sequence of SEQ ID NO: 62 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first repressor domain is N-terminal of the DNA-targeting moiety; and
   the second repressor domain is KRAB, or a functional variant or fragment thereof, e.g., wherein the second repressor domain comprises an amino acid sequence of SEQ ID NO: 61 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second repressor domain is C-terminal of the DNA-targeting moiety.
33. The expression repressor of any of embodiments 18-30, wherein:
   the first repressor domain is FOG1, or a functional variant or fragment thereof, e.g., wherein the first repressor domain comprises an amino acid sequence of SEQ ID NO: 66 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first repressor domain is N-terminal of the DNA-targeting moiety; and
   the second repressor domain is FOG1, or a functional variant or fragment thereof, e.g., wherein the second repressor domain comprises an amino acid sequence of SEQ ID NO: 66 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second repressor domain is C-terminal of the DNA-targeting moiety.
34. The expression repressor of any of embodiments 1-33, wherein the DNA-targeting moiety binds a genomic locus comprising at least 14, 15, 16, 17, 18, 19, or 20 nucleotides of the sequence of any of SEQ ID NO: 1-21.
35. The expression repressor of any of embodiments 1-34, wherein the DNA-targeting moiety comprises a CRISPR/Cas molecule (e.g., a catalytically inactive CRISPR/Cas protein), a zinc finger domain, or a TAL effector molecule.
36. The expression repressor of embodiment 35, wherein the CRISPR/Cas molecule comprises one or more mutations selected from D10A, D839A, H840A, and N863A.
37. The expression repressor of any of embodiments 1-36, wherein the DNA-targeting moiety binds to a promoter region of a target gene, an enhancer region (e.g., a super enhancer region), or an anchor sequence.
38. The expression repressor of any of embodiments 1-37, wherein the repressor domain comprises DNMT1, DNMT3a/3L, MQ1, KRAB, G9A, HDAC8, LSD1, EZH2, or FOG1,
39. The expression repressor of any of embodiments 18-38, wherein the first repressor domain comprising, DNMT1, DNMT3a/31, MQ1, KRAB, G9A, HDAC8, LSD1, EZH2, or FOG1, and the second repressor domain comprises DNMT1, DNMT3a/31, MQ1, KRAB, G9A, HDAC8, LSD1, EZH2, or FOG1,
40. The expression repressor of any of embodiments 1-39, wherein the expression repressor is encoded by a nucleotide sequence chosen from any of SEQ ID NOs: 22-32, 92, 95 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
41. The expression repressor of any of embodiments 1-40, wherein the expression repressor comprises an amino acid sequence chosen from any of SEQ ID Nos: 33-36, 38-44, 67-69, 93, 96, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
42. The expression repressor of any of embodiments 1-41, wherein the DNA-targeting moiety is encoded by a nucleotide sequence chosen from any of SEQ ID NOs: 45 or 89 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
43. The expression repressor of any of embodiments 1-42, wherein the DNA-targeting moiety comprises an amino acid sequence according to any of SEQ ID NOs: 46 or 88, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
44. The expression repressor of any of embodiments 18-43, wherein,
   (i) the first repressor domain is encoded by a nucleotide sequence chosen from any of SEQ ID NOs: 47-56, 90 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   (ii) the second repressor domain is encoded by a nucleotide sequence chosen from any of SEQ ID NOs: 47-56, 90 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
45. The expression repressor of any of embodiments 18-43, wherein,
   (i) the first repressor domain comprises an amino acid sequence according to any of SEQ ID NOs: 57-66, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and
   (ii) the second repressor domain comprises an amino acid sequence according to any of SEQ ID NOs: 57-66, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.
46. The expression repressor of any preceding embodiments, wherein the DNA-targeting moiety comprises a TAL effector molecule, a CRISPR/Cas molecule, a zinc finger domain, a tetR domain, a meganuclease domain, or an oligonucleotide.
47. The expression repressor of any preceding embodiments, wherein the DNA-targeting moiety further comprises a gRNA, e.g., a gRNA that binds a genomic locus comprising at least 14, 15, 16, 17, 18, 19, or 20 nucleotides of the sequence of any of SEQ ID NOs: 1-21, e.g., wherein the gRNA comprises a sequence that comprises at least 14, 15, 16, 17, 18, 19, or 20 nucleotides of the sequence of any of SEQ ID NOs: 1-21.
48. The expression repressor of any of embodiments 35-47, wherein the DNA-targeting domain comprises a CRISPR/Cas molecule, e.g., a catalytically inactive CRISPR/Cas protein, and a gRNA, e.g., a gRNA that binds a genomic locus comprising at least 14, 15, 16, 17, 18, 19, or 20 nucleotides of the sequence of any of SEQ ID NOs: 1-21, e.g., wherein the gRNA comprises a sequence that comprises at least 14, 15, 16, 17, 18, 19, or 20 nucleotides of the sequence of any of SEQ ID NOs: 1-21 and the repressor domain comprises a moiety chosen from DNMT1, DNMt3a/31, MQ1, KRAB, G9A, HDAC8, LSD1, EZH2, or FOG1.
49. The expression repressor of embodiment 35-47, wherein the DNA-targeting domain comprises a CRISPR/Cas molecule, e.g., a catalytically inactive CRISPR/Cas protein, and a gRNA, e.g., a gRNA that binds a genomic locus comprising at least 14, 15, 16, 17, 18, 19, or 20 nucleotides of the sequence of any of SEQ ID NOs: 1-21, e.g., wherein the gRNA comprises a sequence that comprises at least 14, 15, 16, 17, 18, 19, or 20 nucleotides of the sequence of any of SEQ ID NOs: 1-21, the first repressor domain comprises a moiety chosen from DNMT1, DNM T3a/3L, MQ1, KRAB, G9A, HDAC8, LSD1, EZH2, or FOG1 and the second domain comprises a moiety chosen from DNMT1, DNMt3a/3L, MQ1, KRAB, G9A, HDAC8, LSD1, EZH2, or FOG1.
50. The expression repressor of any of embodiments 35-49, wherein the CRISPR/Cas molecule comprises a Cas protein or Cpf1 protein chosen from Table 1 or a variant (e.g., mutant) of any thereof.
51. The expression repressor of any of embodiments 35-50, wherein the CRISPR/Cas molecule comprises a catalytically inactive CRISPR/Cas protein, e.g., dCas9.
52. The expression repressor of any of the preceding embodiments, which: (i) comprises one or more nuclear localization signal sequences (NLS), or (ii) does not comprise an NLS.
53. The expression repressor of any of the preceding embodiments, comprising a first NLS at the N terminus, e.g., wherein the first NLS has a sequence of SEQ ID NO: 86.
54. The expression repressor of any of the preceding embodiments, comprising an NLS, e.g., a second NLS, at the C terminus, e.g., having a sequence of SEQ ID NO: 87.
55. The expression repressor of any of the preceding embodiments, wherein the first and the second NLS have the same sequence.
56. The expression repressor of any of embodiments 52-55, wherein the first and the second NLS have different sequences.
57. The expression repressor of any of the preceding embodiments, which comprises an epitope tag.
58. The expression repressor of embodiment 57, wherein the epitope tag is an HA tag, e.g., an HA tag comprising an amino acid seq of SEQ ID NO: 80.
59. The expression repressor of any of embodiments 37-58, wherein the anchor sequence comprises the sequence of SEQ ID NO: 81 or 82, or a sequence with no more than 8, 7, 6, 5, 4, 3, 2, or 1 alteration relative thereto.
60. The expression repressor of any of embodiments 37-58, wherein the anchor sequence comprises a sequence according to SEQ ID NO: 83 or 84, or a sequence with no more than 8, 7, 6, 5, 4, 3, 2, or 1 alteration relative thereto.
61. The expression repressor of any of embodiments 37-60, wherein the anchor sequence is on the same chromosome as the gene.
62. The expression repressor of any of embodiments 37-61, wherein the anchor sequence is upstream of the target gene (e.g., upstream of the TSS or upstream of the promoter).
63. The expression repressor of any of embodiments 37-61 wherein the anchor sequence is downstream of the target gene (e.g., downstream of the TSS or upstream of the promoter).
64. The expression repressor of any of the preceding embodiments, wherein binding of the expression repressor to the target gene locus decreases expression of the target gene in a cell by 10, 20, 30, 40, 50, 60, 70, 80, 90, or 100% compared to expression of the target gene in the absence of the expression repressor.
65. The expression repressor of any of the preceding embodiments, wherein binding of the expression repressor to the target gene locus appreciably decreases expression of the target gene for a time period of at least 1, 2, 3, 4, 5, 6, 7, 8, 9, 10, 11, 12, 13, 14, 15, 16, 17, 18, 19, 20, 21, or 22 days.
66. The expression repressor of any of embodiments 18-65, wherein the first and/or second repressor domain comprises a DNA methyltransferase, a histone methyltransferase, a histone deacetylase, a histone demethylase, or a recruiter of a histone modifying complex.
67. An expression repression system comprising:
   a first expression repressor comprising a first DNA-targeting moiety and a first repressor domain, and
   a second expression repressor comprising a second DNA-targeting moiety and a second repressor domain,
   wherein the first DNA-targeting moiety specifically binds a first DNA sequence, and the second DNA-targeting moiety specifically binds a second DNA sequence different from the first DNA sequence, and
   wherein the first repressor domain is different from the second repressor domain.
68. An expression repression system comprising:
   a first expression repressor comprising a first DNA-targeting moiety and a first repressor domain, and
   a second expression repressor comprising a second DNA-targeting moiety and a second repressor domain,
   wherein the first or second repressor domain comprises an MQ1 domain or functional variant or fragment thereof,
   wherein the first repressor domain is different from the second repressor domain.
69. An expression repression system of embodiment 67 or 68, wherein the first expression repressor further comprises a third repressor domain.
70. An expression repression system of any of embodiments 67-69, wherein the second expression repressor further comprises a fourth repressor domain.
71. The expression repression system of any of embodiments 67-70, wherein the first DNA-targeting moiety specifically binds a first DNA sequence, and the second DNA-targeting moiety specifically binds a second DNA sequence different from the first DNA sequence.
72. The expression repression system of any of embodiments 67-70, wherein the first DNA-targeting moiety and the second DNA-targeting moiety specifically bind the same DNA sequence.
73. The expression repression system of any of embodiments 67-72,
   wherein the first DNA-targeting moiety comprises a first CRISPR/Cas molecule comprising a first CRISPR/Cas protein and first guide RNA, and the second DNA-targeting moiety comprises a second CRISPR/Cas molecule comprising a second CRISPR/Cas protein and a second guide RNA.
74. The expression repression system of embodiment 73,
   wherein the first CRISPR/Cas protein does not bind the second guide RNA, e.g., binds with a K_{D} of at least 10, 20, 50, 100, 1000, or 10,000 nM, and the second CRISPR/Cas protein does not bind the first guide RNA, e.g., binds with a K_{D} of at least 10, 20, 50, 100, 1000, or 10,000 nM.
75. The expression repression system of either of embodiments 73 or 74:
   wherein the first CRISPR/Cas protein comprises a different amino acid sequence than the second CRISPR/Cas protein.
76. The expression repression system of any of embodiments 67-75, wherein the first or second DNA-targeting moiety comprises a CRISPR/Cas molecule comprising a Cas protein or Cpf1 protein chosen from Table 1 or a variant (e.g., mutant) of any thereof.
77. The expression repression system of any of embodiments 67-76, wherein the first DNA-targeting moiety comprises a first CRISPR/Cas molecule comprising a Cas protein or Cpf1 protein chosen from Table 1 or a variant (e.g., mutant) of any thereof, and the second DNA-targeting moiety comprises a second CRISPR/Cas molecule comprising a different Cas protein or Cpf1 protein chosen from Table 1 or a variant (e.g., mutant) of any thereof.
78. The expression repression system of any of embodiments 70-77, wherein the first, second, third, or fourth repressor domain comprises a histone methyltransferase activity.
79. The expression repression system of embodiment 70-78, wherein the first, second, third, or fourth repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof.
80. The expression repression system of any of embodiments 70-79, wherein the first, second, third, or fourth repressor domain comprises a histone demethylase activity (e.g., a lysine demethylase activity).
81. The expression repression system of any of embodiment 70-80, wherein the first, second, third, or fourth repressor domain comprises a protein chosen from KDM1A (i.e., LSD1), KDM1B (i.e., LSD2), KDM2A, KDM2B, KDM5A, KDM5B, KDM5C, KDM5D, KDM4B, N066 (or a functional variant or fragment of any thereof.
82. The expression repression system of any of embodiments 70-81, wherein the first, second, third, or fourth repressor domain comprises a histone deacetylase activity.
83. The expression repression system of embodiment 70-82, wherein the first, second, third, or fourth repressor domain comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.
84. The expression repression system of any of embodiments 70-83, wherein the first, second, second, third, or fourth repressor domain comprises a DNA methyltransferase activity.
85. The expression repression system of embodiment 70-84, wherein the first, second, third, or fourth repressor domain comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.
86. The expression repression system of any of embodiments 70-85, wherein the first, second, third, or fourth repressor domain comprises a transcription repressor activity.
87. The expression repression system of embodiment 70-86, wherein the first, second, third, or fourth repressor domain comprises a protein chosen from KRAB, MeCP2, HP1, RBBP4, REST, FOG1, SUZ12, or a functional variant or fragment of any thereof.
88. The expression repression system of any of embodiments 67-87, wherein:
   the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof, and
   the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof.
89. The expression repression system of any of embodiments 69-88, wherein:
   (i) the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof, and
   (ii) the third repressor domain comprises a protein (e.g., a different protein) chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof.
90. The expression repression system of any of embodiments 69-89, wherein:
   (i) the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof,
   (ii) the second repressor domain comprises a protein (e.g., different protein) chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof, and
   (iii) the third repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof.
91. The expression repression system of any of embodiments 70-90, wherein:
   (i) the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof,
   (ii) the second repressor domain comprises a protein (e.g., a different protein) chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof,
   (iii) the third repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof and
   (iv) the fourth repressor domain comprises a protein (e.g., a different protein) chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof.
92. The expression repression system of any of embodiments 70-91, wherein:
   (i) the second repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof, and
   (ii) the fourth repressor domain comprises a protein (e.g., a different protein) chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof.
93. The expression repression system of any of embodiments 67-92, wherein the first repressor domain comprises a histone methyltransferase activity and the second repressor domain comprises a histone demethylase activity.
94. The expression repression system of any of embodiments 67-92, wherein the first repressor domain comprises a histone methyltransferase activity and the second repressor domain comprises a histone deacetylase activity.
95. The expression repression system of any of embodiments 67-92, wherein the first repressor domain comprises a histone methyltransferase activity and the second repressor domain comprises a DNA methyltransferase activity.
96. The expression repression system of any of embodiments 67-92, wherein the first repressor domain comprises a histone methyltransferase activity and the second repressor domain comprises a DNA demethylase activity.
97. The expression repression system of any of embodiments 67-92, wherein the first repressor domain comprises a histone methyltransferase activity and the second repressor domain comprises a transcription repressor activity.
98. The expression repression system of any of embodiments 67-92, wherein the first repressor domain comprises a histone methyltransferase activity and the second repressor domain comprises a histone methyltransferase activity (e.g., wherein the histone methyltransferase activities are the same or different from each other).
99. The expression repression system of any of embodiments 67-92, wherein the first repressor domain comprises a histone demethylase activity and the second repressor domain comprises a histone deacetylase activity.
100. The expression repression system of any of embodiments 67-92, wherein the first repressor domain comprises a histone demethylase activity and the second repressor domain comprises a DNA methyltransferase activity.
101. The expression repression system of any of embodiments 67-92, wherein the first repressor domain comprises a histone demethylase activity and the second repressor domain comprises a DNA demethylase activity.
102. The expression repression system of any of embodiments 67-92, wherein the first repressor domain comprises a histone demethylase activity and the second repressor domain comprises a transcription repressor activity.
103. The expression repression system of any of embodiments 67-92, wherein the first repressor domain comprises a histone demethylase activity and the second repressor domain comprises a histone demethylase activity (e.g., wherein the histone demethylase activities are the same or different from each other).
104. The expression repression system of any of embodiments 67-92, wherein the first repressor domain comprises a histone deacetylase activity and the second repressor domain comprises a DNA methyltransferase activity.
105. The expression repression system of any of embodiments 67-92, wherein the first repressor domain comprises a histone deacetylase activity and the second repressor domain comprises a DNA demethylase activity.
106. The expression repression system of any of embodiments 67-92, wherein the first repressor domain comprises a histone deacetylase activity and the second repressor domain comprises a transcription repressor activity.
107. The expression repression system of any of embodiments 67-92, wherein the first repressor domain comprises a histone deacetylase activity and the second repressor domain comprises a histone deacetylase activity (e.g., wherein the histone deacetylase activities are the same or different from each other).
108. The expression repression system of any of embodiments 67-92, wherein the first repressor domain comprises a DNA methyltransferase activity and the second repressor domain comprises a DNA demethylase activity.
109. The expression repression system of any of embodiments 67-92, wherein the first repressor domain comprises a DNA methyltransferase activity and the second repressor domain comprises a transcription repressor activity.
110. The expression repression system of any of embodiments 67-92, wherein the first repressor domain comprises a DNA methyltransferase activity and the second repressor domain comprises a DNA methyltransferase activity (e.g., wherein the DNA methyltransferase activities are the same or different from each other).
111. The expression repression system of any of embodiments 67-92, wherein the first repressor domain comprises a DNA demethylase activity and the second repressor domain comprises a transcription repressor activity.
112. The expression repression system of any of embodiments 67-92, wherein the first repressor domain comprises a DNA demethylase activity and the second repressor domain comprises a DNA demethylase activity (e.g., wherein the DNA demethylase activities are the same or different from each other).
113. The expression repression system of any of embodiments 67-92, wherein the first repressor domain comprises a transcription repressor activity and the second repressor domain comprises a transcription repressor activity (e.g., wherein the transcription repressor activities are the same or different from each other).
114. The expression repression system of any of embodiments 67-113, wherein:
   the first repressor domain comprises a protein selected from: KRAB, a SET domain (e.g., the SET domain of SETDB1, EZH2, G9A, or SUV39H1), histone demethylase LSD1, FOG1 (e.g., the N-terminal residues of FOG1), KAP1, or a functional variant or fragment of any thereof; and
   the second repressor domain comprises a protein selected from: DNMT3A (e.g., human DNMT3A), DNMT3B, DNMT3L, DNMT3A/3L complex, bacterial MQ1, or a functional variant or fragment of any thereof.
115. The expression repression system of any of embodiments 67-114, wherein:
   the first repressor domain comprises a protein selected from: EZH2, G9A, SUV39H1, FOG1, or a functional variant or fragment of any thereof; and
   the second repressor domain comprises a protein selected from: DNMT3A (e.g., human DNMT3A), DNMT3B, DNMT3L, DNMT3A/3L, DNMT1, LSD1, FOG1, KRAB, HDAC8, bacterial MQ1, or a functional variant or fragment of any thereof.
116. The expression repression system of any of embodiments 67-115, wherein:
   (i) the first repressor domain comprises a protein selected from: KRAB, MeCP2, HP1, RBBP4, REST, FOG1, SUZ12, KDM1A (i.e., LSD1), KDM1B (i.e., LSD2), KDM2A, KDM2B, KDM5A, KDM5B, KDM5C, KDM5D, KDM4B, N066, SETDB1, SETDB2, EHMT2 (i.e., G9A), EHMT1 (i.e., GLP), SUV39H1, EZH2, EZH1, SUV39H2, SETD8, SUV420H1, SUV420H2, KAP1, HDAC8, MQ1, DNMT1, DNMT3A, DNMT3a/31, or a functional variant or fragment of any thereof; and
   (ii) the second repressor domain comprises a protein selected from: KRAB, FOG1, G9A, LSD1, HDAC8, EZH2, DNMT3a/31, MQ1, DNMT1, DBMT3A, DNMT3A1, DNMT3A2, DNMT3B1, DNMT3B2, DNMT3B3, DNMT3B4, DNMT3B5, DNMT3B6, DNMT3L or a functional variant or fragment of any thereof.
117. The expression repression system of any of embodiments 67-116, wherein the first repressor domain comprises KRAB, and the second repressor domain comprises DNMT3A (e.g., human DNMT3A).
118. The expression repression system of any of embodiments 67-115, wherein the first repressor domain comprises G9A or a functional variant or fragment thereof, and the second repressor domain comprises KRAB or a functional variant or fragment thereof.
119. The expression repression system of any of embodiments 67-118, wherein the first repressor domain comprises LSD1 or a functional variant or fragment thereof and the second repressor domain comprises KRAB or a functional variant or fragment thereof.
120. The expression repression system of any of embodiments 67-119, wherein the first repressor domain comprises KRAB or a functional variant or fragment thereof and the second repressor domain comprises bacterial MQ1 or a functional variant or fragment thereof.
121. The expression repression system of any of embodiments 67-120, wherein:
   the first repressor domain comprises G9A or a functional variant or fragment thereof, wherein optionally the first repressor domain is N-terminal of the first DNA-targeting moiety; and
   the second repressor domain comprises EZH2 or a functional variant or fragment thereof, wherein optionally the second repressor domain is N-terminal of the second DNA-targeting moiety.
122. The expression repression system of any of embodiments 67-120, wherein:
   the first repressor domain of comprises LSD1 or a functional variant or fragment thereof, wherein optionally the first repressor domain is C-terminal of the first DNA-targeting moiety; and
   the second repressor domain comprises G9A or a functional variant or fragment thereof, wherein optionally the second repressor domain is N-terminal of the second DNA-targeting moiety.
123. The expression repression system of any of embodiments 67-120, wherein:
   the first repressor domain comprises MQ1 or a functional variant or fragment thereof, wherein optionally the first repressor domain is C-terminal of the first DNA-targeting moiety; and
   the second repressor domain comprises HDAC8 or a functional variant or fragment thereof, wherein optionally the second repressor domain is C-terminal of the second DNA-targeting moiety.
124. The expression repression system of any of embodiments 67-120, wherein:
   the first repressor domain comprises LSD1 or a functional variant or fragment thereof, wherein optionally the first repressor domain is C-terminal of the first DNA-targeting moiety; and
   the second repressor domain comprises HDAC8 or a functional variant or fragment thereof, wherein optionally the second repressor domain is C-terminal of the second DNA-targeting moiety.
125. The expression repression system of any of embodiments 67-120, wherein:
   the first repressor domain comprises LSD1 or a functional variant or fragment thereof, wherein optionally the first repressor domain is C-terminal of the first DNA-targeting moiety; and
   the second repressor domain comprises EZH2 or a functional variant or fragment thereof, wherein optionally the second repressor domain is N-terminal of the second DNA-targeting moiety.
126. The expression repression system of any of embodiments 67-120, wherein:
   the first repressor domain comprises EZH2 or a functional variant or fragment thereof, wherein optionally the first repressor domain is N-terminal of the first DNA-targeting moiety; and
   the second repressor domain comprises HDAC8 or a functional variant or fragment thereof, wherein optionally the second repressor domain is C-terminal of the second DNA-targeting moiety.
127. The expression repression system of any of embodiments 67-120, wherein:
   the first repressor domain comprises G9A or a functional variant or fragment thereof, wherein optionally the first repressor domain is N-terminal of the first DNA-targeting moiety; and
   the second repressor domain comprises HDAC8 or a functional variant or fragment thereof, wherein optionally the second repressor domain is C-terminal of the second DNA-targeting moiety.
128. The expression repression system of any of embodiments 69-120, wherein:
   the first repressor domain comprises EZH2 or a functional variant or fragment thereof, wherein optionally the first repressor domain is N-terminal of the first DNA-targeting moiety,
   the third repressor domain comprises KRAB or a functional variant or fragment thereof, wherein optionally the third repressor domain is C-terminal of the first DNA-targeting moiety; and
   the second repressor domain comprises MQ1 or a functional variant or fragment thereof, wherein optionally the second repressor domain is C-terminal of the second DNA-targeting moiety.
129. The expression repression system of any of embodiments 69-120, wherein:
   the first repressor domain comprises G9A or a functional variant or fragment thereof, wherein optionally the first repressor domain is N-terminal of the first DNA-targeting moiety,
   the third repressor domain comprises KRAB or a functional variant or fragment thereof, wherein optionally the third repressor domain is C-terminal of the first DNA-targeting moiety, and
   the second repressor domain comprises MQ1 or a functional variant or fragment thereof, wherein optionally the second repressor domain is C-terminal of the second DNA-targeting moiety
130. The expression repression system of any of embodiments 69-120, wherein:
   the first repressor domain comprises EZH2 or a functional variant or fragment thereof, wherein optionally the first repressor domain is N-terminal of the first DNA-targeting moiety,
   the third repressor domain comprises KRAB or a functional variant or fragment thereof, wherein optionally the third repressor domain is C-terminal of the first DNA-targeting moiety, and
   the second repressor domain comprises HDAC8 or a functional variant or fragment thereof, wherein optionally the second repressor domain is C-terminal of the second DNA-targeting moiety.
131. The expression repression system of any of embodiments 69-120, wherein:
   the first repressor domain comprises FOG1 or a functional variant or fragment thereof, wherein optionally the first repressor domain is N-terminal of the first DNA-targeting moiety,
   the third repressor domain comprises FOG1 or a functional variant or fragment thereof, wherein optionally the third repressor domain is C-terminal of the first DNA-targeting moiety, and
   the second repressor domain comprises KRAB or a functional variant or fragment thereof, wherein optionally the second repressor domain is C-terminal of the second DNA-targeting moiety.
132. The expression repression system of any of embodiments 69-120, wherein:
   the first repressor domain comprises EZH2 or a functional variant or fragment thereof, wherein optionally the first repressor domain is N-terminal of the first DNA-targeting moiety,
   the third repressor domain comprises KRAB or a functional variant or fragment thereof, wherein optionally the third repressor domain is C-terminal of the first DNA-targeting moiety, and
   the second repressor domain comprises LSD1 or a functional variant or fragment thereof, wherein optionally the second repressor domain is C-terminal of the second DNA-targeting moiety.
133. The expression repression system of any of embodiments 69-130, wherein:
   the first repressor domain comprises G9A or a functional variant or fragment thereof, wherein optionally the first repressor domain is N-terminal of the first DNA-targeting moiety,
   the third repressor domain comprises KRAB or a functional variant or fragment thereof, wherein optionally the third repressor domain is C-terminal of the first DNA-targeting moiety, and
   the second repressor domain comprises LSD1 or a functional variant or fragment thereof, wherein optionally the second repressor domain is C-terminal of the second DNA-targeting moiety.
134. The expression repression system of any of embodiments 67-133, wherein the first DNA-targeting moiety is selected from a TAL effector molecule, a CRISPR/Cas molecule, a zinc finger domain, a tetR domain, a meganuclease, or an oligonucleotide.
135. The expression repression system of any of embodiments 67-134, wherein the second DNA-targeting moiety is selected from a TAL effector molecule, a CRISPR/Cas molecule, a zinc finger domain, a tetR domain, a meganuclease, or an oligonucleotide.
136. The expression repression system of any of embodiments 67-135, wherein the first DNA-targeting moiety is or comprises a CRISPR/Cas molecule comprising a CRISPR/Cas protein (e.g., that is or comprises a Cas protein or Cpf1 protein chosen from Table 1 or a variant (e.g., mutant) of any thereof) and a guide RNA.
137. The expression repression system of any of embodiments 67-136, wherein the second DNA-targeting moiety is or comprises a CRISPR/Cas molecule comprising a CRISPR/Cas protein (e.g., that is or comprises a Cas protein or Cpf1 protein chosen from Table 1 or a variant (e.g., mutant) of any thereof) and a guide RNA.
138. The expression repression system of any of embodiments 67-137, wherein:
   the first DNA-targeting moiety is or comprises a CRISPR/Cas molecule comprising a CRISPR/Cas protein that is or comprises a Cas protein or Cpf1 protein chosen from Table 1 or a variant (e.g., mutant) of any thereof), and
   the second DNA-targeting moiety is or comprises a CRISPR/Cas molecule comprising a different CRISPR/Cas protein that is or comprises a Cas protein or Cpf1 protein chosen from Table 1 or a variant (e.g., mutant) of any thereof).
139. The expression repression system of any of embodiments 135-138, wherein:
   the first DNA-targeting moiety is or comprises a CRISPR/Cas molecule encoded by a nucleic acid sequence of SEQ ID NO: 45 or 89, and
   the second DNA-targeting moiety is or comprises a CRISPR/Cas molecule encoded by a nucleic acid sequence of SEQ ID NO: 45 or 89.
140. The expression repression system of any of embodiments 135-138, wherein:
   the first DNA-targeting moiety is or comprises a CRISPR/Cas molecule comprising an amino acid sequence of SEQ ID NO: 46 or 88, and
the second DNA-targeting moiety is or comprises a CRISPR/Cas molecule comprising an amino acid sequence of SEQ ID NO: 46 or 88.
141. The expression repression system of any of embodiments 67-140, wherein the first or the second DNA-targeting moiety specifically binds to a promoter sequence or to DNA proximal to a promoter sequence, e.g., a promoter sequence operably linked to a target gene.
142. The expression repression system of any of embodiments 67-141, wherein the first or the second DNA-targeting moiety specifically binds to an enhancer sequence or to DNA proximal to an enhancer sequence, e.g., an enhancer sequence that affects expression of and/or is operably linked to a target gene.
143. The expression repression system of any of embodiments 67-142, wherein the first or the second DNA targeting moiety specifically binds to a target gene, e.g., sequence comprised between the transcription start site and stop codon of a target gene.
144. The expression repression system of any of embodiments 67-143, wherein the first or the second DNA targeting moiety specifically binds to the transcription start site of a target gene.
145. The expression repression system of any of embodiments 67-144, wherein the first or the second DNA targeting moiety specifically binds to an intron, e.g., a splice site associated with an intron, of a target gene.
146. The expression repression system of any of embodiments 67-145, wherein the first or the second DNA targeting moiety specifically binds to an exon of a target gene.
147. The expression repression system of any of embodiments 67-146, wherein first or the second DNA targeting moiety specifically binds to an anchor sequence.
148. The expression repression system of embodiment 147, wherein the anchor sequence comprises the sequence of SEQ ID NO: 81 or 82, or a sequence with no more than 8, 7, 6, 5, 4, 3, 2, or 1 alterations relative thereto.
149. The expression repression system of embodiment 147, wherein the anchor sequence comprises the sequence of SEQ ID NO: 83 or 84, or a sequence with no more than 8, 7, 6, 5, 4, 3, 2, or 1 alterations relative thereto.
150. The expression repression system of any of embodiments 147-149, wherein the anchor sequence is on the same chromosome as the gene.
151. The expression repression system of any of embodiments 147-149, wherein the anchor sequence is on a different chromosome as the gene.
152. The expression repression system of any of embodiments 147-151, wherein the anchor sequence is upstream of the target gene (e.g., upstream of the TSS or upstream of the promoter).
153. The expression repression system of any of embodiments 148-151, wherein the anchor sequence is downstream of the target gene (e.g., downstream of the TSS or upstream of the promoter).
154. The expression repression system of any of embodiments 67-141, wherein the first DNA sequence is a first site within or proximal to a promoter operably linked to a target gene and the second DNA sequence is a second site within or proximal to the promoter operably linked to the target gene.
155. The expression repression system of any of embodiments 67-142, wherein the first DNA sequence is within or proximal to a promoter operably linked to a target gene and the second DNA sequence is a within or proximal to an enhancer that affects expression of or is operably linked to the target gene.
156. The expression repression system of any of embodiments 67-142, wherein the first DNA sequence is within or proximal to an enhancer that affects expression of or is operably linked to a target gene and the second DNA sequence is a within or proximal to a promoter operably linked to the target gene.
157. The expression repression system of any of embodiments 67-141 or 143, wherein the first DNA sequence comprises the transcription start site of a target gene and the second DNA sequence is within or proximal to a promoter operably linked to the target gene.
158. The expression repression system of any of embodiments 67-140, 142 or 143, wherein the first DNA sequence comprises the transcription start site of a target gene and the second DNA sequence is within or proximal to an enhancer that affects expression of or is operably linked to the target gene.
159. The expression repression system of any of embodiments 67-141 or 143, wherein the first DNA sequence within or proximal to a promoter operably linked to a target gene and the second DNA sequence is the transcription start site of the target gene.
160. The expression repression system of any of embodiments 67-140, 142, or 143, wherein the first DNA sequence is within or proximal to an enhancer that affects expression of or is operably linked to a target gene and the second DNA sequence is the transcription start site of the target gene.
161. The expression repression system of any of embodiments 67-160, wherein the distance between the first DNA sequence and the second DNA sequence is at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 base pairs or any size therebetween, e.g., 20-500 base pairs.
162. The expression repression system of any of embodiments 67-161, wherein the distance between the first DNA sequence and the second DNA sequence is no more than 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 base pairs or any size therebetween, e.g., 20-500 base pairs.
163. The expression repression system of any of embodiments 67-162, wherein the first DNA sequence and the second DNA sequence are situated on different nucleic acids, e.g., different chromosomes.
164. The expression repression system of any of embodiments 67-163, wherein the target gene is β-2-microglobulin, MYC, HSPA1B, GATA1, APOB, FOXP3, CXCL1, CXCL2, CXCL3, CXCL4, CXCL5, CXCL6, CXCL6, CXCL7, and/or CXCL8. 165. The expression repression system of any of embodiments 73-164, wherein the first guide RNA comprises a sequence specific to, e.g., complementary to, the first DNA sequence and the second guide RNA comprises a sequence specific to, e.g., complementary to, the second DNA sequence.
166. The expression repression system of any of embodiments 67-165, wherein the first DNA-targeting moiety does not appreciably bind to the second DNA sequence and the second DNA-targeting moiety does not appreciably bind to the first DNA sequence.
167. The expression repression system of any of embodiments 67-166, wherein the first DNA-targeting moiety binds the second DNA sequence with a K_{D} of at least 500, 600, 700, 800, 900, 1000, 2000, 5000, 10,000, or 100,000 nM, and the second DNA-targeting moiety binds the first DNA sequence with binds with a K_{D} of at least 500, 600, 700, 800, 900, 1000, 2000, 5000, 10,000, or 100,000 nM.
168. The expression repression system of any of embodiments 67-167, wherein binding of the first expression repressor to the first DNA sequence decreases expression of a target gene, e.g., a target gene operably linked to the first DNA sequence, in a cell.
169. The expression repression system of embodiment 168, wherein expression is decreased by 10, 20, 30, 40, 50, 60, 70, 80, 90, or 100% compared to expression in the absence of the first expression repressor, e.g., as measured by ELISA or as described in Examples 2-4.
170. The expression repression system of any of embodiments 67-169, wherein binding of the first expression repressor to the first DNA sequence appreciably decreases expression of a target gene, e.g., a target gene operably linked to the first DNA sequence, for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in Examples 2-4.
171. The expression repression system of any of embodiments 67-170, wherein binding of the second expression repressor to the second DNA sequence decreases expression of a target gene, e.g., a target gene operably linked to the second DNA sequence, in a cell.
172. The expression repression system of embodiment 171, wherein expression is decreased by 10, 20, 30, 40, 50, 60, 70, 80, 90, or 100% compared to expression in the absence of the second expression repressor, e.g., as measured by ELISA or as described in Examples 2-4.
173. The expression repression system of any of embodiments 67-172, wherein binding of the second expression repressor to the second DNA sequence appreciably decreases expression of a target gene, e.g., a target gene operably linked to the second DNA sequence, for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in Examples 2-4.
174. The expression repression system of any of embodiments 67-173, wherein binding of the first expression repressor to the first DNA sequence and the second expression repressor to the second DNA sequence decreases expression of a target gene, e.g., a target gene operably linked to the first DNA sequence and/or the second DNA sequence, in a cell.
175. The expression repression system of embodiment 174, wherein expression is decreased by 10, 20, 30, 40, 50, 60, 70, 80, 90, or 100% compared to expression in the absence of the first and second expression repressors, e.g., as measured by ELISA or as described in Examples 2-4.
176. The expression repression system of any of embodiments 1-175, wherein binding of the first expression repressor to the first DNA sequence and the second expression repressor to the second DNA sequence appreciably decreases expression of a target gene, e.g., a target gene operably linked to the first DNA sequence, for a time period of at least 1, 2, 3, 4, 5, 6, 7, 8, 9, 10, 11, or 12 hours, or at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in Examples 2-4.
177. The expression repression system of any of embodiments 174-176, wherein the decrease in expression resulting from the binding of the first expression repressor to the first DNA sequence and the second expression repressor to the second DNA sequence is greater than the decrease in expression resulting from the binding of the first expression repressor to the first DNA sequence or the binding of the second expression repressor to the second DNA sequence individually.
178. The expression repression system of embodiment 177, wherein the binding of the first expression repressor to the first DNA sequence and the second expression repressor to the second DNA sequence decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the first DNA sequence or the binding of the second expression repressor to the second DNA sequence individually, e.g., as measured by ELISA or as described in Examples 2-4.
179. The expression repression system of embodiment 174-178, wherein the decrease in expression resulting from the binding of the first expression repressor to the first DNA sequence and the second expression repressor to the second DNA sequence persists for a longer time (e.g., more hours, days, or cell divisions) than the decrease in expression resulting from the binding of the first expression repressor to the first DNA sequence or the binding of the second expression repressor to the second DNA sequence individually.
180. The expression repression system of embodiment 179, wherein the binding of the first expression repressor to the first DNA sequence and the second expression repressor to the second DNA sequence decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× longer (e.g., as measured in hours, days, or cell divisions) than either the binding of the first expression repressor to the first DNA sequence or the binding of the second expression repressor to the second DNA sequence individually, e.g., as measured by ELISA or as described in Examples 2-4.
181. The expression repression system of any of embodiments 174-180, wherein the cell is dividing at a rate of no more than once per 24, 36, 48, 60, or 72 hours (and optionally, at least once per 24, 36, 48, 60, or 72 hours).
182. The expression repression system of any of embodiments 174-181, wherein the cell is dividing at a rate of at least once per 24, 36, 48, 60, or 72 hours (and optionally, no more than once per 24, 36, 48, 60, or 72 hours).
183. The expression repression system of any of embodiments 174-182, wherein expression is appreciably decreased indefinitely (e.g., for a time period greater than can be experimentally measured).
184. The expression repression system of any of embodiments 67-183, wherein
   the first DNA-targeting moiety comprises an S. pyogenes CRISPR/Cas protein selected from Table 1 or variant (e.g., mutant) thereof, e.g., an S. pyogenes dCas9, e.g., a dCas9 comprising an amino acid sequence of SEQ ID NO: 46; or a S. aureus CRISPR/Cas protein selected from Table 1 or variant (e.g., mutant) thereof, e.g., an S. aureus dCas9, e.g., a dCas9 comprising an amino acid sequence of SEQ ID NO: 88 and
   the second DNA-targeting moiety comprises an S. pyogenes CRISPR/Cas protein selected from Table 1 or variant (e.g., mutant) thereof, e.g., an S. pyogenes dCas9, e.g., a dCas9 comprising an amino acid sequence of SEQ ID NO: 46; or a CRISPR/Cas molecule comprising a S. aureus CRISPR/Cas protein selected from Table 1 or variant (e.g., mutant) thereof, e.g., an S. aureus dCas9, e.g., a dCas9 comprising an amino acid sequence of SEQ ID NO: 88.
185. The expression repression system of any of embodiments 67-184, wherein
   the first DNA-targeting moiety comprises a CRISPR/Cas molecule comprising a CRISPR/Cas protein, e.g., a catalytically inactive dCas9, e.g., a dCas9, e.g., a dCas9 comprising one or more mutations selected from D10A, D839A, H840A, and N863A mutations; and
   the second DNA-targeting moiety comprises a CRISPR/Cas molecule comprising a CRISPR/Cas protein, e.g., a catalytically inactive dCas9, e.g., a dCas9, e.g., a dCas9 comprising one or more mutations selected from D10A, D839A, H840A, and N863A mutations.
186. The expression repression system of any of embodiments 67-185, wherein
   the first expression repressor comprises a first DNA-targeting moiety comprising a CRISPR/Cas molecule comprising an S. pyogenes CRISPR/Cas protein selected from Table 1 or variant (e.g., mutant) thereof, e.g., an S. pyogenes dCas9, and a first repressor domain comprising KRAB or a functional variant or fragment thereof; and
   the second expression repressor comprises a second DNA-targeting moiety comprising a CRISPR/Cas molecule comprising a S. aureus CRISPR/Cas protein selected from Table 1 or variant (e.g., mutant) thereof, e.g., an S. aureus dCas9, and a second repressor domain comprising bacterial MQ1 or a functional variant or fragment thereof.
187. A polypeptide comprising:
   a DNA-targeting moiety; and
   a repressor domain comprising bacterial MQ1 or a functional variant or fragment thereof.
188. A polypeptide comprising:
   a DNA-targeting moiety;
   a first repressor domain comprising MQ1, DNMT1, DNMT3a/31, KRAB, G9A, HDAC8, LSD1, EZH2, FOG1 or a functional variant or fragment thereof, and
   a second repressor domain comprising MQ1, DNMT1, DNMT3a/31, KRAB, G9A, HDAC8, LSD1, EZH2, FOG1 or a functional variant or fragment thereof.
189. The polypeptide of embodiment 88a, wherein the DNA-targeting moiety is situated between the first repressor domain and the second repressor domain.
190. A nucleic acid encoding the first expression repressor of the expression repression system of any of embodiments 67-189.
191. A nucleic acid encoding the second expression repressor of the expression repression system of any of embodiments 67-189.
192. A nucleic acid encoding the expression repression system, e.g., the first expression repressor and the second expression repressor, of any of embodiments 67-189.
193. The nucleic acid of any of embodiments 190-192, which is an RNA, e.g., an mRNA.
194. An expression repression system comprising:
   a first nucleic acid comprising a sequence encoding the first expression repressor of the expression repression system of any of embodiments 67-189; and
   a second nucleic acid comprising a sequence encoding the second expression repressor of the expression repression system of any of embodiments 67-189.
195. A nucleic acid encoding the polypeptide of any of embodiments 187-189.
196. The nucleic acid any of embodiments 190-193 or 195, wherein the nucleic acid comprises mRNA.
197. A vector comprising the nucleic acid encoding the expression repressor or the expression repression system of any preceding embodiment.
198. A lipid nanoparticle comprising the expression repressor, expression repression system, nucleic acid, mRNA, or vector of any of the preceding embodiments.
199. The lipid nanoparticle of embodiment 198 comprising an ionizable lipid, e.g., a cationic lipid, e.g., MC3, SSOP.
200. The lipid nanoparticle of embodiment 198 or 199, further comprising one or more of neutral lipids, ionizable amine-containing lipids, biodegradable alkyn lipids, steroids, phospholipids, polyunsaturated lipids, structural lipids (e.g., sterols), PEG, cholesterol, or polymer conjugated lipids.
201. A reaction mixture comprising the expression repressor, expression repression system, nucleic acid, vector, or lipid nanoparticle of any of the preceding embodiments.
202. The reaction mixture of embodiment 201 further comprising a cell.
203. A cell comprising the expression repressor, expression repression system, nucleic acid, mRNA, vector, or nanoparticle of any of embodiments 1-202.
204. The cell of embodiment 203, wherein the cell is selected from a hepatocyte, a neuronal cell, an endothelial cell, a myocyte, and a lymphocyte.
205. A cell produced by a method comprising:
   providing i) a cell and ii) an expression repressor, expression repression system, polypeptide, nucleic acid, vector, lipid nanoparticle, or reaction mixture of any of embodiments 1-202; and
   contacting the cell with the expression repressor, expression repression system, polypeptide, nucleic acid, vector, lipid nanoparticle, or reaction mixture.
206. A pharmaceutical composition comprising the expression repressor, expression repression system, nucleic acid, polypeptide, vector, lipid nanoparticle, or reaction mixture of any of embodiments 1-202, and at least one pharmaceutically acceptable excipient or carrier.
207. The expression repression system of any of embodiments 1-202, wherein the first expression repressor further comprises an additional moiety selected from: a tagging or monitoring moiety, a cleavable moiety (e.g., a cleavable moiety positioned between a DNA-targeting moiety and a repressor domain or at the N- or C-terminal end of a polypeptide), a small molecule, a membrane translocating polypeptide, or a pharmacoagent moiety.
208. The expression repression system of any of embodiments 1-202, wherein the second expression repressor further comprises an additional moiety selected from: a tagging or monitoring moiety, a cleavable moiety (e.g., a cleavable moiety positioned between a DNA-targeting moiety and a repressor domain or at the N- or C-terminal end of a polypeptide), a small molecule, a membrane translocating polypeptide, or a pharmacoagent moiety.
209. A method of decreasing expression of a target genomic sequence in a cell, comprising:
   providing the expression repressor, expression repression system, nucleic acid, polypeptide, vector, lipid nanoparticle, reaction mixture, cell, or pharmaceutical composition of any of embodiments 1-204 or 206; and
   contacting the cell with the expression repressor, expression repression system, nucleic acid, polypeptide, vector, lipid nanoparticle, reaction mixture, cell, or pharmaceutical composition thereby decreasing expression of the target genomic sequence.
210. A method of epigenetically modifying a target genomic sequence in a cell, comprising:
   providing an expression repressor, expression repression system, nucleic acid, vector, polypeptide, lipid nanoparticle, reaction mixture, cell, or pharmaceutical composition of any of embodiments 1-204 or 206; and
   contacting the cell with the expression repressor, expression repression system, nucleic acid, vector, polypeptide, lipid nanoparticle, reaction mixture, cell, or pharmaceutical composition,
   thereby epigenetically modifying the target genomic sequence.
211. The method of f embodiment 209 or 210, wherein the target genomic sequence is:
   a target gene (e.g., a site within a target gene, e.g., an exon, intron, or splice site), e.g., the gene encoding β-2-microglobulin (β2M), the gene encoding MYC, the gene encoding HSPA1B, or the gene encoding GATA1,
   a transcription control element operably linked to a target gene, or
   an anchor sequence proximal to a target gene or associated with an anchor sequence-mediated conjunction operably linked to the target gene.
212. The method of embodiment 211, wherein the epigenetic modification results in a decrease in expression of the target gene.
213. The expression repressor, expression repression system, cell, pharmaceutical composition, nucleic acid, vector, or method of any of embodiments 174-212, wherein the cell is a mammalian cell, e.g., a human cell.
214. The method of embodiment 213, wherein the method is performed ex vivo (e.g., in a sample from a subject) or in vivo (e.g., by administering the expression repression system to a subject).
215. The method of any of embodiments 209-214, wherein expression is decreased by 10, 20, 30, 40, 50, 60, 70, 80, 90, or 100% compared to expression in an otherwise similar cell in the absence of the expression repression system.
216. The method of any of embodiments 209-214, wherein expression is decreased by 10, 20, 30, 40, 50, 60, 70, 80, 90, or 100% compared to expression in an otherwise similar cell that has not been contacted with the expression repression system.
217. The method of any of embodiments 209-216, wherein expression is decreased for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in Examples 2-4.
218. The method of any of embodiments 215-217, wherein the decrease in expression comprises a decrease in the level of RNA, e.g., mRNA, encoded by the target gene.
219. The method of any of embodiments 215-218, wherein the decrease in expression comprises a decrease in the level of a protein encoded by the target gene.
220. The method of any of embodiments 209-219, wherein providing comprises contacting the cell with a nucleic acid, e.g., vector, encoding the expression repression system to the cell comprising the target gene.
221. The method of any of embodiments 209-220, wherein the nucleic acid encoding the first expression repressor is disposed on a first nucleic acid molecule and the nucleic acid encoding the second expression repressor is disposed on a second nucleic acid molecule.
222. The method of any of embodiments 209-221, wherein the nucleic acid encoding the first expression repressor is disposed on the same nucleic acid molecule as the nucleic acid encoding the second expression repressor.
223. The method of any of embodiments 209-222, wherein administering comprises contacting the cell with the first nucleic acid molecule and the second nucleic acid molecule together, e.g., simultaneously.
224. The method of any of embodiments 209-223, wherein administering comprises contacting the cell with the first nucleic acid molecule and the second nucleic acid molecule separately, e.g., sequentially.
225. The method of any of embodiments 209-224, wherein providing comprises contacting the cell with a vesicle comprising the expression repression system or a nucleic acid, e.g., vector, encoding the expression repression system.
226. The method of any of embodiments 209-225, wherein providing comprises contacting the cell with a lipid nanoparticle (LNP) comprising the expression repression system or a nucleic acid, e.g., vector, encoding the expression repression system.
227. The method of any of embodiments 209-226, wherein the expression repression system comprises a first expression repressor and a second expression repressor, and providing comprises administering the first expression repressor and, separately, administering the second expression repressor.
228. The method of any of embodiments 209-227, wherein the expression repression system comprises a first expression repressor and a second expression repressor and providing comprises administering the first expression repressor and the second expression repressor together, e.g., simultaneously.
229. A method of treating a condition associated with over-expression of a target gene in a subject, comprising:
   administering the expression repressor, expression repression system, nucleic acid, vector, polypeptide, lipid nanoparticle, reaction mixture, cell, or pharmaceutical composition of any of embodiments 1-204 or 206-208 to the subject,
   thereby treating a condition associated with over-expression of a target gene in a subject.
230. A method of treating a condition associated with mis-regulation of a target gene in a subject, comprising:
   administering the expression repressor, expression repression system, nucleic acid, vector, polypeptide, lipid nanoparticle, reaction mixture, cell, or pharmaceutical composition of any of embodiments 1-204 or 206-208 to the subject,
   thereby treating a condition associated with mis-regulation of a target gene in a subject.
231. A method of making a cell comprising an expression repression system, the method comprising:
   providing the expression repressor, expression repression system, nucleic acid, vector, polypeptide, or pharmaceutical composition of any of embodiments 1-201 or 206-208; and
   contacting the cell with the expression repressor, expression repression system, nucleic acid, vector, or pharmaceutical composition,
   thereby making a cell comprising an expression repression system.
232. A method of making a nucleic acid encoding an expression repression system, the method comprising:
   providing a first nucleic acid encoding the first expression repressor of the expression repression system of any of embodiments 67-186, 207, or 208;
   providing a second nucleic acid encoding the second expression repressor of the expression repression system of any of embodiments 67-186, 9207, or 208;
   combining (e.g., ligating or recombining) the first nucleic acid and the second nucleic acid into a single nucleic acid molecule,
   thereby making a nucleic acid encoding an expression repression system.
233. A cell treated or produced by the method of any of embodiments 209-228 or 230.
234. The cell of embodiment 233, wherein the cell does not comprise the expression repression system, e.g., at least one component (e.g., all components) of the expression repression system.
235. The cell of either of embodiments 233 or 234, wherein expression of the target gene is decreased relative to a cell not contacted, treated, or produced by the method of any of embodiments 209-228 or 230.
236. A kit comprising a container comprising a composition comprising an expression repressor, an expression repression system, one or more nucleic acids encoding said expression repressor or an expression repression system, a vector, a lipid nanoparticle, or a pharmaceutical composition of any of embodiments 1-208 and a set of instructions comprising at least one method for modulating, e.g., decreasing the expression of a gene within a cell with said composition.

### Definitions

Anchor Sequence: The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.

Anchor Sequence-Mediated Conjunction: The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences (see, e.g. FIG. 1).

Associated with: Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic or transcription complex when the nucleic acid is at least partially within the target genomic or transcription complex, and expression of a gene in the DNA sequence is affected by formation or disruption of the target genomic or transcription complex.

Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively, or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

Expression repressor: As used herein, the term “expression repressor” refers to an agent or entity with one or more functionalities that decrease expression of a target gene in a cell and that specifically binds to a DNA sequence (e.g., a DNA sequence associated with a target gene, or a transcription control element operably linked to a target gene). An expression repressor comprises at least one DNA-targeting moiety and at least one repressor domain.

Expression repression system: As used herein, the term “expression repression system” refers to a plurality of expression repressors which decrease expression of a target gene in a cell. In some embodiments, an expression repression system comprises a first expression repressor and a second expression repressor, wherein the first expression repressor and second expression repressor (or nucleic acids encoding the first expression repressor and second expression repressor) are present together in a single composition, mixture, or pharmaceutical composition. In some embodiments, an expression repression system comprises a first expression repressor and a second expression repressor, wherein the first expression repressor and second expression repressor (or nucleic acids encoding the first expression repressor and second expression repressor) are present in separate compositions or pharmaceutical compositions. In some embodiments, the first expression repressor and the second expression repressor are present in the same cell at the same time. In some embodiments, the first expression repressor and the second expression repressor are not present in the same cell at the same time, e.g., they are present sequentially. For example, the first expression repressor may be present in a cell for a first time period, and then the second expression repressor may be present in the cell for a second time period, wherein the first and second time periods may be overlapping or non-overlapping.

Genomic complex: As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that it does not adopt when the complex is not formed.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present disclosure. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, the phrase “operably linked” refers to a juxtaposition wherein the components described are in a relationship permitting them to function in their intended manner. A transcription control element “operably linked” to a functional element, e.g., gene, is associated in such a way that expression and/or activity of the functional element, e.g., gene, is achieved under conditions compatible with the transcription control element. In some embodiments, “operably linked” transcription control elements are contiguous (e.g., covalently linked) with coding elements, e.g., genes, of interest; in some embodiments, operably linked transcription control elements act in trans to or otherwise at a distance from the functional element, e.g., gene, of interest. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other.

Peptide, Polypeptide, Protein: As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides, and oligomers, for example, and to longer chains, which generally are referred to in the art as proteins, of which there are many types.

Repressor domain: As used herein, the term “repressor domain” refers to a domain with one or more functionalities that decrease expression of a target gene in a cell when appropriately localized in the nucleus of a cell. In some embodiments, a repressor domain is or comprises a polypeptide. In some embodiments, a repressor domain is or comprises a polypeptide and a nucleic acid. A functionality associated with a repressor domain may directly affect expression of a target gene, e.g., blocking recruitment of a transcription factor that would stimulate expression of the gene. A functionality associated with a repressor domain may indirectly affect expression of a target gene, e.g., introducing epigenetic modifications or recruiting other factors that introduce epigenetic modifications that induce a change in chromosomal topology that inhibits expression of a target gene.

Specific binding: As used herein, the term “specific binding” refers to an ability to discriminate between possible binding partners in the environment in which binding is to occur. In some embodiments, a binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex. In some embodiments, specific binding is assessed by detecting or determining ability of the binding agent to compete with an alternative interaction between its partner and another entity. In some embodiments, specific binding is assessed by performing such detections or determinations across a range of concentrations.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

Symptoms are reduced: As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.

Target: An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence.

Target gene: As used herein, the term “target gene” means a gene that is targeted for modulation, e.g., of expression. In some embodiments, a target gene is part of a targeted genomic complex (e.g., a gene that has at least part of its genomic sequence as part of a target genomic complex, e.g. inside an anchor sequence-mediated conjunction), which genomic complex is targeted by one or more modulating agents as described herein. In some embodiments, modulation comprises inhibition of expression of the target gene. In some embodiments, a target gene is modulated by contacting the target gene or a transcription control element operably linked to the target gene with an expression repression system, e.g., expression repressor(s), described herein. In some embodiments, a target gene is aberrantly expressed (e.g., over-expressed) in a cell, e.g., a cell in a subject (e.g., patient).

DNA-targeting moiety: As used herein, in the context of expression repressors, the term “DNA-targeting moiety” means a domain that specifically binds to a DNA sequence (e.g., a DNA sequence associated with a target gene, or a transcription control element operably linked to a target gene).

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent comprises an expression repression system, e.g., an expression repressor, described herein. In some embodiments, a therapeutic agent comprises a nucleic acid encoding an expression repression system, e.g., an expression repressor, described herein. In some embodiments, a therapeutic agent comprises a pharmaceutical composition described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

### BRIEF DESCRIPTION OF THE DRAWING

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 shows a Genome Browser view showing key features of the region near the transcription start site of β2M. RNAseq (top level) shows mRNA expression from the β2M gene. A previously described positive control (Cas9 targeting, GD-21547) is shown in green. Blue guides (GD-28228 and GD-28229) were used to target Sp-dCas9-KRAB and orange guides (GD-28171, GD-28172 and GD-28173) were used to target Sa-dCas9-MQ1 to the CpG Island in the β2M promoter region (second level from the top). Guides were chosen based on hypomethylation of the promoter region (depicted in the CpG Methylation track, bottom level), where methylation runs from 0 to 1 (0 to 100%) and where higher methylation is depicted by red bars. Each bar represents a CpG site.

FIG. 2 shows a graph of percent change in β-2-microglobulin expression (mRNA) in HepG2 cells with treatment with a variety of expression repressors or controls. β2M mRNA levels shown are relative to the untreated control (gray). Points show biological replicates. Bars show the average and error bars indicate standard deviations. The guide ID number is shown on the x-axis. Samples treated with dCas9 are shown in magenta; dCas9-KRAB in green; sa-dCas9-MQ1 in light blue; a combination of dCas9-KRAB and sa-dCas9-MQ1 in dark blue. A β2M-knockout positive control with Cas9 is shown in orange.

FIG. 3 shows a graph of percent change in β-2-microglobulin mRNA with treatment with a variety of expression repressors or controls after 72 hours (left), and a graph of flow cytometry data of β-2-microglobulin protein levels with treatment with a variety of expression repressors or controls after 72 hours (right). (Left) Treatment with dCas9-KRAB (green) and Sa-dCas9-MQ1 (light blue) effectors, separately and together (dark blue) results in a decrease in β2M mRNA relative to untreated cells (grey) and cells treated with dCas9 (magenta). (Right) Flow cytometry revealed populations with decreased β2M immunofluorescence in cells treated with dCas9-KRAB and Sa-dCas9-MQ1 relative to untreated cells and cells treated with dCas9.

FIG. 4 shows a graph of the fold change in β-2-microglobulin mRNA with treatment with a variety of expression repressors or controls over a 22-day period. Over a 22-day period, non-transfected (blue line) and dCas9 transfected (red line) cells don't change β2M expression, whereas dCas9-KRAB (green line) transfected cells have β2M repressed in the initial days post-transfection, with maximum effect on day 2, but the repression is lost after day 5. SadCas9-MQ1 (purple line) transfected cells show significant repression (up to 85% by day 4) and the repression is retained and then slowly lost as seen by line remaining just under the dotted line in the figure. dCas9-KRAB+SadCas9-MQ1 combined transfection (orange line) did not give any added repression than what is already observed for SadCas9 alone (purple line).

FIG. 5 shows a graph of the fold change in P-2-microglobulin mRNA with treatment with a variety of expression repressors or controls over an 18-day period. Over an 18-day period untreated cells do not change β2M expression, whereas cells transfected with dCas9-MQ1, dCas9-DNMT3a/3L (m), dCas9-DNMT3a/3L (hs), dCas9-DNMT1 and dCas9-DNMT3B, show significant β2M repression post-transfection.

FIG. 6 shows a graph of the fold change in MYC expression in K-562 cells when treated with different expression repressors or expression repression systems that target the CTCF site located upstream of MYC gene. The data showed that, at least, cells treated with dCas9-DNMT3a/3L (h), dCas9-KRAB, FOG1-dCas9-FOG1, G9A-dCas9+EZH2-dCas9, dCas9-MQ1+EZH2-dCas9-KRAB, dCas9-LSD1+G9A-dCas9, dCas9-MQ1+dCas9-HDAC8, and dCas9-MQ1+G9A-dCas9-KRAB showed repression of MYC expression at 48 hour and/or 72-hour time point.

FIG. 7 shows a graph of the fold change in β2M expression in K-562 cells when treated with different expression repressors or expression repression systems that target a region upstream of the β2M promoter. The data showed that, at least, cells treated with dCas9-HDAC8, dCas9-LSD1, dCas9-MQ1, FOG1-dCas9-FOG1, G9A-dCas9-KRAB, dCas9-DNMT3a/3L (h), G9A-dCas9, EZH2-dCas9-KRAB, dCas9-KRAB, EZH2-dCas9-KRAB+dCas9-HDAC8, dCas9-LSD1+EZH2-dCas9-KRAB, dCas9-LSD1+dCas9-HDAC8, dCas9-KRAB+FOG1-dCas9-FOG1, dCas9-MQ1+G9A-dCas9-KRAB, G9A-dCas9+EZH2-dCas9, dCas9-MQ1+EZH2-dCas9-KRAB, and dCas9-MQ1+dCas9-HDAC8 showed repression of β2M expression at 48 hour, 72 hour and/or 144 hour time point.

FIG. 8 shows a graph of the fold change in HSPA1B expression in K-562 cells when treated with different expression repressors or expression repression systems that target a region downstream of the HSPA1B promoter. The data showed that, at least, cells treated with dCas9-HDAC8, EZH2-dCas9, dCas9-MQ1, dCas9-DNMT1, dCas9-DNMT3a/3L (h), dCas9-DNMT3a/3L (m), G9A-dCas9-KRAB, FOG1-dCas9-FOG1, G9A-dCas9, dCas9-KRAB, G9A-dCas9+dCas9-HDAC8, dCas9-LSD1+G9A-dCas9-KRAB, dCas9-LSD1+EZH2-dCas9, G9A-dCas9+EZH2-dCas9, dCas9-LSD1+EZH2-dCas9-KRAB, EZH2-dCas9+dCas9-HDAC8, dCas9-LSD1+dCas9-HDAC8, dCas9-MQ1+EZH2-dCas9-KRAB, dCas9-MQ1+dCas9-HDAC8, G9A-dCas9-KRAB+EZH2-dCas9-KRAB, dCas9-LSD1+G9A-dCas9 and dCas9-MQ1+G9A-dCas9-KRAB showed repression of HSPA1B expression at 48 hour, 72 hour and/or 144 hour time point.

FIG. 9 shows a graph of the fold change in GATA1 expression in K-562 cells when treated with different expression repressors or expression repression systems. The data showed that, at least, cells treated with dCas9-HDAC8, dCas9-LSD1+dCas9-HDAC8, dCas9-MQ1+dCas9-HDAC8, and G9A-dCas9+dCas9-HDAC8 showed repression of GATA1 expression at 48-hour, 72 hour and/or 144 hour time point.

### DETAILED DESCRIPTION OF CERTAIN EMBODIMENTS

The present disclosure provides technologies for modulating, e.g., decreasing, expression of a target gene in cell, e.g., in a subject or patient, through the use of an expression repressor or an expression repression system. In some embodiments, an expression repressor comprises a DNA binding moiety and at least one repressor domain. In some embodiments, an expression repressor comprises two repressor domains. In some embodiments an expression repressor comprises two identical repressor domains. In some embodiments an expression repressor comprises two non-identical repressor domains.

In some embodiments, an expression repression system comprises two or more expression repressors, each comprising a DNA-targeting moiety and at least one repressor domain. In some embodiments, the DNA-targeting moieties target two or more different DNA sequences (e.g., each expression repressor may target a different DNA sequence). Without wishing to be bound by theory, the use of an expression repression system comprising expression repressors targeting two or more different DNA sequences may be advantageous over similar systems targeting a single DNA sequence because individual expression repressors will not compete with one another or compete less with one another for binding to their target DNA sequences, thereby achieving superior localization of the various functionalities of the repressor domains. In some embodiments, it can be advantageous to use two different DNA-targeting moieties to precisely target two different repressors to two different defined locations, e.g., a first expression repressor to a first location upstream of the transcription start site and a second expression repressor to a second location comprising the transcription start site. In some embodiments, the expression repressors comprise two or more different repressor domains (e.g., each expression repressor comprises a different repressor domain from each other expression repressor). Without wishing to be bound by theory, the use of an expression repression system comprising expression repressors comprising two or more repressor domains may be advantageous over similar systems comprising a single repressor domain due to synergistic effects on expression of a target gene associated with the concerted action of the two or more repressor domains.

### Expression Repressors

Expression repressors of the present disclosure may comprise a DNA-targeting moiety and at least one repressor domain. In some embodiments, an expression repressor may comprise 1, 2, 3, 4, 5, 6, or more repressor domains. In some embodiments, an expression repressor targets two or more different DNA sequences (e.g., a 1^{st} and 2^{nd}, 3^{rd}, 4^{th}, 5^{th}, 6^{th}, 7^{th}, 8^{th}, 9^{th}, 10^{th}, 11h, 12^{th}, and/or further DNA sequence, and optionally no more than a 20^{th}, 19^{th}, 18^{th}, 17^{th}, 16^{th}, 15^{th}, 14^{th}, 13^{th}, 12^{th}, 11h, 10^{th}, 9^{th}, 8^{th}, 6^{th}, 5^{th}, 4^{th}, 3^{rd}, or 2^{nd} DNA sequence). In some embodiments, an expression repressor comprises a DNA-targeting moiety and a plurality of repressor domains (e.g., 1, 2, 3, 4, 5, 6, 7, 8, 9, 10, 11, 12, 13, 14, 15, 16, 17, 18, 19, 20, or more repressor domains (and optionally, less than 20, 19, 18, 17, 16, 15, 14, 13, 12, 11, 10, 9, 8, 7, 6, 5, 4, 3, or 2 repressor domains)) each of which may be the same or different from another of the more than one repressor domains.

In some embodiments, an expression repressor comprises a first repressor domain and a second repressor domain, wherein the first repressor domain is not identical to the second repressor domain. In some embodiments, an expression repressor comprises a first repressor domain and a second repressor domain, wherein the first repressor domain is identical to the second repressor domain. In some embodiments, the DNA-targeting moiety is situated between the first repressor domain and the second repressor domain.

An expression repressor may comprise a plurality of repressor domains, where each repressor domain comprises a different functionality than the other repressor domains. For example, an expression repressor may comprise two repressor domains, where the first repressor domain comprises DNA methylase functionality and the second repressor domain comprises a transcriptional repressor functionality. In some embodiments, an expression repressor comprises repressor domains whose functionalities are complementary to one another with regard to decreasing expression of a target gene, where the functionalities together enable inhibition of expression and, optionally, do not inhibit or negligibly inhibit expression when present individually. In some embodiments, an expression repressor comprises a plurality of repressor domains, wherein each repressor domain complements other repressor domains, each repressor domain decreases expression of a target gene.

In some embodiments, an expression repressor comprises a combination of repressor domains whose functionalities synergize with one another with regard to decreasing expression of a target gene. Without wishing to be bound by theory, in some embodiments, epigenetic modifications to a genomic locus are cumulative, in that multiple transcription activating epigenetic markers (e.g., multiple different types of epigenetic markers and/or more extensive marking of a given type) individually together inhibit expression more effectively than individual modifications alone (e.g., producing a greater decrease in expression and/or a longer-lasting decrease in expression). In some embodiments, an expression repressor comprises a plurality of repressor domains, wherein each repressor domain synergizes with other repressor domains, e.g., each repressor domain decreases expression of a target gene. In some embodiments, an expression repressor (comprising a plurality of repressor domains which synergize with one another) is more effective at inhibiting expression of a target gene than an expression repressor comprising an individual repressor domain. In some embodiments, an expression repressor comprising said plurality of repressor domains is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at decreasing expression of a target gene than an expression repressor comprising an individual repressor domain.

In some embodiments, an expression repressor comprises a DNA-targeting moiety and a repressor domain that are covalently linked, e.g., by a peptide bond. In some embodiments, the DNA-targeting moiety and the repressor domain are situated on the same polypeptide chain, e.g., connected by one or more peptide bonds and/or a linker. In some embodiments, the expression repressor is or comprises a fusion molecule, e.g., comprising the DNA-targeting moiety and the repressor domain linked by a peptide bond and/or a linker. In some embodiments, an expression repressor comprises a targeting moiety and a plurality of effector moieties, wherein the targeting moiety and the plurality of effector moieties are covalently linked, e.g., by peptide bonds (e.g., the targeting moiety and plurality of effector moieties are all connected by a series of covalent bonds, although each individual moiety may not share a covalent bond with every other effector moiety). In some embodiments, the expression repressor comprises a DNA-targeting moiety that is linked to the N-terminal of a repressor domain on the same polypeptide chain. In some embodiments, the expression repressor comprises a DNA-targeting moiety that is linked to the C-terminal of a repressor domain on the same polypeptide chain. In some embodiments, the expression repressor comprises a DNA-targeting moiety that is linked to the C-terminal of a first repressor domain and is linked to the N-terminal of a second repressor domain on the same polypeptide chain. In some embodiments, the expression repressor comprises a DNA-targeting moiety that is disposed N-terminal of a repressor domain on the same polypeptide chain. In some embodiments, the expression repressor comprises a DNA-targeting moiety that is disposed C-terminal of a repressor domain on the same polypeptide chain. In some embodiments, an expression repressor comprises a DNA-targeting moiety and a repressor domain that are covalently linked by a non-peptide bond. In some embodiments, a DNA-targeting moiety is conjugated to a repressor domain by a non-peptide bond. In some embodiments, an expression repressor comprises a DNA-targeting moiety and a plurality of repressor domains, wherein the DNA-targeting moiety and the plurality of repressor domains are covalently linked, e.g., by peptide bonds (e.g., the DNA-targeting moiety and plurality of repressor domains are all connected by a series of covalent bonds, although each individual domain or moiety may not share a covalent bond with every other domain or moiety).

In other embodiments, an expression repressor comprises a DNA-targeting moiety and a repressor domain that are not covalently linked, e.g., that are non-covalently associated with one another. In some embodiments, an expression repressor comprises a DNA-targeting moiety that non-covalently binds to a repressor domain or vice versa. In some embodiments, an expression repressor comprises a DNA-targeting moiety and a plurality of repressor domains, wherein the DNA-targeting moiety and at least one repressor domain are not covalently linked, e.g., are non-covalently associated with one another, and wherein the DNA-targeting moiety and at least one other repressor domain are covalently linked, e.g., by a peptide bond.

In general, an expression repressor as described herein binds (e.g., via a DNA-targeting moiety) a genomic sequence element proximal to and/or operably linked to a target gene. In some embodiments, binding of the expression repressor to the genomic sequence element modulates (e.g., decreases) expression of the target gene. For example, binding of an expression repressor comprising a repressor domain that recruits or inhibits recruitment of components of the transcription machinery to the genomic sequence element may modulate (e.g., decrease) expression of the target gene. As a further example, binding of an expression repressor comprising a repressor domain with an enzymatic activity (e.g., an epigenetic modifying moiety) may modulate (e.g., decrease) expression of the target gene) through the localized enzymatic activity of the repressor domain. As a further example, both binding of an expression repressor to a genomic sequence element and the localized enzymatic activity of an expression repressor may contribute to the resulting modulation (e.g., decrease) in expression of the target gene.

In some embodiments, an expression repressor comprises a repressor domain wherein the repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, and the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof.

In some embodiments, an expression repressor comprises a first repressor domain wherein the repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, and the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof and a second repressor domain wherein the repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, and the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof.

In some embodiments, an expression repressor comprises a DNA-targeting moiety and a repressor domain wherein the C-terminal end of the repressor domain, e.g., a repressor domain chosen from EZH1, EZH2, G9A, SUV39H1, FOG1, SETDB1, or SETDB2, and the N-terminal end of the DNA-targeting moiety are covalently linked. In some embodiments, an expression repressor comprises a DNA-targeting moiety and a repressor domain wherein the N-terminal end of the repressor domain, e.g., a repressor domain chosen from LSD1, HDAC8, MQ1, DNMT1, DNMT3a/3L, FOG1, or KRAB, and the C-terminal end of the DNA-targeting moiety are covalently linked. In some embodiments, an expression repressor comprises a DNA-targeting moiety, a first repressor domain and second repressor domain, wherein, the C-terminal end of the first repressor domain, e.g., a repressor domain chosen from EZH1, EZH2, G9A, SUV39H1, FOG1, SETDB1, or SETDB2, and the N-terminal end of the DNA-targeting moiety are covalently linked and the C-terminal end of the DNA-targeting moiety and the N-terminal end of the second repressor domain, e.g., a repressor domain chosen from LSD1, HDAC8, MQ1, DNMT1, DNMT3a/3L, FOG1, or KRAB are covalently linked.

In some embodiments, an expression repressor as disclosed herein is present in a composition, pharmaceutical composition, or mixture.

While several of the embodiments herein describe systems comprising a plurality of expression repressors, it is understood that the present application also provides the expression repressors individually, e.g., for use as a single agent, or for use in a combination with a generic second therapy that need not be specified herein.

In some embodiments, an expression repressor described herein is part of an expression repression system, e.g., as described below.

### Expression Repression Systems

Expression repression systems of the present disclosure may comprise two or more expression repressors. In some embodiments, an expression repression system comprises 2, 3, 4, 5, 6, 7, 8, 9, 10, 11, 12, or more expression repressors (and optionally no more than 15, 14, 13, 12, 11, 10, 9, 8, 7, 6, 5, 4, 3, or 2). In some embodiments, an expression repression system targets two or more different DNA sequences (e.g., a 1^{st} and 2^{nd}, 3^{rd}, 4^{th}, 5^{th}, 6^{th}, 7^{th}, 8^{th}, 9^{th}, 10^{th}, 11h, 12^{th}, and/or further DNA sequence, and optionally no more than a 20^{th}, 19^{th}, 18^{th}, 17^{th}, 16^{th}, 15^{th}, 14^{th}, 13^{th}, 12^{th}, 11h, 10^{th}, 9^{th}, 8^{th}, 6^{th}, 5^{th}, 4^{th}, 3^{rd}, or 2^{nd} DNA sequence). In some embodiments, an expression repression system comprises a plurality of expression repressors, wherein each member of the plurality of expression repressors does not detectably bind, e.g., does not bind, to another member of the plurality of expression repressors. In some embodiments, an expression repression system comprises a first expression repressor and a second expression repressor, wherein the first expression repressor does not detectably bind, e.g., does not bind, to the second expression repressor.

In some embodiments, an expression repression system of the present disclosure comprises two or more expression repressors, wherein the expression repressors are present together in a composition, pharmaceutical composition, or mixture. In some embodiments, an expression repression system of the present disclosure comprises two or more expression repressors, wherein one or more expression repressors is not admixed with at least one other expression repressor. For example, an expression repression system may comprise a first expression repressor and a second expression repressor, wherein the presence of the first expression repressor in the nucleus of a cell does not overlap with the presence of the second expression repressor in the nucleus of the same cell, wherein the expression repression system achieves a decrease in expression of a target gene via the non-overlapping presences of the first and second expression repressors.

In some embodiments, the expression repressors of an expression repression system each comprise a different DNA-targeting moiety (e.g., the first, second, third, or further expression repressors each comprise different DNA-targeting moieties from one another). For example, an expression repression system may comprise a first expression repressor and a second expression repressor wherein the first expression repressor comprises a first DNA-targeting moiety (e.g., a Cas9 molecule, TAL effector molecule, or Zn Finger domain), and the second expression repressor comprises a second DNA-targeting moiety (e.g., a Cas9 molecule, TAL effector molecule, or Zn Finger domain) different from the first DNA-targeting moiety. In some embodiments, different can mean comprising distinct types of DNA-targeting moiety, e.g., the first DNA-targeting moiety comprises a Cas9 molecule, and the second DNA-targeting moiety comprises a TAL effector molecule. In other embodiments, different can mean comprising distinct variants of the same type of DNA-targeting moiety, e.g., the first DNA-targeting moiety comprises a first Cas9 molecule (e.g., from a first species) and the second DNA-targeting moiety comprises a second Cas9 molecule (e.g., from a second species). In an embodiment, when an expression repression system comprises two or more DNA-targeting moieties of the same type, e.g., two or more Cas9 molecules, the DNA-targeting moieties specifically bind two or more different DNA sequences. For example, in an expression repression system comprising two or more Cas9 molecules, the two or more Cas9 molecules may be chosen or altered such that they only appreciably bind the gRNA corresponding to their target DNA sequence (e.g., and do not appreciably bind the gRNA corresponding to the target of another Cas9 molecule). In a further example, in an expression repression system comprising two or more TAL effector molecules, the two or more TAL effector molecules may be chosen or altered such that they only appreciably bind to their target DNA sequence (e.g., and do not appreciably bind the target DNA sequence of another TAL effector molecule).

In some embodiments, an expression repression system comprises three or more expression repressors and two or more expression repressors comprise the same DNA-targeting moiety. For example, an expression repression system may comprise three expression repressors, wherein the first and second expression repressors both comprise a first DNA-targeting moiety and the third expression repressor comprises a second different DNA-targeting moiety. For a further example, an expression repression system may comprise four expression repressors, wherein the first and second expression repressors both comprise a first DNA-targeting moiety and the third and fourth expression repressors comprises a second different DNA-targeting moiety. For a further example, an expression repression system may comprise five expression repressors, wherein the first and second expression repressors both comprise a first DNA-targeting moiety, the third and fourth expression repressors both comprise a second different DNA-targeting moiety, and the fifth expression repressor comprises a third different DNA-targeting moiety. As described above, different can mean comprising different types of DNA-targeting moieties or comprising distinct variants of the same type of DNA-targeting moiety.

In some embodiments, the expression repressors of an expression repression system each bind to a different DNA sequence (e.g., the first, second, third, or further expression repressors each bind DNA sequences that are different from one another). For example, an expression repression system may comprise a first expression repressor and a second expression repressor wherein the first expression repressor binds to a first DNA sequence, and the second expression repressor binds to a second DNA sequence. In some embodiments, different can mean that: there is at least one position that is not identical between the DNA sequence bound by one expression repressor and the DNA sequence bound by another expression repressor, or that there is at least one position present in the DNA sequence bound by one expression repressor that is not present in the DNA sequence bound by another expression repressor. For example, a first expression repressor may bind to a first exemplary DNA sequence 5′-ATGATTGGATTTA-3′ (SEQ ID NO: 97), and a second expression repressor may bind to a second exemplary DNA sequence 5′-TGATTGGATTTAG-3′ (SEQ ID NO: 98); in said example, the first and second exemplary DNA sequences are different. For a further example, a first expression repressor may bind to a first exemplary DNA sequence 5′-ATGATTgGATTTA-3′ (SEQ ID NO: 99), and a second expression repressor may bind to a second exemplary DNA sequence 5′-ATGATTcGATTTA-3′ (SEQ ID NO: 100); in said example, the first and second exemplary DNA sequences are different. In some embodiments, the first DNA sequence may be situated on a first genomic DNA strand and the second DNA sequence may be situated on a second genomic DNA strand. In some embodiments, the first DNA sequence may be situated on the same genomic DNA strand as the second DNA sequence.

In some embodiments, an expression repression system comprises three or more expression repressors and two or more expression repressors bind the same DNA sequence. For example, an expression repression system may comprise three expression repressors, wherein the first and second expression repressors both bind a first DNA sequence, and the third expression repressor binds a second different DNA sequence. For a further example, an expression repression system may comprise four expression repressors, wherein the first and second expression repressors both bind a first DNA sequence and the third and fourth expression repressors both bind a second DNA sequence. For a further example, an expression repression system may comprise five expression repressors, wherein the first and second expression repressors both bind a first DNA sequence, the third and fourth expression repressors both bind a second DNA sequence, and the fifth expression repressor binds a third DNA sequence. As described above, different can mean that there is at least one position that is not identical between the DNA sequence bound by one expression repressor and the DNA sequence bound by another expression repressor, or that there is at least one position present in the DNA sequence bound by one expression repressor that is not present in the DNA sequence bound by another expression repressor.

In some embodiments, an expression repression system comprises two or more (e.g., two) expression repressors and a plurality (e.g., two) of the expression repressors comprise DNA-targeting moieties that bind to different DNA sequences. In such embodiments, a first DNA-targeting moiety may bind to a first DNA sequence and a second DNA-targeting moiety may bind to a second DNA sequence, wherein the first and the second DNA sequences are different and do not overlap. In some such embodiments, the first DNA sequence is separated from the second DNA sequence by at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no more than 500, 400, 300, 200, 100, 95, 90, 85, 80, 75, 70, 65, 60, 55, or 50 base pairs). In some such embodiments, the first DNA sequence is separated from the second DNA sequence by no more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no base pairs, e.g., the first and second sequence are directly adjacent one another).

In some embodiments, the expression repressors of an expression repression system each comprise a different repressor domain (e.g., the first, second, third, or further expression repressors each comprise a different repressor domain from one another). For example, an expression repression system may comprise a first expression repressor and a second expression repressor wherein the first expression repressor comprises a first repressor domain (e.g., comprising a histone methyltransferase or functional fragment thereof), and the second expression repressor comprises a second repressor domain (e.g., comprising a DNA methyltransferase or functional fragment thereof) different from the first repressor domain. In some embodiments, different can mean comprising distinct types of repressor domain, e.g., the first repressor domain comprises a histone methyltransferase and the second repressor domain comprises a DNA methyltransferase, or the first repressor domain comprises a histone methyltransferase and the second repressor domain comprises a small molecule inhibitor of an enzyme. In other embodiments, different can mean comprising distinct variants of the same type of repressor domain, e.g., the first repressor domain comprises a first histone methyltransferase (e.g., having a first site specificity or amino acid sequence) and the second repressor domain comprises a second histone methyltransferase (e.g., having a second site specificity or amino acid sequence).

In some embodiments, an expression repression system comprises a first expression repressor comprising a first repressor domain and a second expression repressor comprising a second repressor domain, wherein the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, and the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof.

In some embodiments, an expression repression system comprises a first expression repressor comprising a first repressor domain and a second expression repressor comprising a second repressor domain, wherein the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, and the third repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof.

In some embodiments, an expression repression system comprises: (i) a first expression repressor comprising a first repressor domain and a third repressor domain, and (ii) a second expression repressor comprising a second repressor domain and optionally a fourth repressor domain, wherein the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof, the third repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof and the fourth repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, KRAB, MeCP2, HP1, RBBP4, REST, FOG1, SUZ12, or a functional variant or fragment thereof.

In some embodiments, the first repressor domain comprises a histone methyltransferase activity (e.g., SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof) and the second repressor domain comprises a histone demethylase activity (e.g., KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof). In some embodiments, the first repressor domain comprises a histone methyltransferase activity and the second repressor domain comprises a histone deacetylase activity (e.g., HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, the first repressor domain comprises a histone methyltransferase activity and the second repressor domain comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, or a functional variant or fragment of any thereof). In some embodiments, the first repressor domain comprises a histone methyltransferase activity and the second repressor domain comprises a DNA demethylase activity. In some embodiments the first repressor domain comprises a histone methyltransferase activity and the second repressor domain comprises a transcription repressor activity (e.g., KRAB, MeCP2, HP1, RBBP4, REST, FOG1, SUZ12, or a functional variant or fragment of any thereof). In some embodiments, the first repressor domain comprises a histone methyltransferase activity and the second repressor domain comprises a different histone methyltransferase activity. In some embodiments, the first repressor domain comprises a histone methyltransferase activity and the second repressor domain comprises the same histone methyltransferase activity. In some embodiments, the first repressor domain comprises a histone demethylase activity and the second repressor domain comprises a histone deacetylase activity. In some embodiments, the first repressor domain comprises a histone demethylase activity and the second repressor domain comprises a DNA methyltransferase activity. In some embodiments, the first repressor domain comprises a histone demethylase activity and the second repressor domain comprises a DNA demethylase activity. In some embodiments, the first repressor domain comprises a histone demethylase activity and the second repressor domain comprises a transcription repressor activity. In some embodiments, the first repressor domain comprises a histone demethylase activity and the second repressor domain comprises a different histone demethylase activity. In some embodiments, the first repressor domain comprises a histone demethylase activity and the second repressor domain comprises the same histone demethylase activity. In some embodiments, the first repressor domain comprises a histone deacetylase activity and the second repressor domain comprises a DNA methyltransferase activity. In some embodiments, the first repressor domain comprises a histone deacetylase activity and the second repressor domain comprises a DNA demethylase activity. In some embodiments, the first repressor domain comprises a histone deacetylase activity and the second repressor domain comprises a transcription repressor activity. In some embodiments, the first repressor domain comprises a histone deacetylase activity and the second repressor domain comprises a different histone deacetylase activity. In some embodiments, the first repressor domain comprises a histone deacetylase activity and the second repressor domain comprises the same histone deacetylase activity. In some embodiments, the first repressor domain comprises a DNA methyltransferase activity and the second repressor domain comprises a DNA demethylase activity. In some embodiments, the first repressor domain comprises a DNA methyltransferase activity and the second repressor domain comprises a transcription repressor activity. In some embodiments, the first repressor domain comprises a DNA methyltransferase activity and the second repressor domain comprises a different DNA methyltransferase activity. In some embodiments, the first repressor domain comprises a DNA methyltransferase activity and the second repressor domain comprises the same DNA methyltransferase activity. In some embodiments, the first repressor domain comprises a DNA demethylase activity and the second repressor domain comprises a transcription repressor activity. In some embodiments, the first repressor domain comprises a DNA demethylase activity and the second repressor domain comprises a different DNA demethylase activity. In some embodiments, the first repressor domain comprises a DNA demethylase activity and the second repressor domain comprises the same DNA demethylase activity. In some embodiments, the first repressor domain comprises a transcription repressor activity and the second repressor domain comprises a different transcription repressor activity. In some embodiments, the first repressor domain comprises a transcription repressor activity and the second repressor domain comprises the same transcription repressor activity.

In some embodiments, an expression repression system comprises three or more expression repressors and two or more expression repressors comprise the same DNA-targeting moiety. For example, an expression repression system may comprise three expression repressors, wherein the first and second expression repressors both comprise a first repressor domain and the third expression repressor comprises a second different repressor domain. For a further example, an expression repression system may comprise four expression repressors, wherein the first and second expression repressors both comprise a first repressor domain and the third and fourth expression repressors comprises a second different repressor domain. For a further example, an expression repression system may comprise five expression repressors, wherein the first and second expression repressors both comprise a first repressor domain, the third and fourth expression repressors both comprise a second different repressor domain, and the fifth expression repressor comprises a third different repressor domain. As described above, different can mean comprising different types of repressor domain or comprising distinct variants of the same type of repressor domain.

In some embodiments, two repressor domains comprise a histone methyltransferase activity (e.g., SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof) and the other repressor domain comprises a histone demethylase activity (e.g., KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof). In some embodiments, two repressor domains comprise a histone methyltransferase activity and the other repressor domain comprises a histone deacetylase activity (e.g., HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof). In some embodiments, two repressor domains comprise a histone methyltransferase activity and the other repressor domain comprises a DNA methyltransferase activity (e.g., MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L, or a functional variant or fragment of any thereof). In some embodiments, two repressor domains comprise a histone methyltransferase activity and the other repressor domain comprises a DNA demethylase activity. In some embodiments, two repressor domains comprise a histone methyltransferase activity and the other repressor domain comprises a transcription repressor activity (e.g., KRAB, MeCP2, HP1, RBBP4, REST, FOG1, SUZ12, or a functional variant or fragment of any thereof). In some embodiments, two repressor domains comprise a histone methyltransferase activity and the other repressor domain comprises a different histone methyltransferase activity. In some embodiments, two repressor domains comprise a histone methyltransferase activity and the other repressor domain comprises the same histone methyltransferase activity. In some embodiments, two repressor domains comprise a histone demethylase activity and the other repressor domain comprises a histone deacetylase activity. In some embodiments, two repressor domains comprise a histone demethylase activity and the other repressor domain comprises a DNA methyltransferase activity. In some embodiments, two repressor domains comprise a histone demethylase activity and the other repressor domain comprises a DNA demethylase activity. In some embodiments, two repressor domains comprise a histone demethylase activity and the other repressor domain comprises a transcription repressor activity. In some embodiments, two repressor domains comprise a histone demethylase activity and the other repressor domain comprises a different histone demethylase activity. In some embodiments, two repressor domains comprise a histone demethylase activity and the other repressor domain comprises the same histone demethylase activity. In some embodiments, two repressor domains comprise a histone deacetylase activity and the other repressor domain comprises a DNA methyltransferase activity. In some embodiments, two repressor domains comprise a histone deacetylase activity and the other repressor domain comprises a DNA demethylase activity. In some embodiments, two repressor domains comprise a histone deacetylase activity and the other repressor domain comprises a transcription repressor activity. In some embodiments, two repressor domains comprise a histone deacetylase activity and the other repressor domain comprises a different histone deacetylase activity. In some embodiments, two repressor domains comprise a histone deacetylase activity and the other repressor domain comprises the same histone deacetylase activity. In some embodiments, two repressor domains comprise a DNA methyltransferase activity and the other repressor domain comprises a DNA demethylase activity. In some embodiments, two repressor domains comprise a DNA methyltransferase activity and the other repressor domain comprises a transcription repressor activity. In some embodiments, two repressor domains comprise a DNA methyltransferase activity and the other repressor domain comprises a different DNA methyltransferase activity. In some embodiments, two repressor domains comprise a DNA methyltransferase activity and the other repressor domain comprises the same DNA methyltransferase activity. In some embodiments, two repressor domains comprise a DNA demethylase activity and the other repressor domain comprises a transcription repressor activity. In some embodiments, two repressor domains comprise a DNA demethylase activity and the other repressor domain comprises a different DNA demethylase activity. In some embodiments, two repressor domains comprise a DNA demethylase activity and the other repressor domain comprises the same DNA demethylase activity. In some embodiments, two repressor domains comprise a transcription repressor activity and the other repressor domain comprises a different transcription repressor activity. In some embodiments, two repressor domains comprise a transcription repressor activity and the other repressor domain comprises the same transcription repressor activity.

In some embodiments, two or more (e.g., all) expression repressors of an expression repression system are not covalently associated with each other, e.g., each expression repressor is not covalently associated with any other expression repressor. In another embodiment, two or more expression repressors of an expression repression system are covalently associated with one another. In an embodiment, an expression repression system comprises a first expression repressor and a second expression repressor disposed on the same polypeptide, e.g., as a fusion molecule, e.g., connected by a peptide bond and optionally a linker. In an embodiment, an expression repression system comprises a first expression repressor and a second expression repressor that are connected by a non-peptide bond, e.g., are conjugated to one another.

### Linkers

An expression repressor or an expression repression system as disclosed herein may comprise one or more linkers. A linker may connect a DNA-targeting moiety to a repressor domain, a repressor domain to another repressor domain, or a DNA-targeting moiety to another DNA-targeting moiety. A linker may be a chemical bond, e.g., one or more covalent bonds or non-covalent bonds. In some embodiments, a linker is covalent. In some embodiments, a linker is non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first domain or moiety from a second domain or moiety, e.g., a DNA-targeting moiety from a repressor domain. In some embodiments, for example, a linker can be positioned between a DNA-targeting moiety and a repressor domain, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g., polypeptide) from a modulating agent, but sufficiently stable to prevent premature cleavage.

In some embodiments, one or more moieties and/or domains of an expression repressor described herein are linked with one or more linkers. In some embodiments, an expression repression may comprise a linker situated between the DNA-targeting moiety and the repressor domain. In some embodiments, an expression repressor may comprise a first linker situated between the DNA-targeting moiety and the first repressor domain, and a second linker situated between the DNA-targeting moiety and the second repressor domain. In some embodiments, the first and the second linker may be identical. In some embodiments, the first and the second linker may be different.

As will be known by one of skill in the art, commonly used flexible linkers have sequences consisting primarily of stretches of Gly and Ser residues (“GS” linker). Flexible linkers may be useful for joining domains/moieties that require a certain degree of movement or interaction and may include small, non-polar (e.g., Gly) or polar (e.g., Ser or Thr) amino acids. Incorporation of Ser or Thr can also maintain the stability of a linker in aqueous solutions by forming hydrogen bonds with water molecules, and therefore reduce unfavorable interactions between a linker and moieties/domains.

Rigid linkers are useful to keep a fixed distance between domains/moieties and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv. Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of molecules suitable for use in linkers described herein include a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof; noncarbon linkers; carbohydrate linkers; phosphodiester linkers, or other molecule capable of covalently linking two or more components of an expression repressor. Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine, or other hydrophobic residue. Components of an expression repressor may be linked using charge-based chemistry, such that a positively charged component of an expression repressor is linked to a negative charge of another component.

### Nucleic Acids

In one aspect, the disclosure provides nucleic acid sequences encoding an expression repressor, an expression repression system, a DNA-targeting moiety and/or a repressor domain as described herein. A skilled artisan is aware that the nucleic acid sequences of RNA are identical to the corresponding DNA sequences, except that typically thymine (T) is replaced by uracil (U). It will be understood that when a nucleotide sequence is represented by a DNA sequence (e.g., comprising, A, T, G, C), this disclosure also provides the corresponding RNA sequence (e.g., comprising, A, U, G, C) in which “U” replaces “T.” Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

It will be appreciated by those skilled in the art that due to the degeneracy of the genetic code, a multitude of nucleotide sequences encoding an expression repressor comprising DNA-targeting moiety and/or a repressor domain as described herein may be produced, some of which have similarity, e.g., 90%, 95%, 96%, 97%, 98%, or 99% identity to the nucleic acid sequences disclosed herein. For instance, codons AGA, AGG, CGA, CGC, CGG, and CGU all encode the amino acid arginine. Thus, at every position in the nucleic acid molecules of the disclosure where an arginine is specified by a codon, the codon can be altered to any of the corresponding codons described above without altering the encoded polypeptide.

In some embodiments a nucleic acid sequence encoding an expression repressor comprising a DNA-targeting moiety and/or one or more repressor domains may be part or all of a codon-optimized coding region, optimized according to codon usage in mammals, e.g., humans. In some embodiments, a nucleic acid sequence encoding a DNA-targeting moiety and/or one or more repressor domains is codon optimized for increasing the protein expression and/or increasing the duration of protein expression. In some embodiments, a protein produced by the codon optimized nucleic acid sequence is at least 1%, at least 2%, at least 5%, at least 10%, at least 15%, at least 20%, at least 30%, at least 40%, or at least 50% higher compared to levels of the protein when encoded by a nucleic acid sequence that is not codon optimized.

In one aspect, the disclosure is directed to a polypeptide comprising one or more (e.g., one) DNA-targeting moiety and one or more repressor domain, e.g., wherein the repressor domain is or comprises MQ1, e.g., bacterial MQ1, or a functional variant or fragment thereof. In some embodiments, MQ1 is Spiroplasma monobiae MQ1, e.g., MQ1 from strain ATCC 33825 and/or corresponding to Uniprot ID P15840. In some embodiments, MQ1 repressor domain is encoded by a nucleotide sequence of SEQ ID NO: 47. In some embodiments, a nucleotide sequence described herein comprises a sequence of SEQ ID NO: 47 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 47)
AGCAAGGTGGAGAACAAGACCAAGAAGCTGCGGGTGTTCG
AGGCCTTCGCCGGCATCGGCGCCCAGCGGAAGGCCCTGGA
GAAGGTGCGGAAGGACGAGTACGAGATCGTGGGCCTGGCC
GAGTGGTACGTGCCCGCCATCGTGATGTACCAGGCCATCC
ACAACAACTTCCACACCAAGCTGGAGTACAAGAGCGTGAG
CCGGGAGGAGATGATCGACTACCTGGAGAACAAGACCCTG
AGCTGGAACAGCAAGAACCCCGTGAGCAACGGCTACTGGA
AGCGGAAGAAGGACGACGAGCTGAAGATCATCTACAACGC
CATCAAGCTGAGCGAGAAGGAGGGCAACATCTTCGACATC
CGGGACCTGTACAAGCGGACCCTGAAGAACATCGACCTGC
TGACCTACAGCTTCCCCTGCCAGGACCTGAGCCAGCAGGG
CATCCAGAAGGGCATGAAGCGGGGCAGCGGCACCCGGAGC
GGCCTGCTGTGGGAGATCGAGCGGGCCCTGGACAGCACCG
AGAAGAACGACCTGCCCAAGTACCTGCTGATGGAGAACGT
GGGCGCCCTGCTGCACAAGAAGAACGAGGAGGAGCTGAAC
CAGTGGAAGCAGAAGCTGGAGAGCCTGGGCTACCAGAACA
GCATCGAGGTGCTGAACGCCGCCGACTTCGGCAGCAGCCA
GGCCCGGCGGCGGGTGTTCATGATCAGCACCCTGAACGAG
TTCGTGGAGCTGCCCAAGGGCGACAAGAAGCCCAAGAGCA
TCAAGAAGGTGCTGAACAAGATCGTGAGCGAGAAGGACAT
CCTGAACAACCTGCTGAAGTACAACCTGACCGAGTTCAAG
AAAACCAAGAGCAACATCAACAAGGCCAGCCTGATCGGCT
ACAGCAAGTTCAACAGCGAGGGCTACGTGTACGACCCCGA
GTTCACCGGCCCCACCCTGACCGCCAGCGGCGCCAACAGC
CGGATCAAGATCAAGGACGGCAGCAACATCCGGAAGATGA
ACAGCGACGAGACCTTCCTGTACATCGGCTTCGACAGCCA
GGACGGCAAGCGGGTGAACGAGATCGAGTTCCTGACCGAG
AACCAGAAGATCTTCGTGTGCGGCAACAGCATCAGCGTGG
AGGTGCTGGAGGCCATCATCGACAAGATCGGCGGC

In some embodiments, MQ1 comprises an amino acid sequence of SEQ ID NO: 90. In some embodiments, MQ1 comprises an amino acid sequence of SEQ ID NO: 57. In some embodiments, an effector domain described herein comprises SEQ ID NO: 90 or 57, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 90)
MSKVENKTKKLRVFEAFAGIGAQRKALEKVRKDEYEIVGL
AEWYVPAIVMYQAIHNNFHTKLEYKSVSREEMIDYLENKT
LSWNSKNPVSNGYWKRKKDDELKIIYNAIKLSEKEGNIFD
IRDLYKRTLKNIDLLTYSFPCQDLSQQGIQKGMKRGSGTR
SGLLWEIERALDSTEKNDLPKYLLMENVGALLHKKNEEEL
NQWKQKLESLGYQNSIEVLNAADFGSSQARRRVFMISTLN
EFVELPKGDKKPKSIKKVLNKIVSEKDILNNLLKYNLTEF
KKTKSNINKASLIGYSKFNSEGYVYDPEFTGPTLTASGAN
SRIKIKDGSNIRKMNSDETFLYIGFDSQDGKRVNEIEFLT
ENQKIFVCGNSISVEVLEAIIDKIGG
(SEQ ID NO: 57)
SKVENKTKKLRVFEAFAGIGAQRKALEKVRKDEYEIVGLA
EWYVPAIVMYQAIHNNFHTKLEYKSVSREEMIDYLENKTL
SWNSKNPVSNGYWKRKKDDELKIIYNAIKLSEKEGNIFDI
RDLYKRTLKNIDLLTYSFPCQDLSQQGIQKGMKRGSGTRS
GLLWEIERALDSTEKNDLPKYLLMENVGALLHKKNEEELN
QWKQKLESLGYQNSIEVLNAADFGSSQARRRVFMISTLNE
FVELPKGDKKPKSIKKVLNKIVSEKDILNNLLKYNLTEFK
KTKSNINKASLIGYSKFNSEGYVYDPEFTGPTLTASGANS
RIKIKDGSNIRKMNSDETFLYIGFDSQDGKRVNEIEFLTE
NQKIFVCGNSISVEVLEAIIDKIGG

In some embodiments, MQ1 for use in a polypeptide described herein is a variant, e.g., comprising one or more mutations, relative to wildtype MQ1 (e.g., SEQ ID NO: 90 or SEQ ID NO: 57). In some embodiments, an MQ1 variant comprises one or more amino acid substitutions, deletions, or insertions relative to wildtype MQ1. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1). In some embodiments, the polypeptide comprises one or more linkers described herein, e.g., connecting a moiety/domain to another moiety/domain. In some embodiments, the polypeptide comprises a DNA-targeting moiety that is or comprises a CRISPR/Cas molecule, e.g., comprising a CRISPR/Cas protein, e.g., a dCas9 protein. In some embodiments, the polypeptide is a fusion protein comprising a repressor domain that is or comprises MQ1 and a DNA-targeting moiety that is or comprises a CRISPR/Cas molecule, e.g., comprising a CRISPR/Cas protein, e.g., a dCas9 protein. In some embodiments, the polypeptide comprises an additional moiety described herein. In some embodiments, the polypeptide decreases expression of a target gene (e.g., a target gene described herein). In some embodiments, the polypeptide may be used in methods of modulating, e.g., decreasing, gene expression, methods of treating a condition, or methods of epigenetically modifying a target gene or transcription control element described herein, e.g., in place of an expression repression system. In some embodiments, an expression repression system comprises two or more (e.g., two, three, or four) expression repressors, wherein the first expression repressor comprises a repressor domain comprising MQ1, e.g., bacterial MQ1, or a functional variant or fragment thereof.

In another aspect, the disclosure is directed to an expression repressor or a polypeptide comprising one or more (e.g., one) targeting moiety and one or more effector moiety, wherein the effector moiety is or comprises Krueppel-associated box (KRAB) e.g., as according to NP_056209.2 or the protein encoded by NM_015394.5 or a functional variant or fragment thereof. In some embodiments, KRAB is a synthetic KRAB construct. In some embodiments, KRAB comprises an amino acid sequence of SEQ ID NO: 61:

(SEQ ID NO: 61)
DAKSLTAWSRTLVTFKDVFVDFTREEWKLLDTAQQILYRN
VMLENYKNLVSLGYQLTKPDVILRLEKGEEPWLVEREIHQ
ETHPDSETAFEIKSSV

In some embodiments, the KRAB repressor domain is encoded by a nucleotide sequence of SEQ ID NO: 51. In some embodiments, a nucleotide sequence described herein comprises a sequence of SEQ ID NO: 51 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 51)
GACGCCAAGAGCCTGACCGCCTGGAGCCGGACCCTGGTGA
CCTTCAAGGACGTGTTCGTGGACTTCACCCGGGAGGAGTG
GAAGCTGCTGGACACCGCCCAGCAGATCCTGTACCGGAAC
GTGATGCTGGAGAACTACAAGAACCTGGTGAGCCTGGGCT
ACCAGCTGACCAAGCCCGACGTGATCCTGCGGCTGGAGAA
GGGCGAGGAGCCCTGGCTGGTGGAGCGGGAGATCCACCAG
GAGACCCACCCCGACAGCGAGACCGCCTTCGAGATCAAGA
GCAGCGTG

In some embodiments, KRAB for use in a polypeptide or an expression repressor described herein is a variant, e.g., comprising one or more mutations, relative to the KRAB sequence of SEQ ID NO: 61. In some embodiments, an KRAB variant comprises one or more amino acid substitutions, deletions, or insertions relative to SEQ ID NO: 61.

In some embodiments, the polypeptide or the expression repressor is a fusion protein comprising a repressor domain that is or comprises KRAB and a DNA-targeting moiety. In some embodiments, the polypeptide or the expression repressor comprises an additional moiety described herein. In some embodiments, the polypeptide or the expression repressor decreases expression of a target gene. In some embodiments, the polypeptide or the expression repressor may be used in methods of modulating, e.g., decreasing, gene expression, methods of treating a condition, or methods of epigenetically modifying a target gene, e.g., a transcription control element described herein, e.g., in place of an expression repression system. In some embodiments, an expression repression system comprises two or more (e.g., two, three, or four) expression repressors, wherein the first expression repressor comprises a repressor domain comprising the KRAB sequence of SEQ ID NO: 61, or a functional variant or fragment thereof.

In another aspect, the disclosure is directed to an expression repressor or a polypeptide comprising one or more (e.g., one) DNA-targeting moiety and one or more repressor domain, wherein the repressor domain is or comprises DNMT1, e.g., human DNMT1, or a functional variant or fragment thereof. In some embodiments, DNMT1 is human DNMT1, e.g., corresponding to Gene ID 1786, e.g., corresponding to UniPort ID P26358.2. In some embodiments, DNMT1 comprises an amino acid sequence of SEQ ID NO: 58. In some embodiments, a repressor domain described herein comprises a sequence according to SEQ ID NO: 58 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto:

(SEQ ID NO: 58)
VDLRTLDVFSGCGGLSEGFHQAGISDTLWAIEMWDPAAQA
FRLNNPGSTVFTEDCNILLKLVMAGETTNSRGQRLPQKGD
VEMLCGGPPCQGFSGMNRFNSRTYSKFKNSLVVSFLSYCD
YYRPRFFLLENVRNFVSFKRSMVLKLTLRCLVRMGYQCTF
GVLQAGQYGVAQTRRRAIILAAAPGEKLPLFPEPLHVFAP
RACQLSVVVDDKKFVSNITRLSSGPFRTITVRDTMSDLPE
VRNGASALEISYNGEPQSWFQRQLRGAQYQPILRDHICKD
MSALVAARMRHIPLAPGSDWRDLPNIEVRLSDGTMARKLR
YTHHDRKNGRSSSGALRGVCSCVEAGKACDPAARQFNTLI
PWCLPHTGNRHNHWAGLYGRLEWDGFFSTTVTNPEPMGKQ
GRVLHPEQHRVVSVRECARSQGFPDTYRLFGNILDKHRQV
GNAVPPPLAKAIGLEIKLCMLAKARESASAKIKEEEAAKD

In some embodiments, DNMT1 is encoded by a nucleotide sequence of SEQ ID NO: 48. In some embodiments, a nucleic acid described herein comprises a sequence of SEQ ID NO: 48 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto:

(SEQ ID NO: 48)
GTGGATCTGAGGACACTCGACGTGTTTAGCGGATGCGGCG
GACTCTCCGAAGGCTTCCACCAAGCCGGAATTTCCGACAC
ACTCTGGGCCATTGAGATGTGGGACCCCGCCGCTCAAGCC
TTCAGACTGAATAATCCCGGCTCCACCGTGTTCACCGAGG
ACTGCAACATTCTGCTGAAGCTGGTGATGGCTGGCGAAAC
CACCAACTCTAGAGGCCAGAGGCTGCCCCAGAAGGGAGAT
GTGGAAATGCTCTGTGGAGGCCCTCCTTGCCAAGGCTTCT
CCGGCATGAACAGGTTCAACTCTAGAACATACAGCAAGTT
CAAGAACTCTCTGGTCGTGAGCTTTCTGAGCTACTGCGAC
TACTATAGACCTAGGTTCTTTCTGCTGGAGAACGTGAGAA
ATTTCGTGTCCTTCAAGAGGAGCATGGTGCTGAAGCTGAC
ACTGAGGTGTCTGGTGAGGATGGGCTACCAGTGCACATTC
GGAGTGCTGCAAGCTGGCCAGTACGGCGTGGCCCAGACCA
GAAGGAGGGCCATCATTCTGGCTGCTGCCCCCGGCGAGAA
ACTCCCTCTGTTCCCCGAGCCCCTCCACGTGTTCGCCCCT
AGAGCTTGCCAGCTGAGCGTGGTGGTCGACGATAAGAAGT
TCGTGAGCAACATCACAAGGCTGTCCAGCGGACCCTTCAG
AACCATTACCGTGAGGGATACCATGTCCGACCTCCCCGAG
GTGAGGAATGGCGCCAGCGCTCTGGAGATTTCCTACAACG
GCGAACCTCAGAGCTGGTTCCAAAGGCAGCTGAGAGGCGC
TCAGTATCAGCCCATTCTGAGGGACCACATCTGCAAAGAT
ATGAGCGCTCTGGTGGCCGCTAGAATGAGACATATTCCTC
TGGCCCCCGGCAGCGACTGGAGAGATCTGCCCAATATTGA
GGTGAGACTCAGCGACGGAACAATGGCTAGAAAACTGAGG
TACACCCATCATGATAGAAAGAACGGAAGGAGCAGCAGCG
GCGCTCTGAGAGGAGTGTGTAGCTGCGTGGAAGCTGGCAA
GGCTTGCGATCCCGCCGCTAGGCAGTTCAATACCCTCATC
CCTTGGTGTCTGCCTCACACCGGCAACAGACACAATCATT
GGGCTGGACTGTATGGAAGGCTCGAATGGGACGGCTTTTT
CAGCACCACCGTGACCAATCCCGAACCTATGGGCAAGCAA
GGAAGGGTGCTCCACCCCGAGCAGCATAGAGTCGTGTCCG
TGAGAGAATGCGCTAGAAGCCAAGGCTTCCCCGACACCTA
TAGACTGTTCGGCAACATTCTGGATAAGCACAGACAAGTG
GGAAATGCTGTCCCTCCTCCTCTGGCCAAGGCTATCGGAC
TGGAGATCAAGCTGTGTATGCTCGCCAAAGCTAGGGAGAG
CGCTTCCGCCAAGATTAAGGAGGAGGAGGCCGCCAAGGAC

In some embodiments, DNMT1 for use in a polypeptide or an expression repressor described herein is a variant, e.g., comprising one or more mutations, relative to a DNMT sequence of SEQ ID NO: 58. In some embodiments, the effector domain comprises one or more amino acid substitutions, deletions, or insertions relative to wild type DNMT1. In some embodiments, the polypeptide is a fusion protein comprising a repressor domain that is or comprises DNMT1 and a targeting moiety. In some embodiments, an expression repression system comprises two or more (e.g., two, three, or four) expression repressors, wherein the first expression repressor comprises a repressor domain comprising DNMT1, or a functional variant or fragment thereof.

In another aspect, the disclosure is directed to an expression repressor or a polypeptide comprising one or more (e.g., one) DNA-targeting moiety and one or more repressor domain, wherein the repressor domain is or comprises DNMT3a/3L complex, or a functional variant or fragment thereof. In some embodiments, the DNMT3a/3L complex is a fusion construct. In some embodiments the DNMT3a/3L complex comprises DNMT3A, e.g., human DNMT3A, e.g., as according to NP_072046.2 or the protein encoded by NM_022552.4). In some embodiments the DNMT3a/3L complex comprises mouse DNMT3A, e.g., as according to NP_031898 or the protein encoded by NM_007872. In some embodiments the DNMT3a/3L complex comprises human DNMT3L (e.g., as according to NP_787063.1 or the protein encoded by NM_175867.3). In some embodiments the DNMT3a/3L complex comprises mouse DNMT3L (e.g., as according to NP_001075164 or the protein encoded by NM_001081695). In some embodiments, DNMT3a/3L comprises an amino acid sequence of SEQ ID NO:59 or 60. In some embodiments, a repressor domain described herein comprises SEQ ID NO: 59 or SEQ ID NO: 60, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 59)
NHDQEFDPPKVYPPVPAEKRKPIRVLSLFDGIATGLLVLKDLGIQVDRYIASEVCEDSITVGMVRH
QGKIMYVGDVRSVTQKHIQEWGPFDLVIGGSPCNDLSIVNPARKGLYEGTGRLFFEFYRLLHDA
RPKEGDDRPFFWLFENVVAMGVSDKRDISRFLESNPVMIDAKEVSAAHRARYFWGNLPGMNRP
LASTVNDKLELQECLEHGRIAKFSKVRTITTRSNSIKQGKDQHFPVFMNEKEDILWCTEMERVFG
FPVHYTDVSNMSRLARQRLLGRSWSVPVIRHLFAPLKEYFACVSSGNSNANSRGPSFSSGLVPLS
LRGSHMNPLEMFETVPVWRRQPVRVLSLFEDIKKELTSLGFLESGSDPGQLKHVVDVTDTVRKD
VEEWGPFDLVYGATPPLGHTCDRPPSWYLFQFHRLLQYARPKPGSPRPFFWMFVDNLVLNKEDL
DVASRFLEMEPVTIPDVHGGSLQNAVRVWSNIPAIRSRHWALVSEEELSLLAQNKQSSKLAAKW
PTKLVKNCFLPLREYFKYFSTELTSS
(SEQ ID NO: 60)
NHDQEFDPPKVYPPVPAEKRKPIRVLSLFDGIATGLLVLKDLGIQVDRYIASEVCEDSITVGMVRH
QGKIMYVGDVRSVTQKHIQEWGPFDLVIGGSPCNDLSIVNPARKGLYEGTGRLFFEFYRLLHDA
RPKEGDDRPFFWLFENVVAMGVSDKRDISRFLESNPVMIDAKEVSAAHRARYFWGNLPGMNRP
LASTVNDKLELQECLEHGRIAKFSKVRTITTRSNSIKQGKDQHFPVFMNEKEDILWCTEMERVFG
FPVHYTDVSNMSRLARQRLLGRSWSVPVIRHLFAPLKEYFACVSSGNSNANSRGPSFSSGLVPLS
LRGSHMGPMEIYKTVSAWKRQPVRVLSLFRNIDKVLKSLGFLESGSGSGGGTLKYVEDVTNVVR
RDVEKWGPFDLVYGSTQPLGSSCDRCPGWYMFQFHRILQYALPRQESQRPFFWIFMDNLLLTED
DQETTTRFLQTEAVTLQDVRGRDYQNAMRVWSNIPGLKSKHAPLTPKEEEYLQAQVRSRSKLD
APKVDLLVKNCLLPLREYFKYFSQNSLPL

In some embodiments, DNMT3a/3L is encoded by a nucleotide sequence of SEQ ID NO: 49 or SEQ ID NO: 50. In some embodiments, a nucleic acid described herein comprises a sequence of SEQ ID NO: 49 or 50 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 49)
AACCACGACCAGGAGTTCGACCCCCCCAAGGTGTACCCCCCCGTGCCCGCCGAGAAGCGGA
AGCCCATCCGGGTGCTGAGCCTGTTCGACGGCATCGCCACCGGCCTGCTGGTGCTGAAGGAC
CTGGGCATCCAGGTGGACCGGTACATCGCCAGCGAGGTGTGCGAGGACAGCATCACCGTGG
GCATGGTGCGGCACCAGGGCAAGATCATGTACGTGGGCGACGTGCGGAGCGTGACCCAGAA
GCACATCCAGGAGTGGGGCCCCTTCGACCTGGTGATCGGCGGCAGCCCCTGCAACGACCTG
AGCATCGTGAACCCCGCCCGGAAGGGCCTGTACGAGGGCACCGGCCGGCTGTTCTTCGAGTT
CTACCGGCTGCTGCACGACGCCCGGCCCAAGGAGGGCGACGACCGGCCCTTCTTCTGGCTGT
TCGAGAACGTGGTGGCCATGGGCGTGAGCGACAAGCGGGACATCAGCCGGTTCCTGGAGAG
CAACCCCGTGATGATCGACGCCAAGGAGGTGAGCGCCGCCCACCGGGCCCGGTACTTCTGG
GGCAACCTGCCCGGCATGAACCGGCCCCTGGCCAGCACCGTGAACGACAAGCTGGAGCTGC
AGGAGTGCCTGGAGCACGGCCGGATCGCCAAGTTCAGCAAGGTGCGGACCATCACCACCCG
GAGCAACAGCATCAAGCAGGGCAAGGACCAGCACTTCCCCGTGTTCATGAACGAGAAGGAG
GACATCCTGTGGTGCACCGAGATGGAGCGGGTGTTCGGCTTCCCCGTGCACTACACCGACGT
GAGCAACATGAGCCGGCTGGCCCGGCAGCGGCTGCTGGGCCGGAGCTGGAGCGTGCCCGTG
ATCCGGCACCTGTTCGCCCCCCTGAAGGAGTACTTCGCCTGCGTGAGCAGCGGCAACAGCAA
CGCCAACAGCCGGGGCCCCAGCTTCAGCAGCGGCCTGGTGCCCCTGAGCCTGCGGGGCAGC
CACATGAATCCTCTGGAGATGTTCGAGACAGTGCCCGTGTGGAGAAGGCAACCCGTGAGGG
TGCTGAGCCTCTTCGAGGACATTAAGAAGGAGCTGACCTCTCTGGGCTTTCTGGAATCCGGC
AGCGACCCCGGCCAGCTGAAACACGTGGTGGACGTGACCGACACAGTGAGGAAGGACGTGG
AAGAGTGGGGCCCCTTTGACCTCGTGTATGGAGCCACACCTCCTCTCGGCCACACATGCGAT
AGGCCTCCCAGCTGGTATCTCTTCCAGTTCCACAGACTGCTCCAGTACGCCAGACCTAAGCC
CGGCAGCCCCAGACCCTTCTTCTGGATGTTCGTGGACAATCTGGTGCTGAACAAGGAGGATC
TGGATGTGGCCAGCAGATTTCTGGAGATGGAACCCGTGACAATCCCCGACGTGCATGGCGG
CTCTCTGCAGAACGCCGTGAGAGTGTGGTCCAACATCCCCGCCATTAGAAGCAGACACTGGG
CTCTGGTGAGCGAGGAGGAACTGTCTCTGCTGGCCCAGAATAAGCAGTCCTCCAAGCTGGCC
GCCAAGTGGCCCACCAAGCTGGTGAAGAACTGCTTTCTGCCTCTGAGGGAGTATTTCAAGTA
TTTCAGCACCGAACTGACCAGCAGCCTG
(SEQ ID NO: 50)
AACCACGACCAGGAGTTCGACCCCCCCAAGGTGTACCCCCCCGTGCCCGCCGAGAAGCGGA
AGCCCATCCGGGTGCTGAGCCTGTTCGACGGCATCGCCACCGGCCTGCTGGTGCTGAAGGAC
CTGGGCATCCAGGTGGACCGGTACATCGCCAGCGAGGTGTGCGAGGACAGCATCACCGTGG
GCATGGTGCGGCACCAGGGCAAGATCATGTACGTGGGCGACGTGCGGAGCGTGACCCAGAA
GCACATCCAGGAGTGGGGCCCCTTCGACCTGGTGATCGGCGGCAGCCCCTGCAACGACCTG
AGCATCGTGAACCCCGCCCGGAAGGGCCTGTACGAGGGCACCGGCCGGCTGTTCTTCGAGTT
CTACCGGCTGCTGCACGACGCCCGGCCCAAGGAGGGCGACGACCGGCCCTTCTTCTGGCTGT
TCGAGAACGTGGTGGCCATGGGCGTGAGCGACAAGCGGGACATCAGCCGGTTCCTGGAGAG
CAACCCCGTGATGATCGACGCCAAGGAGGTGAGCGCCGCCCACCGGGCCCGGTACTTCTGG
GGCAACCTGCCCGGCATGAACCGGCCCCTGGCCAGCACCGTGAACGACAAGCTGGAGCTGC
AGGAGTGCCTGGAGCACGGCCGGATCGCCAAGTTCAGCAAGGTGCGGACCATCACCACCCG
GAGCAACAGCATCAAGCAGGGCAAGGACCAGCACTTCCCCGTGTTCATGAACGAGAAGGAG
GACATCCTGTGGTGCACCGAGATGGAGCGGGTGTTCGGCTTCCCCGTGCACTACACCGACGT
GAGCAACATGAGCCGGCTGGCCCGGCAGCGGCTGCTGGGCCGGAGCTGGAGCGTGCCCGTG
ATCCGGCACCTGTTCGCCCCCCTGAAGGAGTACTTCGCCTGCGTGAGCAGCGGCAACAGCAA
CGCCAACAGCCGGGGCCCCAGCTTCAGCAGCGGCCTGGTGCCCCTGAGCCTGCGGGGCAGC
CACATGGGCCCCATGGAGATCTACAAGACCGTGAGCGCCTGGAAGCGGCAGCCCGTGCGGG
TGCTGAGCCTGTTCCGGAACATCGACAAGGTGCTGAAATCCCTGGGCTTCCTGGAGAGCGGC
AGCGGCAGCGGCGGCGGCACCCTGAAGTACGTGGAGGACGTGACCAACGTGGTGCGGCGGG
ACGTGGAGAAGTGGGGCCCCTTCGACCTGGTGTACGGCAGCACCCAGCCCCTGGGCAGCAG
CTGCGACCGGTGCCCCGGCTGGTACATGTTCCAGTTCCACCGGATCCTGCAGTACGCCCTGC
CCCGGCAGGAGAGCCAGCGGCCCTTCTTCTGGATCTTCATGGACAACCTGCTGCTGACCGAG
GACGACCAGGAGACCACCACCCGGTTCCTGCAGACCGAGGCCGTGACCCTGCAGGACGTGC
GGGGCCGGGACTACCAGAACGCCATGCGGGTGTGGAGCAACATCCCCGGCCTGAAATCCAA
GCACGCCCCCCTGACCCCCAAGGAGGAGGAGTACCTGCAGGCCCAGGTGCGGAGCCGGAGC
AAGCTGGACGCCCCCAAGGTGGACCTGCTGGTGAAGAACTGCCTGCTGCCCCTGCGGGAGT
ACTTCAAGTACTTCAGCCAGAACAGCCTGCCCCTG

In some embodiments, DNMT3a/3L for use in a polypeptide or an expression repressor described herein is a variant, e.g., comprising one or more mutations, relative to the DNMT3a/3L of SEQ ID NO: 59 or SEQ ID NO: 60. In some embodiments, an DNMT3a/3L variant comprises one or more amino acid substitutions, deletions, or insertions relative to SEQ ID NO: 59 or SEQ ID NO: 60. In some embodiments, the polypeptide or the expression repressor is a fusion protein comprising a repressor domain that is or comprises DNMT3a/3L and a DNA-targeting moiety. In some embodiments, an expression repression system comprises two or more (e.g., two, three, or four) expression repressors, wherein the first expression repressor comprises a repressor domain comprising DNMT3a/3L, or a functional variant or fragment thereof.

In another aspect, the disclosure is directed to an expression repressor or a polypeptide comprising one or more (e.g., one) DNA-targeting moiety and one or more repressor domain, wherein the repressor domain is or comprises DNMT3b, e.g., human DNMT3b, or a functional variant or fragment thereof. In some embodiments the DNMT3b is human DNMT3b e.g., as according to NP_008823.1 or AOX21819.1, or the protein encoded by NM_006892.4 or KX447429.) In some embodiments, DNMT3b comprises an amino acid sequence of SEQ ID NO: 85. In some embodiments, a repressor domain described herein comprises SEQ ID NO: 85, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

DNMT3b
(SEQ ID NO: 85)
MKGDTRHLNGEEDAGGREDSILVNGACSDQSSDSPPILEAIRTPEIRGRRSSSRLSKREVSSLLSYT
QDLTGDGDGEDGDGSDTPVMPKLFRETRTRSESPAVRTRNNNSVSSRERHRPSPRSTRGRQGRN
HVDESPVEFPATRSLRRRATASAGTPWPSPPSSYLTIDLTDDTEDTHGTPQSSSTPYARLAQDSQQ
GGMESPQVEADSGDGDSSEYQVSADKLVALGLFSQHFNLATFNKLVSYRKAMYHALEKARVR
AGKTFPSSPGDSLEDQLKPMLEWAHGGFKPTGIEGLKPNNTQPENKTRRRTADDSATSDYCPAP
KRLKTNCYNNGKDRGDEDQSREQMASDVANNKSSLEDGCLSCGRKNPVSFHPLFEGGLCQTCR
DRFLELFYMYDDDGYQSYCTVCCEGRELLLCSNTSCCRCFCVECLEVLVGTGTAAEAKLQEPWS
CYMCLPQRCHGVLRRRKDWNVRLQAFFTSDTGLEYEAPKLYPAIPAARRRPIRVLSLFDGIATGY
LVLKELGIKVGKYVASEVCEESIAVGTVKHEGNIKYVNDVRNITKKNIEEWGPFDLVIGGSPCND
LSNVNPARKGLYEGTGRLFFEFYHLLNYSRPKEGDDRPFFWMFENVVAMKVGDKRDISRFLECN
PVMIDAIKVSAAHRARYFWGNLPGMNRPVIASKNDKLELQDCLEYNRIAKLKKVQTITTKSNSIK
QGKNQLFPVVMNGKEDVLWCTELERIFGFPVHYTDVSNMGRGARQKLLGRSWSVPVIRHLFAP
LKDYFACE

In another aspect, the disclosure is directed to an expression repressor or a polypeptide comprising one or more (e.g., one) targeting moiety and one or more effector moiety, wherein the effector moiety is or comprises G9A e.g., as according to NP_001350618.1 or the protein encoded by NM_001363689.1 or a functional variant or fragment thereof. In some embodiments, G9A comprises an amino acid sequence of SEQ ID NO: 62:

(SEQ ID NO: 62)
GNRAIRTEKIICRDVARGYENVPIPCVNGVDGEPCPEDYKYISENCETSTMNIDRNITHLQHCTCV
DDCSSSNCLCGQLSIRCWYDKDGRLLQEFNKIEPPLIFECNQACSCWRNCKNRVVQSGIKVRLQL
YRTAKMGWGVRALQTIPQGTFICEYVGELISDAEADVREDDSYLFDLDNKDGEVYCIDARYYG
NISRFINHLCDPNIIPVRVFMLHQDLRFPRIAFFSSRDIRTGEELGFDYGDRFWDIKSKYFTCQCGS
EKCKHSAEAIALEQSRLARLD

In some embodiments, the G9A repressor domain is encoded by a nucleotide sequence of SEQ ID NO: 52. In some embodiments, a nucleotide sequence described herein comprises a sequence of SEQ ID NO: 52 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 52)
GGAAATAGGGCTATCAGAACCGAGAAGATCATCTGTAGGGACGTGGCTAGAGGCTACGAGA
ACGTGCCCATTCCTTGCGTGAATGGCGTGGATGGCGAACCTTGCCCCGAGGACTACAAATAC
ATCTCCGAGAACTGCGAAACCAGCACAATGAACATCGACAGAAACATCACCCACCTCCAGC
ACTGCACATGTGTGGATGACTGCTCCTCCAGCAACTGTCTGTGCGGCCAGCTCTCCATCAGA
TGCTGGTACGACAAGGACGGCAGACTGCTGCAAGAGTTCAACAAGATCGAACCCCCTCTCA
TCTTCGAGTGTAACCAAGCTTGCAGCTGCTGGAGAAACTGCAAGAATAGAGTGGTCCAGAG
CGGCATCAAGGTGAGACTGCAACTGTACAGAACCGCCAAGATGGGATGGGGAGTGAGGGCT
CTGCAAACCATTCCCCAAGGCACCTTCATCTGCGAATACGTGGGCGAACTGATCTCCGACGC
CGAAGCTGACGTGAGAGAGGACGACAGCTATCTCTTCGATCTGGACAATAAGGACGGCGAG
GTGTACTGCATCGACGCTAGATATTACGGCAACATCTCTAGATTCATCAACCACCTCTGCGA
TCCCAACATCATTCCCGTGAGGGTGTTCATGCTGCACCAAGATCTGAGGTTCCCTAGAATCG
CCTTCTTCAGCTCTAGAGACATCAGAACCGGCGAGGAGCTGGGCTTCGATTACGGCGATAGA
TTCTGGGACATCAAGTCCAAGTACTTCACATGCCAGTGCGGCAGCGAGAAGTGTAAGCACA
GCGCTGAGGCCATTGCTCTGGAGCAGTCTAGACTGGCCAGACTGGAT

In some embodiments, G9A for use in a polypeptide or an expression repressor described herein is a variant, e.g., comprising one or more mutations, relative to the G9A sequence of SEQ ID NO: 62. In some embodiments, an G9A variant comprises one or more amino acid substitutions, deletions, or insertions relative to SEQ ID NO: 62.

In some embodiments, the polypeptide or the expression repressor is a fusion protein comprising a repressor domain that is or comprises G9A and a DNA-targeting moiety. In some embodiments, the polypeptide or the expression repressor comprises an additional moiety described herein. In some embodiments, the polypeptide or the expression repressor decreases expression of a target gene. In some embodiments, the polypeptide or the expression repressor may be used in methods of modulating, e.g., decreasing, gene expression, methods of treating a condition, or methods of epigenetically modifying a target gene, e.g., a transcription control element described herein, e.g., in place of an expression repression system. In some embodiments, an expression repression system comprises two or more (e.g., two, three, or four) expression repressors, wherein the first expression repressor comprises a repressor domain comprising the G9A sequence of SEQ ID NO: 62, or a functional variant or fragment thereof. In another aspect, the disclosure is directed to an expression repressor or a polypeptide comprising one or more (e.g., one) targeting moiety and one or more effector moiety, wherein the effector moiety is or comprises HDAC8, e.g., as according to NP_001159890 or the protein encoded by NM_001166418 or a functional variant or fragment thereof. In some embodiments, HDAC8 comprises an amino acid sequence of SEQ ID NO: 63:

(SEQ ID NO: 63)
EEPEEPADSGQSLVPVYIYSPEYVSMCDSLAKIPKRASMVHSLIEAYALHKQMRIVKPKVASMEE
MATFHTDAYLQHLQKVSQEGDDDHPDSIEYGLGYDCPATEGIFDYAAAIGGATITAAQCLIDGM
CKVAINWSGGWHHAKKDEASGFCYLNDAVLGILRLRRKFERILYVDLDLHHGDGVEDAFSFTS
KVMTVSLHKFSPGFFPGTGDVSDVGLGKGRYYSVNVPIQDGIQDEKYYQICESVLKEVYQAFNP
KAVVLQLGADTIAGDPMCSFNMTPVGIGKCLKYILQWQLATLILGGGGYNLANTARCWTYLTG
VILGKTLSSEIPDHEFFTAYGPDYVLEITPSCRPDRNEPHRIQQILNYIKGNLKHVV

In some embodiments, the HDAC8 repressor domain is encoded by a nucleotide sequence of SEQ ID NO: 53. In some embodiments, a nucleotide sequence described herein comprises a sequence of SEQ ID NO: 53 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 53)
GAGGAGCCCGAGGAGCCCGCCGATAGCGGACAATCTCTGGTGCCCGTCTACATCTACAGCC
CCGAATATGTGAGCATGTGTGATTCCCTCGCCAAGATCCCTAAGAGAGCCAGCATGGTGCAT
TCTCTGATCGAGGCCTACGCTCTGCATAAGCAAATGAGGATCGTGAAGCCCAAGGTCGCCAG
CATGGAAGAGATGGCCACCTTTCACACCGATGCCTACCTCCAACATCTCCAGAAGGTGTCCC
AAGAGGGCGACGACGACCACCCCGACTCCATTGAGTACGGACTGGGCTATGATTGCCCCGC
CACCGAGGGCATCTTTGACTATGCCGCCGCTATCGGCGGAGCTACCATCACAGCCGCCCAGT
GTCTGATTGATGGCATGTGCAAGGTCGCCATCAACTGGTCCGGAGGCTGGCATCATGCCAAG
AAGGATGAGGCCTCCGGCTTCTGTTATCTGAATGACGCCGTGCTGGGCATTCTGAGACTGAG
GAGGAAATTCGAGAGGATTCTGTACGTGGATCTGGATCTGCATCACGGAGATGGAGTCGAA
GATGCCTTCAGCTTCACCAGCAAGGTGATGACAGTCTCTCTGCACAAGTTCTCCCCCGGCTTC
TTTCCCGGAACCGGCGACGTGTCCGACGTGGGACTGGGCAAGGGAAGGTACTACAGCGTGA
ACGTGCCCATTCAAGACGGCATCCAAGACGAGAAGTACTACCAGATCTGCGAGTCCGTGCTC
AAGGAGGTCTACCAAGCCTTCAATCCTAAGGCTGTCGTGCTCCAACTGGGAGCTGATACCAT
TGCTGGCGATCCCATGTGCAGCTTCAATATGACACCCGTCGGAATCGGCAAGTGCCTCAAGT
ACATCCTCCAGTGGCAGCTCGCCACCCTCATTCTCGGAGGAGGCGGATACAATCTGGCTAAT
ACCGCCAGATGCTGGACCTATCTGACCGGCGTGATTCTGGGCAAAACACTGAGCAGCGAAA
TCCCCGACCACGAGTTTTTCACCGCTTACGGCCCCGACTACGTGCTGGAGATCACCCCCAGC
TGCAGACCCGATAGAAACGAACCCCATAGAATCCAGCAAATTCTGAACTATATCAAGGGCA
ACCTCAAGCACGTCGTG

In some embodiments, HDAC8 for use in a polypeptide or an expression repressor described herein is a variant, e.g., comprising one or more mutations, relative to the HDAC8 sequence of SEQ ID NO: 63. In some embodiments, an HDAC8 variant comprises one or more amino acid substitutions, deletions, or insertions relative to SEQ ID NO: 63.

In some embodiments, the polypeptide or the expression repressor is a fusion protein comprising a repressor domain that is or comprises HDAC8 and a DNA-targeting moiety. In some embodiments, the polypeptide or the expression repressor comprises an additional moiety described herein. In some embodiments, the polypeptide or the expression repressor decreases expression of a target gene. In some embodiments, the polypeptide or the expression repressor may be used in methods of modulating, e.g., decreasing, gene expression, methods of treating a condition, or methods of epigenetically modifying a target gene, e.g., a transcription control element described herein, e.g., in place of an expression repression system. In some embodiments, an expression repression system comprises two or more (e.g., two, three, or four) expression repressors, wherein the first expression repressor comprises a repressor domain comprising the HDAC8 sequence of SEQ ID NO: 63, or a functional variant or fragment thereof.

In another aspect, the disclosure is directed to an expression repressor or a polypeptide comprising one or more (e.g., one) targeting moiety and one or more effector moiety, wherein the effector moiety is or comprises LSD1 e.g., as according to NP_055828.2 or the protein encoded by NM_015013.4 or a functional variant or fragment thereof. In some embodiments, KRAB comprises an amino acid sequence of SEQ ID NO: 64:

(SEQ ID NO: 64)
LSGKKAAAAAAAAAAAATGTEAGPGTAGGSENGSEVAAQPAGLSGPAEVGPGAVGERTPRKK
EPPRASPPGGLAEPPGSAGPQAGPTVVPGSATPMETGIAETPEGRRTSRRKRAKVEYREMDESLA
NLSEDEYYSEEERNAKAEKEKKLPPPPPQAPPEEENESEPEEPSGVEGAAFQSRLPHDRMTSQEA
ACFPDIISGPQQTQKVFLFIRNRTLQLWLDNPKIQLTFEATLQQLEAPYNSDTVLVHRVHSYLERH
GLINFGIYKRIKPLPTKKTGKVIIIGSGVSGLAAARQLQSFGMDVTLLEARDRVGGRVATFRKGN
YVADLGAMVVTGLGGNPMAVVSKQVNMELAKIKQKCPLYEANGQAVPKEKDEMVEQEFNRL
LEATSYLSHQLDFNVLNNKHVSLGQALEVVIQLQEKHVKDEQIEHWKKIVKTQEELKELLNKM
VNLKEKIKELHQQYKEASEVKPPRDITAEFLVKSKHRDLTALCKEYDELAETQGKLEEKLQELEA
NPPSDVYLSSRDRQILDWHFANLEFANATPLSTLSLKHWDQDDDFEFTGSHLTVRNGYSCVPVA
LAEGLDIKLNTAVRQVRYTASGCEVIAVNTRSTSQTFIYKCDAVLCTLPLGVLKQQPPAVQFVPP
LPEWKTSAVQRMGFGNLNKVVLCFDRVFWDPSVNLFGHVGSTTASRGELFLFWNLYKAPILLA
LVAGEAAGIMENISDDVIVGRCLAILKGIFGSSAVPQPKETVVSRWRADPWARGSYSYVAAGSS
GNDYDLMAQPITPGPSIPGAPQPIPRLFFAGEHTIRNYPATVHGALLSGLREAGRIADQFLGAMYT
LPRQATPGVPAQQSPSM

In some embodiments, the LSD1 repressor domain is encoded by a nucleotide sequence of SEQ ID NO: 54. In some embodiments, a nucleotide sequence described herein comprises a sequence of SEQ ID NO: 54 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 54)
CTCTCCGGAAAGAAAGCCGCCGCTGCCGCTGCCGCCGCTGCTGCTGCCGCTACCGGCACAGA
AGCTGGCCCCGGAACAGCTGGCGGAAGCGAAAACGGAAGCGAAGTGGCTGCCCAGCCCGCC
GGACTGTCCGGACCCGCCGAGGTGGGACCCGGCGCTGTCGGCGAAAGGACCCCCAGAAAAA
AGGAGCCTCCTAGAGCCAGCCCTCCCGGCGGACTCGCCGAACCTCCCGGCAGCGCTGGACCT
CAAGCCGGACCTACAGTGGTGCCCGGCAGCGCTACACCTATGGAGACCGGCATCGCTGAGA
CCCCCGAGGGAAGGAGAACCAGCAGAAGGAAGAGAGCCAAGGTGGAGTACAGAGAGATGG
ATGAGTCTCTGGCTAACCTCAGCGAGGACGAGTACTACTCCGAGGAGGAAAGGAATGCCAA
GGCCGAGAAGGAGAAGAAGCTGCCCCCTCCTCCCCCTCAAGCCCCCCCCGAGGAGGAGAAC
GAGTCCGAGCCCGAGGAACCCAGCGGAGTGGAAGGAGCCGCCTTTCAGTCCAGACTGCCCC
ACGACAGAATGACATCCCAAGAGGCCGCTTGCTTTCCCGACATTATTTCCGGCCCTCAGCAG
ACCCAGAAGGTGTTTCTGTTCATTAGAAATAGAACACTCCAGCTGTGGCTCGACAACCCCAA
GATCCAGCTGACCTTCGAGGCTACCCTCCAACAGCTGGAGGCCCCCTACAATAGCGATACCG
TGCTGGTGCACAGAGTGCACAGCTATCTGGAGAGGCACGGCCTCATTAACTTCGGCATTTAC
AAGCGGATCAAGCCCCTGCCCACCAAGAAAACAGGCAAGGTGATCATCATCGGCAGCGGCG
TGAGCGGCCTGGCCGCCGCCCGGCAGCTGCAGAGCTTCGGCATGGACGTGACCCTGCTGGA
GGCCCGGGACCGGGTGGGCGGCCGGGTGGCCACCTTCCGGAAGGGCAACTACGTGGCCGAC
CTGGGCGCCATGGTGGTGACCGGCCTGGGCGGCAACCCCATGGCCGTGGTGAGCAAGCAGG
TGAACATGGAGCTGGCCAAGATCAAGCAGAAGTGCCCCCTGTACGAGGCCAACGGCCAGGC
CGTGCCCAAGGAGAAGGACGAGATGGTGGAGCAGGAGTTCAACCGGCTGCTGGAGGCCACC
AGCTACCTGAGCCACCAGCTGGACTTCAACGTGCTGAACAACAAGCACGTGAGCCTGGGCC
AGGCCCTGGAGGTGGTGATCCAGCTGCAGGAGAAGCACGTGAAGGACGAGCAGATCGAGC
ACTGGAAGAAGATCGTGAAAACACAGGAGGAGCTGAAGGAGCTGCTGAACAAGATGGTGA
ACCTGAAGGAGAAGATCAAGGAGCTGCACCAGCAGTACAAGGAGGCCAGCGAGGTGAAGC
CCCCCCGGGACATCACCGCCGAGTTCCTGGTCAAAAGCAAGCACCGGGACCTGACCGCCCT
GTGCAAGGAGTACGACGAGCTGGCCGAGACCCAGGGCAAGCTGGAGGAGAAGCTGCAGGA
GCTGGAGGCCAACCCCCCCAGCGACGTGTACCTGAGCAGCCGGGACCGGCAGATCCTGGAC
TGGCACTTCGCCAACCTGGAGTTCGCCAACGCCACCCCCCTGAGCACCCTGAGCCTGAAGCA
CTGGGACCAGGACGACGACTTCGAGTTCACCGGCAGCCACCTGACCGTGCGGAACGGCTAC
AGCTGCGTGCCCGTGGCCCTGGCCGAGGGCCTGGACATCAAGCTGAACACCGCCGTGCGGC
AGGTGCGGTACACCGCCAGCGGCTGCGAGGTGATCGCCGTGAACACCCGGAGCACCAGCCA
GACCTTCATCTACAAGTGCGACGCCGTGCTGTGCACCCTGCCCCTGGGCGTGCTGAAGCAGC
AGCCCCCCGCCGTGCAGTTCGTGCCCCCCCTGCCCGAGTGGAAAACAAGCGCCGTGCAGCG
GATGGGCTTCGGCAACCTGAACAAGGTGGTGCTGTGCTTCGACCGGGTGTTCTGGGACCCCA
GCGTGAACCTGTTCGGCCACGTGGGCAGCACCACCGCCAGCCGGGGCGAGCTGTTCCTGTTC
TGGAACCTGTACAAGGCCCCCATCCTGCTGGCCCTGGTGGCCGGCGAGGCCGCCGGCATCAT
GGAGAACATCAGCGACGACGTGATCGTGGGCCGGTGCCTGGCCATCCTGAAGGGCATCTTC
GGCAGCAGCGCCGTGCCCCAGCCCAAGGAGACCGTGGTGAGCCGGTGGCGGGCCGACCCCT
GGGCCCGGGGCAGCTACAGCTACGTGGCCGCCGGCAGCAGCGGCAACGACTACGACCTGAT
GGCCCAGCCCATCACCCCCGGCCCCAGCATCCCCGGCGCCCCCCAGCCCATCCCCCGGCTGT
TCTTCGCCGGCGAGCACACCATCCGGAACTACCCCGCCACCGTGCACGGCGCCCTGCTGAGC
GGCCTGCGGGAGGCAGGACGGATCGCCGACCAGTTCCTGGGCGCCATGTACACCCTGCCCC
GGCAGGCCACCCCCGGCGTGCCCGCCCAGCAGAGCCCCAGCATG

In some embodiments, LSD1 for use in a polypeptide or an expression repressor described herein is a variant, e.g., comprising one or more mutations, relative to the LSD1 sequence of SEQ ID NO: 64. In some embodiments, an LSD1 variant comprises one or more amino acid substitutions, deletions, or insertions relative to SEQ ID NO: 64.

In some embodiments, the polypeptide or the expression repressor is a fusion protein comprising a repressor domain that is or comprises LSD1 and a DNA-targeting moiety. In some embodiments, the polypeptide or the expression repressor comprises an additional moiety described herein. In some embodiments, the polypeptide or the expression repressor decreases expression of a target gene. In some embodiments, the polypeptide or the expression repressor may be used in methods of modulating, e.g., decreasing, gene expression, methods of treating a condition, or methods of epigenetically modifying a target gene, e.g., a transcription control element described herein, e.g., in place of an expression repression system. In some embodiments, an expression repression system comprises two or more (e.g., two, three, or four) expression repressors, wherein the first expression repressor comprises a repressor domain comprising the LSD1 sequence of SEQ ID NO: 64, or a functional variant or fragment thereof.

In another aspect, the disclosure is directed to an expression repressor or a polypeptide comprising one or more (e.g., one) targeting moiety and one or more effector moiety, wherein the effector moiety is or comprises EZH2, e.g., as according to NP-004447.2 or the protein encoded by NM_004456.5 or a functional variant or fragment thereof. In some embodiments, EZH2 comprises an amino acid sequence of SEQ ID NO: 65:

(SEQ ID NO: 65)
GQTGKKSEKGPVCWRKRVKSEYMRLRQLKRFRRADEVKSMFSSNRQKILERTEILNQEWKQRRI
QPVHILTSVSSLRGTRECSVTSDLDFPTQVIPLKTLNAVASVPIMYSWSPLQQNFMVEDETVLHNI
PYMGDEVLDQDGTFIEELIKNYDGKVHGDRECGFINDEIFVELVNALGQYNDDDDDDDGDDPEE
REEKQKDLEDHRDDKESRPPRKFPSDKIFEAISSMFPDKGTAEELKEKYKELTEQQLPGALPPECT
PNIDGPNAKSVQREQSLHSFHTLFCRRCFKYDCFLHPFHATPNTYKRKNTETALDNKPCGPQCYQ
HLEGAKEFAAALTAERIKTPPKRPGGRRRGRLPNNSSRPSTPTINVLESKDTDSDREAGTETGGEN
NDKEEEEKKDETSSSSEANSRCQTPIKMKPNIEPPENVEWSGAEASMFRVLIGTYYDNFCAIARLI
GTKTCRQVYEFRVKESSIIAPAPAEDVDTPPRKKKRKHRLWAAHCRKIQLKKDGSSNHVYNYQP
CDHPRQPCDSSCPCVIAQNFCEKFCQCSSECQNRFPGCRCKAQCNTKQCPCYLAVRECDPDLCLT
CGAADHWDSKNVSCKNCSIQRGSKKHLLLAPSDVAGWGIFIKDPVQKNEFISEYCGEIISQDEAD
RRGKVYDKYMCSFLFNLNNDFVVDATRKGNKIRFANHSVNPNCYAKVMMVNGDHRIGIFAKR
AIQTGEELFFDYRYSQADALKYVGIEREMEIP

In some embodiments, the EZH2 repressor domain is encoded by a nucleotide sequence of SEQ ID NO: 55. In some embodiments, a nucleotide sequence described herein comprises a sequence of SEQ ID NO: 55 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 55)
GGCCAGACCGGCAAGAAGAGCGAGAAGGGCCCCGTGTGCTGGCGGAAGCGGGTGAAGAGC
GAGTACATGCGGCTGCGGCAGCTGAAGCGGTTCCGGCGGGCCGACGAGGTGAAGAGCATGT
TCAGCAGCAACCGGCAGAAGATCCTGGAGCGGACCGAGATCCTGAACCAGGAGTGGAAGCA
GCGGCGAATCCAGCCCGTGCACATCCTGACCAGCGTGAGCAGCCTGCGGGGCACCCGGGAG
TGCAGCGTGACCAGCGACCTGGACTTCCCCACCCAGGTGATCCCCCTAAAGACCCTGAACGC
CGTGGCCAGCGTGCCCATCATGTACAGCTGGAGCCCCCTGCAGCAGAACTTCATGGTGGAGG
ACGAGACCGTGCTGCACAACATCCCCTACATGGGCGACGAGGTGCTGGACCAGGACGGCAC
CTTCATCGAGGAGCTGATCAAGAACTACGACGGCAAGGTGCACGGCGACCGGGAGTGCGGC
TTCATCAACGACGAGATCTTCGTGGAGCTGGTGAACGCCCTGGGCCAGTACAACGACGACG
ACGACGACGACGACGGCGACGACCCCGAGGAGCGGGAGGAGAAGCAGAAGGACCTGGAGG
ACCACCGGGACGACAAGGAGAGCCGGCCCCCCCGGAAGTTCCCCAGCGACAAGATCTTCGA
GGCCATCAGCAGCATGTTCCCCGACAAGGGCACCGCCGAGGAGCTGAAGGAGAAGTACAAG
GAGCTGACCGAGCAGCAGCTGCCCGGCGCCCTGCCCCCCGAGTGCACCCCCAACATCGACG
GCCCCAACGCCAAGAGCGTGCAGCGGGAGCAGAGCCTGCACAGCTTCCACACCCTGTTCTG
CCGGCGGTGCTTCAAGTACGACTGCTTCCTGCACCCCTTCCACGCCACCCCCAACACCTACA
AGCGGAAGAACACCGAGACCGCCCTGGACAACAAGCCCTGCGGCCCCCAGTGCTACCAGCA
CCTGGAGGGCGCCAAGGAGTTCGCCGCCGCCCTGACCGCCGAGCGGATCAAGACCCCCCCC
AAGCGGCCCGGCGGCCGGCGGCGGGGCCGGCTGCCCAACAACAGCAGCCGGCCCAGCACCC
CCACCATCAACGTGCTGGAGAGCAAGGACACCGACAGCGACCGGGAGGCCGGCACCGAGA
CCGGCGGCGAGAACAACGACAAGGAGGAGGAGGAGAAGAAGGACGAGACCAGCAGCAGC
AGCGAGGCCAACAGCCGGTGCCAGACCCCCATCAAGATGAAGCCCAACATCGAGCCCCCCG
AGAACGTGGAGTGGAGCGGCGCCGAGGCCAGCATGTTCCGGGTGCTGATCGGCACCTACTA
CGACAACTTCTGCGCCATCGCCCGGCTGATCGGCACCAAGACCTGCCGGCAGGTGTACGAGT
TCCGGGTGAAGGAGAGCAGCATCATCGCCCCCGCCCCCGCCGAGGACGTGGACACCCCCCC
CCGGAAGAAGAAGCGGAAGCACCGGCTGTGGGCCGCCCACTGCCGGAAGATCCAGCTGAAG
AAGGACGGCAGCAGCAACCACGTGTACAACTACCAGCCCTGCGACCACCCCCGGCAGCCCT
GCGACAGCAGCTGCCCCTGCGTGATCGCCCAGAACTTCTGCGAGAAGTTCTGCCAGTGCAGC
AGCGAGTGCCAGAACCGGTTCCCCGGCTGCCGGTGCAAGGCCCAGTGCAACACCAAGCAGT
GCCCCTGCTACCTGGCCGTGCGGGAGTGCGACCCCGACCTGTGCCTGACCTGCGGCGCCGCC
GACCACTGGGACAGCAAGAACGTGAGCTGCAAGAACTGCAGCATCCAGCGGGGCAGCAAG
AAGCACCTGCTGCTGGCCCCCAGCGACGTGGCCGGCTGGGGCATCTTCATCAAGGACCCCGT
GCAGAAGAACGAGTTCATCAGCGAGTACTGCGGCGAGATCATCAGCCAGGACGAGGCCGAC
CGGCGGGGCAAGGTGTACGACAAGTACATGTGCAGCTTCCTGTTCAACCTGAACAACGACTT
CGTGGTGGACGCCACCCGGAAGGGCAACAAGATCCGGTTCGCCAACCACAGCGTGAACCCC
AACTGCTACGCCAAGGTGATGATGGTGAACGGCGACCACCGGATCGGCATCTTCGCCAAGC
GGGCCATCCAGACCGGCGAGGAGCTGTTCTTCGACTACCGGTACAGCCAGGCCGACGCCCT
GAAGTACGTGGGCATCGAGCGGGAGATGGAGATCCCC

In some embodiments, EZH2 for use in a polypeptide or an expression repressor described herein is a variant, e.g., comprising one or more mutations, relative to the EZH2 sequence of SEQ ID NO: 65. In some embodiments, an EZH2 variant comprises one or more amino acid substitutions, deletions, or insertions relative to SEQ ID NO: 65.

In some embodiments, the polypeptide or the expression repressor is a fusion protein comprising a repressor domain that is or comprises EZH2 and a DNA-targeting moiety. In some embodiments, the polypeptide or the expression repressor comprises an additional moiety described herein. In some embodiments, the polypeptide or the expression repressor decreases expression of a target gene. In some embodiments, the polypeptide or the expression repressor may be used in methods of modulating, e.g., decreasing, gene expression, methods of treating a condition, or methods of epigenetically modifying a target gene, e.g., a transcription control element described herein, e.g., in place of an expression repression system. In some embodiments, an expression repression system comprises two or more (e.g., two, three, or four) expression repressors, wherein the first expression repressor comprises a repressor domain comprising the EZH2 sequence of SEQ ID NO: 65, or a functional variant or fragment thereof.

In another aspect, the disclosure is directed to an expression repressor or a polypeptide comprising one or more (e.g., one) targeting moiety and one or more effector moiety, wherein the effector moiety is or comprises FOG1 e.g., as according to NP_722520.2 or the protein encoded by NM_153813.3 or a functional variant or fragment thereof. In some embodiments, FOG1 comprises an amino acid sequence of SEQ ID NO: 66:

(SEQ ID NO: 66)
SRRKQSNPRQIKRSLGDMEAREEVQLVGASHMEQKATAPEAPSP

In some embodiments, the FOG1 repressor domain is encoded by a nucleotide sequence of SEQ ID NO: 56. In some embodiments, a nucleotide sequence described herein comprises a sequence of SEQ ID NO: 56 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 56)
AGCAGAAGGAAGCAGAGCAACCCCAGACAAATCAAGAGATCTCTGGGCG
ACATGGAGGCCAGAGAGGAAGTGCAGCTGGTGGGCGCCAGCCACATGGA
GCAGAAGGCTACAGCCCCCGAGGCCCCCAGCCCC

In some embodiments, FOG1 for use in a polypeptide or an expression repressor described herein is a variant, e.g., comprising one or more mutations, relative to the FOG 1 sequence of SEQ ID NO: 66. In some embodiments, an FOG1 variant comprises one or more amino acid substitutions, deletions, or insertions relative to SEQ ID NO: 66.

In some embodiments, the polypeptide or the expression repressor is a fusion protein comprising a repressor domain that is or comprises FOG1 and a DNA-targeting moiety. In some embodiments, the polypeptide or the expression repressor comprises an additional moiety described herein. In some embodiments, the polypeptide or the expression repressor decreases expression of a target gene. In some embodiments, the polypeptide or the expression repressor may be used in methods of modulating, e.g., decreasing, gene expression, methods of treating a condition, or methods of epigenetically modifying a target gene, e.g., a transcription control element described herein, e.g., in place of an expression repression system. In some embodiments, an expression repression system comprises two or more (e.g., two, three, or four) expression repressors, wherein the first expression repressor comprises a repressor domain comprising the FOG1 sequence of SEQ ID NO: 66, or a functional variant or fragment thereof.

In some embodiments, provided technologies are described as comprising a gRNA that specifically targets a target gene. In some embodiments, the target gene is an oncogene, a tumor suppressor, or a MYC mis-regulation disorder related gene. In some embodiments, the target gene is MYC. In some embodiments, the target gene is an MHC class I molecule, e.g., β2M. In some embodiments, the target gene encodes a heat shock protein, e.g., HSPA1B. In some embodiments, the target gene is a transcription factor, e.g., GATA1.

In some embodiments, technologies provided herein include methods of delivering one or more expression repressors or expression repression systems described herein to a subject, e.g., to a nucleus of a cell or tissue of a subject, by linking such a moiety to a DNA-targeting moiety as part of a fusion molecule.

In some embodiments, an expression repressor comprises a nuclear localization sequence (NLS). In some embodiments, the expression repressor comprises an NLS, e.g., an SV40 NLS at the N-terminus. In some embodiments, the expression repressor comprises an NLS, e.g., a nucleoplasmin NLS at the C-terminus. In some embodiments, the expression repressor comprises a first NLS at the N-terminus and a second NLS at the C-terminus. In some embodiments the first and the second NLS have the same sequence. In some embodiments, the first and the second NLS have different sequences. In some embodiments, the expression repression repressor comprises an SV40 NLS, e.g., the expression repressor comprises a sequence according to PKKKRK (SEQ ID NO: 86). In some embodiments, the expression repressor comprises an epitope tag, e.g., an HA tag: YPYDVPDYA (SEQ ID NO: 80). For example, the expression repressor may comprise two copies of the epitope tag.

While an epitope tag is useful in many research contexts, it is sometimes desirable to omit an epitope tag in a therapeutic context. Accordingly, in some embodiments, the expression repressor lacks an epitope tag. In some embodiments, an expression repressor described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the HA tag of SEQ ID NO: 80. In some embodiments, a nucleic acid described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking a region encoding the HA tag of SEQ ID NO: 80. In some embodiments, the expression repressor comprises a nucleoplasmin NLS, e.g., the expression repressor comprises a sequence of KRPAATKKAGQAKKK (SEQ ID NO: 87). In some embodiments, the expression repressor does not comprise an NLS. In some embodiments, the expression repressor does not comprise an epitope tag. In some embodiments the expression repressor does not comprise an HA tag. In some embodiments, the expression repressor does not comprise an HA tag sequence according to SEQ ID NO: 80.

### DNA-Targeting Moieties

DNA-targeting moieties may specifically bind a DNA sequence, e.g., a DNA sequence associated with a target gene, e.g., binds, a genomic sequence element (e.g., a promoter, a TSS, or an anchor sequence) in, proximal to, and/or operably linked to a target gene. Any molecule or compound that specifically binds a DNA sequence may be used as a DNA-targeting moiety.

In some embodiments, a DNA-targeting moiety targets, e.g., binds, a component of a genomic complex (e.g., ASMC). In some embodiments, a DNA-targeting moiety targets, e.g., binds, an expression control sequence (e.g., a promoter or enhancer) operably linked to a target gene. In some embodiments, a DNA-targeting moiety targets, e.g., binds, a target gene, or a part of a target gene. The target of a DNA-targeting moiety may be referred to as its targeted component. A targeted component may be any genomic sequence element operably linked to a target gene, or the target gene itself, including but not limited to a promoter, enhancer, anchor sequence, exon, intron, UTR encoding sequence, a splice site, or a transcription start site. In some embodiments, a DNA-targeting moiety binds specifically to one or more target anchor sequences (e.g., within a cell) and not to non-targeted anchor sequences (e.g., within the same cell).

In some embodiments, a DNA-targeting moiety may be or comprise a CRISPR/Cas molecule, a TAL effector molecule, a Zn finger domain, peptide nucleic acid (PNA) or a nucleic acid molecule. In some embodiments, an expression repressor comprises one DNA-targeting moiety. In some embodiments, an expression repression system comprises a plurality of expression repressors, wherein each member of the plurality of expression repressors comprises a DNA-targeting moiety, wherein each DNA-targeting moiety does not detectably bind, e.g., does not bind, to another DNA-targeting moiety. In some embodiments, an expression repression system comprises a first expression repressor comprising a first DNA-targeting moiety and a second expression repressor comprising a second DNA-targeting moiety, wherein the first DNA-targeting moiety does not detectably bind, e.g., does not bind, to the second DNA-targeting moiety. In some embodiments, an expression repression system comprises a first expression repressor comprising a first DNA-targeting moiety and a second expression repressor comprising a second DNA-targeting moiety, wherein the first DNA-targeting moiety does not detectably bind, e.g., does not bind, to another first DNA-targeting moiety, and the second DNA-targeting moiety does not detectably bind, e.g., does not bind, to another second DNA-targeting moiety. In some embodiments, a DNA-targeting moiety for use in the compositions and methods described herein is functional (e.g., binds to a DNA sequence) in a monomeric, e.g., non-dimeric, state.

In some embodiments, binding of a targeting moiety to a targeted component decreases binding affinity of the targeted component for another transcription factor, genomic complex component, or genomic sequence element. In some embodiments, a DNA-targeting moiety binds to its target sequence with a K_{D} of less than or equal to 500, 450, 400, 350, 300, 250, 200, 150, 100, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM (and optionally, a K_{D} of at least 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM). In some embodiments, a DNA-targeting moiety binds to its target sequence with a K_{D} of 0.001 nM to 500 nM, e.g., 0.1 nM to 5 nM, e.g., about 0.5 nM. In some embodiments, a DNA-targeting moiety binds to a non-target sequence with a K_{D} of at least 500, 600, 700, 800, 900, 1000, 2000, 5000, 10,000, or 100,000 nM (and optionally, does not appreciably bind to a non-target sequence). In some embodiments, a DNA-targeting moiety does not bind to a non-target sequence.

In some embodiments, a DNA-targeting moiety comprises a nucleic acid sequence complementary to a targeted component, e.g., a promoter of a target gene. In some embodiments, a targeting moiety comprises a nucleic acid sequence that is at least 10%, 15%, 20%, 25%, 30%, 35%, 40%, 45%, 50%, 55%, 60%, 65%, 70%, 75%, 80%, 85%, 90%, 95%, 99%, or 100% complementary to a targeted component.

In some embodiments, the DNA-targeting moiety of an expression repressor comprises no more than 100, 90, 80, 70, 60, 50, 40, 30, or 20 nucleotides (and optionally at least 10, 20, 30, 40, 50, 60, 70, 80, or 90 nucleotides). In some embodiments, an expression repressor or a repressor domain of a fusion molecule, comprises no more than 2000, 1900, 1800, 1700, 1600, 1500, 1400, 1300, 1200, 1100, 1000, 900, 800, 700, 600, 500, 400, 300, 200, or 100 amino acids (and optionally at least 50, 100, 200, 300, 400, 500, 600, 700, 800, 900, 1000, 1100, 1200, 1300, 1400, 1500, 1600, 1700, 1800, or 1900 amino acids). In some embodiments, an expression repressor or the effector moiety of a fusion molecule, comprises 100-2000, 100-1900, 100-1800, 100-1700, 100-1600, 100-1500, 100-1400, 100-1300, 100-1200, 100-1100, 100-1000, 100-900, 100-800, 100-700, 100-600, 100-500, 100-400, 100-300, 100-200, 200-2000, 200-1900, 200-1800, 200-1700, 200-1600, 200-1500, 200-1400, 200-1300, 200-1200, 200-1100, 200-1000, 200-900, 200-800, 200-700, 200-600, 200-500, 200-400, 200-300, 300-2000, 300-1900, 300-1800, 300-1700, 300-1600, 300-1500, 300-1400, 300-1300, 300-1200, 300-1100, 300-1000, 300-900, 300-800, 300-700, 300-600, 300-500, 300-400, 400-2000, 400-1900, 400-1800, 400-1700, 400-1600, 400-1500, 400-1400, 400-1300, 400-1200, 400-1100, 400-1000, 400-900, 400-800, 400-700, 400-600, 400-500, 500-2000, 500-1900, 500-1800, 500-1700, 500-1600, 500-1500, 500-1400, 500-1300, 500-1200, 500-1100, 500-1000, 500-900, 500-800, 500-700, 500-600, 600-2000, 600-1900, 600-1800, 600-1700, 600-1600, 600-1500, 600-1400, 600-1300, 600-1200, 600-1100, 600-1000, 600-900, 600-800, 600-700, 700-2000, 700-1900, 700-1800, 700-1700, 700-1600, 700-1500, 700-1400, 700-1300, 700-1200, 700-1100, 700-1000, 700-900, 700-800, 800-2000, 800-1900, 800-1800, 800-1700, 800-1600, 800-1500, 800-1400, 800-1300, 800-1200, 800-1100, 800-1000, 800-900, 900-2000, 900-1900, 900-1800, 900-1700, 900-1600, 900-1500, 900-1400, 900-1300, 900-1200, 900-1100, 900-1000, 1000-2000, 1000-1900, 1000-1800, 1000-1700, 1000-1600, 1000-1500, 1000-1400, 1000-1300, 1000-1200, 1000-1100, 1100-2000, 1100-1900, 1100-1800, 1100-1700, 1100-1600, 1100-1500, 1100-1400, 1100-1300, 1100-1200, 1200-2000, 1200-1900, 1200-1800, 1200-1700, 1200-1600, 1200-1500, 1200-1400, 1200-1300, 1300-2000, 1300-1900, 1300-1800, 1300-1700, 1300-1600, 1300-1500, 1300-1400, 1400-2000, 1400-1900, 1400-1800, 1400-1700, 1400-1600, 1400-1500, 1500-2000, 1500-1900, 1500-1800, 1500-1700, 1500-1600, 1600-2000, 1600-1900, 1600-1800, 1600-1700, 1700-2000, 1700-1900, 1700-1800, 1800-2000, 1800-1900, or 1900-2000 amino acids.

An expression repressor or an expression repression system as disclosed herein, may comprise nucleic acid, e.g., one or more nucleic acids. In some embodiments, a nucleic acid is or comprises RNA; in some embodiments, a nucleic acid is or comprises DNA. In some embodiments, a nucleic acid is or comprises more than 50% ribonucleotides. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more peptide nucleic acids. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, nucleic acids may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

In some embodiments, a DNA-targeting moiety comprises or is nucleic acid.

In some embodiments, a nucleic acid that may be included in a moiety may be or comprise DNA, RNA, and/or an artificial or synthetic nucleic acid or nucleic acid analog or mimic. For example, in some embodiments, a nucleic acid may be or include one or more of genomic DNA (gDNA), complementary DNA (cDNA), a peptide nucleic acid (PNA), a peptide-nucleic acid mixmer, a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. A nucleic acid sequence may include modified oligonucleotides (e.g., chemical modifications, such as modifications that alter backbone linkages, sugar molecules, and/or nucleic acid bases) and/or artificial nucleic acids. In some embodiments, a nucleic acid sequence includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules. In some embodiments, a nucleic acid may include one or more residues that is not a naturally occurring DNA or RNA residue, may include one or more linkages that is/are not phosphodiester bonds (e.g., that may be, for example, phosphorothioate bonds, etc.), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

In some embodiments, a nucleic acid described herein comprises one or more nucleoside analogs. In some embodiments, a nucleic acid sequence may include in addition or as an alternative to one or more natural nucleosides, e.g., purines or pyrimidines, e.g., adenine, cytosine, guanine, thymine, and uracil, one or more nucleoside analogs. In some embodiments, a nucleic acid sequence includes one or more nucleoside analogs. A nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

### CRISPR/Cas Domains

In some embodiments, a DNA-targeting moiety is or comprises a CRISPR/Cas molecule. A CRISPR/Cas molecule comprises a protein involved in the clustered regulatory interspaced short palindromic repeat (CRISPR) system, e.g., a Cas protein, and optionally a guide RNA, e.g., single guide RNA (sgRNA). In some embodiments, the gRNA comprised by the CRISPR/Cas molecule is noncovalently bound by the CRISPR/Cas protein.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. For example, in a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (1-111) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by RNase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, CasIO, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-targeting moiety includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram-positive bacterium or a gram-negative bacterium. In certain embodiments, a Cas protein may be from a Streptococcus (e.g., S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 1. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions.

TABLE 1

| | | | | | Mutations to | |
|---|---|---|---|---|---|---|
| | | | # of | | alter PAM | Mutations to make |
| Name | Enzyme | Species | AAs | PAM | recognition | catalytically dead |
| FnCas9 | Cas9 | Francisella | 1629 | 5′-NGG-3′ | Wt | D11A/H969A/N995A |
| | | novicida | | | | |
| FnCas9 | Cas9 | Francisella | 1629 | 5′-YG-3′ | E1369R/E1449H/ | D11A/H969A/N995A |
| RHA | | novicida | | | R1556A | |
| SaCas9 | Cas9 | Staphylococcus | 1053 | 5′-NNGRRT-3′ | Wt | D10A/H557A |
| | | aureus | | | | |
| SaCas9 | Cas9 | Staphylococcus | 1053 | 5′-NNNRRT-3′ | E782K/N968K/ | D10A/H557A |
| KKH | | aureus | | | R1015H | |
| SpCas9 | Cas9 | Streptococcus | 1368 | 5′-NGG-3′ | Wt | D10A/D839A/H840A/ |
| | | pyogenes | | | | N863A |
| SpCas9 | Cas9 | Streptococcus | 1368 | 5′-NGA-3′ | D1135V/R1335Q/ | D10A/D839A/H840A/ |
| VQR | | pyogenes | | | T1337R | N863A |
| AsCpf1 | Cpf1 | Acidaminococcus sp. | 1307 | 5′-TYCV-3′ | S542R/K607R | E993A |
| RR | | BV3L6 | | | | |
| AsCpf1 | Cpf1 | Acidaminococcus sp. | 1307 | 5′-TATV-3′ | S542R/K548V/ | E993A |
| RVR | | BV3L6 | | | N552R | |
| FnCpf1 | Cpf1 | Francisella | 1300 | 5′-NTTN-3′ | Wt | D917A/E1006A/D1255A |
| | | novicida | | | | |
| NmCas9 | Cas9 | Neisseria | 1082 | 5′-NNNGATT-3′ | Wt | D16A/D587A/H588A/ |
| | | meningitidis | | | | N611A |

In some embodiments, the Cas protein is modified to deactivate the nuclease, e.g., nuclease deficient Cas9. Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas9 binding to a DNA sequence may interfere with transcription at that site by steric hindrance. In some embodiments, a DNA-targeting moiety is or comprises a catalytically inactive Cas9, e.g., dCas9. In some embodiments, a DNA-targeting moiety is or comprises a catalytically inactive mutant Cas9, e.g., Cas9m4. Many catalytically inactive Cas9 proteins are known in the art. In some embodiments, dCas9 comprises mutations in each endonuclease domain of the Cas protein, e.g., D10A and H840A mutations.

In some embodiments, a catalytically inactive Cas9 protein, e.g., dCas9, comprises a D11A mutation or an analogous substitution to the amino acid corresponding to said position. In some embodiments, a catalytically inactive Cas9 protein, e.g., dCas9, comprises a H969A mutation or an analogous substitution to the amino acid corresponding to said position. In some embodiments, a catalytically inactive Cas9 protein, e.g., dCas9, comprises a N995A mutation or an analogous substitution to the amino acid corresponding to said position. In some embodiments, a catalytically inactive Cas9 protein, e.g., dCas9, comprises D11A, H969A, and N995A mutations or analogous substitutions to the amino acids corresponding to said positions.

In some embodiments, a catalytically inactive Cas9 protein, e.g., dCas9, comprises a D10A mutation or an analogous substitution to the amino acid corresponding to said position. In some embodiments, a catalytically inactive Cas9 protein, e.g., dCas9, comprises a H557A mutation or an analogous substitution to the amino acid corresponding to said position. In some embodiments, a catalytically inactive Cas9 protein, e.g., dCas9, comprises D10A and H557A mutations or analogous substitutions to the amino acids corresponding to said positions.

In some embodiments, a catalytically inactive Cas9 protein, e.g., dCas9, comprises a D839A mutation or an analogous substitution to the amino acid corresponding to said position. In some embodiments, a catalytically inactive Cas9 protein, e.g., dCas9, comprises a H840A mutation or an analogous substitution to the amino acid corresponding to said position. In some embodiments, a catalytically inactive Cas9 protein, e.g., dCas9, comprises a N863A mutation or an analogous substitution to the amino acid corresponding to said position. In some embodiments, a catalytically inactive Cas9 protein, e.g., dCas9, comprises D10A and D839A mutations or analogous substitutions to the amino acids corresponding to said positions. In some embodiments, a catalytically inactive Cas9 protein, e.g., dCas9, comprises D10A, D839A, and H840A mutations or analogous substitutions to the amino acids corresponding to said positions. In some embodiments, a catalytically inactive Cas9 protein, e.g., dCas9, comprises D10A, D839A, H840A, and N863A mutations or analogous substitutions to the amino acids corresponding to said positions.

In some embodiments, a catalytically inactive Cas9 protein, e.g., dCas9, comprises a E993A mutation or an analogous substitution to the amino acid corresponding to said position.

In some embodiments, a catalytically inactive Cas9 protein, e.g., dCas9, comprises a D917A mutation or an analogous substitution to the amino acid corresponding to said position. In some embodiments, a catalytically inactive Cas9 protein, e.g., dCas9, comprises a E1006A mutation or an analogous substitution to the amino acid corresponding to said position. In some embodiments, a catalytically inactive Cas9 protein, e.g., dCas9, comprises a D1255A mutation or an analogous substitution to the amino acid corresponding to said position. In some embodiments, a catalytically inactive Cas9 protein, e.g., dCas9, comprises D917A, E1006A, and D1255A mutations or analogous substitutions to the amino acids corresponding to said positions.

In some embodiments, a catalytically inactive Cas9 protein, e.g., dCas9, comprises a D16A mutation or an analogous substitution to the amino acid corresponding to said position. In some embodiments, a catalytically inactive Cas9 protein, e.g., dCas9, comprises a D587A mutation or an analogous substitution to the amino acid corresponding to said position. In some embodiments, a catalytically inactive Cas9 protein, e.g., dCas9, comprises a H588A mutation or an analogous substitution to the amino acid corresponding to said position. In some embodiments, a catalytically inactive Cas9 protein, e.g., dCas9, comprises a N611A mutation or an analogous substitution to the amino acid corresponding to said position. In some embodiments, a catalytically inactive Cas9 protein, e.g., dCas9, comprises D16A, D587A, H588A, and N611A mutations or analogous substitutions to the amino acids corresponding to said positions.

In another aspect, the disclosure is directed to an expression repressor or a polypeptide comprising one or more (e.g., one) DNA-targeting moiety and one or more repressor domain, wherein the one or more DNA-targeting moiety is or comprises a CRISPR/Cas molecule comprising a Cas protein, e.g., catalytically inactive Cas9 protein, e.g., dCas9, e.g., dCas9m4, or a functional variant or fragment thereof. In some embodiments, dCas9 comprises an amino acid sequence of SEQ ID NO: 46 or 88:

(SEQ ID NO: 46)
DKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRLSDYDVAAIV
PQSFLKDDSIDNKVLTRSDKARGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQEIGKAT
AKYFFYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAY
NKHRDKPIREQAENIIHLFTLTNLGAPAAFKYFDTTIDRKRYTSTKEVLDATLIHQSITGLYETRID
LSQLGGD
(SEQ ID NO: 46)
AKRNYILGLAIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVHNVNEVE
EDTGNELSTKEQISRNSKALEEKYVAELQLERLKKDGEVRGSINRFKTSDYVKEAKQLLKVQKA
YHQLDQSFIDTYIDLLETRRTYYEGPGEGSPFGWKDIKEWYEMLMGHCTYFPEELRSVKYAYNA
DLYNALNDLNNLVITRDENEKLEYYEKFQIIENVFKQKKKPTLKQIAKEILVNEEDIKGYRVTSTG
KPEFTNLKVYHDIKDITARKEIIENAELLDQIAKILTIYQSSEDIQEELTNLNSELTQEEIEQISNLKG
YTGTHNLSLKAINLILDELWHTNDNQIAIFNRLKLVPKKVDLSQQKEIPTTLVDDFILSPVVKRSFI
QSIKVINAIIKKYGLPNDIIIELAREKNSKDAQKMINEMQKRNRQTNERIEEIIRTTGKENAKYLIEK
IKLHDMQEGKCLYSLEAIPLEDLLNNPFNYEVDAIIPRSVSFDNSFNNKVLVKQEENSKKGNRTPF
QYLSSSDSKISYETFKKHILNLAKGKGRISKTKKEYLLEERDINRFSVQKDFINRNLVDTRYATRG
LMNLLRSYFRVNNLDVKVKSINGGFTSFLRRKWKFKKERNKGYKHHAEDALIIANADFIFKEWK
KLDKAKKVMENQMFEEKQAESMPEIETEQEYKEIFITPHQIKHIKDFKDYKYSHRVDKKPNRKLI
NDTLYSTRKDDKGNTLIVNNLNGLYDKDNDKLKKLINKSPEKLLMYHHDPQTYQKLKLIMEQY
GDEKNPLYKYYEETGNYLTKYSKKDNGPVIKKIKYYGNKLNAHLDITDDYPNSRNKVVKLSLKP
YRFDVYLDNGVYKFVTVKNLDVIKKENYYEVNSKCYEEAKKLKKISNQAEFIASFYKNDLIKIN
GELYRVIGVNNDLLNRIEVNMIDITYREYLENMNDKRPPHIIKTIASKTQSIKKYSTDILGNLYEVK
SKKHPQIIKKG

In some embodiments, the dCas9 is encoded by a nucleic acid sequence of SEQ ID NO: 45 or 89:

(SEQ ID NO: 45)
GACAAGAAGTACAGCATCGGCCTGGCCATCGGCACCAACAGCGTGGGCTGGGCCGTGATCA
CCGACGAGTACAAGGTGCCCAGCAAGAAGTTCAAGGTGCTGGGCAACACCGACCGGCACAG
CATCAAGAAGAACCTGATCGGCGCCCTGCTGTTCGACAGCGGCGAGACCGCCGAGGCCACC
CGGCTGAAGCGGACCGCCCGGCGGCGGTACACCCGGCGGAAGAACCGGATCTGCTACCTGC
AGGAGATCTTCAGCAACGAGATGGCCAAGGTGGACGACAGCTTCTTCCACCGGCTGGAGGA
GAGCTTCCTGGTGGAGGAGGACAAGAAGCACGAGCGGCACCCCATCTTCGGCAACATCGTG
GACGAGGTGGCCTACCACGAGAAGTACCCCACCATCTACCACCTGCGGAAGAAGCTGGTGG
ACAGCACCGACAAGGCCGACCTGCGGCTGATCTACCTGGCCCTGGCCCACATGATCAAGTTC
CGGGGCCACTTCCTGATCGAGGGCGACCTGAACCCCGACAACAGCGACGTGGACAAGCTGT
TCATCCAGCTGGTGCAGACCTACAACCAGCTGTTCGAGGAGAACCCCATCAACGCCAGCGG
CGTGGACGCCAAGGCCATCCTGAGCGCCCGGCTGAGCAAGAGCCGGCGGCTGGAGAACCTG
ATCGCCCAGCTGCCCGGCGAGAAGAAGAACGGCCTGTTCGGCAACCTGATCGCCCTGAGCC
TGGGCCTGACCCCCAACTTCAAGAGCAACTTCGACCTGGCCGAGGACGCCAAGCTGCAGCT
GAGCAAGGACACCTACGACGACGACCTGGACAACCTGCTGGCCCAGATCGGCGACCAGTAC
GCCGACCTGTTCCTGGCCGCCAAGAACCTGAGCGACGCCATCCTGCTGAGCGACATCCTGCG
GGTGAACACCGAGATCACCAAGGCCCCCCTGAGCGCCAGCATGATCAAGCGGTACGACGAG
CACCACCAGGACCTGACCCTGCTGAAGGCCCTGGTGCGGCAGCAGCTGCCCGAGAAGTACA
AGGAGATCTTCTTCGACCAGAGCAAGAACGGCTACGCCGGCTACATCGACGGCGGCGCCAG
CCAGGAGGAGTTCTACAAGTTCATCAAGCCCATCCTGGAGAAGATGGACGGCACCGAGGAG
CTGCTGGTGAAGCTGAACCGGGAGGACCTGCTGCGGAAGCAGCGGACCTTCGACAACGGCA
GCATCCCCCACCAGATCCACCTGGGCGAGCTGCACGCCATCCTGCGGCGGCAGGAGGACTTC
TACCCCTTCCTGAAGGACAACCGGGAGAAGATCGAGAAGATCCTGACCTTCCGGATCCCCTA
CTACGTGGGCCCCCTGGCCCGGGGCAACAGCCGGTTCGCCTGGATGACCCGGAAATCCGAG
GAGACCATCACCCCCTGGAACTTCGAGGAGGTGGTGGACAAGGGCGCCAGCGCCCAGAGCT
TCATCGAGCGGATGACCAACTTCGACAAGAACCTGCCCAACGAGAAGGTGCTGCCCAAGCA
CAGCCTGCTGTACGAGTACTTCACCGTGTACAACGAGCTGACCAAGGTGAAGTACGTGACCG
AGGGCATGCGGAAGCCCGCCTTCCTGAGCGGCGAGCAGAAGAAGGCCATCGTGGACCTGCT
GTTCAAGACCAACCGGAAGGTGACCGTGAAGCAGCTGAAGGAGGACTACTTCAAGAAGATC
GAGTGCTTCGACAGCGTGGAGATCAGCGGCGTGGAGGACCGGTTCAACGCCAGCCTGGGCA
CCTACCACGACCTGCTGAAGATCATCAAGGACAAGGACTTCCTGGACAACGAGGAGAACGA
GGACATCCTGGAGGACATCGTGCTGACCCTGACCCTGTTCGAGGACCGGGAGATGATCGAG
GAGCGGCTGAAAACCTACGCCCACCTGTTCGACGACAAGGTGATGAAGCAGCTGAAGCGGC
GGCGGTACACCGGCTGGGGCCGGCTGAGCCGGAAGCTGATCAACGGCATCCGGGACAAGCA
GAGCGGCAAGACCATCCTGGACTTCCTGAAATCCGACGGCTTCGCCAACCGGAACTTCATGC
AGCTGATCCACGACGACAGCCTGACCTTCAAGGAGGACATCCAGAAGGCCCAGGTGAGCGG
CCAGGGCGACAGCCTGCACGAGCACATCGCCAACCTGGCCGGCAGCCCCGCCATCAAGAAG
GGCATCCTGCAGACCGTGAAGGTGGTGGACGAGCTGGTGAAGGTGATGGGCCGGCACAAGC
CCGAGAACATCGTGATCGAGATGGCCCGGGAGAACCAGACCACCCAGAAGGGCCAGAAGA
ACAGCCGGGAGCGGATGAAGCGGATCGAGGAGGGCATCAAGGAGCTGGGCAGCCAGATCC
TGAAGGAGCACCCCGTGGAGAACACCCAGCTGCAGAACGAGAAGCTGTACCTGTACTACCT
GCAGAACGGCCGGGACATGTACGTGGACCAGGAGCTGGACATCAACCGGCTGAGCGACTAC
GACGTGGCCGCCATCGTGCCCCAGAGCTTCCTGAAGGACGACAGCATCGACAACAAGGTGC
TGACCCGGAGCGACAAGGCCCGGGGCAAGAGCGACAACGTGCCCAGCGAGGAGGTGGTGA
AGAAGATGAAGAACTACTGGCGGCAGCTGCTGAACGCCAAGCTGATCACCCAGCGGAAGTT
CGACAACCTGACCAAGGCCGAGCGGGGCGGCCTGAGCGAGCTGGACAAGGCCGGCTTCATC
AAGCGGCAGCTGGTGGAGACCCGGCAGATCACCAAGCACGTGGCCCAGATCCTGGACAGCC
GGATGAACACCAAGTACGACGAGAACGACAAGCTGATCCGGGAGGTGAAGGTGATCACCCT
GAAATCCAAGCTGGTGAGCGACTTCCGGAAGGACTTCCAGTTCTACAAGGTGCGGGAGATC
AACAACTACCACCACGCCCACGACGCCTACCTGAACGCCGTGGTGGGCACCGCCCTGATCA
AGAAGTACCCCAAGCTGGAGAGCGAGTTCGTGTACGGCGACTACAAGGTGTACGACGTGCG
GAAGATGATCGCCAAGAGCGAGCAGGAGATCGGCAAGGCCACCGCCAAGTACTTCTTCTAC
AGCAACATCATGAACTTCTTCAAGACCGAGATCACCCTGGCCAACGGCGAGATCCGGAAGC
GGCCCCTGATCGAGACCAACGGCGAGACCGGCGAGATCGTGTGGGACAAGGGCCGGGACTT
CGCCACCGTGCGGAAGGTGCTGAGCATGCCCCAGGTGAACATCGTGAAGAAAACCGAGGTG
CAGACCGGCGGCTTCAGCAAGGAGAGCATCCTGCCCAAGCGGAACAGCGACAAGCTGATCG
CCCGGAAGAAGGACTGGGACCCCAAGAAGTACGGCGGCTTCGACAGCCCCACCGTGGCCTA
CAGCGTGCTGGTGGTGGCCAAGGTGGAGAAGGGCAAGAGCAAGAAGCTGAAATCCGTGAA
GGAGCTGCTGGGCATCACCATCATGGAGCGGAGCAGCTTCGAGAAGAACCCCATCGACTTC
CTGGAGGCCAAGGGCTACAAGGAGGTGAAGAAGGACCTGATCATCAAGCTGCCCAAGTACA
GCCTGTTCGAGCTGGAGAACGGCCGGAAGCGGATGCTGGCCAGCGCCGGCGAGCTGCAGAA
GGGCAACGAGCTGGCCCTGCCCAGCAAGTACGTGAACTTCCTGTACCTGGCCAGCCACTACG
AGAAGCTGAAGGGCAGCCCCGAGGACAACGAGCAGAAGCAGCTGTTCGTGGAGCAGCACA
AGCACTACCTGGACGAGATCATCGAGCAGATCAGCGAGTTCAGCAAGCGGGTGATCCTGGC
CGACGCCAACCTGGACAAGGTGCTGAGCGCCTACAACAAGCACCGGGACAAGCCCATCCGG
GAGCAGGCCGAGAACATCATCCACCTGTTCACCCTGACCAACCTGGGCGCCCCCGCCGCCTT
CAAGTACTTCGACACCACCATCGACCGGAAGCGGTACACCAGCACCAAGGAGGTGCTGGAC
GCCACCCTGATCCACCAGAGCATCACCGGCCTGTACGAGACCCGGATCGACCTGAGCCAGCT
GGGCGGCGAC
(SEQ ID NO: 89)
GCCAAGCGGAACTACATCCTGGGCCTGGCCATCGGCATCACCAGCGTGGGCTACGGCATCAT
CGACTACGAGACCCGGGACGTGATCGACGCCGGCGTGCGGCTGTTCAAGGAGGCCAACGTG
GAGAACAACGAGGGCCGGCGGAGCAAGCGGGGCGCCCGGCGGCTGAAGCGGCGGCGGCGG
CACCGGATCCAGCGGGTGAAGAAGCTGCTGTTCGACTACAACCTGCTGACCGACCACAGCG
AGCTGAGCGGCATCAACCCCTACGAGGCCCGGGTGAAGGGCCTGAGCCAGAAGCTGAGCGA
GGAGGAGTTCAGCGCCGCCCTGCTGCACCTGGCCAAGCGGCGGGGCGTGCACAACGTGAAC
GAGGTGGAGGAGGACACCGGCAACGAGCTGAGCACCAAGGAGCAGATCAGCCGGAACAGC
AAGGCCCTGGAGGAGAAGTACGTGGCCGAGCTGCAGCTGGAGCGGCTGAAGAAGGACGGC
GAGGTGCGGGGCAGCATCAACCGGTTCAAGACCAGCGACTACGTGAAGGAGGCCAAGCAGC
TGCTGAAGGTGCAGAAGGCCTACCACCAGCTGGACCAGAGCTTCATCGACACCTACATCGA
CCTGCTGGAGACCCGGCGGACCTACTACGAGGGCCCCGGCGAGGGCAGCCCCTTCGGCTGG
AAGGACATCAAGGAGTGGTACGAGATGCTGATGGGCCACTGCACCTACTTCCCCGAGGAGC
TGCGGAGCGTGAAGTACGCCTACAACGCCGACCTGTACAACGCCCTGAACGACCTGAACAA
CCTGGTGATCACCCGGGACGAGAACGAGAAGCTGGAGTACTACGAGAAGTTCCAGATCATC
GAGAACGTGTTCAAGCAGAAGAAGAAGCCCACCCTGAAGCAGATCGCCAAGGAGATCCTGG
TGAACGAGGAGGACATCAAGGGCTACCGGGTGACCAGCACCGGCAAGCCCGAGTTCACCAA
CCTGAAGGTGTACCACGACATCAAGGACATCACCGCCCGGAAGGAGATCATCGAGAACGCC
GAGCTGCTGGACCAGATCGCCAAGATCCTGACCATCTACCAGAGCAGCGAGGACATCCAGG
AGGAGCTGACCAACCTGAACAGCGAGCTGACCCAGGAGGAGATCGAGCAGATCAGCAACCT
GAAGGGCTACACCGGCACCCACAACCTGAGCCTGAAGGCCATCAACCTGATCCTGGACGAG
CTGTGGCACACCAACGACAACCAGATCGCCATCTTCAACCGGCTGAAGCTGGTGCCCAAGA
AGGTGGACCTGAGCCAGCAGAAGGAGATCCCCACCACCCTGGTGGACGACTTCATCCTGAG
CCCCGTGGTGAAGCGGAGCTTCATCCAGAGCATCAAGGTGATCAACGCCATCATCAAGAAG
TACGGCCTGCCCAACGACATCATCATCGAGCTGGCCCGGGAGAAGAACAGCAAGGACGCCC
AGAAGATGATCAACGAGATGCAGAAGCGGAACCGGCAGACCAACGAGCGGATCGAGGAGA
TCATCCGGACCACCGGCAAGGAGAACGCCAAGTACCTGATCGAGAAGATCAAGCTGCACGA
CATGCAGGAGGGCAAGTGCCTGTACAGCCTGGAGGCCATCCCCCTGGAGGACCTGCTGAAC
AACCCCTTCAACTACGAGGTGGACGCCATCATCCCCCGGAGCGTGAGCTTCGACAACAGCTT
CAACAACAAGGTGCTGGTGAAGCAGGAGGAGAACAGCAAGAAGGGCAACCGGACCCCCTT
CCAGTACCTGAGCAGCAGCGACAGCAAGATCAGCTACGAGACCTTCAAGAAGCACATCCTG
AACCTGGCCAAGGGCAAGGGCCGGATCAGCAAGACCAAGAAGGAGTACCTGCTGGAGGAG
CGGGACATCAACCGGTTCAGCGTGCAGAAGGACTTCATCAACCGGAACCTGGTGGACACCC
GGTACGCCACCCGGGGCCTGATGAACCTGCTGCGGAGCTACTTCCGGGTGAACAACCTGGA
CGTGAAGGTGaaatccATCAACGGCGGCTTCACCAGCTTCCTGCGGCGGAAGTGGAAGTTCAAG
AAGGAGCGGAACAAGGGCTACAAGCACCACGCCGAGGACGCCCTGATCATCGCCAACGCCG
ACTTCATCTTCAAGGAGTGGAAGAAGCTGGACAAGGCCAAGAAGGTGATGGAGAACCAGAT
GTTCGAGGAGAAGCAGGCCGAGAGCATGCCCGAGATCGAGACCGAGCAGGAGTACAAGGA
GATCTTCATCACCCCCCACCAGATCAAGCACATCAAGGACTTCAAGGACTACAAGTACAGCC
ACCGGGTGGACAAGAAGCCCAACCGGAAGCTGATCAACGACACCCTGTACAGCACCCGGAA
GGACGACAAGGGCAACACCCTGATCGTGAACAACCTGAACGGCCTGTACGACAAGGACAAC
GACAAGCTGAAGAAGCTGATCAACAAGAGCCCCGAGAAGCTGCTGATGTACCACCACGACC
CCCAGACCTACCAGAAGCTGAAGCTGATCATGGAGCAGTACGGCGACGAGAAGAACCCCCT
GTACAAGTACTACGAGGAGACCGGCAACTACCTGACCAAGTACAGCAAGAAGGACAACGGC
CCCGTGATCAAGAAGATCAAGTACTACGGCAACAAGCTGAACGCCCACCTGGACATCACCG
ACGACTACCCCAACAGCCGGAACAAGGTGGTGAAGCTGAGCCTGAAGCCCTACCGGTTCGA
CGTGTACCTGGACAACGGCGTGTACAAGTTCGTGACCGTGAAGAACCTGGACGTGATCAAG
AAGGAGAACTACTACGAGGTGAACAGCAAGTGCTACGAGGAGGCCAAGAAGCTGAAGAAG
ATCAGCAACCAGGCCGAGTTCATCGCCAGCTTCTACAAGAACGACCTGATCAAGATCAACG
GCGAGCTGTACCGGGTGATCGGCGTGAACAACGACCTGCTGAACCGGATCGAGGTGAACAT
GATCGACATCACCTACCGGGAGTACCTGGAGAACATGAACGACAAGCGGCCCCCCCACATC
ATCAAGACCATCGCCAGCAAGACCCAGAGCATCAAGAAGTACAGCACCGACATCCTGGGCA
ACCTGTACGAGGTGaaatccAAGAAGCACCCCCAGATCATCAAGAAGGGC
Guide RNA (gRNA)

In some embodiments, a DNA-targeting moiety may comprise a Cas molecule comprising or linked (e.g., covalently) to a gRNA. A gRNA is a short synthetic RNA composed of a “scaffold” sequence necessary for Cas-protein binding and a user-defined ˜20 nucleotide targeting sequence for a genomic target. In practice, guide RNA sequences are generally designed to have a length of between 17-24 nucleotides (e.g., 19, 20, or 21 nucleotides) and be complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective for use with Cas proteins; see, for example, Hendel et al. (2015) Nature Biotechnol., 985-991.

In some embodiments, a gRNA comprises a nucleic acid sequence that is complementary to a DNA sequence associated with a target gene. In some embodiments, the DNA sequence is, comprises, or overlaps an expression control element that is operably linked to the target gene. In some embodiments, a gRNA comprises a nucleic acid sequence that is at least 90, 95, 99, or 100% complementary to a DNA sequence associated with a target gene. In some embodiments, a gRNA for use with a DNA-targeting moiety that comprises a Cas molecule is an sgRNA.

In some embodiments, a gRNA for use with a CRISPR/Cas molecule specifically binds a target sequence associated with β-2-microglobulin expression. Such a gRNA may comprise a target-binding sequence selected from:

| | GD-28228: |
|---|---|
| (SEQ ID NO: 1) | |
| | TCTCCTTGGTGGCCCGCCGT, |
| | GD-28229: |
| (SEQ ID NO: 2) | |
| | GTCCCAAAGGCGCGGCGCTG, |
| | GD-28171: |
| (SEQ ID NO: 3) | |
| | CCCTGCTCCCCGCCGAAAGGG, |
| | GD-28172: |
| (SEQ ID NO: 4) | |
| | CTCTGGCTCCCCCAGCGCAGC, |
| | or |
| | GD-28173: |
| (SEQ ID NO: 5) | |
| | GTGAACGCGTGGAGGGGCGCT |

In some embodiments, a gRNA for use with a CRISPR/Cas domain specifically binds a target sequence associated with CTCF. In some embodiments, a gRNA for use with a CRISPR/Cas domain specifically binds a target sequence associated with the promoter. In some embodiments the gRNA binds a target sequence listed in Table 3.

TABLE 3
Exemplary gRNA sequences

| | | | | SEQ |
|---|---|---|---|---|
| | | | Genomic | ID |
| Guide | Target | Target sequence | Coordinates | NO: |
| GD- | MYC | AGGGTGATGTTCATT | chr8:128746082- | 11 |
| 29639 | | AGCAG | 128746104 | |
| GD- | MYC | TGCAGAAGGTCCGAA | chr8:128746177- | 12 |
| 29640 | | GAAAG | 128746199 | |
| GD- | β2M | GAGACAGGTGACGGT | chr15:45003632- | 13 |
| 27634 | | CCCTG | 45003654 | |
| GD- | β2M | GAGTCTCGTGATGTT | chr15:45003545- | 14 |
| 29500 | | TAAGA | 45003567 | |
| GD- | HSPA1B | CCTGCCCTCTGATTG | chr6:31795352- | 17 |
| 29542 | | GTCCA | 31795374 | |
| GD- | HSPA1B | GCCTCATCGAGCTTG | chr6:31795429- | 18 |
| 29544 | | GTGAT | 31795451 | |
| GD- | GATA1 | CTGTGGTGTCAAAAG | chrX:48641126- | 19 |
| 29536 | | CACCA | 48641148 | |
| GD- | GATA1 | GAACCCCAGAAGATG | chrX:48641245- | 20 |
| 29693 | | CCAGG | 48641267 | |
| GD- | β2M | GGCGCGCACCCCAGA | chr15:45003481- | 21 |
| 29499 | promoter | TCGGA | 45003503 | |

In some embodiments, an expression repressor system comprises a first expression repressor comprising a first DNA-targeting moiety and a second expression repressor comprising a second DNA-targeting moiety, wherein the first DNA-targeting moiety comprises or is a first CRISPR/Cas molecule and the second DNA-targeting moiety comprises or is a second CRISPR/Cas molecule. In some embodiments, the first CRISPR/Cas molecule comprises a first CRISPR/Cas protein and first guide RNA, and the second CRISPR/Cas molecule comprises a second CRISPR/Cas protein and a second guide RNA. In some embodiments, the first CRISPR/Cas protein does not appreciably bind (e.g., does not bind) the second guide RNA, e.g., binds with a K_{D} of at least 10, 20, 50, 100, 1000, or 10,000 nM, and the second CRISPR/Cas protein does not appreciably bind (e.g., does not bind) the first guide RNA, e.g., binds with a K_{D} of at least 10, 20, 50, 100, 1000, or 10,000 nM.

### TAL Effector Domains

In some embodiments, a DNA-targeting moiety is or comprises a TAL effector molecule. A TAL effector molecule, e.g., a TAL effector molecule that specifically binds a DNA sequence, comprises a plurality of TAL effector domains or fragments thereof, and optionally one or more additional portions of naturally occurring TAL effectors (e.g., N- and/or C-terminal of the plurality of TAL effector domains). Many TAL effectors are known to those of skill in the art and are commercially available, e.g., from Thermo Fisher Scientific.

TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector feature a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 2 listing exemplary repeat variable di-residues (RVD) and their correspondence to nucleic acid base targets.

TABLE 2
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of the TAL effector molecule of the present invention may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicola strain BLS256 (Bogdanove et al. 2011). As used herein, the TAL effector domain in accordance with the present invention comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL effector molecule of the present invention is designed to target a given DNA sequence based on the above code and others known in the art. The number of TAL effector domains (e.g., repeats (monomers or modules)) and their specific sequence are selected based on the desired DNA target sequence. For example, TAL effector domains, e.g., repeats, may be removed or added in order to suit a specific target sequence. In an embodiment, the TAL effector molecule of the present invention comprises between 6.5 and 33.5 TAL effector domains, e.g., repeats. In an embodiment, TAL effector molecule of the present invention comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the expression repression system, e.g., the expression repressor comprising the TAL effector molecule. In general, TALE binding is inversely correlated with the number of mismatches. In some embodiments, the TAL effector molecule of an expression repressor of the present invention comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the expression repression system, e.g., the expression repressor comprising the TAL effector molecule. The binding affinity is thought to depend on the sum of matching repeat-DNA combinations. For example, TAL effector molecules having 25 TAL effector domains or more may be able to tolerate up to 7 mismatches.

In addition to the TAL effector domains, the TAL effector molecule of the present invention may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule of an expression repressor of the present invention. Accordingly, in an embodiment, a TAL effector molecule of the present invention comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector.

### Zn Finger Domains

In some embodiments, a DNA-targeting moiety is or comprises a Zn finger domain. A Zn finger domain comprises a Zn finger protein, e.g., a naturally occurring Zn finger protein or engineered Zn finger protein, or fragment thereof. Many Zn finger proteins are known to those of skill in the art and are commercially available, e.g., from Sigma-Aldrich.

In some embodiments, a Zn finger domain comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger domains described herein may include any combination of suitable linkers between the individual zinc finger proteins and/or multi-fingered Zn finger proteins of the Zn finger domain.

In certain embodiments, the DNA-targeting moiety comprises a Zn finger domain comprising an engineered zinc finger protein that binds (in a sequence-specific manner) to a target DNA sequence. In some embodiments, the Zn finger domain comprises one Zn finger protein or fragment thereof. In other embodiments, the Zn finger domain comprises a plurality of Zn finger proteins (or fragments thereof), e.g., 2, 3, 4, 5, 6 or more Zn finger proteins (and optionally no more than 12, 11, 10, 9, 8, 7, 6, 5, 4, 3, or 2 Zn finger proteins). In some embodiments, the Zn finger domain comprises at least three Zn finger proteins. In some embodiments, the Zn finger domain comprises four, five or six fingers. In some embodiments, the Zn finger domain comprises 8, 9, 10, 11 or 12 fingers. In some embodiments, a Zn finger domain comprising three Zn finger proteins recognizes a target DNA sequence comprising 9 or 10 nucleotides. In some embodiments, a Zn finger domain comprising four Zn finger proteins recognizes a target DNA sequence comprising 12 to 14 nucleotides. In some embodiments, a Zn finger domain comprising six Zn finger proteins recognizes a target DNA sequence comprising 18 to 21 nucleotides.

In some embodiments, a Zn finger domain comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two-handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18(18):5073-5084). Each cluster of zinc fingers in these proteins is able to bind to a unique target sequence and the spacing between the two target sequences can comprise many nucleotides.

### Nucleic Acid Molecule

In some embodiments, a DNA-targeting moiety is or comprises a DNA-binding domain from a nuclease. For example, the recognition sequences of homing endonucleases and meganucleases such as T-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996) J. Mol. Biol. 263:163-180; Argast, et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Patent Publication No. 2007/0117128.

In some embodiments, a DNA-targeting moiety comprises or is nucleic acid. In some embodiments, a nucleic acid that may be included in a DNA-targeting moiety, may be or comprise DNA, RNA, and/or an artificial or synthetic nucleic acid or nucleic acid analog or mimic. For example, in some embodiments, a nucleic acid may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. In some embodiments, a nucleic acid may include one or more residues that is not a naturally occurring DNA or RNA residue, may include one or more linkages that is/are not phosphodiester bonds (e.g., that may be, for example, phosphorothioate bonds, etc.), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

A nucleic acid suitable for use in an expression repressor, e.g., in the DNA-targeting moiety, may include, but is not limited to, DNA, RNA, modified oligonucleotides (e.g., chemical modifications, such as modifications that alter backbone linkages, sugar molecules, and/or nucleic acid bases), and artificial nucleic acids. In some embodiments, a nucleic acid includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules.

In some embodiments, a DNA-targeting moiety comprises a nucleic acid with a length from about 15-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 215-190, 20-190, 30-190, 40-190, 50-190, 60-190, 70-190, 80-190, 90-190, 100-190, 110-190, 120-190, 130-190, 140-190, 150-190, 160-190, 170-190, 180-190, 15-180, 20-180, 30-180, 40-180, 50-180, 60-180, 70-180, 80-180, 90-180, 100-180, 110-180, 120-180, 130-180, 140-180, 150-180, 160-180, 170-180, 15-170, 20-170, 30-170, 40-170, 50-170, 60-170, 70-170, 80-170, 90-170, 100-170, 110-170, 120-170, 130-170, 140-170, 150-170, 160-170, 15-160, 20-160, 30-160, 40-160, 50-160, 60-160, 70-160, 80-160, 90-160, 100-160, 110-160, 120-160, 130-160, 140-160, 150-160, 215-150, 20-150, 30-150, 40-150, 50-150, 60-150, 70-150, 80-150, 90-150, 100-150, 110-150, 120-150, 130-150, 140-150, 15-140, 20-140, 30-140, 40-140, 50-140, 60-140, 70-140, 80-140, 90-140, 100-140, 110-140, 120-140, 130-140, 15-130, 20-130, 30-130, 40-130, 50-130, 60-130, 70-130, 80-130, 90-130, 100-130, 110-130, 120-130, 215-120, 20-120, 30-120, 40-120, 50-120, 60-120, 70-120, 80-120, 90-120, 100-120, 110-120, 15-110, 20-110, 30-110, 40-110, 50-110, 60-110, 70-110, 80-110, 90-110, 100-110, 15-100, 20-100, 30-100, 40-100, 50-100, 60-100, 70-100, 80-100, 90-100, 15-90, 20-90, 30-90, 40-90, 50-90, 60-90, 70-90, 80-90, 15-80, 20-80, 30-80, 40-80, 50-80, 60-80, 70-80, 15-70, 20-70, 30-70, 40-70, 50-70, 60-70, 15-60, 20-60, 30-60, 40-60, 50-60, 15-50, 20-50, 30-50, 40-50, 15-40, 20-40, 30-40, 15-30, 20-30, or 15-20 nucleotides, or any range therebetween.

### Repressor Domains

Expression repressors of the present disclosure comprise one or more repressor domains. A repressor domain has one or more functionality that, when used as part of an expressor repressor or an expression repression system described herein, decreases expression of a target gene in a cell. In some embodiments, a repressor domain comprises a histone modifying functionality, e.g., a histone methyltransferase, histone demethylase, or histone deacetylase activity. In some embodiments, a histone methyltransferase functionality comprises H3K9 targeting methyltransferase activity. In some embodiments, a histone methyltransferase functionality comprises H3K56 targeting methyltransferase activity. In some embodiments, a histone methyltransferase functionality comprises H3K27 targeting methyltransferase activity. In some embodiments, a histone methyltransferase or demethylase functionality transfers one, two, or three methyl groups. In some embodiments, a histone demethylase functionality comprises H3K4 targeting demethylase activity. In some embodiments, a repressor domain is or comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof. In some embodiments, a repressor domain is or comprises a protein chosen from KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof. In some embodiments, a repressor domain is or comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

In some embodiments, a repressor domain comprises an epigenetic modifying moiety. In some embodiments, a repressor domain comprises a DNA modifying functionality, e.g., a DNA methyltransferase. In some embodiments, a repressor domain is or comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, DNMT3a/3L or a functional variant or fragment of any thereof.

In some embodiments, a repressor domain comprises a transcription repressor. In some embodiments the transcription repressor blocks recruitment of a factor that stimulates or promotes transcription, e.g., of the target gene. In some embodiments, the transcription repressor recruits a factor that inhibits transcription, e.g., of the target gene. In some embodiments, a repressor domain, e.g., transcription repressor, is or comprises a protein chosen from KRAB, MeCP2, HP1, RBBP4, REST, FOG1, SUZ12, or a functional variant or fragment of any thereof.

In some embodiments a repressor domain promotes epigenetic modification, e.g., directly, or indirectly. For example, a repressor domain can indirectly promote epigenetic modification by recruiting an endogenous protein that epigenetically modifies the chromatin. In some embodiments, a repressor domain can directly promote epigenetic modification by catalyzing epigenetic modification, wherein the repressor domain comprises enzymatic activity and directly places an epigenetic mark on the chromatin.

In some embodiments, a repressor domain comprises a protein having a functionality described herein. In some embodiments, a repressor domain is or comprises a protein selected from:
KRAB (e.g., as according to NP_056209.2 or the protein encoded by NM_015394.5, e.g., as according to SEQ ID NO: 61);
a SET domain (e.g., the SET domain of:
SETDB1 (e.g., as according to NP_001353347.1 or the protein encoded by NM_001366418.1);
EZH2 (e.g., as according to NP-004447.2 or the protein encoded by NM_004456.5, e.g., as according to SEQ ID NO: 65);
G9A (e.g., as according to NP_001350618.1 or the protein encoded by NM_001363689.1; e.g., as according to SEQ ID NO: 62); or
SUV39H1 (e.g., as according to NP_003164.1 or the protein encoded by NM_003173.4)); histone demethylase LSD1 (e.g., as according to NP_055828.2 or the protein encoded by NM_015013.4, e.g., as according to SEQ ID NO: 64);
FOG1 (e.g., the N-terminal residues of FOG1) (e.g., as according to NP_722520.2 or the protein encoded by NM_153813.3, e.g., as according to SEQ ID NO: 66);
KAP1 (e.g., as according to NP_005753.1 or the protein encoded by NM_005762.3); or
HDAC8 (e.g., as according to NP_001159890 or the protein encoded by NM_001166418, e.g., as according to SEQ ID NO: 63);
a functional fragment or variant of any thereof, or
a polypeptide with a sequence that has at least 80, 85, 90, 91, 92, 93, 94, 95, 96, 97, 98, or 99% identity to any of the above-referenced sequences. In some embodiments, a repressor domain is or comprises a protein selected from:
DNMT3A (e.g., human DNMT3A) (e.g., as according to NP_072046.2 or the protein encoded by NM_022552.4, e.g., as according to SEQ ID NO: 58);
DNMT3B (e.g., as according to or AOX21819.1 or the protein encoded by NM_006892.4 or KX447429);
DNMT3L (e.g., as according to NP_787063.1 or the protein encoded by NM_175867.3);
DNMT3A/3L complex (e.g., as according to SEQ ID NO: 59 or 60), bacterial MQ1 (e.g., as according to CAA35058.1 or P15840.3, e.g., as according to SEQ ID NO: 57 or 90);
a functional fragment of any thereof, or
a polypeptide with a sequence that has at least 80, 85, 90, 91, 92, 93, 94, 95, 96, 97, 98, or 99% identity to any of the above-referenced sequences.

In some embodiments, a repressor domain is or comprises a polypeptide. In some embodiments, a repressor domain has enzymatic activity.

In some embodiments, a DNA-binding domain comprises a helix-hairpin-helix (HhH) motif. DNA-binding proteins with a HhH structural motif may be involved in non-sequence-specific DNA binding that occurs via the formation of hydrogen bonds between protein backbone nitrogens and DNA phosphate groups.

In some embodiments, a DNA-binding domain comprises a helix-loop-helix (HLH) motif. DNA-binding proteins with an HLH structural motif are transcriptional regulatory proteins and are principally related to a wide array of developmental processes. An HLH structural motif is longer, in terms of residues, than HTH or HhH motifs. Many of these proteins interact to form homo- and hetero-dimers. A structural motif is composed of two long helix regions, with an N-terminal helix binding to DNA, while a complex region allows the protein to dimerize.

In some embodiments, a DNA-binding domain comprises a leucine zipper motif. In some transcription factors, a dimer binding site with DNA forms a leucine zipper. This motif includes two amphipathic helices, one from each subunit, interacting with each other resulting in a left-handed coiled-coil super secondary structure. A leucine zipper is an interdigitation of regularly spaced leucine residues in one helix with leucines from an adjacent helix. Mostly, helices involved in leucine zippers exhibit a heptad sequence (abcdefg) with residues a and d being hydrophobic and other residues being hydrophilic. Leucine zipper motifs can mediate either homo- or heterodimer formation.

In some embodiments, a DNA-binding domain comprises a Zn finger domain, where a Zn++ ion is coordinated by 2 Cys and 2 His residues. Such a transcription factor includes a trimer with the stoichiometry ββ′α. An apparent effect of Zn++ coordination is stabilization of a small complex structure instead of hydrophobic core residues. Each Zn-finger interacts in a conformationally identical manner with successive triple base pair segments in the major groove of the double helix. Protein-DNA interaction is determined by two factors: (i) H-bonding interaction between α-helix and DNA segment, mostly between Arg residues and Guanine bases. (ii) H-bonding interaction with DNA phosphate backbone, mostly with Arg and His. An alternative Zn-finger motif chelates Zn++ with 6 Cys.

An exemplary repressor domain may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3A, DNMT3B, DNMT3L), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9A), histone-lysine N-methyltransferase (SUV39H1), and G9a), histone deacetylase (e.g., HDAC1, HDAC2, HDAC3, HDAC8), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), transcription repressors (e.g., KRAB, FOG1), helicases such as DHX9, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl 0-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as a KRAB domain.

In some embodiments, a candidate domain may be determined to be suitable for use as a repressor domain by methods known to those of skill in the art. For example, a candidate repressor domain may be tested by assaying whether, when the candidate repressor domain is present in the nucleus of a cell and appropriately localized (e.g., to a target gene or transcription control element operably linked to said target gene, e.g., via a DNA-targeting moiety), the candidate repressor domain decreases expression of the target gene in the cell, e.g., decreases the level of RNA transcript encoded by the target gene (e.g., as measured by RNASeq or Northern blot) or decreases the level of protein encoded by the target gene (e.g., as measured by ELISA).

In some embodiments, an expression repression system comprises a plurality of expression repressors, wherein each member of the plurality of expression repressors comprises a repressor domain, wherein each repressor domain does not detectably bind, e.g., does not bind, to another repressor domain. In some embodiments, an expression repression system comprises a first expression repressor comprising a first repressor domain and a second expression repressor comprising a second repressor domain, wherein the first repressor domain does not detectably bind, e.g., does not bind, to the second repressor domain. In some embodiments, an expression repression system comprises a first expression repressor comprising a first repressor domain and a second expression repressor comprising a second repressor domain, wherein the first repressor domain does not detectably bind, e.g., does not bind, to another first repressor domain, and the second repressor domain does not detectably bind, e.g., does not bind, to another second repressor domain. In some embodiments, a repressor domain for use in the compositions and methods described herein is functional in a monomeric, e.g., non-dimeric, state.

In some embodiments, a repressor domain is or comprises an epigenetic modifying moiety, e.g., that modulates the two-dimensional structure of chromatin (i.e., that modulate structure of chromatin in a way that would alter its two-dimensional representation).

Epigenetic modifying moieties useful in methods and compositions of the present disclosure include agents that affect epigenetic markers, e.g., DNA methylation, histone methylation, histone acetylation, histone sumoylation, histone phosphorylation, and RNA-associated silencing. Exemplary epigenetic enzymes that can be targeted to a genomic sequence element as described herein include DNA methylases (e.g., DNMT3a, DNMT3b, DNMTL), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, an expression repressor, e.g., comprising an epigenetic modifying moiety, useful herein comprises or is a construct described in Koferle et al. Genome Medicine 7.59 (2015):1-3 incorporated herein by reference. For example, in some embodiments, an expression repressor comprises or is a construct found in Table 1 of Koferle et al., e.g., histone deacetylase, histone methyltransferase, DNA demethylation, or H3K4 and/or H3K9 histone demethylase described in Table 1 (e.g., dCas9-β300, TALE-TET1, ZF-DNMT3A, or TALE-LSD1).

### Additional Moieties

An expression repressor may further comprise one or more additional moieties (e.g., in addition to one or more DNA-targeting moieties and one or more repressor domains). In some embodiments, an additional moiety is selected from a tagging or monitoring moiety, a cleavable moiety (e.g., a cleavable moiety positioned between a DNA-targeting moiety and a repressor domain or at the N- or C-terminal end of a polypeptide), a small molecule, a membrane translocating polypeptide, or a pharmacoagent moiety.

### Exemplary Expression Repressors

The following exemplary expression repressors are presented for illustration purposes only and are not intended to be limiting.

In some embodiments, an expression repressor comprises a DNA-targeting moiety comprising dCas9, e.g., an S. aureus dCas9, and a repressor domain comprising MQ1, e.g., bacterial MQ1. In some embodiments, the expression repressor is encoded by the nucleic acid sequence of SEQ ID NO: 91 (e.g., a plasmid encoding the expression repressor), SEQ ID NO: 22 (e.g., a nucleic acid (e.g., cDNA) encoding the expression repressor) and/or SEQ ID NO: 92 (e.g., a nucleic acid (e.g., cDNA) encoding the expression repressor). In some embodiments, a nucleic acid described herein comprises a nucleic acid sequence of SEQ ID NO: 22, 91 or 92 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the DNA-targeting moiety is encoded by the nucleic acid sequence of SEQ ID NO: 45 or 89 and/or the repressor domain is encoded by the nucleic acid sequence of SEQ ID NO: 47.

dCas9-MQ1 (MR-28125) mRNA sequence:
(SEQ ID NO: 22)
AGGAAATAAGAGAGAAAAGAAGAGTAAGAAGAAATATAAGAGCCACCATGGCCCCCAAGAA
GAAGCGGAAGGTGGGCATCCACGGCGTGCCCGCCGCCGACAAGAAGTACAGCATCGGCCTG
GCCATCGGCACCAACAGCGTGGGCTGGGCCGTGATCACCGACGAGTACAAGGTGCCCAGCA
AGAAGTTCAAGGTGCTGGGCAACACCGACCGGCACAGCATCAAGAAGAACCTGATCGGCGC
CCTGCTGTTCGACAGCGGCGAGACCGCCGAGGCCACCCGGCTGAAGCGGACCGCCCGGCGG
CGGTACACCCGGCGGAAGAACCGGATCTGCTACCTGCAGGAGATCTTCAGCAACGAGATGG
CCAAGGTGGACGACAGCTTCTTCCACCGGCTGGAGGAGAGCTTCCTGGTGGAGGAGGACAA
GAAGCACGAGCGGCACCCCATCTTCGGCAACATCGTGGACGAGGTGGCCTACCACGAGAAG
TACCCCACCATCTACCACCTGCGGAAGAAGCTGGTGGACAGCACCGACAAGGCCGACCTGC
GGCTGATCTACCTGGCCCTGGCCCACATGATCAAGTTCCGGGGCCACTTCCTGATCGAGGG
CGACCTGAACCCCGACAACAGCGACGTGGACAAGCTGTTCATCCAGCTGGTGCAGACCTAC
AACCAGCTGTTCGAGGAGAACCCCATCAACGCCAGCGGCGTGGACGCCAAGGCCATCCTGA
GCGCCCGGCTGAGCAAGAGCCGGCGGCTGGAGAACCTGATCGCCCAGCTGCCCGGCGAGAA
GAAGAACGGCCTGTTCGGCAACCTGATCGCCCTGAGCCTGGGCCTGACCCCCAACTTCAAG
AGCAACTTCGACCTGGCCGAGGACGCCAAGCTGCAGCTGAGCAAGGACACCTACGACGACG
ACCTGGACAACCTGCTGGCCCAGATCGGCGACCAGTACGCCGACCTGTTCCTGGCCGCCAA
GAACCTGAGCGACGCCATCCTGCTGAGCGACATCCTGCGGGTGAACACCGAGATCACCAAG
GCCCCCCTGAGCGCCAGCATGATCAAGCGGTACGACGAGCACCACCAGGACCTGACCCTGC
TGAAGGCCCTGGTGCGGCAGCAGCTGCCCGAGAAGTACAAGGAGATCTTCTTCGACCAGAG
CAAGAACGGCTACGCCGGCTACATCGACGGCGGCGCCAGCCAGGAGGAGTTCTACAAGTTC
ATCAAGCCCATCCTGGAGAAGATGGACGGCACCGAGGAGCTGCTGGTGAAGCTGAACCGGG
AGGACCTGCTGCGGAAGCAGCGGACCTTCGACAACGGCAGCATCCCCCACCAGATCCACCT
GGGCGAGCTGCACGCCATCCTGCGGCGGCAGGAGGACTTCTACCCCTTCCTGAAGGACAAC
CGGGAGAAGATCGAGAAGATCCTGACCTTCCGGATCCCCTACTACGTGGGCCCCCTGGCCC
GGGGCAACAGCCGGTTCGCCTGGATGACCCGGAAATCCGAGGAGACCATCACCCCCTGGAA
CTTCGAGGAGGTGGTGGACAAGGGCGCCAGCGCCCAGAGCTTCATCGAGCGGATGACCAAC
TTCGACAAGAACCTGCCCAACGAGAAGGTGCTGCCCAAGCACAGCCTGCTGTACGAGTACT
TCACCGTGTACAACGAGCTGACCAAGGTGAAGTACGTGACCGAGGGCATGCGGAAGCCCGC
CTTCCTGAGCGGCGAGCAGAAGAAGGCCATCGTGGACCTGCTGTTCAAGACCAACCGGAAG
GTGACCGTGAAGCAGCTGAAGGAGGACTACTTCAAGAAGATCGAGTGCTTCGACAGCGTGG
AGATCAGCGGCGTGGAGGACCGGTTCAACGCCAGCCTGGGCACCTACCACGACCTGCTGAA
GATCATCAAGGACAAGGACTTCCTGGACAACGAGGAGAACGAGGACATCCTGGAGGACATC
GTGCTGACCCTGACCCTGTTCGAGGACCGGGAGATGATCGAGGAGCGGCTGAAAACCTACG
CCCACCTGTTCGACGACAAGGTGATGAAGCAGCTGAAGCGGCGGCGGTACACCGGCTGGGG
CCGGCTGAGCCGGAAGCTGATCAACGGCATCCGGGACAAGCAGAGCGGCAAGACCATCCTG
GACTTCCTGAAATCCGACGGCTTCGCCAACCGGAACTTCATGCAGCTGATCCACGACGACA
GCCTGACCTTCAAGGAGGACATCCAGAAGGCCCAGGTGAGCGGCCAGGGCGACAGCCTGCA
CGAGCACATCGCCAACCTGGCCGGCAGCCCCGCCATCAAGAAGGGCATCCTGCAGACCGTG
AAGGTGGTGGACGAGCTGGTGAAGGTGATGGGCCGGCACAAGCCCGAGAACATCGTGATCG
AGATGGCCCGGGAGAACCAGACCACCCAGAAGGGCCAGAAGAACAGCCGGGAGCGGATGAA
GCGGATCGAGGAGGGCATCAAGGAGCTGGGCAGCCAGATCCTGAAGGAGCACCCCGTGGAG
AACACCCAGCTGCAGAACGAGAAGCTGTACCTGTACTACCTGCAGAACGGCCGGGACATGT
ACGTGGACCAGGAGCTGGACATCAACCGGCTGAGCGACTACGACGTGGCCGCCATCGTGCC
CCAGAGCTTCCTGAAGGACGACAGCATCGACAACAAGGTGCTGACCCGGAGCGACAAGGCC
CGGGGCAAGAGCGACAACGTGCCCAGCGAGGAGGTGGTGAAGAAGATGAAGAACTACTGGC
GGCAGCTGCTGAACGCCAAGCTGATCACCCAGCGGAAGTTCGACAACCTGACCAAGGCCGA
GCGGGGCGGCCTGAGCGAGCTGGACAAGGCCGGCTTCATCAAGCGGCAGCTGGTGGAGACC
CGGCAGATCACCAAGCACGTGGCCCAGATCCTGGACAGCCGGATGAACACCAAGTACGACG
AGAACGACAAGCTGATCCGGGAGGTGAAGGTGATCACCCTGAAATCCAAGCTGGTGAGCGA
CTTCCGGAAGGACTTCCAGTTCTACAAGGTGCGGGAGATCAACAACTACCACCACGCCCAC
GACGCCTACCTGAACGCCGTGGTGGGCACCGCCCTGATCAAGAAGTACCCCAAGCTGGAGA
GCGAGTTCGTGTACGGCGACTACAAGGTGTACGACGTGCGGAAGATGATCGCCAAGAGCGA
GCAGGAGATCGGCAAGGCCACCGCCAAGTACTTCTTCTACAGCAACATCATGAACTTCTTC
AAGACCGAGATCACCCTGGCCAACGGCGAGATCCGGAAGCGGCCCCTGATCGAGACCAACG
GCGAGACCGGCGAGATCGTGTGGGACAAGGGCCGGGACTTCGCCACCGTGCGGAAGGTGCT
GAGCATGCCCCAGGTGAACATCGTGAAGAAAACCGAGGTGCAGACCGGCGGCTTCAGCAAG
GAGAGCATCCTGCCCAAGCGGAACAGCGACAAGCTGATCGCCCGGAAGAAGGACTGGGACC
CCAAGAAGTACGGCGGCTTCGACAGCCCCACCGTGGCCTACAGCGTGCTGGTGGTGGCCAA
GGTGGAGAAGGGCAAGAGCAAGAAGCTGAAATCCGTGAAGGAGCTGCTGGGCATCACCATC
ATGGAGCGGAGCAGCTTCGAGAAGAACCCCATCGACTTCCTGGAGGCCAAGGGCTACAAGG
AGGTGAAGAAGGACCTGATCATCAAGCTGCCCAAGTACAGCCTGTTCGAGCTGGAGAACGG
CCGGAAGCGGATGCTGGCCAGCGCCGGCGAGCTGCAGAAGGGCAACGAGCTGGCCCTGCCC
AGCAAGTACGTGAACTTCCTGTACCTGGCCAGCCACTACGAGAAGCTGAAGGGCAGCCCCG
AGGACAACGAGCAGAAGCAGCTGTTCGTGGAGCAGCACAAGCACTACCTGGACGAGATCAT
CGAGCAGATCAGCGAGTTCAGCAAGCGGGTGATCCTGGCCGACGCCAACCTGGACAAGGTG
CTGAGCGCCTACAACAAGCACCGGGACAAGCCCATCCGGGAGCAGGCCGAGAACATCATCC
ACCTGTTCACCCTGACCAACCTGGGCGCCCCCGCCGCCTTCAAGTACTTCGACACCACCAT
CGACCGGAAGCGGTACACCAGCACCAAGGAGGTGCTGGACGCCACCCTGATCCACCAGAGC
ATCACCGGCCTGTACGAGACCCGGATCGACCTGAGCCAGCTGGGCGGCGACAAGCGGCCCG
CCGCCACCAAGAAGGCCGGCCAGGCCAAGAAGAAGAAGGCCCGGGACAGCAAGGTGGAGAA
CAAGACCAAGAAGCTGCGGGTGTTCGAGGCCTTCGCCGGCATCGGCGCCCAGCGGAAGGCC
CTGGAGAAGGTGCGGAAGGACGAGTACGAGATCGTGGGCCTGGCCGAGTGGTACGTGCCCG
CCATCGTGATGTACCAGGCCATCCACAACAACTTCCACACCAAGCTGGAGTACAAGAGCGT
GAGCCGGGAGGAGATGATCGACTACCTGGAGAACAAGACCCTGAGCTGGAACAGCAAGAAC
CCCGTGAGCAACGGCTACTGGAAGCGGAAGAAGGACGACGAGCTGAAGATCATCTACAACG
CCATCAAGCTGAGCGAGAAGGAGGGCAACATCTTCGACATCCGGGACCTGTACAAGCGGAC
CCTGAAGAACATCGACCTGCTGACCTACAGCTTCCCCTGCCAGGACCTGAGCCAGCAGGGC
ATCCAGAAGGGCATGAAGCGGGGCAGCGGCACCCGGAGCGGCCTGCTGTGGGAGATCGAGC
GGGCCCTGGACAGCACCGAGAAGAACGACCTGCCCAAGTACCTGCTGATGGAGAACGTGGG
CGCCCTGCTGCACAAGAAGAACGAGGAGGAGCTGAACCAGTGGAAGCAGAAGCTGGAGAGC
CTGGGCTACCAGAACAGCATCGAGGTGCTGAACGCCGCCGACTTCGGCAGCAGCCAGGCCC
GGCGGCGGGTGTTCATGATCAGCACCCTGAACGAGTTCGTGGAGCTGCCCAAGGGCGACAA
GAAGCCCAAGAGCATCAAGAAGGTGCTGAACAAGATCGTGAGCGAGAAGGACATCCTGAAC
AACCTGCTGAAGTACAACCTGACCGAGTTCAAGAAAACCAAGAGCAACATCAACAAGGCCA
GCCTGATCGGCTACAGCAAGTTCAACAGCGAGGGCTACGTGTACGACCCCGAGTTCACCGG
CCCCACCCTGACCGCCAGCGGCGCCAACAGCCGGATCAAGATCAAGGACGGCAGCAACATC
CGGAAGATGAACAGCGACGAGACCTTCCTGTACATCGGCTTCGACAGCCAGGACGGCAAGC
GGGTGAACGAGATCGAGTTCCTGACCGAGAACCAGAAGATCTTCGTGTGCGGCAACAGCAT
CAGCGTGGAGGTGCTGGAGGCCATCATCGACAAGATCGGCGGCCCCAGCAGCGGCGGCAAG
CGGCCCGCCGCCACCAAGAAGGCCGGCCAGGCCAAGAAGAAGAAGGGCAGCTACCCCTACG
ACGTGCCCGACTACGCCTGAGCGGCCGCTTAATTAAGCTGCCTTCTGCGGGGCTTGCCTTC
TGGCCATGCCCTTCTTCTCTCCCTTGCACCTGTACCTCTTGGTCTTTGAATAAAGCCTGAG
TAGGAAGTCTAGAAAAAAAAAAAAAAAAAAAAAAAAAAAAAAAAAAAAAAAAAAAAAAAAA
AAAAAAAAAAAAAAAAAAAAAAAAAAAAAAA
Sa-dCas9-MQ1 (PL-27695) Plasmid DNA sequence:
(SEQ ID NO: 91)
TCGCGCGTTTCGGTGATGACGGTGAAAACCTCTGACACATGCAGCTCCCGGAGACGGTCAC
AGCTTGTCTGTAAGCGGATGCCGGGAGCAGACAAGCCCGTCAGGGCGCGTCAGCGGGTGTT
GGCGGGTGTCGGGGCTGGCTTAACTATGCGGCATCAGAGCAGATTGTACTGAGAGTGCACC
ATATGCGGTGTGAAATACCGCACAGATGCGTAAGGAGAAAATACCGCATCAGGCGCCATTC
GCCATTCAGGCTGCGCAACTGTTGGGAAGGGCGATCGGTGCGGGCCTCTTCGCTATTACGC
CAGCTGGCGAAAGGGGGATGTGCTGCAAGGCGATTAAGTTGGGTAACGCCAGGGTTTTCCC
AGTCACGACGTTGTAAAACGACGGCCAGTGAATTGACGCGTATTGGGATGGTACCTAATAC
GACTCACTATAAGGAAATAAGAGAGAAAAGAAGAGTAAGAAGAAATATAAGAGCCACCATG
GCCCCCAAGAAGAAGCGGAAGGTGGGCATCCACGGCGTGCCCGCCGCCGCCAAGCGGAACT
ACATCCTGGGCCTGGCCATCGGCATCACCAGCGTGGGCTACGGCATCATCGACTACGAGAC
CCGGGACGTGATCGACGCCGGCGTGCGGCTGTTCAAGGAGGCCAACGTGGAGAACAACGAG
GGCCGGCGGAGCAAGCGGGGCGCCCGGCGGCTGAAGCGGCGGCGGCGGCACCGGATCCAGC
GGGTGAAGAAGCTGCTGTTCGACTACAACCTGCTGACCGACCACAGCGAGCTGAGCGGCAT
CAACCCCTACGAGGCCCGGGTGAAGGGCCTGAGCCAGAAGCTGAGCGAGGAGGAGTTCAGC
GCCGCCCTGCTGCACCTGGCCAAGCGGCGGGGCGTGCACAACGTGAACGAGGTGGAGGAGG
ACACCGGCAACGAGCTGAGCACCAAGGAGCAGATCAGCCGGAACAGCAAGGCCCTGGAGGA
GAAGTACGTGGCCGAGCTGCAGCTGGAGCGGCTGAAGAAGGACGGCGAGGTGCGGGGCAGC
ATCAACCGGTTCAAGACCAGCGACTACGTGAAGGAGGCCAAGCAGCTGCTGAAGGTGCAGA
AGGCCTACCACCAGCTGGACCAGAGCTTCATCGACACCTACATCGACCTGCTGGAGACCCG
GCGGACCTACTACGAGGGCCCCGGCGAGGGCAGCCCCTTCGGCTGGAAGGACATCAAGGAG
TGGTACGAGATGCTGATGGGCCACTGCACCTACTTCCCCGAGGAGCTGCGGAGCGTGAAGT
ACGCCTACAACGCCGACCTGTACAACGCCCTGAACGACCTGAACAACCTGGTGATCACCCG
GGACGAGAACGAGAAGCTGGAGTACTACGAGAAGTTCCAGATCATCGAGAACGTGTTCAAG
CAGAAGAAGAAGCCCACCCTGAAGCAGATCGCCAAGGAGATCCTGGTGAACGAGGAGGACA
TCAAGGGCTACCGGGTGACCAGCACCGGCAAGCCCGAGTTCACCAACCTGAAGGTGTACCA
CGACATCAAGGACATCACCGCCCGGAAGGAGATCATCGAGAACGCCGAGCTGCTGGACCAG
ATCGCCAAGATCCTGACCATCTACCAGAGCAGCGAGGACATCCAGGAGGAGCTGACCAACC
TGAACAGCGAGCTGACCCAGGAGGAGATCGAGCAGATCAGCAACCTGAAGGGCTACACCGG
CACCCACAACCTGAGCCTGAAGGCCATCAACCTGATCCTGGACGAGCTGTGGCACACCAAC
GACAACCAGATCGCCATCTTCAACCGGCTGAAGCTGGTGCCCAAGAAGGTGGACCTGAGCC
AGCAGAAGGAGATCCCCACCACCCTGGTGGACGACTTCATCCTGAGCCCCGTGGTGAAGCG
GAGCTTCATCCAGAGCATCAAGGTGATCAACGCCATCATCAAGAAGTACGGCCTGCCCAAC
GACATCATCATCGAGCTGGCCCGGGAGAAGAACAGCAAGGACGCCCAGAAGATGATCAACG
AGATGCAGAAGCGGAACCGGCAGACCAACGAGCGGATCGAGGAGATCATCCGGACCACCGG
CAAGGAGAACGCCAAGTACCTGATCGAGAAGATCAAGCTGCACGACATGCAGGAGGGCAAG
TGCCTGTACAGCCTGGAGGCCATCCCCCTGGAGGACCTGCTGAACAACCCCTTCAACTACG
AGGTGGACGCCATCATCCCCCGGAGCGTGAGCTTCGACAACAGCTTCAACAACAAGGTGCT
GGTGAAGCAGGAGGAGAACAGCAAGAAGGGCAACCGGACCCCCTTCCAGTACCTGAGCAGC
AGCGACAGCAAGATCAGCTACGAGACCTTCAAGAAGCACATCCTGAACCTGGCCAAGGGCA
AGGGCCGGATCAGCAAGACCAAGAAGGAGTACCTGCTGGAGGAGCGGGACATCAACCGGTT
CAGCGTGCAGAAGGACTTCATCAACCGGAACCTGGTGGACACCCGGTACGCCACCCGGGGC
CTGATGAACCTGCTGCGGAGCTACTTCCGGGTGAACAACCTGGACGTGAAGGTGaaatccA
TCAACGGCGGCTTCACCAGCTTCCTGCGGCGGAAGTGGAAGTTCAAGAAGGAGCGGAACAA
GGGCTACAAGCACCACGCCGAGGACGCCCTGATCATCGCCAACGCCGACTTCATCTTCAAG
GAGTGGAAGAAGCTGGACAAGGCCAAGAAGGTGATGGAGAACCAGATGTTCGAGGAGAAGC
AGGCCGAGAGCATGCCCGAGATCGAGACCGAGCAGGAGTACAAGGAGATCTTCATCACCCC
CCACCAGATCAAGCACATCAAGGACTTCAAGGACTACAAGTACAGCCACCGGGTGGACAAG
AAGCCCAACCGGAAGCTGATCAACGACACCCTGTACAGCACCCGGAAGGACGACAAGGGCA
ACACCCTGATCGTGAACAACCTGAACGGCCTGTACGACAAGGACAACGACAAGCTGAAGAA
GCTGATCAACAAGAGCCCCGAGAAGCTGCTGATGTACCACCACGACCCCCAGACCTACCAG
AAGCTGAAGCTGATCATGGAGCAGTACGGCGACGAGAAGAACCCCCTGTACAAGTACTACG
AGGAGACCGGCAACTACCTGACCAAGTACAGCAAGAAGGACAACGGCCCCGTGATCAAGAA
GATCAAGTACTACGGCAACAAGCTGAACGCCCACCTGGACATCACCGACGACTACCCCAAC
AGCCGGAACAAGGTGGTGAAGCTGAGCCTGAAGCCCTACCGGTTCGACGTGTACCTGGACA
ACGGCGTGTACAAGTTCGTGACCGTGAAGAACCTGGACGTGATCAAGAAGGAGAACTACTA
CGAGGTGAACAGCAAGTGCTACGAGGAGGCCAAGAAGCTGAAGAAGATCAGCAACCAGGCC
GAGTTCATCGCCAGCTTCTACAAGAACGACCTGATCAAGATCAACGGCGAGCTGTACCGGG
TGATCGGCGTGAACAACGACCTGCTGAACCGGATCGAGGTGAACATGATCGACATCACCTA
CCGGGAGTACCTGGAGAACATGAACGACAAGCGGCCCCCCCACATCATCAAGACCATCGCC
AGCAAGACCCAGAGCATCAAGAAGTACAGCACCGACATCCTGGGCAACCTGTACGAGGTGa
aatccAAGAAGCACCCCCAGATCATCAAGAAGGGCAAGCGGCCCGCCGCCACCAAGAAGGC
CGGCCAGGCCAAGAAGAAGAAGGCCCGGGACAGCAAGGTGGAGAACAAGACCAAGAAGCTG
CGGGTGTTCGAGGCCTTCGCCGGCATCGGCGCCCAGCGGAAGGCCCTGGAGAAGGTGCGGA
AGGACGAGTACGAGATCGTGGGCCTGGCCGAGTGGTACGTGCCCGCCATCGTGATGTACCA
GGCCATCCACAACAACTTCCACACCAAGCTGGAGTACAAGAGCGTGAGCCGGGAGGAGATG
ATCGACTACCTGGAGAACAAGACCCTGAGCTGGAACAGCAAGAACCCCGTGAGCAACGGCT
ACTGGAAGCGGAAGAAGGACGACGAGCTGAAGATCATCTACAACGCCATCAAGCTGAGCGA
GAAGGAGGGCAACATCTTCGACATCCGGGACCTGTACAAGCGGACCCTGAAGAACATCGAC
CTGCTGACCTACAGCTTCCCCTGCCAGGACCTGAGCCAGCAGGGCATCCAGAAGGGCATGA
AGCGGGGCAGCGGCACCCGGAGCGGCCTGCTGTGGGAGATCGAGCGGGCCCTGGACAGCAC
CGAGAAGAACGACCTGCCCAAGTACCTGCTGATGGAGAACGTGGGCGCCCTGCTGCACAAG
AAGAACGAGGAGGAGCTGAACCAGTGGAAGCAGAAGCTGGAGAGCCTGGGCTACCAGAACA
GCATCGAGGTGCTGAACGCCGCCGACTTCGGCAGCAGCCAGGCCCGGCGGCGGGTGTTCAT
GATCAGCACCCTGAACGAGTTCGTGGAGCTGCCCAAGGGCGACAAGAAGCCCAAGAGCATC
AAGAAGGTGCTGAACAAGATCGTGAGCGAGAAGGACATCCTGAACAACCTGCTGAAGTACA
ACCTGACCGAGTTCAAGAAaACCAAGAGCAACATCAACAAGGCCAGCCTGATCGGCTACAG
CAAGTTCAACAGCGAGGGCTACGTGTACGACCCCGAGTTCACCGGCCCCACCCTGACCGCC
AGCGGCGCCAACAGCCGGATCAAGATCAAGGACGGCAGCAACATCCGGAAGATGAACAGCG
ACGAGACCTTCCTGTACATCGGCTTCGACAGCCAGGACGGCAAGCGGGTGAACGAGATCGA
GTTCCTGACCGAGAACCAGAAGATCTTCGTGTGCGGCAACAGCATCAGCGTGGAGGTGCTG
GAGGCCATCATCGACAAGATCGGCGGCCCCAGCAGCGGCGGCAAGCGGCCCGCCGCCACCA
AGAAGGCCGGCCAGGCCAAGAAGAAGAAGGGCAGCTACCCCTACGACGTGCCCGACTACGC
CTGAGcggccgcttaattaagctgccttctgcggggcttgccttctggccatgcccttctt
ctctcccttgcacctgtacctcttggtctttgaataaagcctgagtaggaagtctagaaaa
aaaaaaaaaaaaaaaaaaaaaaaaaaaaaaaaaaaaaaaaaaaaaaaaaaaaaaaaaaaaa
aaaaaaaaaaaaaaattgtcttcttcatcgcctgcagATCCCAATGGCGCGCCGAGCTTGG
CTCGAGCATGGTCATAGCTGTTTCCTGTGTGAAATTGTTATCCGCTCACAATTCCACACAA
CATACGAGCCGGAAGCATAAAGTGTAAAGCCTGGGGTGCCTAATGAGTGAGCTAACTCACA
TTAATTGCGTTGCGCTCACTGCCCGCTTTCCAGTCGGGAAACCTGTCGTGCCAGCTGCATT
AATGAATCGGCCAACGCGCGGGGAGAGGCGGTTTGCGTATTGGGCGCTCTTCCGCTTCCTC
GCTCACTGACTCGCTGCGCTCGGTCGTTCGGCTGCGGCGAGCGGTATCAGCTCACTCAAAG
GCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCATAG
CTCACGCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAAC
TTGGTCTGACAGTTAGAAAAACTCATCGAGCATCAAATGAAACTGCAATTTATTCATATCA
GGATTATCAATACCATATTTTTGAAAAAGCCGTTTCTGTAATGAAGGAGAAAACTCACCGA
GGCAGTTCCATAGGATGGCAAGATCCTGGTATCGGTCTGCGATTCCGACTCGTCCAACATC
AATACAACCTATTAATTTCCCCTCGTCAAAAATAAGGTTATCAAGTGAGAAATCACCATGA
GTGACGACTGAATCCGGTGAGAATGGCAAAAGTTTATGCATTTCTTTCCAGACTTGTTCAA
CAGGCCAGCCATTACGCTCGTCATCAAAATCACTCGCATCAACCAAACCGTTATTCATTCG
TGATTGCGCCTGAGCGAGACGAAATACGCGATCGCTGTTAAAAGGACAATTACAAACAGGA
ATCGAATGCAACCGGCGCAGGAACACTGCCAGCGCATCAACAATATTTTCACCTGAATCAG
GATATTCTTCTAATACCTGGAATGCTGTTTTCCCAGGGATCGCAGTGGTGAGTAACCATGC
ATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAA
ACAACTCTGGCGCATCGGGCTTCCCATACAATCGATAGATTGTCGCACCTGATTGCCCGAC
ATTATCGCGAGCCCATTTATACCCATATAAATCAGCATCCATGTTGGAATTTAATCGCGGC
CTAGAGCAAGACGTTTCCCGTTGAATATGGCTCATACTCTTCCTTTTTCAATATTATTGAA
GCATTTATCAGGGTTATTGTCTCATGAGCGGATACATATTTGAATGTATTTAGAAAAATAA
ACAAATAGGGGTTCCGCGCACATTTCCCCGAAAAGTGCCACCTGACGTCTAAGAAACCATT
ATTATCATGACATTAACCTATAAAAATAGGCGTATCACGAGGCCCTTTCGTC
Sa-dCas9-MQ1 (MR-28126) Expressed mRNA sequence:
(SEQ ID NO: 92)
AGGAAATAAGAGAGAAAAGAAGAGTAAGAAGAAATATAAGAGCCACCATGGCCCCCAAGAA
GAAGCGGAAGGTGGGCATCCACGGCGTGCCCGCCGCCGCCAAGCGGAACTACATCCTGGGC
CTGGCCATCGGCATCACCAGCGTGGGCTACGGCATCATCGACTACGAGACCCGGGACGTGA
TCGACGCCGGCGTGCGGCTGTTCAAGGAGGCCAACGTGGAGAACAACGAGGGCCGGCGGAG
CAAGCGGGGCGCCCGGCGGCTGAAGCGGCGGCGGCGGCACCGGATCCAGCGGGTGAAGAAG
CTGCTGTTCGACTACAACCTGCTGACCGACCACAGCGAGCTGAGCGGCATCAACCCCTACG
AGGCCCGGGTGAAGGGCCTGAGCCAGAAGCTGAGCGAGGAGGAGTTCAGCGCCGCCCTGCT
GCACCTGGCCAAGCGGCGGGGCGTGCACAACGTGAACGAGGTGGAGGAGGACACCGGCAAC
GAGCTGAGCACCAAGGAGCAGATCAGCCGGAACAGCAAGGCCCTGGAGGAGAAGTACGTGG
CCGAGCTGCAGCTGGAGCGGCTGAAGAAGGACGGCGAGGTGCGGGGCAGCATCAACCGGTT
CAAGACCAGCGACTACGTGAAGGAGGCCAAGCAGCTGCTGAAGGTGCAGAAGGCCTACCAC
CAGCTGGACCAGAGCTTCATCGACACCTACATCGACCTGCTGGAGACCCGGCGGACCTACT
ACGAGGGCCCCGGCGAGGGCAGCCCCTTCGGCTGGAAGGACATCAAGGAGTGGTACGAGAT
GCTGATGGGCCACTGCACCTACTTCCCCGAGGAGCTGCGGAGCGTGAAGTACGCCTACAAC
GCCGACCTGTACAACGCCCTGAACGACCTGAACAACCTGGTGATCACCCGGGACGAGAACG
AGAAGCTGGAGTACTACGAGAAGTTCCAGATCATCGAGAACGTGTTCAAGCAGAAGAAGAA
GCCCACCCTGAAGCAGATCGCCAAGGAGATCCTGGTGAACGAGGAGGACATCAAGGGCTAC
CGGGTGACCAGCACCGGCAAGCCCGAGTTCACCAACCTGAAGGTGTACCACGACATCAAGG
ACATCACCGCCCGGAAGGAGATCATCGAGAACGCCGAGCTGCTGGACCAGATCGCCAAGAT
CCTGACCATCTACCAGAGCAGCGAGGACATCCAGGAGGAGCTGACCAACCTGAACAGCGAG
CTGACCCAGGAGGAGATCGAGCAGATCAGCAACCTGAAGGGCTACACCGGCACCCACAACC
TGAGCCTGAAGGCCATCAACCTGATCCTGGACGAGCTGTGGCACACCAACGACAACCAGAT
CGCCATCTTCAACCGGCTGAAGCTGGTGCCCAAGAAGGTGGACCTGAGCCAGCAGAAGGAG
ATCCCCACCACCCTGGTGGACGACTTCATCCTGAGCCCCGTGGTGAAGCGGAGCTTCATCC
AGAGCATCAAGGTGATCAACGCCATCATCAAGAAGTACGGCCTGCCCAACGACATCATCAT
CGAGCTGGCCCGGGAGAAGAACAGCAAGGACGCCCAGAAGATGATCAACGAGATGCAGAAG
CGGAACCGGCAGACCAACGAGCGGATCGAGGAGATCATCCGGACCACCGGCAAGGAGAACG
CCAAGTACCTGATCGAGAAGATCAAGCTGCACGACATGCAGGAGGGCAAGTGCCTGTACAG
CCTGGAGGCCATCCCCCTGGAGGACCTGCTGAACAACCCCTTCAACTACGAGGTGGACGCC
ATCATCCCCCGGAGCGTGAGCTTCGACAACAGCTTCAACAACAAGGTGCTGGTGAAGCAGG
AGGAGAACAGCAAGAAGGGCAACCGGACCCCCTTCCAGTACCTGAGCAGCAGCGACAGCAA
GATCAGCTACGAGACCTTCAAGAAGCACATCCTGAACCTGGCCAAGGGCAAGGGCCGGATC
AGCAAGACCAAGAAGGAGTACCTGCTGGAGGAGCGGGACATCAACCGGTTCAGCGTGCAGA
AGGACTTCATCAACCGGAACCTGGTGGACACCCGGTACGCCACCCGGGGCCTGATGAACCT
GCTGCGGAGCTACTTCCGGGTGAACAACCTGGACGTGAAGGTGaaatccATCAACGGCGGC
TTCACCAGCTTCCTGCGGCGGAAGTGGAAGTTCAAGAAGGAGCGGAACAAGGGCTACAAGC
ACCACGCCGAGGACGCCCTGATCATCGCCAACGCCGACTTCATCTTCAAGGAGTGGAAGAA
GCTGGACAAGGCCAAGAAGGTGATGGAGAACCAGATGTTCGAGGAGAAGCAGGCCGAGAGC
ATGCCCGAGATCGAGACCGAGCAGGAGTACAAGGAGATCTTCATCACCCCCCACCAGATCA
AGCACATCAAGGACTTCAAGGACTACAAGTACAGCCACCGGGTGGACAAGAAGCCCAACCG
GAAGCTGATCAACGACACCCTGTACAGCACCCGGAAGGACGACAAGGGCAACACCCTGATC
GTGAACAACCTGAACGGCCTGTACGACAAGGACAACGACAAGCTGAAGAAGCTGATCAACA
AGAGCCCCGAGAAGCTGCTGATGTACCACCACGACCCCCAGACCTACCAGAAGCTGAAGCT
GATCATGGAGCAGTACGGCGACGAGAAGAACCCCCTGTACAAGTACTACGAGGAGACCGGC
AACTACCTGACCAAGTACAGCAAGAAGGACAACGGCCCCGTGATCAAGAAGATCAAGTACT
ACGGCAACAAGCTGAACGCCCACCTGGACATCACCGACGACTACCCCAACAGCCGGAACAA
GGTGGTGAAGCTGAGCCTGAAGCCCTACCGGTTCGACGTGTACCTGGACAACGGCGTGTAC
AAGTTCGTGACCGTGAAGAACCTGGACGTGATCAAGAAGGAGAACTACTACGAGGTGAACA
GCAAGTGCTACGAGGAGGCCAAGAAGCTGAAGAAGATCAGCAACCAGGCCGAGTTCATCGC
CAGCTTCTACAAGAACGACCTGATCAAGATCAACGGCGAGCTGTACCGGGTGATCGGCGTG
AACAACGACCTGCTGAACCGGATCGAGGTGAACATGATCGACATCACCTACCGGGAGTACC
TGGAGAACATGAACGACAAGCGGCCCCCCCACATCATCAAGACCATCGCCAGCAAGACCCA
GAGCATCAAGAAGTACAGCACCGACATCCTGGGCAACCTGTACGAGGTGaaatccAAGAAG
CACCCCCAGATCATCAAGAAGGGCAAGCGGCCCGCCGCCACCAAGAAGGCCGGCCAGGCCA
AGAAGAAGAAGGCCCGGGACAGCAAGGTGGAGAACAAGACCAAGAAGCTGCGGGTGTTCGA
GGCCTTCGCCGGCATCGGCGCCCAGCGGAAGGCCCTGGAGAAGGTGCGGAAGGACGAGTAC
GAGATCGTGGGCCTGGCCGAGTGGTACGTGCCCGCCATCGTGATGTACCAGGCCATCCACA
ACAACTTCCACACCAAGCTGGAGTACAAGAGCGTGAGCCGGGAGGAGATGATCGACTACCT
GGAGAACAAGACCCTGAGCTGGAACAGCAAGAACCCCGTGAGCAACGGCTACTGGAAGCGG
AAGAAGGACGACGAGCTGAAGATCATCTACAACGCCATCAAGCTGAGCGAGAAGGAGGGCA
ACATCTTCGACATCCGGGACCTGTACAAGCGGACCCTGAAGAACATCGACCTGCTGACCTA
CAGCTTCCCCTGCCAGGACCTGAGCCAGCAGGGCATCCAGAAGGGCATGAAGCGGGGCAGC
GGCACCCGGAGCGGCCTGCTGTGGGAGATCGAGCGGGCCCTGGACAGCACCGAGAAGAACG
ACCTGCCCAAGTACCTGCTGATGGAGAACGTGGGCGCCCTGCTGCACAAGAAGAACGAGGA
GGAGCTGAACCAGTGGAAGCAGAAGCTGGAGAGCCTGGGCTACCAGAACAGCATCGAGGTG
CTGAACGCCGCCGACTTCGGCAGCAGCCAGGCCCGGCGGCGGGTGTTCATGATCAGCACCC
TGAACGAGTTCGTGGAGCTGCCCAAGGGCGACAAGAAGCCCAAGAGCATCAAGAAGGTGCT
GAACAAGATCGTGAGCGAGAAGGACATCCTGAACAACCTGCTGAAGTACAACCTGACCGAG
TTCAAGAAaACCAAGAGCAACATCAACAAGGCCAGCCTGATCGGCTACAGCAAGTTCAACA
GCGAGGGCTACGTGTACGACCCCGAGTTCACCGGCCCCACCCTGACCGCCAGCGGCGCCAA
CAGCCGGATCAAGATCAAGGACGGCAGCAACATCCGGAAGATGAACAGCGACGAGACCTTC
CTGTACATCGGCTTCGACAGCCAGGACGGCAAGCGGGTGAACGAGATCGAGTTCCTGACCG
AGAACCAGAAGATCTTCGTGTGCGGCAACAGCATCAGCGTGGAGGTGCTGGAGGCCATCAT
CGACAAGATCGGCGGCCCCAGCAGCGGCGGCAAGCGGCCCGCCGCCACCAAGAAGGCCGGC
CAGGCCAAGAAGAAGAAGGGCAGCTACCCCTACGACGTGCCCGACTACGCCTGAGggccgc
ttaattaagctgccttctgcggggcttgccttctggccatgcccttcttctctcccttgca
cctgtacctcttggtctttgaataaagcctgagtaggaagtctagaaaaaaaaaaaaaaaa
aaaaaaaaaaaaaaaaaaaaaaaaaaaaaaaaaaaaaaaaaaaaaaaaaaaaaaaaaaaaa
aaa

In some embodiments, an expression repressor comprises the amino acid sequence of SEQ ID NOs: 93, 33, 67, or 68. In some embodiments, an expression repressor described herein comprises an amino acid sequence of SEQ ID NO: 93, 33, 67, 68, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Sa-dCas9-MQ1 Protein sequence:
(SEQ ID NO: 93)
MAPKKKRKVGIHGVPAAAKRNYILGLAIGITSVGYGIIDYETRDVIDAGVRLFKEANVENNEGRR
SKRGARRLKRRRRHRIQRVKKLLFDYNLLTDHSELSGINPYEARVKGLSQKLSEEEFSAALLHLA
KRRGVHNVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGHCTYFPEE
LRSVKYAYNADLYNALNDLNNLVITRDENEKLEYYEKFQIIENVFKQKKKPTLKQIAKEILVNEE
DIKGYRVTSTGKPEFTNLKVYHDIKDITARKEIIENAELLDQIAKILTIYQSSEDIQEELTNLNS
ELTQEEIEQISNLKGYTGTHNLSLKAINLILDELWHTNDNQIAIFNRLKLVPKKVDLSQQKEIPT
TLVDDFILSPVVKRSFIQSIKVINAIIKKYGLPNDIIIELAREKNSKDAQKMINEMQKRNRQTNE
RIEEIIRTTGKENAKYLIEKIKLHDMQEGKCLYSLEAIPLEDLLNNPFNYEVDAIIPRSVSFDNS
FNNKVLVKQEENSKKGNRTPFQYLSSSDSKISYETFKKHILNLAKGKGRISKTKKEYLLEERDIN
RFSVQKDFINRNLVDTRYATRGLMNLLRSYFRVNNLDVKVKSINGGFTSFLRRKWKFKKERNKGY
KHHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSPEKLL
MYHHDPQTYQKLKLIMEQYGDEKNPLYKYYEETGNYLTKYSKKDNGPVIKKIKYYGNKLNAHLDI
TDDYPNSRNKVVKLSLKPYRFDVYLDNGVYKFVTVKNLDVIKKENYYEVNSKCYEEAKKLKKISN
QAEFIASFYKNDLIKINGELYRVIGVNNDLLNRIEVNMIDITYREYLENMNDKRPPHIIKTIASK
TQSIKKYSTDILGNLYEVKSKKHPQIIKKGKRPAATKKAGQAKKKKARDSKVENKTKKLRVFEAF
AGIGAQRKALEKVRKDEYEIVGLAEWYVPAIVMYQAIHNNFHTKLEYKSVSREEMIDYLENKTLS
WNSKNPVSNGYWKRKKDDELKIIYNAIKLSEKEGNIFDIRDLYKRTLKNIDLLTYSFPCQDLSQQ
GIQKGMKRGSGTRSGLLWEIERALDSTEKNDLPKYLLMENVGALLHKKNEEELNQWKQKLESLGY
QNSIEVLNAADFGSSQARRRVFMISTLNEFVELPKGDKKPKSIKKVLNKIVSEKDILNNLLKYNL
TEFKKTKSNINKASLIGYSKFNSEGYVYDPEFTGPTLTASGANSRIKIKDGSNIRKMNSDETFLY
IGFDSQDGKRVNEIEFLTENQKIFVCGNSISVEVLEAIIDKIGGPSSGGKRPAATKKAGQAKKKK
GSYPYDVPDYA
dCas9-MQ1 amino acid sequence
(SEQ ID NO: 33)
MAPKKKRKVGIHGVPAADKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQELD
INRLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIH
QSITGLYETRIDLSQLGGDKRPAATKKAGQAKKKKARDSKVENKTKKLRVFEAFAGIGAQRKALE
KVRKDEYEIVGLAEWYVPAIVMYQAIHNNFHTKLEYKSVSREEMIDYLENKTLSWNSKNPVSNGY
WKRKKDDELKIIYNAIKLSEKEGNIFDIRDLYKRTLKNIDLLTYSFPCQDLSQQGIQKGMKRGSG
TRSGLLWEIERALDSTEKNDLPKYLLMENVGALLHKKNEEELNQWKQKLESLGYQNSIEVLNAAD
FGSSQARRRVFMISTLNEFVELPKGDKKPKSIKKVLNKIVSEKDILNNLLKYNLTEFKKTKSNIN
KASLIGYSKFNSEGYVYDPEFTGPTLTASGANSRIKIKDGSNIRKMNSDETFLYIGFDSQDGKRV
NEIEFLTENQKIFVCGNSISVEVLEAIIDKIGGPSSGGKRPAATKKAGQAKKKKGSYPYDVPDYA
Sa-dCas9-MQ1 without HA tag
(SEQ ID NO: 67)
MAPKKKRKVGIHGVPAAAKRNYILGLAIGITSVGYGIIDYETRDVIDAGVRLFKEANVENNEGRR
SKRGARRLKRRRRHRIQRVKKLLFDYNLLTDHSELSGINPYEARVKGLSQKLSEEEFSAALLHLA
KRRGVHNVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGHCTYFPEE
LRSVKYAYNADLYNALNDLNNLVITRDENEKLEYYEKFQIIENVFKQKKKPTLKQIAKEILVNEE
DIKGYRVTSTGKPEFTNLKVYHDIKDITARKEIIENAELLDQIAKILTIYQSSEDIQEELTNLNS
ELTQEEIEQISNLKGYTGTHNLSLKAINLILDELWHTNDNQIAIFNRLKLVPKKVDLSQQKEIPT
TLVDDFILSPVVKRSFIQSIKVINAIIKKYGLPNDIIIELAREKNSKDAQKMINEMQKRNRQTNE
RIEEIIRTTGKENAKYLIEKIKLHDMQEGKCLYSLEAIPLEDLLNNPFNYEVDAIIPRSVSFDNS
FNNKVLVKQEENSKKGNRTPFQYLSSSDSKISYETFKKHILNLAKGKGRISKTKKEYLLEERDIN
RFSVQKDFINRNLVDTRYATRGLMNLLRSYFRVNNLDVKVKSINGGFTSFLRRKWKFKKERNKGY
KHHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSPEKLL
MYHHDPQTYQKLKLIMEQYGDEKNPLYKYYEETGNYLTKYSKKDNGPVIKKIKYYGNKLNAHLDI
TDDYPNSRNKVVKLSLKPYRFDVYLDNGVYKFVTVKNLDVIKKENYYEVNSKCYEEAKKLKKISN
QAEFIASFYKNDLIKINGELYRVIGVNNDLLNRIEVNMIDITYREYLENMNDKRPPHIIKTIASK
TQSIKKYSTDILGNLYEVKSKKHPQIIKKGKRPAATKKAGQAKKKKARDSKVENKTKKLRVFEAF
AGIGAQRKALEKVRKDEYEIVGLAEWYVPAIVMYQAIHNNFHTKLEYKSVSREEMIDYLENKTLS
WNSKNPVSNGYWKRKKDDELKIIYNAIKLSEKEGNIFDIRDLYKRTLKNIDLLTYSFPCQDLSQQ
GIQKGMKRGSGTRSGLLWEIERALDSTEKNDLPKYLLMENVGALLHKKNEEELNQWKQKLESLGY
QNSIEVLNAADFGSSQARRRVFMISTLNEFVELPKGDKKPKSIKKVLNKIVSEKDILNNLLKYNL
TEFKKTKSNINKASLIGYSKFNSEGYVYDPEFTGPTLTASGANSRIKIKDGSNIRKMNSDETFLY
IGFDSQDGKRVNEIEFLTENQKIFVCGNSISVEVLEAIIDKIGGPSSGGKRPAATKKAGQAKKKK
GS
dCas9-MQ1 without HA tag
(SEQ ID NO: 68)
MAPKKKRKVGIHGVPAADKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIAL
SLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLYYLQNGRDMYVDQELD
INRLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIH
QSITGLYETRIDLSQLGGDKRPAATKKAGQAKKKKARDSKVENKTKKLRVFEAFAGIGAQRKALE
KVRKDEYEIVGLAEWYVPAIVMYQAIHNNFHTKLEYKSVSREEMIDYLENKTLSWNSKNPVSNGY
WKRKKDDELKIIYNAIKLSEKEGNIFDIRDLYKRTLKNIDLLTYSFPCQDLSQQGIQKGMKRGSG
TRSGLLWEIERALDSTEKNDLPKYLLMENVGALLHKKNEEELNQWKQKLESLGYQNSIEVLNAAD
FGSSQARRRVFMISTLNEFVELPKGDKKPKSIKKVLNKIVSEKDILNNLLKYNLTEFKKTKSNIN
KASLIGYSKFNSEGYVYDPEFTGPTLTASGANSRIKIKDGSNIRKMNSDETFLYIGFDSQDGKRV
NEIEFLTENQKIFVCGNSISVEVLEAIIDKIGGPSSGGKRPAATKKAGQAKKKKGS

In some embodiments, an expression repressor comprises a DNA-targeting moiety comprising dCas9, e.g., an S. pyogenes dCas9, and a repressor domain comprising KRAB, e.g., a KRAB domain. In some embodiments, the expression repressor is encoded by the nucleic acid sequence of SEQ ID NOs: 94 (e.g., a plasmid encoding the expression repressor) and/or 95 (e.g., a nucleic acid (e.g., cDNA) encoding the expression repressor). In some embodiments, a nucleic acid described herein comprises a nucleic acid sequence of SEQ ID NO: 94 or 95 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Sp-dCas9-KRAB (PL-27687) Plasmid DNA sequence:
(SEQ ID NO: 94)
TCGCGCGTTTCGGTGATGACGGTGAAAACCTCTGACACATGCAGCTCCCGGAGACGGTCACA
GCTTGTCTGTAAGCGGATGCCGGGAGCAGACAAGCCCGTCAGGGCGCGTCAGCGGGTGTTGG
CGGGTGTCGGGGCTGGCTTAACTATGCGGCATCAGAGCAGATTGTACTGAGAGTGCACCATA
TGCGGTGTGAAATACCGCACAGATGCGTAAGGAGAAAATACCGCATCAGGCGCCATTCGCCA
TTCAGGCTGCGCAACTGTTGGGAAGGGCGATCGGTGCGGGCCTCTTCGCTATTACGCCAGCT
GGCGAAAGGGGGATGTGCTGCAAGGCGATTAAGTTGGGTAACGCCAGGGTTTTCCCAGTCAC
GACGTTGTAAAACGACGGCCAGTGAATTGACGCGTATTGGGATGGTACCTAATACGACTCAC
TATAAGGAAATAAGAGAGAAAAGAAGAGTAAGAAGAAATATAAGAGCCACCATGGCCCCCAA
GAAGAAGCGGAAGGTGGGCATCCACGGCGTGCCCGCCGCCGACAAGAAGTACAGCATCGGCC
TGGCCATCGGCACCAACAGCGTGGGCTGGGCCGTGATCACCGACGAGTACAAGGTGCCCAGC
AAGAAGTTCAAGGTGCTGGGCAACACCGACCGGCACAGCATCAAGAAGAACCTGATCGGCGC
CCTGCTGTTCGACAGCGGCGAGACCGCCGAGGCCACCCGGCTGAAGCGGACCGCCCGGCGGC
GGTACACCCGGCGGAAGAACCGGATCTGCTACCTGCAGGAGATCTTCAGCAACGAGATGGCC
AAGGTGGACGACAGCTTCTTCCACCGGCTGGAGGAGAGCTTCCTGGTGGAGGAGGACAAGAA
GCACGAGCGGCACCCCATCTTCGGCAACATCGTGGACGAGGTGGCCTACCACGAGAAGTACC
CCACCATCTACCACCTGCGGAAGAAGCTGGTGGACAGCACCGACAAGGCCGACCTGCGGCTG
ATCTACCTGGCCCTGGCCCACATGATCAAGTTCCGGGGCCACTTCCTGATCGAGGGCGACCT
GAACCCCGACAACAGCGACGTGGACAAGCTGTTCATCCAGCTGGTGCAGACCTACAACCAGC
TGTTCGAGGAGAACCCCATCAACGCCAGCGGCGTGGACGCCAAGGCCATCCTGAGCGCCCGG
CTGAGCAAGAGCCGGCGGCTGGAGAACCTGATCGCCCAGCTGCCCGGCGAGAAGAAGAACGG
CCTGTTCGGCAACCTGATCGCCCTGAGCCTGGGCCTGACCCCCAACTTCAAGAGCAACTTCG
ACCTGGCCGAGGACGCCAAGCTGCAGCTGAGCAAGGACACCTACGACGACGACCTGGACAAC
CTGCTGGCCCAGATCGGCGACCAGTACGCCGACCTGTTCCTGGCCGCCAAGAACCTGAGCGA
CGCCATCCTGCTGAGCGACATCCTGCGGGTGAACACCGAGATCACCAAGGCCCCCCTGAGCG
CCAGCATGATCAAGCGGTACGACGAGCACCACCAGGACCTGACCCTGCTGAAGGCCCTGGTG
CGGCAGCAGCTGCCCGAGAAGTACAAGGAGATCTTCTTCGACCAGAGCAAGAACGGCTACGC
CGGCTACATCGACGGCGGCGCCAGCCAGGAGGAGTTCTACAAGTTCATCAAGCCCATCCTGG
AGAAGATGGACGGCACCGAGGAGCTGCTGGTGAAGCTGAACCGGGAGGACCTGCTGCGGAAG
CAGCGGACCTTCGACAACGGCAGCATCCCCCACCAGATCCACCTGGGCGAGCTGCACGCCAT
CCTGCGGCGGCAGGAGGACTTCTACCCCTTCCTGAAGGACAACCGGGAGAAGATCGAGAAGA
TCCTGACCTTCCGGATCCCCTACTACGTGGGCCCCCTGGCCCGGGGCAACAGCCGGTTCGCC
TGGATGACCCGGaaatccGAGGAGACCATCACCCCCTGGAACTTCGAGGAGGTGGTGGACAA
GGGCGCCAGCGCCCAGAGCTTCATCGAGCGGATGACCAACTTCGACAAGAACCTGCCCAACG
AGAAGGTGCTGCCCAAGCACAGCCTGCTGTACGAGTACTTCACCGTGTACAACGAGCTGACC
AAGGTGAAGTACGTGACCGAGGGCATGCGGAAGCCCGCCTTCCTGAGCGGCGAGCAGAAGAA
GGCCATCGTGGACCTGCTGTTCAAGACCAACCGGAAGGTGACCGTGAAGCAGCTGAAGGAGG
ACTACTTCAAGAAGATCGAGTGCTTCGACAGCGTGGAGATCAGCGGCGTGGAGGACCGGTTC
AACGCCAGCCTGGGCACCTACCACGACCTGCTGAAGATCATCAAGGACAAGGACTTCCTGGA
CAACGAGGAGAACGAGGACATCCTGGAGGACATCGTGCTGACCCTGACCCTGTTCGAGGACC
GGGAGATGATCGAGGAGCGGCTGAAaACCTACGCCCACCTGTTCGACGACAAGGTGATGAAG
CAGCTGAAGCGGCGGCGGTACACCGGCTGGGGCCGGCTGAGCCGGAAGCTGATCAACGGCAT
CCGGGACAAGCAGAGCGGCAAGACCATCCTGGACTTCCTGaaatccGACGGCTTCGCCAACC
GGAACTTCATGCAGCTGATCCACGACGACAGCCTGACCTTCAAGGAGGACATCCAGAAGGCC
CAGGTGAGCGGCCAGGGCGACAGCCTGCACGAGCACATCGCCAACCTGGCCGGCAGCCCCGC
CATCAAGAAGGGCATCCTGCAGACCGTGAAGGTGGTGGACGAGCTGGTGAAGGTGATGGGCC
GGCACAAGCCCGAGAACATCGTGATCGAGATGGCCCGGGAGAACCAGACCACCCAGAAGGGC
CAGAAGAACAGCCGGGAGCGGATGAAGCGGATCGAGGAGGGCATCAAGGAGCTGGGCAGCCA
GATCCTGAAGGAGCACCCCGTGGAGAACACCCAGCTGCAGAACGAGAAGCTGTACCTGTACT
ACCTGCAGAACGGCCGGGACATGTACGTGGACCAGGAGCTGGACATCAACCGGCTGAGCGAC
TACGACGTGGCCGCCATCGTGCCCCAGAGCTTCCTGAAGGACGACAGCATCGACAACAAGGT
GCTGACCCGGAGCGACAAGGCCCGGGGCAAGAGCGACAACGTGCCCAGCGAGGAGGTGGTGA
AGAAGATGAAGAACTACTGGCGGCAGCTGCTGAACGCCAAGCTGATCACCCAGCGGAAGTTC
GACAACCTGACCAAGGCCGAGCGGGGCGGCCTGAGCGAGCTGGACAAGGCCGGCTTCATCAA
GCGGCAGCTGGTGGAGACCCGGCAGATCACCAAGCACGTGGCCCAGATCCTGGACAGCCGGA
TGAACACCAAGTACGACGAGAACGACAAGCTGATCCGGGAGGTGAAGGTGATCACCCTGaaa
tccAAGCTGGTGAGCGACTTCCGGAAGGACTTCCAGTTCTACAAGGTGCGGGAGATCAACAA
CTACCACCACGCCCACGACGCCTACCTGAACGCCGTGGTGGGCACCGCCCTGATCAAGAAGT
ACCCCAAGCTGGAGAGCGAGTTCGTGTACGGCGACTACAAGGTGTACGACGTGCGGAAGATG
ATCGCCAAGAGCGAGCAGGAGATCGGCAAGGCCACCGCCAAGTACTTCTTCTACAGCAACAT
CATGAACTTCTTCAAGACCGAGATCACCCTGGCCAACGGCGAGATCCGGAAGCGGCCCCTGA
TCGAGACCAACGGCGAGACCGGCGAGATCGTGTGGGACAAGGGCCGGGACTTCGCCACCGTG
CGGAAGGTGCTGAGCATGCCCCAGGTGAACATCGTGAAGAAaACCGAGGTGCAGACCGGCGG
CTTCAGCAAGGAGAGCATCCTGCCCAAGCGGAACAGCGACAAGCTGATCGCCCGGAAGAAGG
ACTGGGACCCCAAGAAGTACGGCGGCTTCGACAGCCCCACCGTGGCCTACAGCGTGCTGGTG
GTGGCCAAGGTGGAGAAGGGCAAGAGCAAGAAGCTGaaatccGTGAAGGAGCTGCTGGGCAT
CACCATCATGGAGCGGAGCAGCTTCGAGAAGAACCCCATCGACTTCCTGGAGGCCAAGGGCT
ACAAGGAGGTGAAGAAGGACCTGATCATCAAGCTGCCCAAGTACAGCCTGTTCGAGCTGGAG
AACGGCCGGAAGCGGATGCTGGCCAGCGCCGGCGAGCTGCAGAAGGGCAACGAGCTGGCCCT
GCCCAGCAAGTACGTGAACTTCCTGTACCTGGCCAGCCACTACGAGAAGCTGAAGGGCAGCC
CCGAGGACAACGAGCAGAAGCAGCTGTTCGTGGAGCAGCACAAGCACTACCTGGACGAGATC
ATCGAGCAGATCAGCGAGTTCAGCAAGCGGGTGATCCTGGCCGACGCCAACCTGGACAAGGT
GCTGAGCGCCTACAACAAGCACCGGGACAAGCCCATCCGGGAGCAGGCCGAGAACATCATCC
ACCTGTTCACCCTGACCAACCTGGGCGCCCCCGCCGCCTTCAAGTACTTCGACACCACCATC
GACCGGAAGCGGTACACCAGCACCAAGGAGGTGCTGGACGCCACCCTGATCCACCAGAGCAT
CACCGGCCTGTACGAGACCCGGATCGACCTGAGCCAGCTGGGCGGCGACAAGCGGCCCGCCG
CCACCAAGAAGGCCGGCCAGGCCAAGAAGAAGAAGGCCAGCGACGCCAAGAGCCTGACCGCC
TGGAGCCGGACCCTGGTGACCTTCAAGGACGTGTTCGTGGACTTCACCCGGGAGGAGTGGAA
GCTGCTGGACACCGCCCAGCAGATCCTGTACCGGAACGTGATGCTGGAGAACTACAAGAACC
TGGTGAGCCTGGGCTACCAGCTGACCAAGCCCGACGTGATCCTGCGGCTGGAGAAGGGCGAG
GAGCCCTGGCTGGTGGAGCGGGAGATCCACCAGGAGACCCACCCCGACAGCGAGACCGCCTT
CGAGATCAAGAGCAGCGTGAGCGGCGGCAAGCGGCCCGCCGCCACCAAGAAGGCCGGCCAGG
CCAAGAAGAAGAAGGGCAGCTACCCCTACGACGTGCCCGACTACGCCTGAGcggccgcttaa
ttaagctgccttctgcggggcttgccttctggccatgcccttcttctctcccttgcacctgt
acctcttggtctttgaataaagcctgagtaggaagtctagaaaaaaaaaaaaaaaaaaaaaa
aaaaaaaaaaaaaaaaaaaaaaaaaaaaaaaaaaaaaaaaaaaaaaaaaaaaaaaaaattgt
cttcttcatcgcctgcagATCCCAATGGCGCGCCGAGCTTGGCTCGAGCATGGTCATAGCTG
TTTCCTGTGTGAAATTGTTATCCGCTCACAATTCCACACAACATACGAGCCGGAAGCATAAA
GTGTAAAGCCTGGGGTGCCTAATGAGTGAGCTAACTCACATTAATTGCGTTGCGCTCACTGC
CCGCTTTCCAGTCGGGAAACCTGTCGTGCCAGCTGCATTAATGAATCGGCCAACGCGCGGGG
AGAGGCGGTTTGCGTATTGGGCGCTCTTCCGCTTCCTCGCTCACTGACTCGCTGCGCTCGGT
CGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTGCGCCT
TATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTG
GTGGCCTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTTTTAAATCA
ATCTAAAGTATATATGAGTAAACTTGGTCTGACAGTTAGAAAAACTCATCGAGCATCAAATG
AAACTGCAATTTATTCATATCAGGATTATCAATACCATATTTTTGAAAAAGCCGTTTCTGTA
ATGAAGGAGAAAACTCACCGAGGCAGTTCCATAGGATGGCAAGATCCTGGTATCGGTCTGCG
ATTCCGACTCGTCCAACATCAATACAACCTATTAATTTCCCCTCGTCAAAAATAAGGTTATC
AAGTGAGAAATCACCATGAGTGACGACTGAATCCGGTGAGAATGGCAAAAGTTTATGCATTT
CTTTCCAGACTTGTTCAACAGGCCAGCCATTACGCTCGTCATCAAAATCACTCGCATCAACC
AAACCGTTATTCATTCGTGATTGCGCCTGAGCGAGACGAAATACGCGATCGCTGTTAAAAGG
ACAATTACAAACAGGAATCGAATGCAACCGGCGCAGGAACACTGCCAGCGCATCAACAATAT
TTTCACCTGAATCAGGATATTCTTCTAATACCTGGAATGCTGTTTTCCCAGGGATCGCAGTG
GTGAGTAACCATGCATCATCAGGAGTACGGATAAAATGCTTGATGGTCGGAAGAGGCATAAA
TTCCGTCAGCCAGTTTAGTCTGACCATCTCATCTGTAACATCATTGGCAACGCTACCTTTGC
CATGTTTCAGAAACAACTCTGGCGCATCGGGCTTCCCATACAATCGATAGATTGTCGCACCT
GATTGCCCGACATTATCGCGAGCCCATTTATACCCATATAAATCAGCATCCATGTTGGAATT
TAATCGCGGCCTAGAGCAAGACGTTTCCCGTTGAATATGGCTCATACTCTTCCTTTTTCAAT
ATTATTGAAGCATTTATCAGGGTTATTGTCTCATGAGCGGATACATATTTGAATGTATTTAG
AAAAATAAACAAATAGGGGTTCCGCGCACATTTCCCCGAAAAGTGCCACCTGACGTCTAAGA
AACCATTATTATCATGACATTAACCTATAAAAATAGGCGTATCACGAGGCCCTTTCGTC
Sp-dCas9-KRAB (MR-28122) Expressed mRNA sequence:
(SEQ ID NO: 95)
AGGAAATAAGAGAGAAAAGAAGAGTAAGAAGAAATATAAGAGCCACCATGGCCCCCAAGAAG
AAGCGGAAGGTGGGCATCCACGGCGTGCCCGCCGCCGACAAGAAGTACAGCATCGGCCTGGC
CATCGGCACCAACAGCGTGGGCTGGGCCGTGATCACCGACGAGTACAAGGTGCCCAGCAAGA
AGTTCAAGGTGCTGGGCAACACCGACCGGCACAGCATCAAGAAGAACCTGATCGGCGCCCTG
CTGTTCGACAGCGGCGAGACCGCCGAGGCCACCCGGCTGAAGCGGACCGCCCGGCGGCGGTA
CACCCGGCGGAAGAACCGGATCTGCTACCTGCAGGAGATCTTCAGCAACGAGATGGCCAAGG
TGGACGACAGCTTCTTCCACCGGCTGGAGGAGAGCTTCCTGGTGGAGGAGGACAAGAAGCAC
GAGCGGCACCCCATCTTCGGCAACATCGTGGACGAGGTGGCCTACCACGAGAAGTACCCCAC
CATCTACCACCTGCGGAAGAAGCTGGTGGACAGCACCGACAAGGCCGACCTGCGGCTGATCT
ACCTGGCCCTGGCCCACATGATCAAGTTCCGGGGCCACTTCCTGATCGAGGGCGACCTGAAC
CCCGACAACAGCGACGTGGACAAGCTGTTCATCCAGCTGGTGCAGACCTACAACCAGCTGTT
CGAGGAGAACCCCATCAACGCCAGCGGCGTGGACGCCAAGGCCATCCTGAGCGCCCGGCTGA
GCAAGAGCCGGCGGCTGGAGAACCTGATCGCCCAGCTGCCCGGCGAGAAGAAGAACGGCCTG
TTCGGCAACCTGATCGCCCTGAGCCTGGGCCTGACCCCCAACTTCAAGAGCAACTTCGACCT
GGCCGAGGACGCCAAGCTGCAGCTGAGCAAGGACACCTACGACGACGACCTGGACAACCTGC
TGGCCCAGATCGGCGACCAGTACGCCGACCTGTTCCTGGCCGCCAAGAACCTGAGCGACGCC
ATCCTGCTGAGCGACATCCTGCGGGTGAACACCGAGATCACCAAGGCCCCCCTGAGCGCCAG
CATGATCAAGCGGTACGACGAGCACCACCAGGACCTGACCCTGCTGAAGGCCCTGGTGCGGC
AGCAGCTGCCCGAGAAGTACAAGGAGATCTTCTTCGACCAGAGCAAGAACGGCTACGCCGGC
TACATCGACGGCGGCGCCAGCCAGGAGGAGTTCTACAAGTTCATCAAGCCCATCCTGGAGAA
GATGGACGGCACCGAGGAGCTGCTGGTGAAGCTGAACCGGGAGGACCTGCTGCGGAAGCAGC
GGACCTTCGACAACGGCAGCATCCCCCACCAGATCCACCTGGGCGAGCTGCACGCCATCCTG
CGGCGGCAGGAGGACTTCTACCCCTTCCTGAAGGACAACCGGGAGAAGATCGAGAAGATCCT
GACCTTCCGGATCCCCTACTACGTGGGCCCCCTGGCCCGGGGCAACAGCCGGTTCGCCTGGA
TGACCCGGaaatccGAGGAGACCATCACCCCCTGGAACTTCGAGGAGGTGGTGGACAAGGGC
GCCAGCGCCCAGAGCTTCATCGAGCGGATGACCAACTTCGACAAGAACCTGCCCAACGAGAA
GGTGCTGCCCAAGCACAGCCTGCTGTACGAGTACTTCACCGTGTACAACGAGCTGACCAAGG
TGAAGTACGTGACCGAGGGCATGCGGAAGCCCGCCTTCCTGAGCGGCGAGCAGAAGAAGGCC
ATCGTGGACCTGCTGTTCAAGACCAACCGGAAGGTGACCGTGAAGCAGCTGAAGGAGGACTA
CTTCAAGAAGATCGAGTGCTTCGACAGCGTGGAGATCAGCGGCGTGGAGGACCGGTTCAACG
CCAGCCTGGGCACCTACCACGACCTGCTGAAGATCATCAAGGACAAGGACTTCCTGGACAAC
GAGGAGAACGAGGACATCCTGGAGGACATCGTGCTGACCCTGACCCTGTTCGAGGACCGGGA
GATGATCGAGGAGCGGCTGAAaACCTACGCCCACCTGTTCGACGACAAGGTGATGAAGCAGC
TGAAGCGGCGGCGGTACACCGGCTGGGGCCGGCTGAGCCGGAAGCTGATCAACGGCATCCGG
GACAAGCAGAGCGGCAAGACCATCCTGGACTTCCTGaaatccGACGGCTTCGCCAACCGGAA
CTTCATGCAGCTGATCCACGACGACAGCCTGACCTTCAAGGAGGACATCCAGAAGGCCCAGG
TGAGCGGCCAGGGCGACAGCCTGCACGAGCACATCGCCAACCTGGCCGGCAGCCCCGCCATC
AAGAAGGGCATCCTGCAGACCGTGAAGGTGGTGGACGAGCTGGTGAAGGTGATGGGCCGGCA
CAAGCCCGAGAACATCGTGATCGAGATGGCCCGGGAGAACCAGACCACCCAGAAGGGCCAGA
AGAACAGCCGGGAGCGGATGAAGCGGATCGAGGAGGGCATCAAGGAGCTGGGCAGCCAGATC
CTGAAGGAGCACCCCGTGGAGAACACCCAGCTGCAGAACGAGAAGCTGTACCTGTACTACCT
GCAGAACGGCCGGGACATGTACGTGGACCAGGAGCTGGACATCAACCGGCTGAGCGACTACG
ACGTGGCCGCCATCGTGCCCCAGAGCTTCCTGAAGGACGACAGCATCGACAACAAGGTGCTG
ACCCGGAGCGACAAGGCCCGGGGCAAGAGCGACAACGTGCCCAGCGAGGAGGTGGTGAAGAA
GATGAAGAACTACTGGCGGCAGCTGCTGAACGCCAAGCTGATCACCCAGCGGAAGTTCGACA
ACCTGACCAAGGCCGAGCGGGGCGGCCTGAGCGAGCTGGACAAGGCCGGCTTCATCAAGCGG
CAGCTGGTGGAGACCCGGCAGATCACCAAGCACGTGGCCCAGATCCTGGACAGCCGGATGAA
CACCAAGTACGACGAGAACGACAAGCTGATCCGGGAGGTGAAGGTGATCACCCTGaaatccA
AGCTGGTGAGCGACTTCCGGAAGGACTTCCAGTTCTACAAGGTGCGGGAGATCAACAACTAC
CACCACGCCCACGACGCCTACCTGAACGCCGTGGTGGGCACCGCCCTGATCAAGAAGTACCC
CAAGCTGGAGAGCGAGTTCGTGTACGGCGACTACAAGGTGTACGACGTGCGGAAGATGATCG
CCAAGAGCGAGCAGGAGATCGGCAAGGCCACCGCCAAGTACTTCTTCTACAGCAACATCATG
AACTTCTTCAAGACCGAGATCACCCTGGCCAACGGCGAGATCCGGAAGCGGCCCCTGATCGA
GACCAACGGCGAGACCGGCGAGATCGTGTGGGACAAGGGCCGGGACTTCGCCACCGTGCGGA
AGGTGCTGAGCATGCCCCAGGTGAACATCGTGAAGAAaACCGAGGTGCAGACCGGCGGCTTC
AGCAAGGAGAGCATCCTGCCCAAGCGGAACAGCGACAAGCTGATCGCCCGGAAGAAGGACTG
GGACCCCAAGAAGTACGGCGGCTTCGACAGCCCCACCGTGGCCTACAGCGTGCTGGTGGTGG
CCAAGGTGGAGAAGGGCAAGAGCAAGAAGCTGaaatccGTGAAGGAGCTGCTGGGCATCACC
ATCATGGAGCGGAGCAGCTTCGAGAAGAACCCCATCGACTTCCTGGAGGCCAAGGGCTACAA
GGAGGTGAAGAAGGACCTGATCATCAAGCTGCCCAAGTACAGCCTGTTCGAGCTGGAGAACG
GCCGGAAGCGGATGCTGGCCAGCGCCGGCGAGCTGCAGAAGGGCAACGAGCTGGCCCTGCCC
AGCAAGTACGTGAACTTCCTGTACCTGGCCAGCCACTACGAGAAGCTGAAGGGCAGCCCCGA
GGACAACGAGCAGAAGCAGCTGTTCGTGGAGCAGCACAAGCACTACCTGGACGAGATCATCG
AGCAGATCAGCGAGTTCAGCAAGCGGGTGATCCTGGCCGACGCCAACCTGGACAAGGTGCTG
AGCGCCTACAACAAGCACCGGGACAAGCCCATCCGGGAGCAGGCCGAGAACATCATCCACCT
GTTCACCCTGACCAACCTGGGCGCCCCCGCCGCCTTCAAGTACTTCGACACCACCATCGACC
GGAAGCGGTACACCAGCACCAAGGAGGTGCTGGACGCCACCCTGATCCACCAGAGCATCACC
GGCCTGTACGAGACCCGGATCGACCTGAGCCAGCTGGGCGGCGACAAGCGGCCCGCCGCCAC
CAAGAAGGCCGGCCAGGCCAAGAAGAAGAAGGCCAGCGACGCCAAGAGCCTGACCGCCTGGA
GCCGGACCCTGGTGACCTTCAAGGACGTGTTCGTGGACTTCACCCGGGAGGAGTGGAAGCTG
CTGGACACCGCCCAGCAGATCCTGTACCGGAACGTGATGCTGGAGAACTACAAGAACCTGGT
GAGCCTGGGCTACCAGCTGACCAAGCCCGACGTGATCCTGCGGCTGGAGAAGGGCGAGGAGC
CCTGGCTGGTGGAGCGGGAGATCCACCAGGAGACCCACCCCGACAGCGAGACCGCCTTCGAG
ATCAAGAGCAGCGTGAGCGGCGGCAAGCGGCCCGCCGCCACCAAGAAGGCCGGCCAGGCCAA
GAAGAAGAAGGGCAGCTACCCCTACGACGTGCCCGACTACGCCTGAGcggccgcttaattaa
gctgccttctgcggggcttgccttctggccatgcccttcttctctcccttgcacctgtacct
cttggtctttgaataaagcctgagtaggaagtctagaaaaaaaaaaaaaaaaaaaaaaaaaa
aaaaaaaaaaaaaaaaaaaaaaaaaaaaaaaaaaaaaaaaaaaaaaaaaaaaaa

In some embodiments, an expression repressor comprises the amino acid sequence of SEQ ID NOs: 96 or 72. In some embodiments, an expression repressor described herein comprises a an amino acid sequence of SEQ ID NO: 72 or 96 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Sp-dCas9-KRAB Protein sequence:
(SEQ ID NO: 96)
MAPKKKRKVGIHGVPAADKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGDKRPAATKKAGQAKKKK
ASDAKSLTAWSRTLVTFKDVFVDFTREEWKLLDTAQQILYRNVMLENYKN
LVSLGYQLTKPDVILRLEKGEEPWLVEREIHQETHPDSETAFEIKSSVSG
GKRPAATKKAGQAKKKKGSYPYDVPDYA
(SEQ ID NO: 72)
MAPKKKRKVGIHGVPAADKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGDKRPAATKKAGQAKKKK
ASDAKSLTAWSRTLVTFKDVFVDFTREEWKLLDTAQQILYRNVMLENYKN
LVSLGYQLTKPDVILRLEKGEEPWLVEREIHQETHPDSETAFEIKSSVSG
GKRPAATKKAGQAKKKKGS

In some embodiments, an expression repressor comprises a DNA-targeting moiety comprising dCas9, e.g., an S. pyogenes dCas9, and a repressor domain comprising DNMT1, e.g., a DNMT1 domain. In some embodiments, the expression repressor is encoded by the nucleic acid sequence of SEQ ID NOs: 23 (e.g., a nucleic acid (e.g., cDNA) encoding the expression repressor). In some embodiments, a nucleic acid described herein comprises a nucleic acid sequence of SEQ ID NO: 23 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT1 (MR-29419)
(SEQ ID NO: 23)
AGGAAATAAGAGAGAAAAGAAGAGTAAGAAGAAATATAAG
AGCCACCATGGCCCCCAAGAAGAAGCGGAAGGTGGGCATC
CACGGCGTGCCCGCCGCCGACAAGAAGTACAGCATCGGCC
TGGCCATCGGCACCAACAGCGTGGGCTGGGCCGTGATCAC
CGACGAGTACAAGGTGCCCAGCAAGAAGTTCAAGGTGCTG
GGCAACACCGACCGGCACAGCATCAAGAAGAACCTGATCG
GCGCCCTGCTGTTCGACAGCGGCGAGACCGCCGAGGCCAC
CCGGCTGAAGCGGACCGCCCGGCGGCGGTACACCCGGCGG
AAGAACCGGATCTGCTACCTGCAGGAGATCTTCAGCAACG
AGATGGCCAAGGTGGACGACAGCTTCTTCCACCGGCTGGA
GGAGAGCTTCCTGGTGGAGGAGGACAAGAAGCACGAGCGG
CACCCCATCTTCGGCAACATCGTGGACGAGGTGGCCTACC
ACGAGAAGTACCCCACCATCTACCACCTGCGGAAGAAGCT
GGTGGACAGCACCGACAAGGCCGACCTGCGGCTGATCTAC
CTGGCCCTGGCCCACATGATCAAGTTCCGGGGCCACTTCC
TGATCGAGGGCGACCTGAACCCCGACAACAGCGACGTGGA
CAAGCTGTTCATCCAGCTGGTGCAGACCTACAACCAGCTG
TTCGAGGAGAACCCCATCAACGCCAGCGGCGTGGACGCCA
AGGCCATCCTGAGCGCCCGGCTGAGCAAGAGCCGGCGGCT
GGAGAACCTGATCGCCCAGCTGCCCGGCGAGAAGAAGAAC
GGCCTGTTCGGCAACCTGATCGCCCTGAGCCTGGGCCTGA
CCCCCAACTTCAAGAGCAACTTCGACCTGGCCGAGGACGC
CAAGCTGCAGCTGAGCAAGGACACCTACGACGACGACCTG
GACAACCTGCTGGCCCAGATCGGCGACCAGTACGCCGACC
TGTTCCTGGCCGCCAAGAACCTGAGCGACGCCATCCTGCT
GAGCGACATCCTGCGGGTGAACACCGAGATCACCAAGGCC
CCCCTGAGCGCCAGCATGATCAAGCGGTACGACGAGCACC
ACCAGGACCTGACCCTGCTGAAGGCCCTGGTGCGGCAGCA
GCTGCCCGAGAAGTACAAGGAGATCTTCTTCGACCAGAGC
AAGAACGGCTACGCCGGCTACATCGACGGCGGCGCCAGCC
AGGAGGAGTTCTACAAGTTCATCAAGCCCATCCTGGAGAA
GATGGACGGCACCGAGGAGCTGCTGGTGAAGCTGAACCGG
GAGGACCTGCTGCGGAAGCAGCGGACCTTCGACAACGGCA
GCATCCCCCACCAGATCCACCTGGGCGAGCTGCACGCCAT
CCTGCGGCGGCAGGAGGACTTCTACCCCTTCCTGAAGGAC
AACCGGGAGAAGATCGAGAAGATCCTGACCTTCCGGATCC
CCTACTACGTGGGCCCCCTGGCCCGGGGCAACAGCCGGTT
CGCCTGGATGACCCGGAAATCCGAGGAGACCATCACCCCC
TGGAACTTCGAGGAGGTGGTGGACAAGGGCGCCAGCGCCC
AGAGCTTCATCGAGCGGATGACCAACTTCGACAAGAACCT
GCCCAACGAGAAGGTGCTGCCCAAGCACAGCCTGCTGTAC
GAGTACTTCACCGTGTACAACGAGCTGACCAAGGTGAAGT
ACGTGACCGAGGGCATGCGGAAGCCCGCCTTCCTGAGCGG
CGAGCAGAAGAAGGCCATCGTGGACCTGCTGTTCAAGACC
AACCGGAAGGTGACCGTGAAGCAGCTGAAGGAGGACTACT
TCAAGAAGATCGAGTGCTTCGACAGCGTGGAGATCAGCGG
CGTGGAGGACCGGTTCAACGCCAGCCTGGGCACCTACCAC
GACCTGCTGAAGATCATCAAGGACAAGGACTTCCTGGACA
ACGAGGAGAACGAGGACATCCTGGAGGACATCGTGCTGAC
CCTGACCCTGTTCGAGGACCGGGAGATGATCGAGGAGCGG
CTGAAAACCTACGCCCACCTGTTCGACGACAAGGTGATGA
AGCAGCTGAAGCGGCGGCGGTACACCGGCTGGGGCCGGCT
GAGCCGGAAGCTGATCAACGGCATCCGGGACAAGCAGAGC
GGCAAGACCATCCTGGACTTCCTGAAATCCGACGGCTTCG
CCAACCGGAACTTCATGCAGCTGATCCACGACGACAGCCT
GACCTTCAAGGAGGACATCCAGAAGGCCCAGGTGAGCGGC
CAGGGCGACAGCCTGCACGAGCACATCGCCAACCTGGCCG
GCAGCCCCGCCATCAAGAAGGGCATCCTGCAGACCGTGAA
GGTGGTGGACGAGCTGGTGAAGGTGATGGGCCGGCACAAG
CCCGAGAACATCGTGATCGAGATGGCCCGGGAGAACCAGA
CCACCCAGAAGGGCCAGAAGAACAGCCGGGAGCGGATGAA
GCGGATCGAGGAGGGCATCAAGGAGCTGGGCAGCCAGATC
CTGAAGGAGCACCCCGTGGAGAACACCCAGCTGCAGAACG
AGAAGCTGTACCTGTACTACCTGCAGAACGGCCGGGACAT
GTACGTGGACCAGGAGCTGGACATCAACCGGCTGAGCGAC
TACGACGTGGCCGCCATCGTGCCCCAGAGCTTCCTGAAGG
ACGACAGCATCGACAACAAGGTGCTGACCCGGAGCGACAA
GGCCCGGGGCAAGAGCGACAACGTGCCCAGCGAGGAGGTG
GTGAAGAAGATGAAGAACTACTGGCGGCAGCTGCTGAACG
CCAAGCTGATCACCCAGCGGAAGTTCGACAACCTGACCAA
GGCCGAGCGGGGCGGCCTGAGCGAGCTGGACAAGGCCGGC
TTCATCAAGCGGCAGCTGGTGGAGACCCGGCAGATCACCA
AGCACGTGGCCCAGATCCTGGACAGCCGGATGAACACCAA
GTACGACGAGAACGACAAGCTGATCCGGGAGGTGAAGGTG
ATCACCCTGAAATCCAAGCTGGTGAGCGACTTCCGGAAGG
ACTTCCAGTTCTACAAGGTGCGGGAGATCAACAACTACCA
CCACGCCCACGACGCCTACCTGAACGCCGTGGTGGGCACC
GCCCTGATCAAGAAGTACCCCAAGCTGGAGAGCGAGTTCG
TGTACGGCGACTACAAGGTGTACGACGTGCGGAAGATGAT
CGCCAAGAGCGAGCAGGAGATCGGCAAGGCCACCGCCAAG
TACTTCTTCTACAGCAACATCATGAACTTCTTCAAGACCG
AGATCACCCTGGCCAACGGCGAGATCCGGAAGCGGCCCCT
GATCGAGACCAACGGCGAGACCGGCGAGATCGTGTGGGAC
AAGGGCCGGGACTTCGCCACCGTGCGGAAGGTGCTGAGCA
TGCCCCAGGTGAACATCGTGAAGAAAACCGAGGTGCAGAC
CGGCGGCTTCAGCAAGGAGAGCATCCTGCCCAAGCGGAAC
AGCGACAAGCTGATCGCCCGGAAGAAGGACTGGGACCCCA
AGAAGTACGGCGGCTTCGACAGCCCCACCGTGGCCTACAG
CGTGCTGGTGGTGGCCAAGGTGGAGAAGGGCAAGAGCAAG
AAGCTGAAATCCGTGAAGGAGCTGCTGGGCATCACCATCA
TGGAGCGGAGCAGCTTCGAGAAGAACCCCATCGACTTCCT
GGAGGCCAAGGGCTACAAGGAGGTGAAGAAGGACCTGATC
ATCAAGCTGCCCAAGTACAGCCTGTTCGAGCTGGAGAACG
GCCGGAAGCGGATGCTGGCCAGCGCCGGCGAGCTGCAGAA
GGGCAACGAGCTGGCCCTGCCCAGCAAGTACGTGAACTTC
CTGTACCTGGCCAGCCACTACGAGAAGCTGAAGGGCAGCC
CCGAGGACAACGAGCAGAAGCAGCTGTTCGTGGAGCAGCA
CAAGCACTACCTGGACGAGATCATCGAGCAGATCAGCGAG
TTCAGCAAGCGGGTGATCCTGGCCGACGCCAACCTGGACA
AGGTGCTGAGCGCCTACAACAAGCACCGGGACAAGCCCAT
CCGGGAGCAGGCCGAGAACATCATCCACCTGTTCACCCTG
ACCAACCTGGGCGCCCCCGCCGCCTTCAAGTACTTCGACA
CCACCATCGACCGGAAGCGGTACACCAGCACCAAGGAGGT
GCTGGACGCCACCCTGATCCACCAGAGCATCACCGGCCTG
TACGAGACCCGGATCGACCTGAGCCAGCTGGGCGGCGACA
GCGGCGGCAAGCGGCCCGCCGCCACCAAGAAGGCCGGCCA
GGCCAAGAAGAAGAAGTCGGGCGGGGGTGGCTCAGTGGAT
CTGAGGACACTCGACGTGTTTAGCGGATGCGGCGGACTCT
CCGAAGGCTTCCACCAAGCCGGAATTTCCGACACACTCTG
GGCCATTGAGATGTGGGACCCCGCCGCTCAAGCCTTCAGA
CTGAATAATCCCGGCTCCACCGTGTTCACCGAGGACTGCA
ACATTCTGCTGAAGCTGGTGATGGCTGGCGAAACCACCAA
CTCTAGAGGCCAGAGGCTGCCCCAGAAGGGAGATGTGGAA
ATGCTCTGTGGAGGCCCTCCTTGCCAAGGCTTCTCCGGCA
TGAACAGGTTCAACTCTAGAACATACAGCAAGTTCAAGAA
CTCTCTGGTCGTGAGCTTTCTGAGCTACTGCGACTACTAT
AGACCTAGGTTCTTTCTGCTGGAGAACGTGAGAAATTTCG
TGTCCTTCAAGAGGAGCATGGTGCTGAAGCTGACACTGAG
GTGTCTGGTGAGGATGGGCTACCAGTGCACATTCGGAGTG
CTGCAAGCTGGCCAGTACGGCGTGGCCCAGACCAGAAGGA
GGGCCATCATTCTGGCTGCTGCCCCCGGCGAGAAACTCCC
TCTGTTCCCCGAGCCCCTCCACGTGTTCGCCCCTAGAGCT
TGCCAGCTGAGCGTGGTGGTCGACGATAAGAAGTTCGTGA
GCAACATCACAAGGCTGTCCAGCGGACCCTTCAGAACCAT
TACCGTGAGGGATACCATGTCCGACCTCCCCGAGGTGAGG
AATGGCGCCAGCGCTCTGGAGATTTCCTACAACGGCGAAC
CTCAGAGCTGGTTCCAAAGGCAGCTGAGAGGCGCTCAGTA
TCAGCCCATTCTGAGGGACCACATCTGCAAAGATATGAGC
GCTCTGGTGGCCGCTAGAATGAGACATATTCCTCTGGCCC
CCGGCAGCGACTGGAGAGATCTGCCCAATATTGAGGTGAG
ACTCAGCGACGGAACAATGGCTAGAAAACTGAGGTACACC
CATCATGATAGAAAGAACGGAAGGAGCAGCAGCGGCGCTC
TGAGAGGAGTGTGTAGCTGCGTGGAAGCTGGCAAGGCTTG
CGATCCCGCCGCTAGGCAGTTCAATACCCTCATCCCTTGG
TGTCTGCCTCACACCGGCAACAGACACAATCATTGGGCTG
GACTGTATGGAAGGCTCGAATGGGACGGCTTTTTCAGCAC
CACCGTGACCAATCCCGAACCTATGGGCAAGCAAGGAAGG
GTGCTCCACCCCGAGCAGCATAGAGTCGTGTCCGTGAGAG
AATGCGCTAGAAGCCAAGGCTTCCCCGACACCTATAGACT
GTTCGGCAACATTCTGGATAAGCACAGACAAGTGGGAAAT
GCTGTCCCTCCTCCTCTGGCCAAGGCTATCGGACTGGAGA
TCAAGCTGTGTATGCTCGCCAAAGCTAGGGAGAGCGCTTC
CGCCAAGATTAAGGAGGAGGAGGCCGCCAAGGACGGAGGT
GGCGGATCGGGAAAGCGGCCCGCCGCCACCAAGAAGGCCG
GTCAGGCCAAGAAGAAGAAGGGCAGCTACCCCTACGACGT
GCCCGACTACGCCTGAGCGGCCGCTTAATTAAGCTGCCTT
CTGCGGGGCTTGCCTTCTGGCCATGCCCTTCTTCTCTCCC
TTGCACCTGTACCTCTTGGTCTTTGAATAAAGCCTGAGTA
GGAAGTCTAGAAAAAAAAAAAAAAAAAAAAAAAAAAAAAA
AAAAAAAAAAAAAAAAAAAAAAAAAAAAAAAAAAAAAAAA
AAAAAAAAAA

In some embodiments, an expression repressor comprises the amino acid sequence of SEQ ID NOs: 34 or 69. In some embodiments, an expression repressor described herein comprises an amino acid sequence of SEQ ID NO: 34 or 69, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT
(SEQ ID NO: 34)
MAPKKKRKVGIHGVPAADKKYSIGLAIGTNSVGWAVITDE
YKVPSKKFKVLGNTDRHSIKKNLIGALLFDSGETAEATRL
KRTARRRYTRRKNRICYLQEIFSNEMAKVDDSFFHRLEES
FLVEEDKKHERHPIFGNIVDEVAYHEKYPTIYHLRKKLVD
STDKADLRLIYLALAHMIKFRGHFLIEGDLNPDNSDVDKL
FIQLVQTYNQLFEENPINASGVDAKAILSARLSKSRRLEN
LIAQLPGEKKNGLFGNLIALSLGLTPNFKSNFDLAEDAKL
QLSKDTYDDDLDNLLAQIGDQYADLFLAAKNLSDAILLSD
ILRVNTEITKAPLSASMIKRYDEHHQDLTLLKALVRQQLP
EKYKEIFFDQSKNGYAGYIDGGASQEEFYKFIKPILEKMD
GTEELLVKLNREDLLRKQRTFDNGSIPHQIHLGELHAILR
RQEDFYPFLKDNREKIEKILTFRIPYYVGPLARGNSRFAW
MTRKSEETITPWNFEEVVDKGASAQSFIERMTNFDKNLPN
EKVLPKHSLLYEYFTVYNELTKVKYVTEGMRKPAFLSGEQ
KKAIVDLLFKTNRKVTVKQLKEDYFKKIECFDSVEISGVE
DRFNASLGTYHDLLKIIKDKDFLDNEENEDILEDIVLTLT
LFEDREMIEERLKTYAHLFDDKVMKQLKRRRYTGWGRLSR
KLINGIRDKQSGKTILDFLKSDGFANRNFMQLIHDDSLTF
KEDIQKAQVSGQGDSLHEHIANLAGSPAIKKGILQTVKVV
DELVKVMGRHKPENIVIEMARENQTTQKGQKNSRERMKRI
EEGIKELGSQILKEHPVENTQLQNEKLYLYYLQNGRDMYV
DQELDINRLSDYDVAAIVPQSFLKDDSIDNKVLTRSDKAR
GKSDNVPSEEVVKKMKNYWRQLLNAKLITQRKFDNLTKAE
RGGLSELDKAGFIKRQLVETRQITKHVAQILDSRMNTKYD
ENDKLIREVKVITLKSKLVSDFRKDFQFYKVREINNYHHA
HDAYLNAVVGTALIKKYPKLESEFVYGDYKVYDVRKMIAK
SEQEIGKATAKYFFYSNIMNFFKTEITLANGEIRKRPLIE
TNGETGEIVWDKGRDFATVRKVLSMPQVNIVKKTEVQTGG
FSKESILPKRNSDKLIARKKDWDPKKYGGFDSPTVAYSVL
VVAKVEKGKSKKLKSVKELLGITIMERSSFEKNPIDFLEA
KGYKEVKKDLIIKLPKYSLFELENGRKRMLASAGELQKGN
ELALPSKYVNFLYLASHYEKLKGSPEDNEQKQLFVEQHKH
YLDEIIEQISEFSKRVILADANLDKVLSAYNKHRDKPIRE
QAENIIHLFTLTNLGAPAAFKYFDTTIDRKRYTSTKEVLD
ATLIHQSITGLYETRIDLSQLGGDSGGKRPAATKKAGQAK
KKKSGGGGSVDLRTLDVFSGCGGLSEGFHQAGISDTLWAI
EMWDPAAQAFRLNNPGSTVFTEDCNILLKLVMAGETTNSR
GQRLPQKGDVEMLCGGPPCQGFSGMNRFNSRTYSKFKNSL
VVSFLSYCDYYRPRFFLLENVRNFVSFKRSMVLKLTLRCL
VRMGYQCTFGVLQAGQYGVAQTRRRAIILAAAPGEKLPLF
PEPLHVFAPRACQLSVVVDDKKFVSNITRLSSGPFRTITV
RDTMSDLPEVRNGASALEISYNGEPQSWFQRQLRGAQYQP
ILRDHICKDMSALVAARMRHIPLAPGSDWRDLPNIEVRLS
DGTMARKLRYTHHDRKNGRSSSGALRGVCSCVEAGKACDP
AARQFNTLIPWCLPHTGNRHNHWAGLYGRLEWDGFFSTTV
TNPEPMGKQGRVLHPEQHRVVSVRECARSQGFPDTYRLFG
NILDKHRQVGNAVPPPLAKAIGLEIKLCMLAKARESASAK
IKEEEAAKDGGGGSGKRPAATKKAGQAKKKKGSYPYDVPD
YA
dCas9-DNMT without HA tag
(SEQ ID NO: 69)
MAPKKKRKVGIHGVPAADKKYSIGLAIGTNSVGWAVITDE
YKVPSKKFKVLGNTDRHSIKKNLIGALLFDSGETAEATRL
KRTARRRYTRRKNRICYLQEIFSNEMAKVDDSFFHRLEES
FLVEEDKKHERHPIFGNIVDEVAYHEKYPTIYHLRKKLVD
STDKADLRLIYLALAHMIKFRGHFLIEGDLNPDNSDVDKL
FIQLVQTYNQLFEENPINASGVDAKAILSARLSKSRRLEN
LIAQLPGEKKNGLFGNLIALSLGLTPNFKSNFDLAEDAKL
QLSKDTYDDDLDNLLAQIGDQYADLFLAAKNLSDAILLSD
ILRVNTEITKAPLSASMIKRYDEHHQDLTLLKALVRQQLP
EKYKEIFFDQSKNGYAGYIDGGASQEEFYKFIKPILEKMD
GTEELLVKLNREDLLRKQRTFDNGSIPHQIHLGELHAILR
RQEDFYPFLKDNREKIEKILTFRIPYYVGPLARGNSRFAW
MTRKSEETITPWNFEEVVDKGASAQSFIERMTNFDKNLPN
EKVLPKHSLLYEYFTVYNELTKVKYVTEGMRKPAFLSGEQ
KKAIVDLLFKTNRKVTVKQLKEDYFKKIECFDSVEISGVE
DRFNASLGTYHDLLKIIKDKDFLDNEENEDILEDIVLTLT
LFEDREMIEERLKTYAHLFDDKVMKQLKRRRYTGWGRLSR
KLINGIRDKQSGKTILDFLKSDGFANRNFMQLIHDDSLTF
KEDIQKAQVSGQGDSLHEHIANLAGSPAIKKGILQTVKVV
DELVKVMGRHKPENIVIEMARENQTTQKGQKNSRERMKRI
EEGIKELGSQILKEHPVENTQLQNEKLYLYYLQNGRDMYV
DQELDINRLSDYDVAAIVPQSFLKDDSIDNKVLTRSDKAR
GKSDNVPSEEVVKKMKNYWRQLLNAKLITQRKFDNLTKAE
RGGLSELDKAGFIKRQLVETRQITKHVAQILDSRMNTKYD
ENDKLIREVKVITLKSKLVSDFRKDFQFYKVREINNYHHA
HDAYLNAVVGTALIKKYPKLESEFVYGDYKVYDVRKMIAK
SEQEIGKATAKYFFYSNIMNFFKTEITLANGEIRKRPLIE
TNGETGEIVWDKGRDFATVRKVLSMPQVNIVKKTEVQTGG
FSKESILPKRNSDKLIARKKDWDPKKYGGFDSPTVAYSVL
VVAKVEKGKSKKLKSVKELLGITIMERSSFEKNPIDFLEA
KGYKEVKKDLIIKLPKYSLFELENGRKRMLASAGELQKGN
ELALPSKYVNFLYLASHYEKLKGSPEDNEQKQLFVEQHKH
YLDEIIEQISEFSKRVILADANLDKVLSAYNKHRDKPIRE
QAENIIHLFTLTNLGAPAAFKYFDTTIDRKRYTSTKEVLD
ATLIHQSITGLYETRIDLSQLGGDSGGKRPAATKKAGQAK
KKKSGGGGSVDLRTLDVFSGCGGLSEGFHQAGISDTLWAI
EMWDPAAQAFRLNNPGSTVFTEDCNILLKLVMAGETTNSR
GQRLPQKGDVEMLCGGPPCQGFSGMNRFNSRTYSKFKNSL
VVSFLSYCDYYRPRFFLLENVRNFVSFKRSMVLKLTLRCL
VRMGYQCTFGVLQAGQYGVAQTRRRAIILAAAPGEKLPLF
PEPLHVFAPRACQLSVVVDDKKFVSNITRLSSGPFRTITV
RDTMSDLPEVRNGASALEISYNGEPQSWFQRQLRGAQYQP
ILRDHICKDMSALVAARMRHIPLAPGSDWRDLPNIEVRLS
DGTMARKLRYTHHDRKNGRSSSGALRGVCSCVEAGKACDP
AARQFNTLIPWCLPHTGNRHNHWAGLYGRLEWDGFFSTTV
TNPEPMGKQGRVLHPEQHRVVSVRECARSQGFPDTYRLFG
NILDKHRQVGNAVPPPLAKAIGLEIKLCMLAKARESASAK
IKEEEAAKDGGGGSGKRPAATKKAGQAKKKKGS

In some embodiments, an expression repressor comprises a DNA-targeting moiety comprising dCas9, e.g., an S. pyogenes dCas9, and a repressor domain comprising DNMT3a/3L. In some embodiments, the expression repressor is encoded by the nucleic acid sequence of SEQ ID NOs: 24 (e.g., a nucleic acid (e.g., cDNA) encoding a fully human expression repressor) and/or 25 (e.g., a nucleic acid (e.g., cDNA) encoding a chimeric expression repressor). In some embodiments, a nucleic acid described herein comprises a nucleic acid sequence of SEQ ID NO: 24 or 25 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT 3a/3L (h) (MR-29414)
(SEQ ID NO: 24)
AGGAAATAAGAGAGAAAAGAAGAGTAAGAAGAAATATAAG
AGCCACCATGGCCCCCAAGAAGAAGCGGAAGGTGGGCATC
CACGGCGTGCCCGCCGCCGACAAGAAGTACAGCATCGGCC
TGGCCATCGGCACCAACAGCGTGGGCTGGGCCGTGATCAC
CGACGAGTACAAGGTGCCCAGCAAGAAGTTCAAGGTGCTG
GGCAACACCGACCGGCACAGCATCAAGAAGAACCTGATCG
GCGCCCTGCTGTTCGACAGCGGCGAGACCGCCGAGGCCAC
CCGGCTGAAGCGGACCGCCCGGCGGCGGTACACCCGGCGG
AAGAACCGGATCTGCTACCTGCAGGAGATCTTCAGCAACG
AGATGGCCAAGGTGGACGACAGCTTCTTCCACCGGCTGGA
GGAGAGCTTCCTGGTGGAGGAGGACAAGAAGCACGAGCGG
CACCCCATCTTCGGCAACATCGTGGACGAGGTGGCCTACC
ACGAGAAGTACCCCACCATCTACCACCTGCGGAAGAAGCT
GGTGGACAGCACCGACAAGGCCGACCTGCGGCTGATCTAC
CTGGCCCTGGCCCACATGATCAAGTTCCGGGGCCACTTCC
TGATCGAGGGCGACCTGAACCCCGACAACAGCGACGTGGA
CAAGCTGTTCATCCAGCTGGTGCAGACCTACAACCAGCTG
TTCGAGGAGAACCCCATCAACGCCAGCGGCGTGGACGCCA
AGGCCATCCTGAGCGCCCGGCTGAGCAAGAGCCGGCGGCT
GGAGAACCTGATCGCCCAGCTGCCCGGCGAGAAGAAGAAC
GGCCTGTTCGGCAACCTGATCGCCCTGAGCCTGGGCCTGA
CCCCCAACTTCAAGAGCAACTTCGACCTGGCCGAGGACGC
CAAGCTGCAGCTGAGCAAGGACACCTACGACGACGACCTG
GACAACCTGCTGGCCCAGATCGGCGACCAGTACGCCGACC
TGTTCCTGGCCGCCAAGAACCTGAGCGACGCCATCCTGCT
GAGCGACATCCTGCGGGTGAACACCGAGATCACCAAGGCC
CCCCTGAGCGCCAGCATGATCAAGCGGTACGACGAGCACC
ACCAGGACCTGACCCTGCTGAAGGCCCTGGTGCGGCAGCA
GCTGCCCGAGAAGTACAAGGAGATCTTCTTCGACCAGAGC
AAGAACGGCTACGCCGGCTACATCGACGGCGGCGCCAGCC
AGGAGGAGTTCTACAAGTTCATCAAGCCCATCCTGGAGAA
GATGGACGGCACCGAGGAGCTGCTGGTGAAGCTGAACCGG
GAGGACCTGCTGCGGAAGCAGCGGACCTTCGACAACGGCA
GCATCCCCCACCAGATCCACCTGGGCGAGCTGCACGCCAT
CCTGCGGCGGCAGGAGGACTTCTACCCCTTCCTGAAGGAC
AACCGGGAGAAGATCGAGAAGATCCTGACCTTCCGGATCC
CCTACTACGTGGGCCCCCTGGCCCGGGGCAACAGCCGGTT
CGCCTGGATGACCCGGAAATCCGAGGAGACCATCACCCCC
TGGAACTTCGAGGAGGTGGTGGACAAGGGCGCCAGCGCCC
AGAGCTTCATCGAGCGGATGACCAACTTCGACAAGAACCT
GCCCAACGAGAAGGTGCTGCCCAAGCACAGCCTGCTGTAC
GAGTACTTCACCGTGTACAACGAGCTGACCAAGGTGAAGT
ACGTGACCGAGGGCATGCGGAAGCCCGCCTTCCTGAGCGG
CGAGCAGAAGAAGGCCATCGTGGACCTGCTGTTCAAGACC
AACCGGAAGGTGACCGTGAAGCAGCTGAAGGAGGACTACT
TCAAGAAGATCGAGTGCTTCGACAGCGTGGAGATCAGCGG
CGTGGAGGACCGGTTCAACGCCAGCCTGGGCACCTACCAC
GACCTGCTGAAGATCATCAAGGACAAGGACTTCCTGGACA
ACGAGGAGAACGAGGACATCCTGGAGGACATCGTGCTGAC
CCTGACCCTGTTCGAGGACCGGGAGATGATCGAGGAGCGG
CTGAAAACCTACGCCCACCTGTTCGACGACAAGGTGATGA
AGCAGCTGAAGCGGCGGCGGTACACCGGCTGGGGCCGGCT
GAGCCGGAAGCTGATCAACGGCATCCGGGACAAGCAGAGC
GGCAAGACCATCCTGGACTTCCTGAAATCCGACGGCTTCG
CCAACCGGAACTTCATGCAGCTGATCCACGACGACAGCCT
GACCTTCAAGGAGGACATCCAGAAGGCCCAGGTGAGCGGC
CAGGGCGACAGCCTGCACGAGCACATCGCCAACCTGGCCG
GCAGCCCCGCCATCAAGAAGGGCATCCTGCAGACCGTGAA
GGTGGTGGACGAGCTGGTGAAGGTGATGGGCCGGCACAAG
CCCGAGAACATCGTGATCGAGATGGCCCGGGAGAACCAGA
CCACCCAGAAGGGCCAGAAGAACAGCCGGGAGCGGATGAA
GCGGATCGAGGAGGGCATCAAGGAGCTGGGCAGCCAGATC
CTGAAGGAGCACCCCGTGGAGAACACCCAGCTGCAGAACG
AGAAGCTGTACCTGTACTACCTGCAGAACGGCCGGGACAT
GTACGTGGACCAGGAGCTGGACATCAACCGGCTGAGCGAC
TACGACGTGGCCGCCATCGTGCCCCAGAGCTTCCTGAAGG
ACGACAGCATCGACAACAAGGTGCTGACCCGGAGCGACAA
GGCCCGGGGCAAGAGCGACAACGTGCCCAGCGAGGAGGTG
GTGAAGAAGATGAAGAACTACTGGCGGCAGCTGCTGAACG
CCAAGCTGATCACCCAGCGGAAGTTCGACAACCTGACCAA
GGCCGAGCGGGGCGGCCTGAGCGAGCTGGACAAGGCCGGC
TTCATCAAGCGGCAGCTGGTGGAGACCCGGCAGATCACCA
AGCACGTGGCCCAGATCCTGGACAGCCGGATGAACACCAA
GTACGACGAGAACGACAAGCTGATCCGGGAGGTGAAGGTG
ATCACCCTGAAATCCAAGCTGGTGAGCGACTTCCGGAAGG
ACTTCCAGTTCTACAAGGTGCGGGAGATCAACAACTACCA
CCACGCCCACGACGCCTACCTGAACGCCGTGGTGGGCACC
GCCCTGATCAAGAAGTACCCCAAGCTGGAGAGCGAGTTCG
TGTACGGCGACTACAAGGTGTACGACGTGCGGAAGATGAT
CGCCAAGAGCGAGCAGGAGATCGGCAAGGCCACCGCCAAG
TACTTCTTCTACAGCAACATCATGAACTTCTTCAAGACCG
AGATCACCCTGGCCAACGGCGAGATCCGGAAGCGGCCCCT
GATCGAGACCAACGGCGAGACCGGCGAGATCGTGTGGGAC
AAGGGCCGGGACTTCGCCACCGTGCGGAAGGTGCTGAGCA
TGCCCCAGGTGAACATCGTGAAGAAAACCGAGGTGCAGAC
CGGCGGCTTCAGCAAGGAGAGCATCCTGCCCAAGCGGAAC
AGCGACAAGCTGATCGCCCGGAAGAAGGACTGGGACCCCA
AGAAGTACGGCGGCTTCGACAGCCCCACCGTGGCCTACAG
CGTGCTGGTGGTGGCCAAGGTGGAGAAGGGCAAGAGCAAG
AAGCTGAAATCCGTGAAGGAGCTGCTGGGCATCACCATCA
TGGAGCGGAGCAGCTTCGAGAAGAACCCCATCGACTTCCT
GGAGGCCAAGGGCTACAAGGAGGTGAAGAAGGACCTGATC
ATCAAGCTGCCCAAGTACAGCCTGTTCGAGCTGGAGAACG
GCCGGAAGCGGATGCTGGCCAGCGCCGGCGAGCTGCAGAA
GGGCAACGAGCTGGCCCTGCCCAGCAAGTACGTGAACTTC
CTGTACCTGGCCAGCCACTACGAGAAGCTGAAGGGCAGCC
CCGAGGACAACGAGCAGAAGCAGCTGTTCGTGGAGCAGCA
CAAGCACTACCTGGACGAGATCATCGAGCAGATCAGCGAG
TTCAGCAAGCGGGTGATCCTGGCCGACGCCAACCTGGACA
AGGTGCTGAGCGCCTACAACAAGCACCGGGACAAGCCCAT
CCGGGAGCAGGCCGAGAACATCATCCACCTGTTCACCCTG
ACCAACCTGGGCGCCCCCGCCGCCTTCAAGTACTTCGACA
CCACCATCGACCGGAAGCGGTACACCAGCACCAAGGAGGT
GCTGGACGCCACCCTGATCCACCAGAGCATCACCGGCCTG
TACGAGACCCGGATCGACCTGAGCCAGCTGGGCGGCGACA
GCGCCGGCGGCGGCGGCAGCGGCGGCGGCGGCAGCGGCGG
CGGCGGCAGCGGCCCCAAGAAGAAGCGGAAGGTGGCCGCC
GCCGGCAGCAACCACGACCAGGAGTTCGACCCCCCCAAGG
TGTACCCCCCCGTGCCCGCCGAGAAGCGGAAGCCCATCCG
GGTGCTGAGCCTGTTCGACGGCATCGCCACCGGCCTGCTG
GTGCTGAAGGACCTGGGCATCCAGGTGGACCGGTACATCG
CCAGCGAGGTGTGCGAGGACAGCATCACCGTGGGCATGGT
GCGGCACCAGGGCAAGATCATGTACGTGGGCGACGTGCGG
AGCGTGACCCAGAAGCACATCCAGGAGTGGGGCCCCTTCG
ACCTGGTGATCGGCGGCAGCCCCTGCAACGACCTGAGCAT
CGTGAACCCCGCCCGGAAGGGCCTGTACGAGGGCACCGGC
CGGCTGTTCTTCGAGTTCTACCGGCTGCTGCACGACGCCC
GGCCCAAGGAGGGCGACGACCGGCCCTTCTTCTGGCTGTT
CGAGAACGTGGTGGCCATGGGCGTGAGCGACAAGCGGGAC
ATCAGCCGGTTCCTGGAGAGCAACCCCGTGATGATCGACG
CCAAGGAGGTGAGCGCCGCCCACCGGGCCCGGTACTTCTG
GGGCAACCTGCCCGGCATGAACCGGCCCCTGGCCAGCACC
GTGAACGACAAGCTGGAGCTGCAGGAGTGCCTGGAGCACG
GCCGGATCGCCAAGTTCAGCAAGGTGCGGACCATCACCAC
CCGGAGCAACAGCATCAAGCAGGGCAAGGACCAGCACTTC
CCCGTGTTCATGAACGAGAAGGAGGACATCCTGTGGTGCA
CCGAGATGGAGCGGGTGTTCGGCTTCCCCGTGCACTACAC
CGACGTGAGCAACATGAGCCGGCTGGCCCGGCAGCGGCTG
CTGGGCCGGAGCTGGAGCGTGCCCGTGATCCGGCACCTGT
TCGCCCCCCTGAAGGAGTACTTCGCCTGCGTGAGCAGCGG
CAACAGCAACGCCAACAGCCGGGGCCCCAGCTTCAGCAGC
GGCCTGGTGCCCCTGAGCCTGCGGGGCAGCCACATGAATC
CTCTGGAGATGTTCGAGACAGTGCCCGTGTGGAGAAGGCA
ACCCGTGAGGGTGCTGAGCCTCTTCGAGGACATTAAGAAG
GAGCTGACCTCTCTGGGCTTTCTGGAATCCGGCAGCGACC
CCGGCCAGCTGAAACACGTGGTGGACGTGACCGACACAGT
GAGGAAGGACGTGGAAGAGTGGGGCCCCTTTGACCTCGTG
TATGGAGCCACACCTCCTCTCGGCCACACATGCGATAGGC
CTCCCAGCTGGTATCTCTTCCAGTTCCACAGACTGCTCCA
GTACGCCAGACCTAAGCCCGGCAGCCCCAGACCCTTCTTC
TGGATGTTCGTGGACAATCTGGTGCTGAACAAGGAGGATC
TGGATGTGGCCAGCAGATTTCTGGAGATGGAACCCGTGAC
AATCCCCGACGTGCATGGCGGCTCTCTGCAGAACGCCGTG
AGAGTGTGGTCCAACATCCCCGCCATTAGAAGCAGACACT
GGGCTCTGGTGAGCGAGGAGGAACTGTCTCTGCTGGCCCA
GAATAAGCAGTCCTCCAAGCTGGCCGCCAAGTGGCCCACC
AAGCTGGTGAAGAACTGCTTTCTGCCTCTGAGGGAGTATT
TCAAGTATTTCAGCACCGAACTGACCAGCAGCCTGAGCGG
CGGCAAGCGGCCCGCCGCCACCAAGAAGGCCGGCCAGGCC
AAGAAGAAGAAGGGCAGCTACCCCTACGACGTGCCCGACT
ACGCCTGAGCGGCCGCTTAATTAAGCTGCCTTCTGCGGGG
CTTGCCTTCTGGCCATGCCCTTCTTCTCTCCCTTGCACCT
GTACCTCTTGGTCTTTGAATAAAGCCTGAGTAGGAAGTCT
AGAAAAAAAAAAAAAAAAAAAAAAAAAAAAAAAAAAAAAA
AAAAAAAAAAAAAAAAAAAAAAAAAAAAAAAAAAAAAAAA
AA
dCas9-DNMT3a/3L (m) (MR-28195)
(SEQ ID NO: 25)
AGGAAATAAGAGAGAAAAGAAGAGTAAGAAGAAATATAAG
AGCCACCATGGCCCCCAAGAAGAAGCGGAAGGTGGGCATC
CACGGCGTGCCCGCCGCCGACAAGAAGTACAGCATCGGCC
TGGCCATCGGCACCAACAGCGTGGGCTGGGCCGTGATCAC
CGACGAGTACAAGGTGCCCAGCAAGAAGTTCAAGGTGCTG
GGCAACACCGACCGGCACAGCATCAAGAAGAACCTGATCG
GCGCCCTGCTGTTCGACAGCGGCGAGACCGCCGAGGCCAC
CCGGCTGAAGCGGACCGCCCGGCGGCGGTACACCCGGCGG
AAGAACCGGATCTGCTACCTGCAGGAGATCTTCAGCAACG
AGATGGCCAAGGTGGACGACAGCTTCTTCCACCGGCTGGA
GGAGAGCTTCCTGGTGGAGGAGGACAAGAAGCACGAGCGG
CACCCCATCTTCGGCAACATCGTGGACGAGGTGGCCTACC
ACGAGAAGTACCCCACCATCTACCACCTGCGGAAGAAGCT
GGTGGACAGCACCGACAAGGCCGACCTGCGGCTGATCTAC
CTGGCCCTGGCCCACATGATCAAGTTCCGGGGCCACTTCC
TGATCGAGGGCGACCTGAACCCCGACAACAGCGACGTGGA
CAAGCTGTTCATCCAGCTGGTGCAGACCTACAACCAGCTG
TTCGAGGAGAACCCCATCAACGCCAGCGGCGTGGACGCCA
AGGCCATCCTGAGCGCCCGGCTGAGCAAGAGCCGGCGGCT
GGAGAACCTGATCGCCCAGCTGCCCGGCGAGAAGAAGAAC
GGCCTGTTCGGCAACCTGATCGCCCTGAGCCTGGGCCTGA
CCCCCAACTTCAAGAGCAACTTCGACCTGGCCGAGGACGC
CAAGCTGCAGCTGAGCAAGGACACCTACGACGACGACCTG
GACAACCTGCTGGCCCAGATCGGCGACCAGTACGCCGACC
TGTTCCTGGCCGCCAAGAACCTGAGCGACGCCATCCTGCT
GAGCGACATCCTGCGGGTGAACACCGAGATCACCAAGGCC
CCCCTGAGCGCCAGCATGATCAAGCGGTACGACGAGCACC
ACCAGGACCTGACCCTGCTGAAGGCCCTGGTGCGGCAGCA
GCTGCCCGAGAAGTACAAGGAGATCTTCTTCGACCAGAGC
AAGAACGGCTACGCCGGCTACATCGACGGCGGCGCCAGCC
AGGAGGAGTTCTACAAGTTCATCAAGCCCATCCTGGAGAA
GATGGACGGCACCGAGGAGCTGCTGGTGAAGCTGAACCGG
GAGGACCTGCTGCGGAAGCAGCGGACCTTCGACAACGGCA
GCATCCCCCACCAGATCCACCTGGGCGAGCTGCACGCCAT
CCTGCGGCGGCAGGAGGACTTCTACCCCTTCCTGAAGGAC
AACCGGGAGAAGATCGAGAAGATCCTGACCTTCCGGATCC
CCTACTACGTGGGCCCCCTGGCCCGGGGCAACAGCCGGTT
CGCCTGGATGACCCGGAAATCCGAGGAGACCATCACCCCC
TGGAACTTCGAGGAGGTGGTGGACAAGGGCGCCAGCGCCC
AGAGCTTCATCGAGCGGATGACCAACTTCGACAAGAACCT
GCCCAACGAGAAGGTGCTGCCCAAGCACAGCCTGCTGTAC
GAGTACTTCACCGTGTACAACGAGCTGACCAAGGTGAAGT
ACGTGACCGAGGGCATGCGGAAGCCCGCCTTCCTGAGCGG
CGAGCAGAAGAAGGCCATCGTGGACCTGCTGTTCAAGACC
AACCGGAAGGTGACCGTGAAGCAGCTGAAGGAGGACTACT
TCAAGAAGATCGAGTGCTTCGACAGCGTGGAGATCAGCGG
CGTGGAGGACCGGTTCAACGCCAGCCTGGGCACCTACCAC
GACCTGCTGAAGATCATCAAGGACAAGGACTTCCTGGACA
ACGAGGAGAACGAGGACATCCTGGAGGACATCGTGCTGAC
CCTGACCCTGTTCGAGGACCGGGAGATGATCGAGGAGCGG
CTGAAAACCTACGCCCACCTGTTCGACGACAAGGTGATGA
AGCAGCTGAAGCGGCGGCGGTACACCGGCTGGGGCCGGCT
GAGCCGGAAGCTGATCAACGGCATCCGGGACAAGCAGAGC
GGCAAGACCATCCTGGACTTCCTGAAATCCGACGGCTTCG
CCAACCGGAACTTCATGCAGCTGATCCACGACGACAGCCT
GACCTTCAAGGAGGACATCCAGAAGGCCCAGGTGAGCGGC
CAGGGCGACAGCCTGCACGAGCACATCGCCAACCTGGCCG
GCAGCCCCGCCATCAAGAAGGGCATCCTGCAGACCGTGAA
GGTGGTGGACGAGCTGGTGAAGGTGATGGGCCGGCACAAG
CCCGAGAACATCGTGATCGAGATGGCCCGGGAGAACCAGA
CCACCCAGAAGGGCCAGAAGAACAGCCGGGAGCGGATGAA
GCGGATCGAGGAGGGCATCAAGGAGCTGGGCAGCCAGATC
CTGAAGGAGCACCCCGTGGAGAACACCCAGCTGCAGAACG
AGAAGCTGTACCTGTACTACCTGCAGAACGGCCGGGACAT
GTACGTGGACCAGGAGCTGGACATCAACCGGCTGAGCGAC
TACGACGTGGCCGCCATCGTGCCCCAGAGCTTCCTGAAGG
ACGACAGCATCGACAACAAGGTGCTGACCCGGAGCGACAA
GGCCCGGGGCAAGAGCGACAACGTGCCCAGCGAGGAGGTG
GTGAAGAAGATGAAGAACTACTGGCGGCAGCTGCTGAACG
CCAAGCTGATCACCCAGCGGAAGTTCGACAACCTGACCAA
GGCCGAGCGGGGCGGCCTGAGCGAGCTGGACAAGGCCGGC
TTCATCAAGCGGCAGCTGGTGGAGACCCGGCAGATCACCA
AGCACGTGGCCCAGATCCTGGACAGCCGGATGAACACCAA
GTACGACGAGAACGACAAGCTGATCCGGGAGGTGAAGGTG
ATCACCCTGAAATCCAAGCTGGTGAGCGACTTCCGGAAGG
ACTTCCAGTTCTACAAGGTGCGGGAGATCAACAACTACCA
CCACGCCCACGACGCCTACCTGAACGCCGTGGTGGGCACC
GCCCTGATCAAGAAGTACCCCAAGCTGGAGAGCGAGTTCG
TGTACGGCGACTACAAGGTGTACGACGTGCGGAAGATGAT
CGCCAAGAGCGAGCAGGAGATCGGCAAGGCCACCGCCAAG
TACTTCTTCTACAGCAACATCATGAACTTCTTCAAGACCG
AGATCACCCTGGCCAACGGCGAGATCCGGAAGCGGCCCCT
GATCGAGACCAACGGCGAGACCGGCGAGATCGTGTGGGAC
AAGGGCCGGGACTTCGCCACCGTGCGGAAGGTGCTGAGCA
TGCCCCAGGTGAACATCGTGAAGAAAACCGAGGTGCAGAC
CGGCGGCTTCAGCAAGGAGAGCATCCTGCCCAAGCGGAAC
AGCGACAAGCTGATCGCCCGGAAGAAGGACTGGGACCCCA
AGAAGTACGGCGGCTTCGACAGCCCCACCGTGGCCTACAG
CGTGCTGGTGGTGGCCAAGGTGGAGAAGGGCAAGAGCAAG
AAGCTGAAATCCGTGAAGGAGCTGCTGGGCATCACCATCA
TGGAGCGGAGCAGCTTCGAGAAGAACCCCATCGACTTCCT
GGAGGCCAAGGGCTACAAGGAGGTGAAGAAGGACCTGATC
ATCAAGCTGCCCAAGTACAGCCTGTTCGAGCTGGAGAACG
GCCGGAAGCGGATGCTGGCCAGCGCCGGCGAGCTGCAGAA
GGGCAACGAGCTGGCCCTGCCCAGCAAGTACGTGAACTTC
CTGTACCTGGCCAGCCACTACGAGAAGCTGAAGGGCAGCC
CCGAGGACAACGAGCAGAAGCAGCTGTTCGTGGAGCAGCA
CAAGCACTACCTGGACGAGATCATCGAGCAGATCAGCGAG
TTCAGCAAGCGGGTGATCCTGGCCGACGCCAACCTGGACA
AGGTGCTGAGCGCCTACAACAAGCACCGGGACAAGCCCAT
CCGGGAGCAGGCCGAGAACATCATCCACCTGTTCACCCTG
ACCAACCTGGGCGCCCCCGCCGCCTTCAAGTACTTCGACA
CCACCATCGACCGGAAGCGGTACACCAGCACCAAGGAGGT
GCTGGACGCCACCCTGATCCACCAGAGCATCACCGGCCTG
TACGAGACCCGGATCGACCTGAGCCAGCTGGGCGGCGACA
GCGCCGGCGGCGGCGGCAGCGGCGGCGGCGGCAGCGGCGG
CGGCGGCAGCGGCCCCAAGAAGAAGCGGAAGGTGGCCGCC
GCCGGCAGCAACCACGACCAGGAGTTCGACCCCCCCAAGG
TGTACCCCCCCGTGCCCGCCGAGAAGCGGAAGCCCATCCG
GGTGCTGAGCCTGTTCGACGGCATCGCCACCGGCCTGCTG
GTGCTGAAGGACCTGGGCATCCAGGTGGACCGGTACATCG
CCAGCGAGGTGTGCGAGGACAGCATCACCGTGGGCATGGT
GCGGCACCAGGGCAAGATCATGTACGTGGGCGACGTGCGG
AGCGTGACCCAGAAGCACATCCAGGAGTGGGGCCCCTTCG
ACCTGGTGATCGGCGGCAGCCCCTGCAACGACCTGAGCAT
CGTGAACCCCGCCCGGAAGGGCCTGTACGAGGGCACCGGC
CGGCTGTTCTTCGAGTTCTACCGGCTGCTGCACGACGCCC
GGCCCAAGGAGGGCGACGACCGGCCCTTCTTCTGGCTGTT
CGAGAACGTGGTGGCCATGGGCGTGAGCGACAAGCGGGAC
ATCAGCCGGTTCCTGGAGAGCAACCCCGTGATGATCGACG
CCAAGGAGGTGAGCGCCGCCCACCGGGCCCGGTACTTCTG
GGGCAACCTGCCCGGCATGAACCGGCCCCTGGCCAGCACC
GTGAACGACAAGCTGGAGCTGCAGGAGTGCCTGGAGCACG
GCCGGATCGCCAAGTTCAGCAAGGTGCGGACCATCACCAC
CCGGAGCAACAGCATCAAGCAGGGCAAGGACCAGCACTTC
CCCGTGTTCATGAACGAGAAGGAGGACATCCTGTGGTGCA
CCGAGATGGAGCGGGTGTTCGGCTTCCCCGTGCACTACAC
CGACGTGAGCAACATGAGCCGGCTGGCCCGGCAGCGGCTG
CTGGGCCGGAGCTGGAGCGTGCCCGTGATCCGGCACCTGT
TCGCCCCCCTGAAGGAGTACTTCGCCTGCGTGAGCAGCGG
CAACAGCAACGCCAACAGCCGGGGCCCCAGCTTCAGCAGC
GGCCTGGTGCCCCTGAGCCTGCGGGGCAGCCACATGGGCC
CCATGGAGATCTACAAGACCGTGAGCGCCTGGAAGCGGCA
GCCCGTGCGGGTGCTGAGCCTGTTCCGGAACATCGACAAG
GTGCTGAAATCCCTGGGCTTCCTGGAGAGCGGCAGCGGCA
GCGGCGGCGGCACCCTGAAGTACGTGGAGGACGTGACCAA
CGTGGTGCGGCGGGACGTGGAGAAGTGGGGCCCCTTCGAC
CTGGTGTACGGCAGCACCCAGCCCCTGGGCAGCAGCTGCG
ACCGGTGCCCCGGCTGGTACATGTTCCAGTTCCACCGGAT
CCTGCAGTACGCCCTGCCCCGGCAGGAGAGCCAGCGGCCC
TTCTTCTGGATCTTCATGGACAACCTGCTGCTGACCGAGG
ACGACCAGGAGACCACCACCCGGTTCCTGCAGACCGAGGC
CGTGACCCTGCAGGACGTGCGGGGCCGGGACTACCAGAAC
GCCATGCGGGTGTGGAGCAACATCCCCGGCCTGAAATCCA
AGCACGCCCCCCTGACCCCCAAGGAGGAGGAGTACCTGCA
GGCCCAGGTGCGGAGCCGGAGCAAGCTGGACGCCCCCAAG
GTGGACCTGCTGGTGAAGAACTGCCTGCTGCCCCTGCGGG
AGTACTTCAAGTACTTCAGCCAGAACAGCCTGCCCCTGAG
CGGCGGCAAGCGGCCCGCCGCCACCAAGAAGGCCGGCCAG
GCCAAGAAGAAGAAGGGCAGCTACCCCTACGACGTGCCCG
ACTACGCCTGAGCGGCCGCTTAATTAAGCTGCCTTCTGCG
GGGCTTGCCTTCTGGCCATGCCCTTCTTCTCTCCCTTGCA
CCTGTACCTCTTGGTCTTTGAATAAAGCCTGAGTAGGAAG
TCTAGAAAAAAAAAAAAAAAAAAAAAAAAAAAAAAAAAAA
AAAAAAAAAAAAAAAAAAAAAAAAAAAAAAAAAAAAAAAA
AAAAA

In some embodiments, an expression repressor comprises the amino acid sequence of SEQ ID NOs: 35, 36, 70, or 71. In some embodiments, an expression repressor described herein comprises an amino acid sequence of SEQ ID NO: 35, 36, 70, or 71 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3a/3L (h)
(SEQ ID NO: 35)
MAPKKKRKVGIHGVPAADKKYSIGLAIGTNSVGWAVITDE
YKVPSKKFKVLGNTDRHSIKKNLIGALLFDSGETAEATRL
KRTARRRYTRRKNRICYLQEIFSNEMAKVDDSFFHRLEES
FLVEEDKKHERHPIFGNIVDEVAYHEKYPTIYHLRKKLVD
STDKADLRLIYLALAHMIKFRGHFLIEGDLNPDNSDVDKL
FIQLVQTYNQLFEENPINASGVDAKAILSARLSKSRRLEN
LIAQLPGEKKNGLFGNLIALSLGLTPNFKSNFDLAEDAKL
QLSKDTYDDDLDNLLAQIGDQYADLFLAAKNLSDAILLSD
ILRVNTEITKAPLSASMIKRYDEHHQDLTLLKALVRQQLP
EKYKEIFFDQSKNGYAGYIDGGASQEEFYKFIKPILEKMD
GTEELLVKLNREDLLRKQRTFDNGSIPHQIHLGELHAILR
RQEDFYPFLKDNREKIEKILTFRIPYYVGPLARGNSRFAW
MTRKSEETITPWNFEEVVDKGASAQSFIERMTNFDKNLPN
EKVLPKHSLLYEYFTVYNELTKVKYVTEGMRKPAFLSGEQ
KKAIVDLLFKTNRKVTVKQLKEDYFKKIECFDSVEISGVE
DRFNASLGTYHDLLKIIKDKDFLDNEENEDILEDIVLTLT
LFEDREMIEERLKTYAHLFDDKVMKQLKRRRYTGWGRLSR
KLINGIRDKQSGKTILDFLKSDGFANRNFMQLIHDDSLTF
KEDIQKAQVSGQGDSLHEHIANLAGSPAIKKGILQTVKVV
DELVKVMGRHKPENIVIEMARENQTTQKGQKNSRERMKRI
EEGIKELGSQILKEHPVENTQLQNEKLYLYYLQNGRDMYV
DQELDINRLSDYDVAAIVPQSFLKDDSIDNKVLTRSDKAR
GKSDNVPSEEVVKKMKNYWRQLLNAKLITQRKFDNLTKAE
RGGLSELDKAGFIKRQLVETRQITKHVAQILDSRMNTKYD
ENDKLIREVKVITLKSKLVSDFRKDFQFYKVREINNYHHA
HDAYLNAVVGTALIKKYPKLESEFVYGDYKVYDVRKMIAK
SEQEIGKATAKYFFYSNIMNFFKTEITLANGEIRKRPLIE
TNGETGEIVWDKGRDFATVRKVLSMPQVNIVKKTEVQTGG
FSKESILPKRNSDKLIARKKDWDPKKYGGFDSPTVAYSVL
VVAKVEKGKSKKLKSVKELLGITIMERSSFEKNPIDFLEA
KGYKEVKKDLIIKLPKYSLFELENGRKRMLASAGELQKGN
ELALPSKYVNFLYLASHYEKLKGSPEDNEQKQLFVEQHKH
YLDEIIEQISEFSKRVILADANLDKVLSAYNKHRDKPIRE
QAENIIHLFTLTNLGAPAAFKYFDTTIDRKRYTSTKEVLD
ATLIHQSITGLYETRIDLSQLGGDSAGGGGSGGGGSGGGG
SGPKKKRKVAAAGSNHDQEFDPPKVYPPVPAEKRKPIRVL
SLFDGIATGLLVLKDLGIQVDRYIASEVCEDSITVGMVRH
QGKIMYVGDVRSVTQKHIQEWGPFDLVIGGSPCNDLSIVN
PARKGLYEGTGRLFFEFYRLLHDARPKEGDDRPFFWLFEN
VVAMGVSDKRDISRFLESNPVMIDAKEVSAAHRARYFWGN
LPGMNRPLASTVNDKLELQECLEHGRIAKFSKVRTITTRS
NSIKQGKDQHFPVFMNEKEDILWCTEMERVFGFPVHYTDV
SNMSRLARQRLLGRSWSVPVIRHLFAPLKEYFACVSSGNS
NANSRGPSFSSGLVPLSLRGSHMNPLEMFETVPVWRRQPV
RVLSLFEDIKKELTSLGFLESGSDPGQLKHVVDVTDTVRK
DVEEWGPFDLVYGATPPLGHTCDRPPSWYLFQFHRLLQYA
RPKPGSPRPFFWMFVDNLVLNKEDLDVASRFLEMEPVTIP
DVHGGSLQNAVRVWSNIPAIRSRHWALVSEEELSLLAQNK
QSSKLAAKWPTKLVKNCFLPLREYFKYFSTELTSSLSGGK
RPAATKKAGQAKKKKGSYPYDVPDYA
dCas9-DNMT3a/31 (m)
(SEQ ID NO: 36)
MAPKKKRKVGIHGVPAADKKYSIGLAIGTNSVGWAVITDE
YKVPSKKFKVLGNTDRHSIKKNLIGALLFDSGETAEATRL
KRTARRRYTRRKNRICYLQEIFSNEMAKVDDSFFHRLEES
FLVEEDKKHERHPIFGNIVDEVAYHEKYPTIYHLRKKLVD
STDKADLRLIYLALAHMIKFRGHFLIEGDLNPDNSDVDKL
FIQLVQTYNQLFEENPINASGVDAKAILSARLSKSRRLEN
LIAQLPGEKKNGLFGNLIALSLGLTPNFKSNFDLAEDAKL
QLSKDTYDDDLDNLLAQIGDQYADLFLAAKNLSDAILLSD
ILRVNTEITKAPLSASMIKRYDEHHQDLTLLKALVRQQLP
EKYKEIFFDQSKNGYAGYIDGGASQEEFYKFIKPILEKMD
GTEELLVKLNREDLLRKQRTFDNGSIPHQIHLGELHAILR
RQEDFYPFLKDNREKIEKILTFRIPYYVGPLARGNSRFAW
MTRKSEETITPWNFEEVVDKGASAQSFIERMTNFDKNLPN
EKVLPKHSLLYEYFTVYNELTKVKYVTEGMRKPAFLSGEQ
KKAIVDLLFKTNRKVTVKQLKEDYFKKIECFDSVEISGVE
DRFNASLGTYHDLLKIIKDKDFLDNEENEDILEDIVLTLT
LFEDREMIEERLKTYAHLFDDKVMKQLKRRRYTGWGRLSR
KLINGIRDKQSGKTILDFLKSDGFANRNFMQLIHDDSLTF
KEDIQKAQVSGQGDSLHEHIANLAGSPAIKKGILQTVKVV
DELVKVMGRHKPENIVIEMARENQTTQKGQKNSRERMKRI
EEGIKELGSQILKEHPVENTQLQNEKLYLYYLQNGRDMYV
DQELDINRLSDYDVAAIVPQSFLKDDSIDNKVLTRSDKAR
GKSDNVPSEEVVKKMKNYWRQLLNAKLITQRKFDNLTKAE
RGGLSELDKAGFIKRQLVETRQITKHVAQILDSRMNTKYD
ENDKLIREVKVITLKSKLVSDFRKDFQFYKVREINNYHHA
HDAYLNAVVGTALIKKYPKLESEFVYGDYKVYDVRKMIAK
SEQEIGKATAKYFFYSNIMNFFKTEITLANGEIRKRPLIE
TNGETGEIVWDKGRDFATVRKVLSMPQVNIVKKTEVQTGG
FSKESILPKRNSDKLIARKKDWDPKKYGGFDSPTVAYSVL
VVAKVEKGKSKKLKSVKELLGITIMERSSFEKNPIDFLEA
KGYKEVKKDLIIKLPKYSLFELENGRKRMLASAGELQKGN
ELALPSKYVNFLYLASHYEKLKGSPEDNEQKQLFVEQHKH
YLDEIIEQISEFSKRVILADANLDKVLSAYNKHRDKPIRE
QAENIIHLFTLTNLGAPAAFKYFDTTIDRKRYTSTKEVLD
ATLIHQSITGLYETRIDLSQLGGDSAGGGGSGGGGSGGGG
SGPKKKRKVAAAGSNHDQEFDPPKVYPPVPAEKRKPIRVL
SLFDGIATGLLVLKDLGIQVDRYIASEVCEDSITVGMVRH
QGKIMYVGDVRSVTQKHIQEWGPFDLVIGGSPCNDLSIVN
PARKGLYEGTGRLFFEFYRLLHDARPKEGDDRPFFWLFEN
VVAMGVSDKRDISRFLESNPVMIDAKEVSAAHRARYFWGN
LPGMNRPLASTVNDKLELQECLEHGRIAKFSKVRTITTRS
NSIKQGKDQHFPVFMNEKEDILWCTEMERVFGFPVHYTDV
SNMSRLARQRLLGRSWSVPVIRHLFAPLKEYFACVSSGNS
NANSRGPSFSSGLVPLSLRGSHMNPLEMFETVPVWRRQPV
RVLSLFEDIKKELTSLGFLESGSDPGQLKHVVDVTDTVRK
DVEEWGPFDLVYGATPPLGHTCDRPPSWYLFQFHRLLQYA
RPKPGSPRPFFWMFVDNLVLNKEDLDVASRFLEMEPVTIP
DVHGGSLQNAVRVWSNIPAIRSRHWALVSEEELSLLAQNK
QSSKLAAKWPTKLVKNCFLPLREYFKYFSTELTSSLSGGK
RPAATKKAGQAKKKKGSYPYDVPDYA
dCas-DNMT3a/3L (h) without HA tag
(SEQ ID NO: 70)
MAPKKKRKVGIHGVPAADKKYSIGLAIGTNSVGWAVITDE
YKVPSKKFKVLGNTDRHSIKKNLIGALLFDSGETAEATRL
KRTARRRYTRRKNRICYLQEIFSNEMAKVDDSFFHRLEES
FLVEEDKKHERHPIFGNIVDEVAYHEKYPTIYHLRKKLVD
STDKADLRLIYLALAHMIKFRGHFLIEGDLNPDNSDVDKL
FIQLVQTYNQLFEENPINASGVDAKAILSARLSKSRRLEN
LIAQLPGEKKNGLFGNLIALSLGLTPNFKSNFDLAEDAKL
QLSKDTYDDDLDNLLAQIGDQYADLFLAAKNLSDAILLSD
ILRVNTEITKAPLSASMIKRYDEHHQDLTLLKALVRQQLP
EKYKEIFFDQSKNGYAGYIDGGASQEEFYKFIKPILEKMD
GTEELLVKLNREDLLRKQRTFDNGSIPHQIHLGELHAILR
RQEDFYPFLKDNREKIEKILTFRIPYYVGPLARGNSRFAW
MTRKSEETITPWNFEEVVDKGASAQSFIERMTNFDKNLPN
EKVLPKHSLLYEYFTVYNELTKVKYVTEGMRKPAFLSGEQ
KKAIVDLLFKTNRKVTVKQLKEDYFKKIECFDSVEISGVE
DRFNASLGTYHDLLKIIKDKDFLDNEENEDILEDIVLTLT
LFEDREMIEERLKTYAHLFDDKVMKQLKRRRYTGWGRLSR
KLINGIRDKQSGKTILDFLKSDGFANRNFMQLIHDDSLTF
KEDIQKAQVSGQGDSLHEHIANLAGSPAIKKGILQTVKVV
DELVKVMGRHKPENIVIEMARENQTTQKGQKNSRERMKRI
EEGIKELGSQILKEHPVENTQLQNEKLYLYYLQNGRDMYV
DQELDINRLSDYDVAAIVPQSFLKDDSIDNKVLTRSDKAR
GKSDNVPSEEVVKKMKNYWRQLLNAKLITQRKFDNLTKAE
RGGLSELDKAGFIKRQLVETRQITKHVAQILDSRMNTKYD
ENDKLIREVKVITLKSKLVSDFRKDFQFYKVREINNYHHA
HDAYLNAVVGTALIKKYPKLESEFVYGDYKVYDVRKMIAK
SEQEIGKATAKYFFYSNIMNFFKTEITLANGEIRKRPLIE
TNGETGEIVWDKGRDFATVRKVLSMPQVNIVKKTEVQTGG
FSKESILPKRNSDKLIARKKDWDPKKYGGFDSPTVAYSVL
VVAKVEKGKSKKLKSVKELLGITIMERSSFEKNPIDFLEA
KGYKEVKKDLIIKLPKYSLFELENGRKRMLASAGELQKGN
ELALPSKYVNFLYLASHYEKLKGSPEDNEQKQLFVEQHKH
YLDEIIEQISEFSKRVILADANLDKVLSAYNKHRDKPIRE
QAENIIHLFTLTNLGAPAAFKYFDTTIDRKRYTSTKEVLD
ATLIHQSITGLYETRIDLSQLGGDSAGGGGSGGGGSGGGG
SGPKKKRKVAAAGSNHDQEFDPPKVYPPVPAEKRKPIRVL
SLFDGIATGLLVLKDLGIQVDRYIASEVCEDSITVGMVRH
QGKIMYVGDVRSVTQKHIQEWGPFDLVIGGSPCNDLSIVN
PARKGLYEGTGRLFFEFYRLLHDARPKEGDDRPFFWLFEN
VVAMGVSDKRDISRFLESNPVMIDAKEVSAAHRARYFWGN
LPGMNRPLASTVNDKLELQECLEHGRIAKFSKVRTITTRS
NSIKQGKDQHFPVFMNEKEDILWCTEMERVFGFPVHYTDV
SNMSRLARQRLLGRSWSVPVIRHLFAPLKEYFACVSSGNS
NANSRGPSFSSGLVPLSLRGSHMNPLEMFETVPVWRRQPV
RVLSLFEDIKKELTSLGFLESGSDPGQLKHVVDVTDTVRK
DVEEWGPFDLVYGATPPLGHTCDRPPSWYLFQFHRLLQYA
RPKPGSPRPFFWMFVDNLVLNKEDLDVASRFLEMEPVTIP
DVHGGSLQNAVRVWSNIPAIRSRHWALVSEEELSLLAQNK
QSSKLAAKWPTKLVKNCFLPLREYFKYFSTELTSSLSGGK
RPAATKKAGQAKKKKGS
dCas9-DNMT3a/3L (m) without HA tag
(SEQ ID NO: 71)
MAPKKKRKVGIHGVPAADKKYSIGLAIGTNSVGWAVITDE
YKVPSKKFKVLGNTDRHSIKKNLIGALLFDSGETAEATRL
KRTARRRYTRRKNRICYLQEIFSNEMAKVDDSFFHRLEES
FLVEEDKKHERHPIFGNIVDEVAYHEKYPTIYHLRKKLVD
STDKADLRLIYLALAHMIKFRGHFLIEGDLNPDNSDVDKL
FIQLVQTYNQLFEENPINASGVDAKAILSARLSKSRRLEN
LIAQLPGEKKNGLFGNLIALSLGLTPNFKSNFDLAEDAKL
QLSKDTYDDDLDNLLAQIGDQYADLFLAAKNLSDAILLSD
ILRVNTEITKAPLSASMIKRYDEHHQDLTLLKALVRQQLP
EKYKEIFFDQSKNGYAGYIDGGASQEEFYKFIKPILEKMD
GTEELLVKLNREDLLRKQRTFDNGSIPHQIHLGELHAILR
RQEDFYPFLKDNREKIEKILTFRIPYYVGPLARGNSRFAW
MTRKSEETITPWNFEEVVDKGASAQSFIERMTNFDKNLPN
EKVLPKHSLLYEYFTVYNELTKVKYVTEGMRKPAFLSGEQ
KKAIVDLLFKTNRKVTVKQLKEDYFKKIECFDSVEISGVE
DRFNASLGTYHDLLKIIKDKDFLDNEENEDILEDIVLTLT
LFEDREMIEERLKTYAHLFDDKVMKQLKRRRYTGWGRLSR
KLINGIRDKQSGKTILDFLKSDGFANRNFMQLIHDDSLTF
KEDIQKAQVSGQGDSLHEHIANLAGSPAIKKGILQTVKVV
DELVKVMGRHKPENIVIEMARENQTTQKGQKNSRERMKRI
EEGIKELGSQILKEHPVENTQLQNEKLYLYYLQNGRDMYV
DQELDINRLSDYDVAAIVPQSFLKDDSIDNKVLTRSDKAR
GKSDNVPSEEVVKKMKNYWRQLLNAKLITQRKFDNLTKAE
RGGLSELDKAGFIKRQLVETRQITKHVAQILDSRMNTKYD
ENDKLIREVKVITLKSKLVSDFRKDFQFYKVREINNYHHA
HDAYLNAVVGTALIKKYPKLESEFVYGDYKVYDVRKMIAK
SEQEIGKATAKYFFYSNIMNFFKTEITLANGEIRKRPLIE
TNGETGEIVWDKGRDFATVRKVLSMPQVNIVKKTEVQTGG
FSKESILPKRNSDKLIARKKDWDPKKYGGFDSPTVAYSVL
VVAKVEKGKSKKLKSVKELLGITIMERSSFEKNPIDFLEA
KGYKEVKKDLIIKLPKYSLFELENGRKRMLASAGELQKGN
ELALPSKYVNFLYLASHYEKLKGSPEDNEQKQLFVEQHKH
YLDEIIEQISEFSKRVILADANLDKVLSAYNKHRDKPIRE
QAENIIHLFTLTNLGAPAAFKYFDTTIDRKRYTSTKEVLD
ATLIHQSITGLYETRIDLSQLGGDSAGGGGSGGGGSGGGG
SGPKKKRKVAAAGSNHDQEFDPPKVYPPVPAEKRKPIRVL
SLFDGIATGLLVLKDLGIQVDRYIASEVCEDSITVGMVRH
QGKIMYVGDVRSVTQKHIQEWGPFDLVIGGSPCNDLSIVN
PARKGLYEGTGRLFFEFYRLLHDARPKEGDDRPFFWLFEN
VVAMGVSDKRDISRFLESNPVMIDAKEVSAAHRARYFWGN
LPGMNRPLASTVNDKLELQECLEHGRIAKFSKVRTITTRS
NSIKQGKDQHFPVFMNEKEDILWCTEMERVFGFPVHYTDV
SNMSRLARQRLLGRSWSVPVIRHLFAPLKEYFACVSSGNS
NANSRGPSFSSGLVPLSLRGSHMNPLEMFETVPVWRRQPV
RVLSLFEDIKKELTSLGFLESGSDPGQLKHVVDVTDTVRK
DVEEWGPFDLVYGATPPLGHTCDRPPSWYLFQFHRLLQYA
RPKPGSPRPFFWMFVDNLVLNKEDLDVASRFLEMEPVTIP
DVHGGSLQNAVRVWSNIPAIRSRHWALVSEEELSLLAQNK
QSSKLAAKWPTKLVKNCFLPLREYFKYFSTELTSSLSGGK
RPAATKKAGQAKKKKGS

In some embodiments, an expression repressor comprises a DNA-targeting moiety comprising dCas9, e.g., an S. pyogenes dCas9, and a repressor domain comprising G9A, e.g., a G9A domain. In some embodiments, the expression repressor is encoded by the nucleic acid sequence of SEQ ID NOs: 26 (e.g., a nucleic acid (e.g., cDNA) encoding the expression repressor). In some embodiments, a nucleic acid described herein comprises a nucleic acid sequence of SEQ ID NO: 26 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9 (MR-29387)
(SEQ ID NO: 26)
AGGAAATAAGAGAGAAAAGAAGAGTAAGAAGAAATATAAG
AGCCACCATGGCCCCCAAGAAGAAGCGGAAGGTGGGCATC
CACGGCGTGCCCGCCGCCTCGGGCGGGGGTGGCTCAGGAA
ATAGGGCTATCAGAACCGAGAAGATCATCTGTAGGGACGT
GGCTAGAGGCTACGAGAACGTGCCCATTCCTTGCGTGAAT
GGCGTGGATGGCGAACCTTGCCCCGAGGACTACAAATACA
TCTCCGAGAACTGCGAAACCAGCACAATGAACATCGACAG
AAACATCACCCACCTCCAGCACTGCACATGTGTGGATGAC
TGCTCCTCCAGCAACTGTCTGTGCGGCCAGCTCTCCATCA
GATGCTGGTACGACAAGGACGGCAGACTGCTGCAAGAGTT
CAACAAGATCGAACCCCCTCTCATCTTCGAGTGTAACCAA
GCTTGCAGCTGCTGGAGAAACTGCAAGAATAGAGTGGTCC
AGAGCGGCATCAAGGTGAGACTGCAACTGTACAGAACCGC
CAAGATGGGATGGGGAGTGAGGGCTCTGCAAACCATTCCC
CAAGGCACCTTCATCTGCGAATACGTGGGCGAACTGATCT
CCGACGCCGAAGCTGACGTGAGAGAGGACGACAGCTATCT
CTTCGATCTGGACAATAAGGACGGCGAGGTGTACTGCATC
GACGCTAGATATTACGGCAACATCTCTAGATTCATCAACC
ACCTCTGCGATCCCAACATCATTCCCGTGAGGGTGTTCAT
GCTGCACCAAGATCTGAGGTTCCCTAGAATCGCCTTCTTC
AGCTCTAGAGACATCAGAACCGGCGAGGAGCTGGGCTTCG
ATTACGGCGATAGATTCTGGGACATCAAGTCCAAGTACTT
CACATGCCAGTGCGGCAGCGAGAAGTGTAAGCACAGCGCT
GAGGCCATTGCTCTGGAGCAGTCTAGACTGGCCAGACTGG
ATGGAGGTGGCGGATCGGGAGACAAGAAGTACAGCATCGG
CCTGGCCATCGGCACCAACAGCGTGGGCTGGGCCGTGATC
ACCGACGAGTACAAGGTGCCCAGCAAGAAGTTCAAGGTGC
TGGGCAACACCGACCGGCACAGCATCAAGAAGAACCTGAT
CGGCGCCCTGCTGTTCGACAGCGGCGAGACCGCCGAGGCC
ACCCGGCTGAAGCGGACCGCCCGGCGGCGGTACACCCGGC
GGAAGAACCGGATCTGCTACCTGCAGGAGATCTTCAGCAA
CGAGATGGCCAAGGTGGACGACAGCTTCTTCCACCGGCTG
GAGGAGAGCTTCCTGGTGGAGGAGGACAAGAAGCACGAGC
GGCACCCCATCTTCGGCAACATCGTGGACGAGGTGGCCTA
CCACGAGAAGTACCCCACCATCTACCACCTGCGGAAGAAG
CTGGTGGACAGCACCGACAAGGCCGACCTGCGGCTGATCT
ACCTGGCCCTGGCCCACATGATCAAGTTCCGGGGCCACTT
CCTGATCGAGGGCGACCTGAACCCCGACAACAGCGACGTG
GACAAGCTGTTCATCCAGCTGGTGCAGACCTACAACCAGC
TGTTCGAGGAGAACCCCATCAACGCCAGCGGCGTGGACGC
CAAGGCCATCCTGAGCGCCCGGCTGAGCAAGAGCCGGCGG
CTGGAGAACCTGATCGCCCAGCTGCCCGGCGAGAAGAAGA
ACGGCCTGTTCGGCAACCTGATCGCCCTGAGCCTGGGCCT
GACCCCCAACTTCAAGAGCAACTTCGACCTGGCCGAGGAC
GCCAAGCTGCAGCTGAGCAAGGACACCTACGACGACGACC
TGGACAACCTGCTGGCCCAGATCGGCGACCAGTACGCCGA
CCTGTTCCTGGCCGCCAAGAACCTGAGCGACGCCATCCTG
CTGAGCGACATCCTGCGGGTGAACACCGAGATCACCAAGG
CCCCCCTGAGCGCCAGCATGATCAAGCGGTACGACGAGCA
CCACCAGGACCTGACCCTGCTGAAGGCCCTGGTGCGGCAG
CAGCTGCCCGAGAAGTACAAGGAGATCTTCTTCGACCAGA
GCAAGAACGGCTACGCCGGCTACATCGACGGCGGCGCCAG
CCAGGAGGAGTTCTACAAGTTCATCAAGCCCATCCTGGAG
AAGATGGACGGCACCGAGGAGCTGCTGGTGAAGCTGAACC
GGGAGGACCTGCTGCGGAAGCAGCGGACCTTCGACAACGG
CAGCATCCCCCACCAGATCCACCTGGGCGAGCTGCACGCC
ATCCTGCGGCGGCAGGAGGACTTCTACCCCTTCCTGAAGG
ACAACCGGGAGAAGATCGAGAAGATCCTGACCTTCCGGAT
CCCCTACTACGTGGGCCCCCTGGCCCGGGGCAACAGCCGG
TTCGCCTGGATGACCCGGAAATCCGAGGAGACCATCACCC
CCTGGAACTTCGAGGAGGTGGTGGACAAGGGCGCCAGCGC
CCAGAGCTTCATCGAGCGGATGACCAACTTCGACAAGAAC
CTGCCCAACGAGAAGGTGCTGCCCAAGCACAGCCTGCTGT
ACGAGTACTTCACCGTGTACAACGAGCTGACCAAGGTGAA
GTACGTGACCGAGGGCATGCGGAAGCCCGCCTTCCTGAGC
GGCGAGCAGAAGAAGGCCATCGTGGACCTGCTGTTCAAGA
CCAACCGGAAGGTGACCGTGAAGCAGCTGAAGGAGGACTA
CTTCAAGAAGATCGAGTGCTTCGACAGCGTGGAGATCAGC
GGCGTGGAGGACCGGTTCAACGCCAGCCTGGGCACCTACC
ACGACCTGCTGAAGATCATCAAGGACAAGGACTTCCTGGA
CAACGAGGAGAACGAGGACATCCTGGAGGACATCGTGCTG
ACCCTGACCCTGTTCGAGGACCGGGAGATGATCGAGGAGC
GGCTGAAAACCTACGCCCACCTGTTCGACGACAAGGTGAT
GAAGCAGCTGAAGCGGCGGCGGTACACCGGCTGGGGCCGG
CTGAGCCGGAAGCTGATCAACGGCATCCGGGACAAGCAGA
GCGGCAAGACCATCCTGGACTTCCTGAAATCCGACGGCTT
CGCCAACCGGAACTTCATGCAGCTGATCCACGACGACAGC
CTGACCTTCAAGGAGGACATCCAGAAGGCCCAGGTGAGCG
GCCAGGGCGACAGCCTGCACGAGCACATCGCCAACCTGGC
CGGCAGCCCCGCCATCAAGAAGGGCATCCTGCAGACCGTG
AAGGTGGTGGACGAGCTGGTGAAGGTGATGGGCCGGCACA
AGCCCGAGAACATCGTGATCGAGATGGCCCGGGAGAACCA
GACCACCCAGAAGGGCCAGAAGAACAGCCGGGAGCGGATG
AAGCGGATCGAGGAGGGCATCAAGGAGCTGGGCAGCCAGA
TCCTGAAGGAGCACCCCGTGGAGAACACCCAGCTGCAGAA
CGAGAAGCTGTACCTGTACTACCTGCAGAACGGCCGGGAC
ATGTACGTGGACCAGGAGCTGGACATCAACCGGCTGAGCG
ACTACGACGTGGCCGCCATCGTGCCCCAGAGCTTCCTGAA
GGACGACAGCATCGACAACAAGGTGCTGACCCGGAGCGAC
AAGGCCCGGGGCAAGAGCGACAACGTGCCCAGCGAGGAGG
TGGTGAAGAAGATGAAGAACTACTGGCGGCAGCTGCTGAA
CGCCAAGCTGATCACCCAGCGGAAGTTCGACAACCTGACC
AAGGCCGAGCGGGGCGGCCTGAGCGAGCTGGACAAGGCCG
GCTTCATCAAGCGGCAGCTGGTGGAGACCCGGCAGATCAC
CAAGCACGTGGCCCAGATCCTGGACAGCCGGATGAACACC
AAGTACGACGAGAACGACAAGCTGATCCGGGAGGTGAAGG
TGATCACCCTGAAATCCAAGCTGGTGAGCGACTTCCGGAA
GGACTTCCAGTTCTACAAGGTGCGGGAGATCAACAACTAC
CACCACGCCCACGACGCCTACCTGAACGCCGTGGTGGGCA
CCGCCCTGATCAAGAAGTACCCCAAGCTGGAGAGCGAGTT
CGTGTACGGCGACTACAAGGTGTACGACGTGCGGAAGATG
ATCGCCAAGAGCGAGCAGGAGATCGGCAAGGCCACCGCCA
AGTACTTCTTCTACAGCAACATCATGAACTTCTTCAAGAC
CGAGATCACCCTGGCCAACGGCGAGATCCGGAAGCGGCCC
CTGATCGAGACCAACGGCGAGACCGGCGAGATCGTGTGGG
ACAAGGGCCGGGACTTCGCCACCGTGCGGAAGGTGCTGAG
CATGCCCCAGGTGAACATCGTGAAGAAAACCGAGGTGCAG
ACCGGCGGCTTCAGCAAGGAGAGCATCCTGCCCAAGCGGA
ACAGCGACAAGCTGATCGCCCGGAAGAAGGACTGGGACCC
CAAGAAGTACGGCGGCTTCGACAGCCCCACCGTGGCCTAC
AGCGTGCTGGTGGTGGCCAAGGTGGAGAAGGGCAAGAGCA
AGAAGCTGAAATCCGTGAAGGAGCTGCTGGGCATCACCAT
CATGGAGCGGAGCAGCTTCGAGAAGAACCCCATCGACTTC
CTGGAGGCCAAGGGCTACAAGGAGGTGAAGAAGGACCTGA
TCATCAAGCTGCCCAAGTACAGCCTGTTCGAGCTGGAGAA
CGGCCGGAAGCGGATGCTGGCCAGCGCCGGCGAGCTGCAG
AAGGGCAACGAGCTGGCCCTGCCCAGCAAGTACGTGAACT
TCCTGTACCTGGCCAGCCACTACGAGAAGCTGAAGGGCAG
CCCCGAGGACAACGAGCAGAAGCAGCTGTTCGTGGAGCAG
CACAAGCACTACCTGGACGAGATCATCGAGCAGATCAGCG
AGTTCAGCAAGCGGGTGATCCTGGCCGACGCCAACCTGGA
CAAGGTGCTGAGCGCCTACAACAAGCACCGGGACAAGCCC
ATCCGGGAGCAGGCCGAGAACATCATCCACCTGTTCACCC
TGACCAACCTGGGCGCCCCCGCCGCCTTCAAGTACTTCGA
CACCACCATCGACCGGAAGCGGTACACCAGCACCAAGGAG
GTGCTGGACGCCACCCTGATCCACCAGAGCATCACCGGCC
TGTACGAGACCCGGATCGACCTGAGCCAGCTGGGCGGCGA
CAGCGGCGGCAAGCGGCCCGCCGCCACCAAGAAGGCCGGC
CAGGCCAAGAAGAAGAAGGGCAGCTACCCCTACGACGTGC
CCGACTACGCCTGAGCGGCCGCTTAATTAAGCTGCCTTCT
GCGGGGCTTGCCTTCTGGCCATGCCCTTCTTCTCTCCCTT
GCACCTGTACCTCTTGGTCTTTGAATAAAGCCTGAGTAGG
AAGTCTAGAAAAAAAAAAAAAAAAAAAAAAAAAAAAAAAA
AAAAAAAAAAAAAAAAAAAAAAAAAAAAAAAAAAAAAAAA
AAAAAAAA

In some embodiments, an expression repressor comprises the amino acid sequence of SEQ ID NOs: 38 or 73. In some embodiments, an expression repressor described herein comprises an amino acid sequence of SEQ ID NO: 38 or 73 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9a-dCas9
(SEQ ID NO: 38)
MAPKKKRKVGIHGVPAASGGGGSGNRAIRTEKIICRDVAR
GYENVPIPCVNGVDGEPCPEDYKYISENCETSTMNIDRNI
THLQHCTCVDDCSSSNCLCGQLSIRCWYDKDGRLLQEFNK
IEPPLIFECNQACSCWRNCKNRVVQSGIKVRLQLYRTAKM
GWGVRALQTIPQGTFICEYVGELISDAEADVREDDSYLFD
LDNKDGEVYCIDARYYGNISRFINHLCDPNIIPVRVFMLH
QDLRFPRIAFFSSRDIRTGEELGFDYGDRFWDIKSKYFTC
QCGSEKCKHSAEAIALEQSRLARLDGGGGSGDKKYSIGLA
IGTNSVGWAVITDEYKVPSKKFKVLGNTDRHSIKKNLIGA
LLFDSGETAEATRLKRTARRRYTRRKNRICYLQEIFSNEM
AKVDDSFFHRLEESFLVEEDKKHERHPIFGNIVDEVAYHE
KYPTIYHLRKKLVDSTDKADLRLIYLALAHMIKFRGHFLI
EGDLNPDNSDVDKLFIQLVQTYNQLFEENPINASGVDAKA
ILSARLSKSRRLENLIAQLPGEKKNGLFGNLIALSLGLTP
NFKSNFDLAEDAKLQLSKDTYDDDLDNLLAQIGDQYADLF
LAAKNLSDAILLSDILRVNTEITKAPLSASMIKRYDEHHQ
DLTLLKALVRQQLPEKYKEIFFDQSKNGYAGYIDGGASQE
EFYKFIKPILEKMDGTEELLVKLNREDLLRKQRTFDNGSI
PHQIHLGELHAILRRQEDFYPFLKDNREKIEKILTFRIPY
YVGPLARGNSRFAWMTRKSEETITPWNFEEVVDKGASAQS
FIERMTNFDKNLPNEKVLPKHSLLYEYFTVYNELTKVKYV
TEGMRKPAFLSGEQKKAIVDLLFKTNRKVTVKQLKEDYFK
KIECFDSVEISGVEDRFNASLGTYHDLLKIIKDKDFLDNE
ENEDILEDIVLTLTLFEDREMIEERLKTYAHLFDDKVMKQ
LKRRRYTGWGRLSRKLINGIRDKQSGKTILDFLKSDGFAN
RNFMQLIHDDSLTFKEDIQKAQVSGQGDSLHEHIANLAGS
PAIKKGILQTVKVVDELVKVMGRHKPENIVIEMARENQTT
QKGQKNSRERMKRIEEGIKELGSQILKEHPVENTQLQNEK
LYLYYLQNGRDMYVDQELDINRLSDYDVAAIVPQSFLKDD
SIDNKVLTRSDKARGKSDNVPSEEVVKKMKNYWRQLLNAK
LITQRKFDNLTKAERGGLSELDKAGFIKRQLVETRQITKH
VAQILDSRMNTKYDENDKLIREVKVITLKSKLVSDFRKDF
QFYKVREINNYHHAHDAYLNAVVGTALIKKYPKLESEFVY
GDYKVYDVRKMIAKSEQEIGKATAKYFFYSNIMNFFKTEI
TLANGEIRKRPLIETNGETGEIVWDKGRDFATVRKVLSMP
QVNIVKKTEVQTGGFSKESILPKRNSDKLIARKKDWDPKK
YGGFDSPTVAYSVLVVAKVEKGKSKKLKSVKELLGITIME
RSSFEKNPIDFLEAKGYKEVKKDLIIKLPKYSLFELENGR
KRMLASAGELQKGNELALPSKYVNFLYLASHYEKLKGSPE
DNEQKQLFVEQHKHYLDEIIEQISEFSKRVILADANLDKV
LSAYNKHRDKPIREQAENIIHLFTLTNLGAPAAFKYFDTT
IDRKRYTSTKEVLDATLIHQSITGLYETRIDLSQLGGDSG
GKRPAATKKAGQAKKKKGSYPYDVPDYA
G9A-dCas9 without HA tag
(SEQ ID NO: 73)
MAPKKKRKVGIHGVPAASGGGGSGNRAIRTEKIICRDVAR
GYENVPIPCVNGVDGEPCPEDYKYISENCETSTMNIDRNI
THLQHCTCVDDCSSSNCLCGQLSIRCWYDKDGRLLQEFNK
IEPPLIFECNQACSCWRNCKNRVVQSGIKVRLOLYRTAKM
GWGVRALQTIPQGTFICEYVGELISDAEADVREDDSYLFD
LDNKDGEVYCIDARYYGNISRFINHLCDPNIIPVRVFMLH
QDLRFPRIAFFSSRDIRTGEELGFDYGDRFWDIKSKYFTC
QCGSEKCKHSAEAIALEQSRLARLDGGGGSGDKKYSIGLA
IGTNSVGWAVITDEYKVPSKKFKVLGNTDRHSIKKNLIGA
LLFDSGETAEATRLKRTARRRYTRRKNRICYLQEIFSNEM
AKVDDSFFHRLEESFLVEEDKKHERHPIFGNIVDEVAYHE
KYPTIYHLRKKLVDSTDKADLRLIYLALAHMIKFRGHFLI
EGDLNPDNSDVDKLFIQLVQTYNQLFEENPINASGVDAKA
ILSARLSKSRRLENLIAQLPGEKKNGLFGNLIALSLGLTP
NFKSNFDLAEDAKLQLSKDTYDDDLDNLLAQIGDQYADLF
LAAKNLSDAILLSDILRVNTEITKAPLSASMIKRYDEHHQ
DLTLLKALVRQQLPEKYKEIFFDQSKNGYAGYIDGGASQE
EFYKFIKPILEKMDGTEELLVKLNREDLLRKQRTFDNGSI
PHQIHLGELHAILRRQEDFYPFLKDNREKIEKILTFRIPY
YVGPLARGNSRFAWMTRKSEETITPWNFEEVVDKGASAQS
FIERMTNFDKNLPNEKVLPKHSLLYEYFTVYNELTKVKYV
TEGMRKPAFLSGEQKKAIVDLLFKTNRKVTVKQLKEDYFK
KIECFDSVEISGVEDRFNASLGTYHDLLKIIKDKDFLDNE
ENEDILEDIVLTLTLFEDREMIEERLKTYAHLFDDKVMKQ
LKRRRYTGWGRLSRKLINGIRDKQSGKTILDFLKSDGFAN
RNFMQLIHDDSLTFKEDIQKAQVSGQGDSLHEHIANLAGS
PAIKKGILQTVKVVDELVKVMGRHKPENIVIEMARENQTT
QKGQKNSRERMKRIEEGIKELGSQILKEHPVENTQLQNEK
LYLYYLQNGRDMYVDQELDINRLSDYDVAAIVPQSFLKDD
SIDNKVLTRSDKARGKSDNVPSEEVVKKMKNYWRQLLNAK
LITQRKFDNLTKAERGGLSELDKAGFIKRQLVETRQITKH
VAQILDSRMNTKYDENDKLIREVKVITLKSKLVSDFRKDF
QFYKVREINNYHHAHDAYLNAVVGTALIKKYPKLESEFVY
GDYKVYDVRKMIAKSEQEIGKATAKYFFYSNIMNFFKTEI
TLANGEIRKRPLIETNGETGEIVWDKGRDFATVRKVLSMP
QVNIVKKTEVQTGGFSKESILPKRNSDKLIARKKDWDPKK
YGGFDSPTVAYSVLVVAKVEKGKSKKLKSVKELLGITIME
RSSFEKNPIDFLEAKGYKEVKKDLIIKLPKYSLFELENGR
KRMLASAGELQKGNELALPSKYVNFLYLASHYEKLKGSPE
DNEQKQLFVEQHKHYLDEIIEQISEFSKRVILADANLDKV
LSAYNKHRDKPIREQAENIIHLFTLTNLGAPAAFKYFDTT
IDRKRYTSTKEVLDATLIHQSITGLYETRIDLSQLGGDSG
GKRPAATKKAGQAKKKKGS

In some embodiments, an expression repressor comprises a DNA-targeting moiety comprising dCas9, e.g., an S. pyogenes dCas9, and a repressor domain comprising HDAC8, e.g., a HDAC8 domain. In some embodiments, the expression repressor is encoded by the nucleic acid sequence of SEQ ID NOs: 27 (e.g., a nucleic acid (e.g., cDNA) encoding the expression repressor). In some embodiments, a nucleic acid described herein comprises a nucleic acid sequence of SEQ ID NO: 27 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-HDAC8 (MR-29439)
(SEQ ID NO: 27)
AGGAAATAAGAGAGAAAAGAAGAGTAAGAAGAAATATAAGAGCCACCATGGCCCCCAAGA
AGAAGCGGAAGGTGGGCATCCACGGCGTGCCCGCCGCCGACAAGAAGTACAGCATCGGCCT
GGCCATCGGCACCAACAGCGTGGGCTGGGCCGTGATCACCGACGAGTACAAGGTGCCCAGC
AAGAAGTTCAAGGTGCTGGGCAACACCGACCGGCACAGCATCAAGAAGAACCTGATCGGCG
CCCTGCTGTTCGACAGCGGCGAGACCGCCGAGGCCACCCGGCTGAAGCGGACCGCCCGGCG
GCGGTACACCCGGCGGAAGAACCGGATCTGCTACCTGCAGGAGATCTTCAGCAACGAGATG
GCCAAGGTGGACGACAGCTTCTTCCACCGGCTGGAGGAGAGCTTCCTGGTGGAGGAGGACA
AGAAGCACGAGCGGCACCCCATCTTCGGCAACATCGTGGACGAGGTGGCCTACCACGAGAA
GTACCCCACCATCTACCACCTGCGGAAGAAGCTGGTGGACAGCACCGACAAGGCCGACCTG
CGGCTGATCTACCTGGCCCTGGCCCACATGATCAAGTTCCGGGGCCACTTCCTGATCGAGGG
CGACCTGAACCCCGACAACAGCGACGTGGACAAGCTGTTCATCCAGCTGGTGCAGACCTAC
AACCAGCTGTTCGAGGAGAACCCCATCAACGCCAGCGGCGTGGACGCCAAGGCCATCCTGA
GCGCCCGGCTGAGCAAGAGCCGGCGGCTGGAGAACCTGATCGCCCAGCTGCCCGGCGAGAA
GAAGAACGGCCTGTTCGGCAACCTGATCGCCCTGAGCCTGGGCCTGACCCCCAACTTCAAGA
GCAACTTCGACCTGGCCGAGGACGCCAAGCTGCAGCTGAGCAAGGACACCTACGACGACGA
CCTGGACAACCTGCTGGCCCAGATCGGCGACCAGTACGCCGACCTGTTCCTGGCCGCCAAGA
ACCTGAGCGACGCCATCCTGCTGAGCGACATCCTGCGGGTGAACACCGAGATCACCAAGGC
CCCCCTGAGCGCCAGCATGATCAAGCGGTACGACGAGCACCACCAGGACCTGACCCTGCTG
AAGGCCCTGGTGCGGCAGCAGCTGCCCGAGAAGTACAAGGAGATCTTCTTCGACCAGAGCA
AGAACGGCTACGCCGGCTACATCGACGGCGGCGCCAGCCAGGAGGAGTTCTACAAGTTCAT
CAAGCCCATCCTGGAGAAGATGGACGGCACCGAGGAGCTGCTGGTGAAGCTGAACCGGGAG
GACCTGCTGCGGAAGCAGCGGACCTTCGACAACGGCAGCATCCCCCACCAGATCCACCTGG
GCGAGCTGCACGCCATCCTGCGGCGGCAGGAGGACTTCTACCCCTTCCTGAAGGACAACCG
GGAGAAGATCGAGAAGATCCTGACCTTCCGGATCCCCTACTACGTGGGCCCCCTGGCCCGGG
GCAACAGCCGGTTCGCCTGGATGACCCGGAAATCCGAGGAGACCATCACCCCCTGGAACTT
CGAGGAGGTGGTGGACAAGGGCGCCAGCGCCCAGAGCTTCATCGAGCGGATGACCAACTTC
GACAAGAACCTGCCCAACGAGAAGGTGCTGCCCAAGCACAGCCTGCTGTACGAGTACTTCA
CCGTGTACAACGAGCTGACCAAGGTGAAGTACGTGACCGAGGGCATGCGGAAGCCCGCCTT
CCTGAGCGGCGAGCAGAAGAAGGCCATCGTGGACCTGCTGTTCAAGACCAACCGGAAGGTG
ACCGTGAAGCAGCTGAAGGAGGACTACTTCAAGAAGATCGAGTGCTTCGACAGCGTGGAGA
TCAGCGGCGTGGAGGACCGGTTCAACGCCAGCCTGGGCACCTACCACGACCTGCTGAAGAT
CATCAAGGACAAGGACTTCCTGGACAACGAGGAGAACGAGGACATCCTGGAGGACATCGTG
CTGACCCTGACCCTGTTCGAGGACCGGGAGATGATCGAGGAGCGGCTGAAAACCTACGCCC
ACCTGTTCGACGACAAGGTGATGAAGCAGCTGAAGCGGCGGCGGTACACCGGCTGGGGCCG
GCTGAGCCGGAAGCTGATCAACGGCATCCGGGACAAGCAGAGCGGCAAGACCATCCTGGAC
TTCCTGAAATCCGACGGCTTCGCCAACCGGAACTTCATGCAGCTGATCCACGACGACAGCCT
GACCTTCAAGGAGGACATCCAGAAGGCCCAGGTGAGCGGCCAGGGCGACAGCCTGCACGAG
CACATCGCCAACCTGGCCGGCAGCCCCGCCATCAAGAAGGGCATCCTGCAGACCGTGAAGG
TGGTGGACGAGCTGGTGAAGGTGATGGGCCGGCACAAGCCCGAGAACATCGTGATCGAGAT
GGCCCGGGAGAACCAGACCACCCAGAAGGGCCAGAAGAACAGCCGGGAGCGGATGAAGCG
GATCGAGGAGGGCATCAAGGAGCTGGGCAGCCAGATCCTGAAGGAGCACCCCGTGGAGAA
CACCCAGCTGCAGAACGAGAAGCTGTACCTGTACTACCTGCAGAACGGCCGGGACATGTAC
GTGGACCAGGAGCTGGACATCAACCGGCTGAGCGACTACGACGTGGCCGCCATCGTGCCCC
AGAGCTTCCTGAAGGACGACAGCATCGACAACAAGGTGCTGACCCGGAGCGACAAGGCCCG
GGGCAAGAGCGACAACGTGCCCAGCGAGGAGGTGGTGAAGAAGATGAAGAACTACTGGCG
GCAGCTGCTGAACGCCAAGCTGATCACCCAGCGGAAGTTCGACAACCTGACCAAGGCCGAG
CGGGGCGGCCTGAGCGAGCTGGACAAGGCCGGCTTCATCAAGCGGCAGCTGGTGGAGACCC
GGCAGATCACCAAGCACGTGGCCCAGATCCTGGACAGCCGGATGAACACCAAGTACGACGA
GAACGACAAGCTGATCCGGGAGGTGAAGGTGATCACCCTGAAATCCAAGCTGGTGAGCGAC
TTCCGGAAGGACTTCCAGTTCTACAAGGTGCGGGAGATCAACAACTACCACCACGCCCACG
ACGCCTACCTGAACGCCGTGGTGGGCACCGCCCTGATCAAGAAGTACCCCAAGCTGGAGAG
CGAGTTCGTGTACGGCGACTACAAGGTGTACGACGTGCGGAAGATGATCGCCAAGAGCGAG
CAGGAGATCGGCAAGGCCACCGCCAAGTACTTCTTCTACAGCAACATCATGAACTTCTTCAA
GACCGAGATCACCCTGGCCAACGGCGAGATCCGGAAGCGGCCCCTGATCGAGACCAACGGC
GAGACCGGCGAGATCGTGTGGGACAAGGGCCGGGACTTCGCCACCGTGCGGAAGGTGCTGA
GCATGCCCCAGGTGAACATCGTGAAGAAAACCGAGGTGCAGACCGGCGGCTTCAGCAAGGA
GAGCATCCTGCCCAAGCGGAACAGCGACAAGCTGATCGCCCGGAAGAAGGACTGGGACCCC
AAGAAGTACGGCGGCTTCGACAGCCCCACCGTGGCCTACAGCGTGCTGGTGGTGGCCAAGG
TGGAGAAGGGCAAGAGCAAGAAGCTGAAATCCGTGAAGGAGCTGCTGGGCATCACCATCAT
GGAGCGGAGCAGCTTCGAGAAGAACCCCATCGACTTCCTGGAGGCCAAGGGCTACAAGGAG
GTGAAGAAGGACCTGATCATCAAGCTGCCCAAGTACAGCCTGTTCGAGCTGGAGAACGGCC
GGAAGCGGATGCTGGCCAGCGCCGGCGAGCTGCAGAAGGGCAACGAGCTGGCCCTGCCCAG
CAAGTACGTGAACTTCCTGTACCTGGCCAGCCACTACGAGAAGCTGAAGGGCAGCCCCGAG
GACAACGAGCAGAAGCAGCTGTTCGTGGAGCAGCACAAGCACTACCTGGACGAGATCATCG
AGCAGATCAGCGAGTTCAGCAAGCGGGTGATCCTGGCCGACGCCAACCTGGACAAGGTGCT
GAGCGCCTACAACAAGCACCGGGACAAGCCCATCCGGGAGCAGGCCGAGAACATCATCCAC
CTGTTCACCCTGACCAACCTGGGCGCCCCCGCCGCCTTCAAGTACTTCGACACCACCATCGA
CCGGAAGCGGTACACCAGCACCAAGGAGGTGCTGGACGCCACCCTGATCCACCAGAGCATC
ACCGGCCTGTACGAGACCCGGATCGACCTGAGCCAGCTGGGCGGCGACAGCGGCGGCAAGC
GGCCCGCCGCCACCAAGAAGGCCGGCCAGGCCAAGAAGAAGAAGTCGGGCGGGGGTGGCT
CAGAGGAGCCCGAGGAGCCCGCCGATAGCGGACAATCTCTGGTGCCCGTCTACATCTACAG
CCCCGAATATGTGAGCATGTGTGATTCCCTCGCCAAGATCCCTAAGAGAGCCAGCATGGTGC
ATTCTCTGATCGAGGCCTACGCTCTGCATAAGCAAATGAGGATCGTGAAGCCCAAGGTCGCC
AGCATGGAAGAGATGGCCACCTTTCACACCGATGCCTACCTCCAACATCTCCAGAAGGTGTC
CCAAGAGGGCGACGACGACCACCCCGACTCCATTGAGTACGGACTGGGCTATGATTGCCCC
GCCACCGAGGGCATCTTTGACTATGCCGCCGCTATCGGCGGAGCTACCATCACAGCCGCCCA
GTGTCTGATTGATGGCATGTGCAAGGTCGCCATCAACTGGTCCGGAGGCTGGCATCATGCCA
AGAAGGATGAGGCCTCCGGCTTCTGTTATCTGAATGACGCCGTGCTGGGCATTCTGAGACTG
AGGAGGAAATTCGAGAGGATTCTGTACGTGGATCTGGATCTGCATCACGGAGATGGAGTCG
AAGATGCCTTCAGCTTCACCAGCAAGGTGATGACAGTCTCTCTGCACAAGTTCTCCCCCGGC
TTCTTTCCCGGAACCGGCGACGTGTCCGACGTGGGACTGGGCAAGGGAAGGTACTACAGCG
TGAACGTGCCCATTCAAGACGGCATCCAAGACGAGAAGTACTACCAGATCTGCGAGTCCGT
GCTCAAGGAGGTCTACCAAGCCTTCAATCCTAAGGCTGTCGTGCTCCAACTGGGAGCTGATA
CCATTGCTGGCGATCCCATGTGCAGCTTCAATATGACACCCGTCGGAATCGGCAAGTGCCTC
AAGTACATCCTCCAGTGGCAGCTCGCCACCCTCATTCTCGGAGGAGGCGGATACAATCTGGC
TAATACCGCCAGATGCTGGACCTATCTGACCGGCGTGATTCTGGGCAAAACACTGAGCAGCG
AAATCCCCGACCACGAGTTTTTCACCGCTTACGGCCCCGACTACGTGCTGGAGATCACCCCC
AGCTGCAGACCCGATAGAAACGAACCCCATAGAATCCAGCAAATTCTGAACTATATCAAGG
GCAACCTCAAGCACGTCGTGGGAGGTGGCGGATCGGGAAAGCGGCCCGCCGCCACCAAGAA
GGCCGGTCAGGCCAAGAAGAAGAAGGGCAGCTACCCCTACGACGTGCCCGACTACGCCTGA
GCGGCCGCTTAATTAAGCTGCCTTCTGCGGGGCTTGCCTTCTGGCCATGCCCTTCTTCTCTCC
CTTGCACCTGTACCTCTTGGTCTTTGAATAAAGCCTGAGTAGGAAGTCTAGAAAAAAAAAAA
AAAAAAAAAAAAAAAAAAAAAAAAAAAAAAAAAAAAAAAAAAAAAAAAAAAAAAAAAA
AAAAAAAAAAA

In some embodiments, an expression repressor comprises the amino acid sequence of SEQ ID NOs: 39 or 74. In some embodiments, an expression repressor described herein comprises an amino acid sequence of 25 SEQ ID NO: 39 or 74 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-HDAC8
(SEQ ID NO: 39)
MAPKKKRKVGIHGVPAADKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
KLALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGDSGGKRPAATKKAGQAK
KKKSGGGGSEEPEEPADSGQSLVPVYIYSPEYVSMCDSLAKIPKRASMVH
SLIEAYALHKQMRIVKPKVASMEEMATFHTDAYLQHLQKVSQEGDDDHPD
SIEYGLGYDCPATEGIFDYAAAIGGATITAAQCLIDGMCKVAINWSGGWH
HAKKDEASGFCYLNDAVLGILRLRRKFERILYVDLDLHHGDGVEDAFSFT
SKVMTVSLHKFSPGFFPGTGDVSDVGLGKGRYYSVNVPIQDGIQDEKYYQ
ICESVLKEVYQAFNPKAVVLQLGADTIAGDPMCSFNMTPVGIGKCLKYIL
QWQLATLILGGGGYNLANTARCWTYLTGVILGKTLSSEIPDHEFFTAYGP
DYVLEITPSCRPDRNEPHRIQQILNYIKGNLKHVVGGGGSGKRPAATKKA
GQAKKKKGSYPYDVPDYA
dCas9-HDAC8 without HA tag
(SEQ ID NO: 74)
MAPKKKRKVGIHGVPAADKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGDSGGKRPAATKKAGQAK
KKKSGGGGSEEPEEPADSGQSLVPVYIYSPEYVSMCDSLAKIPKRASMVH
SLIEAYALHKQMRIVKPKVASMEEMATFHTDAYLQHLQKVSQEGDDDHPD
SIEYGLGYDCPATEGIFDYAAAIGGATITAAQCLIDGMCKVAINWSGGWH
HAKKDEASGFCYLNDAVLGILRLRRKFERILYVDLDLHHGDGVEDAFSFT
SKVMTVSLHKFSPGFFPGTGDVSDVGLGKGRYYSVNVPIQDGIQDEKYYQ
ICESVLKEVYQAFNPKAVVLQLGADTIAGDPMCSFNMTPVGIGKCLKYIL
QWQLATLILGGGGYNLANTARCWTYLTGVILGKTLSSEIPDHEFFTAYGP
DYVLEITPSCRPDRNEPHRIQQILNYIKGNLKHVVGGGGSGKRPAATKKA
GQAKKKKGS

In some embodiments, an expression repressor comprises a DNA-targeting moiety comprising dCas9, e.g., an S. pyogenes dCas9, and a repressor domain comprising LSD1, e.g., a LSD1 domain. In some embodiments, the expression repressor is encoded by the nucleic acid sequence of SEQ ID NOs: 28 (e.g., a nucleic acid (e.g., cDNA) encoding the expression repressor). In some embodiments, a nucleic acid described herein comprises a nucleic acid sequence of SEQ ID NO: 28 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-LSD1 (MR-29129)
(SEQ ID NO: 28)
AGGAAATAAGAGAGAAAAGAAGAGTAAGAAGAAATATAAGAGCCACCATGGCCCCCAAGA
AGAAGCGGAAGGTGGGCATCCACGGCGTGCCCGCCGCCGACAAGAAGTACAGCATCGGCCT
GGCCATCGGCACCAACAGCGTGGGCTGGGCCGTGATCACCGACGAGTACAAGGTGCCCAGC
AAGAAGTTCAAGGTGCTGGGCAACACCGACCGGCACAGCATCAAGAAGAACCTGATCGGCG
CCCTGCTGTTCGACAGCGGCGAGACCGCCGAGGCCACCCGGCTGAAGCGGACCGCCCGGCG
GCGGTACACCCGGCGGAAGAACCGGATCTGCTACCTGCAGGAGATCTTCAGCAACGAGATG
GCCAAGGTGGACGACAGCTTCTTCCACCGGCTGGAGGAGAGCTTCCTGGTGGAGGAGGACA
AGAAGCACGAGCGGCACCCCATCTTCGGCAACATCGTGGACGAGGTGGCCTACCACGAGAA
GTACCCCACCATCTACCACCTGCGGAAGAAGCTGGTGGACAGCACCGACAAGGCCGACCTG
CGGCTGATCTACCTGGCCCTGGCCCACATGATCAAGTTCCGGGGCCACTTCCTGATCGAGGG
CGACCTGAACCCCGACAACAGCGACGTGGACAAGCTGTTCATCCAGCTGGTGCAGACCTAC
AACCAGCTGTTCGAGGAGAACCCCATCAACGCCAGCGGCGTGGACGCCAAGGCCATCCTGA
GCGCCCGGCTGAGCAAGAGCCGGCGGCTGGAGAACCTGATCGCCCAGCTGCCCGGCGAGAA
GAAGAACGGCCTGTTCGGCAACCTGATCGCCCTGAGCCTGGGCCTGACCCCCAACTTCAAGA
GCAACTTCGACCTGGCCGAGGACGCCAAGCTGCAGCTGAGCAAGGACACCTACGACGACGA
CCTGGACAACCTGCTGGCCCAGATCGGCGACCAGTACGCCGACCTGTTCCTGGCCGCCAAGA
ACCTGAGCGACGCCATCCTGCTGAGCGACATCCTGCGGGTGAACACCGAGATCACCAAGGC
CCCCCTGAGCGCCAGCATGATCAAGCGGTACGACGAGCACCACCAGGACCTGACCCTGCTG
AAGGCCCTGGTGCGGCAGCAGCTGCCCGAGAAGTACAAGGAGATCTTCTTCGACCAGAGCA
AGAACGGCTACGCCGGCTACATCGACGGCGGCGCCAGCCAGGAGGAGTTCTACAAGTTCAT
CAAGCCCATCCTGGAGAAGATGGACGGCACCGAGGAGCTGCTGGTGAAGCTGAACCGGGAG
GACCTGCTGCGGAAGCAGCGGACCTTCGACAACGGCAGCATCCCCCACCAGATCCACCTGG
GCGAGCTGCACGCCATCCTGCGGCGGCAGGAGGACTTCTACCCCTTCCTGAAGGACAACCG
GGAGAAGATCGAGAAGATCCTGACCTTCCGGATCCCCTACTACGTGGGCCCCCTGGCCCGGG
GCAACAGCCGGTTCGCCTGGATGACCCGGAAATCCGAGGAGACCATCACCCCCTGGAACTT
CGAGGAGGTGGTGGACAAGGGCGCCAGCGCCCAGAGCTTCATCGAGCGGATGACCAACTTC
GACAAGAACCTGCCCAACGAGAAGGTGCTGCCCAAGCACAGCCTGCTGTACGAGTACTTCA
CCGTGTACAACGAGCTGACCAAGGTGAAGTACGTGACCGAGGGCATGCGGAAGCCCGCCTT
CCTGAGCGGCGAGCAGAAGAAGGCCATCGTGGACCTGCTGTTCAAGACCAACCGGAAGGTG
ACCGTGAAGCAGCTGAAGGAGGACTACTTCAAGAAGATCGAGTGCTTCGACAGCGTGGAGA
TCAGCGGCGTGGAGGACCGGTTCAACGCCAGCCTGGGCACCTACCACGACCTGCTGAAGAT
CATCAAGGACAAGGACTTCCTGGACAACGAGGAGAACGAGGACATCCTGGAGGACATCGTG
CTGACCCTGACCCTGTTCGAGGACCGGGAGATGATCGAGGAGCGGCTGAAAACCTACGCCC
ACCTGTTCGACGACAAGGTGATGAAGCAGCTGAAGCGGCGGCGGTACACCGGCTGGGGCCG
GCTGAGCCGGAAGCTGATCAACGGCATCCGGGACAAGCAGAGCGGCAAGACCATCCTGGAC
TTCCTGAAATCCGACGGCTTCGCCAACCGGAACTTCATGCAGCTGATCCACGACGACAGCCT
GACCTTCAAGGAGGACATCCAGAAGGCCCAGGTGAGCGGCCAGGGCGACAGCCTGCACGAG
CACATCGCCAACCTGGCCGGCAGCCCCGCCATCAAGAAGGGCATCCTGCAGACCGTGAAGG
TGGTGGACGAGCTGGTGAAGGTGATGGGCCGGCACAAGCCCGAGAACATCGTGATCGAGAT
GGCCCGGGAGAACCAGACCACCCAGAAGGGCCAGAAGAACAGCCGGGAGCGGATGAAGCG
GATCGAGGAGGGCATCAAGGAGCTGGGCAGCCAGATCCTGAAGGAGCACCCCGTGGAGAA
CACCCAGCTGCAGAACGAGAAGCTGTACCTGTACTACCTGCAGAACGGCCGGGACATGTAC
GTGGACCAGGAGCTGGACATCAACCGGCTGAGCGACTACGACGTGGCCGCCATCGTGCCCC
AGAGCTTCCTGAAGGACGACAGCATCGACAACAAGGTGCTGACCCGGAGCGACAAGGCCCG
GGGCAAGAGCGACAACGTGCCCAGCGAGGAGGTGGTGAAGAAGATGAAGAACTACTGGCG
GCAGCTGCTGAACGCCAAGCTGATCACCCAGCGGAAGTTCGACAACCTGACCAAGGCCGAG
CGGGGCGGCCTGAGCGAGCTGGACAAGGCCGGCTTCATCAAGCGGCAGCTGGTGGAGACCC
GGCAGATCACCAAGCACGTGGCCCAGATCCTGGACAGCCGGATGAACACCAAGTACGACGA
GAACGACAAGCTGATCCGGGAGGTGAAGGTGATCACCCTGAAATCCAAGCTGGTGAGCGAC
TTCCGGAAGGACTTCCAGTTCTACAAGGTGCGGGAGATCAACAACTACCACCACGCCCACG
ACGCCTACCTGAACGCCGTGGTGGGCACCGCCCTGATCAAGAAGTACCCCAAGCTGGAGAG
CGAGTTCGTGTACGGCGACTACAAGGTGTACGACGTGCGGAAGATGATCGCCAAGAGCGAG
CAGGAGATCGGCAAGGCCACCGCCAAGTACTTCTTCTACAGCAACATCATGAACTTCTTCAA
GACCGAGATCACCCTGGCCAACGGCGAGATCCGGAAGCGGCCCCTGATCGAGACCAACGGC
GAGACCGGCGAGATCGTGTGGGACAAGGGCCGGGACTTCGCCACCGTGCGGAAGGTGCTGA
GCATGCCCCAGGTGAACATCGTGAAGAAAACCGAGGTGCAGACCGGCGGCTTCAGCAAGGA
GAGCATCCTGCCCAAGCGGAACAGCGACAAGCTGATCGCCCGGAAGAAGGACTGGGACCCC
AAGAAGTACGGCGGCTTCGACAGCCCCACCGTGGCCTACAGCGTGCTGGTGGTGGCCAAGG
TGGAGAAGGGCAAGAGCAAGAAGCTGAAATCCGTGAAGGAGCTGCTGGGCATCACCATCAT
GGAGCGGAGCAGCTTCGAGAAGAACCCCATCGACTTCCTGGAGGCCAAGGGCTACAAGGAG
GTGAAGAAGGACCTGATCATCAAGCTGCCCAAGTACAGCCTGTTCGAGCTGGAGAACGGCC
GGAAGCGGATGCTGGCCAGCGCCGGCGAGCTGCAGAAGGGCAACGAGCTGGCCCTGCCCAG
CAAGTACGTGAACTTCCTGTACCTGGCCAGCCACTACGAGAAGCTGAAGGGCAGCCCCGAG
GACAACGAGCAGAAGCAGCTGTTCGTGGAGCAGCACAAGCACTACCTGGACGAGATCATCG
AGCAGATCAGCGAGTTCAGCAAGCGGGTGATCCTGGCCGACGCCAACCTGGACAAGGTGCT
GAGCGCCTACAACAAGCACCGGGACAAGCCCATCCGGGAGCAGGCCGAGAACATCATCCAC
CTGTTCACCCTGACCAACCTGGGCGCCCCCGCCGCCTTCAAGTACTTCGACACCACCATCGA
CCGGAAGCGGTACACCAGCACCAAGGAGGTGCTGGACGCCACCCTGATCCACCAGAGCATC
ACCGGCCTGTACGAGACCCGGATCGACCTGAGCCAGCTGGGCGGCGACAGCGGCGGCAAGC
GGCCCGCCGCCACCAAGAAGGCCGGCCAGGCCAAGAAGAAGAAGGAGAGCGGCGGCGGCA
GCCTCTCCGGAAAGAAAGCCGCCGCTGCCGCTGCCGCCGCTGCTGCTGCCGCTACCGGCACA
GAAGCTGGCCCCGGAACAGCTGGCGGAAGCGAAAACGGAAGCGAAGTGGCTGCCCAGCCC
GCCGGACTGTCCGGACCCGCCGAGGTGGGACCCGGCGCTGTCGGCGAAAGGACCCCCAGAA
AAAAGGAGCCTCCTAGAGCCAGCCCTCCCGGCGGACTCGCCGAACCTCCCGGCAGCGCTGG
ACCTCAAGCCGGACCTACAGTGGTGCCCGGCAGCGCTACACCTATGGAGACCGGCATCGCT
GAGACCCCCGAGGGAAGGAGAACCAGCAGAAGGAAGAGAGCCAAGGTGGAGTACAGAGAG
ATGGATGAGTCTCTGGCTAACCTCAGCGAGGACGAGTACTACTCCGAGGAGGAAAGGAATG
CCAAGGCCGAGAAGGAGAAGAAGCTGCCCCCTCCTCCCCCTCAAGCCCCCCCCGAGGAGGA
GAACGAGTCCGAGCCCGAGGAACCCAGCGGAGTGGAAGGAGCCGCCTTTCAGTCCAGACTG
CCCCACGACAGAATGACATCCCAAGAGGCCGCTTGCTTTCCCGACATTATTTCCGGCCCTCA
GCAGACCCAGAAGGTGTTTCTGTTCATTAGAAATAGAACACTCCAGCTGTGGCTCGACAACC
CCAAGATCCAGCTGACCTTCGAGGCTACCCTCCAACAGCTGGAGGCCCCCTACAATAGCGAT
ACCGTGCTGGTGCACAGAGTGCACAGCTATCTGGAGAGGCACGGCCTCATTAACTTCGGCAT
TTACAAGCGGATCAAGCCCCTGCCCACCAAGAAAACAGGCAAGGTGATCATCATCGGCAGC
GGCGTGAGCGGCCTGGCCGCCGCCCGGCAGCTGCAGAGCTTCGGCATGGACGTGACCCTGC
TGGAGGCCCGGGACCGGGTGGGCGGCCGGGTGGCCACCTTCCGGAAGGGCAACTACGTGGC
CGACCTGGGCGCCATGGTGGTGACCGGCCTGGGCGGCAACCCCATGGCCGTGGTGAGCAAG
CAGGTGAACATGGAGCTGGCCAAGATCAAGCAGAAGTGCCCCCTGTACGAGGCCAACGGCC
AGGCCGTGCCCAAGGAGAAGGACGAGATGGTGGAGCAGGAGTTCAACCGGCTGCTGGAGG
CCACCAGCTACCTGAGCCACCAGCTGGACTTCAACGTGCTGAACAACAAGCACGTGAGCCT
GGGCCAGGCCCTGGAGGTGGTGATCCAGCTGCAGGAGAAGCACGTGAAGGACGAGCAGATC
GAGCACTGGAAGAAGATCGTGAAAACACAGGAGGAGCTGAAGGAGCTGCTGAACAAGATG
GTGAACCTGAAGGAGAAGATCAAGGAGCTGCACCAGCAGTACAAGGAGGCCAGCGAGGTG
AAGCCCCCCCGGGACATCACCGCCGAGTTCCTGGTCAAAAGCAAGCACCGGGACCTGACCG
CCCTGTGCAAGGAGTACGACGAGCTGGCCGAGACCCAGGGCAAGCTGGAGGAGAAGCTGCA
GGAGCTGGAGGCCAACCCCCCCAGCGACGTGTACCTGAGCAGCCGGGACCGGCAGATCCTG
GACTGGCACTTCGCCAACCTGGAGTTCGCCAACGCCACCCCCCTGAGCACCCTGAGCCTGAA
GCACTGGGACCAGGACGACGACTTCGAGTTCACCGGCAGCCACCTGACCGTGCGGAACGGC
TACAGCTGCGTGCCCGTGGCCCTGGCCGAGGGCCTGGACATCAAGCTGAACACCGCCGTGC
GGCAGGTGCGGTACACCGCCAGCGGCTGCGAGGTGATCGCCGTGAACACCCGGAGCACCAG
CCAGACCTTCATCTACAAGTGCGACGCCGTGCTGTGCACCCTGCCCCTGGGCGTGCTGAAGC
AGCAGCCCCCCGCCGTGCAGTTCGTGCCCCCCCTGCCCGAGTGGAAAACAAGCGCCGTGCA
GCGGATGGGCTTCGGCAACCTGAACAAGGTGGTGCTGTGCTTCGACCGGGTGTTCTGGGACC
CCAGCGTGAACCTGTTCGGCCACGTGGGCAGCACCACCGCCAGCCGGGGCGAGCTGTTCCTG
TTCTGGAACCTGTACAAGGCCCCCATCCTGCTGGCCCTGGTGGCCGGCGAGGCCGCCGGCAT
CATGGAGAACATCAGCGACGACGTGATCGTGGGCCGGTGCCTGGCCATCCTGAAGGGCATC
TTCGGCAGCAGCGCCGTGCCCCAGCCCAAGGAGACCGTGGTGAGCCGGTGGCGGGCCGACC
CCTGGGCCCGGGGCAGCTACAGCTACGTGGCCGCCGGCAGCAGCGGCAACGACTACGACCT
GATGGCCCAGCCCATCACCCCCGGCCCCAGCATCCCCGGCGCCCCCCAGCCCATCCCCCGGC
TGTTCTTCGCCGGCGAGCACACCATCCGGAACTACCCCGCCACCGTGCACGGCGCCCTGCTG
AGCGGCCTGCGGGAGGCAGGACGGATCGCCGACCAGTTCCTGGGCGCCATGTACACCCTGC
CCCGGCAGGCCACCCCCGGCGTGCCCGCCCAGCAGAGCCCCAGCATGAGCGGCGGCAAGCG
GCCCGCCGCCACCAAGAAGGCCGGCCAGGCCAAGAAGAAGAAGGGCAGCTACCCCTACGA
CGTGCCCGACTACGCCTGAGCGGCCGCTTAATTAAGCTGCCTTCTGCGGGGCTTGCCTTCTG
GCCATGCCCTTCTTCTCTCCCTTGCACCTGTACCTCTTGGTCTTTGAATAAAGCCTGAGTAGG
AAGTCTAGAAAAAAAAAAAAAAAAAAAAAAAAAAAAAAAAAAAAAAAAAAAAAAAAAAA
AAAAAAAAAAAAAAAAAAAAAAAAAAAAA

In some embodiments, an expression repressor comprises the amino acid sequence of SEQ ID NOs: 40 or 75. In some embodiments, an expression repressor described herein comprises an amino acid sequence of SEQ ID NO: 40 or 75, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-LSD1
(SEQ ID NO: 40)
MAPKKKRKVGIHGVPAADKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGDSGGKRPAATKKAGQAK
KKKESGGGSLSGKKAAAAAAAAAAAATGTEAGPGTAGGSENGSEVAAQPA
GLSGPAEVGPGAVGERTPRKKEPPRASPPGGLAEPPGSAGPQAGPTVVPG
SATPMETGIAETPEGRRTSRRKRAKVEYREMDESLANLSEDEYYSEEERN
AKAEKEKKLPPPPPQAPPEEENESEPEEPSGVEGAAFQSRLPHDRMTSQE
AACFPDIISGPQQTQKVFLFIRNRTLQLWLDNPKIQLTFEATLQQLEAPY
NSDTVLVHRVHSYLERHGLINFGIYKRIKPLPTKKTGKVIIIGSGVSGLA
AARQLQSFGMDVTLLEARDRVGGRVATFRKGNYVADLGAMVVTGLGGNPM
AVVSKQVNMELAKIKQKCPLYEANGQAVPKEKDEMVEQEFNRLLEATSYL
SHQLDFNVLNNKHVSLGQALEVVIQLQEKHVKDEQIEHWKKIVKTQEELK
ELLNKMVNLKEKIKELHQQYKEASEVKPPRDITAEFLVKSKHRDLTALCK
EYDELAETQGKLEEKLQELEANPPSDVYLSSRDRQILDWHFANLEFANAT
PLSTLSLKHWDQDDDFEFTGSHLTVRNGYSCVPVALAEGLDIKLNTAVRQ
VRYTASGCEVIAVNTRSTSQTFIYKCDAVLCTLPLGVLKQQPPAVQFVPP
LPEWKTSAVQRMGFGNLNKVVLCFDRVFWDPSVNLFGHVGSTTASRGELF
LFWNLYKAPILLALVAGEAAGIMENISDDVIVGRCLAILKGIFGSSAVPQ
PKETVVSRWRADPWARGSYSYVAAGSSGNDYDLMAQPITPGPSIPGAPQP
IPRLFFAGEHTIRNYPATVHGALLSGLREAGRIADQFLGAMYTLPRQATP
GVPAQQSPSMSGGKRPAATKKAGQAKKKKGSYPYDVPDYA
dCas9-LSD1 without HA tag
(SEQ ID NO: 75)
MAPKKKRKVGIHGVPAADKKYSIGLAIGTNSVGWAVITDEYKVPSKKFKV
LGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRI
EEGIKELGSQILKEHPVENTQLQNEKLYLYYLQNGRDMYVDQELDINRLS
DYDVAAIVPQSFLKDDSIDNKVLTRSDKARGKSDNVPSEEVVKKMKNYWR
QLLNAKLITQRKFDNLTKAERGGLSELDKAGFIKRQLVETRQITKHVAQI
LDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGDSGGKRPAATKKAGQAK
KKKESGGGSLSGKKAAAAAAAAAAAATGTEAGPGTAGGSENGSEVAAQPA
GLSGPAEVGPGAVGERTPRKKEPPRASPPGGLAEPPGSAGPQAGPTVVPG
SATPMETGIAETPEGRRTSRRKRAKVEYREMDESLANLSEDEYYSEEERN
AKAEKEKKLPPPPPQAPPEEENESEPEEPSGVEGAAFQSRLPHDRMTSQE
AACFPDIISGPQQTQKVFLFIRNRTLQLWLDNPKIQLTFEATLQQLEAPY
NSDTVLVHRVHSYLERHGLINFGIYKRIKPLPTKKTGKVIIIGSGVSGLA
AARQLQSFGMDVTLLEARDRVGGRVATFRKGNYVADLGAMVVTGLGGNPM
AVVSKQVNMELAKIKQKCPLYEANGQAVPKEKDEMVEQEFNRLLEATSYL
SHQLDFNVLNNKHVSLGQALEVVIQLQEKHVKDEQIEHWKKIVKTQEELK
ELLNKMVNLKEKIKELHQQYKEASEVKPPRDITAEFLVKSKHRDLTALCK
EYDELAETQGKLEEKLQELEANPPSDVYLSSRDRQILDWHFANLEFANAT
PLSTLSLKHWDQDDDFEFTGSHLTVRNGYSCVPVALAEGLDIKLNTAVRQ
VRYTASGCEVIAVNTRSTSQTFIYKCDAVLCTLPLGVLKQQPPAVQFVPP
LPEWKTSAVQRMGFGNLNKVVLCFDRVFWDPSVNLFGHVGSTTASRGELF
LFWNLYKAPILLALVAGEAAGIMENISDDVIVGRCLAILKGIFGSSAVPQ
PKETVVSRWRADPWARGSYSYVAAGSSGNDYDLMAQPITPGPSIPGAPQP
IPRLFFAGEHTIRNYPATVHGALLSGLREAGRIADQFLGAMYTLPRQATP
GVPAQQSPSMSGGKRPAATKKAGQAKKKKGS

In some embodiments, an expression repressor comprises a DNA-targeting moiety comprising dCas9, e.g., an S. pyogenes dCas9, and a repressor domain comprising EZH2, e.g., a EZH2 domain. In some embodiments, the expression repressor is encoded by the nucleic acid sequence of SEQ ID NOs: 29 (e.g., a nucleic acid (e.g., cDNA) encoding the expression repressor). In some embodiments, a nucleic acid described herein comprises a nucleic acid sequence of SEQ ID NO: 29 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9 (MR-28938)
(SEQ ID NO: 29)
AGGAAATAAGAGAGAAAAGAAGAGTAAGAAGAAATATAAGAGCCACCATGGCCCCCAAGA
AGAAGCGGAAGGTGGGCGGCAGCGGCGGCAGCGGCCAGACCGGCAAGAAGAGCGAGAAGG
GCCCCGTGTGCTGGCGGAAGCGGGTGAAGAGCGAGTACATGCGGCTGCGGCAGCTGAAGCG
GTTCCGGCGGGCCGACGAGGTGAAGAGCATGTTCAGCAGCAACCGGCAGAAGATCCTGGAG
CGGACCGAGATCCTGAACCAGGAGTGGAAGCAGCGGCGAATCCAGCCCGTGCACATCCTGA
CCAGCGTGAGCAGCCTGCGGGGCACCCGGGAGTGCAGCGTGACCAGCGACCTGGACTTCCC
CACCCAGGTGATCCCCCTAAAGACCCTGAACGCCGTGGCCAGCGTGCCCATCATGTACAGCT
GGAGCCCCCTGCAGCAGAACTTCATGGTGGAGGACGAGACCGTGCTGCACAACATCCCCTA
CATGGGCGACGAGGTGCTGGACCAGGACGGCACCTTCATCGAGGAGCTGATCAAGAACTAC
GACGGCAAGGTGCACGGCGACCGGGAGTGCGGCTTCATCAACGACGAGATCTTCGTGGAGC
TGGTGAACGCCCTGGGCCAGTACAACGACGACGACGACGACGACGACGGCGACGACCCCGA
GGAGCGGGAGGAGAAGCAGAAGGACCTGGAGGACCACCGGGACGACAAGGAGAGCCGGCC
CCCCCGGAAGTTCCCCAGCGACAAGATCTTCGAGGCCATCAGCAGCATGTTCCCCGACAAGG
GCACCGCCGAGGAGCTGAAGGAGAAGTACAAGGAGCTGACCGAGCAGCAGCTGCCCGGCG
CCCTGCCCCCCGAGTGCACCCCCAACATCGACGGCCCCAACGCCAAGAGCGTGCAGCGGGA
GCAGAGCCTGCACAGCTTCCACACCCTGTTCTGCCGGCGGTGCTTCAAGTACGACTGCTTCC
TGCACCCCTTCCACGCCACCCCCAACACCTACAAGCGGAAGAACACCGAGACCGCCCTGGA
CAACAAGCCCTGCGGCCCCCAGTGCTACCAGCACCTGGAGGGCGCCAAGGAGTTCGCCGCC
GCCCTGACCGCCGAGCGGATCAAGACCCCCCCCAAGCGGCCCGGCGGCCGGCGGCGGGGCC
GGCTGCCCAACAACAGCAGCCGGCCCAGCACCCCCACCATCAACGTGCTGGAGAGCAAGGA
CACCGACAGCGACCGGGAGGCCGGCACCGAGACCGGCGGCGAGAACAACGACAAGGAGGA
GGAGGAGAAGAAGGACGAGACCAGCAGCAGCAGCGAGGCCAACAGCCGGTGCCAGACCCC
CATCAAGATGAAGCCCAACATCGAGCCCCCCGAGAACGTGGAGTGGAGCGGCGCCGAGGCC
AGCATGTTCCGGGTGCTGATCGGCACCTACTACGACAACTTCTGCGCCATCGCCCGGCTGAT
CGGCACCAAGACCTGCCGGCAGGTGTACGAGTTCCGGGTGAAGGAGAGCAGCATCATCGCC
CCCGCCCCCGCCGAGGACGTGGACACCCCCCCCCGGAAGAAGAAGCGGAAGCACCGGCTGT
GGGCCGCCCACTGCCGGAAGATCCAGCTGAAGAAGGACGGCAGCAGCAACCACGTGTACAA
CTACCAGCCCTGCGACCACCCCCGGCAGCCCTGCGACAGCAGCTGCCCCTGCGTGATCGCCC
AGAACTTCTGCGAGAAGTTCTGCCAGTGCAGCAGCGAGTGCCAGAACCGGTTCCCCGGCTGC
CGGTGCAAGGCCCAGTGCAACACCAAGCAGTGCCCCTGCTACCTGGCCGTGCGGGAGTGCG
ACCCCGACCTGTGCCTGACCTGCGGCGCCGCCGACCACTGGGACAGCAAGAACGTGAGCTG
CAAGAACTGCAGCATCCAGCGGGGCAGCAAGAAGCACCTGCTGCTGGCCCCCAGCGACGTG
GCCGGCTGGGGCATCTTCATCAAGGACCCCGTGCAGAAGAACGAGTTCATCAGCGAGTACT
GCGGCGAGATCATCAGCCAGGACGAGGCCGACCGGCGGGGCAAGGTGTACGACAAGTACAT
GTGCAGCTTCCTGTTCAACCTGAACAACGACTTCGTGGTGGACGCCACCCGGAAGGGCAACA
AGATCCGGTTCGCCAACCACAGCGTGAACCCCAACTGCTACGCCAAGGTGATGATGGTGAA
CGGCGACCACCGGATCGGCATCTTCGCCAAGCGGGCCATCCAGACCGGCGAGGAGCTGTTC
TTCGACTACCGGTACAGCCAGGCCGACGCCCTGAAGTACGTGGGCATCGAGCGGGAGATGG
AGATCCCCAGCACCGGCGGCAGCGGCGGCAGCGGCGGCAGCGGCGGCAGCGGCGGCAGCG
GCCGACCCGACAAGAAGTACAGCATCGGCCTGGCCATCGGCACCAACAGCGTGGGCTGGGC
CGTGATCACCGACGAGTACAAGGTGCCCAGCAAGAAGTTCAAGGTGCTGGGCAACACCGAC
CGGCACAGCATCAAGAAGAACCTGATCGGCGCCCTGCTGTTCGACAGCGGCGAGACCGCCG
AGGCCACCCGGCTGAAGCGGACCGCCCGGCGGCGGTACACCCGGCGGAAGAACCGGATCTG
CTACCTGCAGGAGATCTTCAGCAACGAGATGGCCAAGGTGGACGACAGCTTCTTCCACCGGC
TGGAGGAGAGCTTCCTGGTGGAGGAGGACAAGAAGCACGAGCGGCACCCCATCTTCGGCAA
CATCGTGGACGAGGTGGCCTACCACGAGAAGTACCCCACCATCTACCACCTGCGGAAGAAG
CTGGTGGACAGCACCGACAAGGCCGACCTGCGGCTGATCTACCTGGCCCTGGCCCACATGAT
CAAGTTCCGGGGCCACTTCCTGATCGAGGGCGACCTGAACCCCGACAACAGCGACGTGGAC
AAGCTGTTCATCCAGCTGGTGCAGACCTACAACCAGCTGTTCGAGGAGAACCCCATCAACGC
CAGCGGCGTGGACGCCAAGGCCATCCTGAGCGCCCGGCTGAGCAAGAGCCGGCGGCTGGAG
AACCTGATCGCCCAGCTGCCCGGCGAGAAGAAGAACGGCCTGTTCGGCAACCTGATCGCCC
TGAGCCTGGGCCTGACCCCCAACTTCAAGAGCAACTTCGACCTGGCCGAGGACGCCAAGCT
GCAGCTGAGCAAGGACACCTACGACGACGACCTGGACAACCTGCTGGCCCAGATCGGCGAC
CAGTACGCCGACCTGTTCCTGGCCGCCAAGAACCTGAGCGACGCCATCCTGCTGAGCGACAT
CCTGCGGGTGAACACCGAGATCACCAAGGCCCCCCTGAGCGCCAGCATGATCAAGCGGTAC
GACGAGCACCACCAGGACCTGACCCTGCTGAAGGCCCTGGTGCGGCAGCAGCTGCCCGAGA
AGTACAAGGAGATCTTCTTCGACCAGAGCAAGAACGGCTACGCCGGCTACATCGACGGCGG
CGCCAGCCAGGAGGAGTTCTACAAGTTCATCAAGCCCATCCTGGAGAAGATGGACGGCACC
GAGGAGCTGCTGGTGAAGCTGAACCGGGAGGACCTGCTGCGGAAGCAGCGGACCTTCGACA
ACGGCAGCATCCCCCACCAGATCCACCTGGGCGAGCTGCACGCCATCCTGCGGCGGCAGGA
GGACTTCTACCCCTTCCTGAAGGACAACCGGGAGAAGATCGAGAAGATCCTGACCTTCCGG
ATCCCCTACTACGTGGGCCCCCTGGCCCGGGGCAACAGCCGGTTCGCCTGGATGACCCGGAA
ATCCGAGGAGACCATCACCCCCTGGAACTTCGAGGAGGTGGTGGACAAGGGCGCCAGCGCC
CAGAGCTTCATCGAGCGGATGACCAACTTCGACAAGAACCTGCCCAACGAGAAGGTGCTGC
CCAAGCACAGCCTGCTGTACGAGTACTTCACCGTGTACAACGAGCTGACCAAGGTGAAGTA
CGTGACCGAGGGCATGCGGAAGCCCGCCTTCCTGAGCGGCGAGCAGAAGAAGGCCATCGTG
GACCTGCTGTTCAAGACCAACCGGAAGGTGACCGTGAAGCAGCTGAAGGAGGACTACTTCA
AGAAGATCGAGTGCTTCGACAGCGTGGAGATCAGCGGCGTGGAGGACCGGTTCAACGCCAG
CCTGGGCACCTACCACGACCTGCTGAAGATCATCAAGGACAAGGACTTCCTGGACAACGAG
GAGAACGAGGACATCCTGGAGGACATCGTGCTGACCCTGACCCTGTTCGAGGACCGGGAGA
TGATCGAGGAGCGGCTGAAAACCTACGCCCACCTGTTCGACGACAAGGTGATGAAGCAGCT
GAAGCGGCGGCGGTACACCGGCTGGGGCCGGCTGAGCCGGAAGCTGATCAACGGCATCCGG
GACAAGCAGAGCGGCAAGACCATCCTGGACTTCCTGAAATCCGACGGCTTCGCCAACCGGA
ACTTCATGCAGCTGATCCACGACGACAGCCTGACCTTCAAGGAGGACATCCAGAAGGCCCA
GGTGAGCGGCCAGGGCGACAGCCTGCACGAGCACATCGCCAACCTGGCCGGCAGCCCCGCC
ATCAAGAAGGGCATCCTGCAGACCGTGAAGGTGGTGGACGAGCTGGTGAAGGTGATGGGCC
GGCACAAGCCCGAGAACATCGTGATCGAGATGGCCCGGGAGAACCAGACCACCCAGAAGG
GCCAGAAGAACAGCCGGGAGCGGATGAAGCGGATCGAGGAGGGCATCAAGGAGCTGGGCA
GCCAGATCCTGAAGGAGCACCCCGTGGAGAACACCCAGCTGCAGAACGAGAAGCTGTACCT
GTACTACCTGCAGAACGGCCGGGACATGTACGTGGACCAGGAGCTGGACATCAACCGGCTG
AGCGACTACGACGTGGCCGCCATCGTGCCCCAGAGCTTCCTGAAGGACGACAGCATCGACA
ACAAGGTGCTGACCCGGAGCGACAAGGCCCGGGGCAAGAGCGACAACGTGCCCAGCGAGG
AGGTGGTGAAGAAGATGAAGAACTACTGGCGGCAGCTGCTGAACGCCAAGCTGATCACCCA
GCGGAAGTTCGACAACCTGACCAAGGCCGAGCGGGGCGGCCTGAGCGAGCTGGACAAGGCC
GGCTTCATCAAGCGGCAGCTGGTGGAGACCCGGCAGATCACCAAGCACGTGGCCCAGATCC
TGGACAGCCGGATGAACACCAAGTACGACGAGAACGACAAGCTGATCCGGGAGGTGAAGG
TGATCACCCTGAAATCCAAGCTGGTGAGCGACTTCCGGAAGGACTTCCAGTTCTACAAGGTG
CGGGAGATCAACAACTACCACCACGCCCACGACGCCTACCTGAACGCCGTGGTGGGCACCG
CCCTGATCAAGAAGTACCCCAAGCTGGAGAGCGAGTTCGTGTACGGCGACTACAAGGTGTA
CGACGTGCGGAAGATGATCGCCAAGAGCGAGCAGGAGATCGGCAAGGCCACCGCCAAGTA
CTTCTTCTACAGCAACATCATGAACTTCTTCAAGACCGAGATCACCCTGGCCAACGGCGAGA
TCCGGAAGCGGCCCCTGATCGAGACCAACGGCGAGACCGGCGAGATCGTGTGGGACAAGGG
CCGGGACTTCGCCACCGTGCGGAAGGTGCTGAGCATGCCCCAGGTGAACATCGTGAAGAAA
ACCGAGGTGCAGACCGGCGGCTTCAGCAAGGAGAGCATCCTGCCCAAGCGGAACAGCGACA
AGCTGATCGCCCGGAAGAAGGACTGGGACCCCAAGAAGTACGGCGGCTTCGACAGCCCCAC
CGTGGCCTACAGCGTGCTGGTGGTGGCCAAGGTGGAGAAGGGCAAGAGCAAGAAGCTGAA
ATCCGTGAAGGAGCTGCTGGGCATCACCATCATGGAGCGGAGCAGCTTCGAGAAGAACCCC
ATCGACTTCCTGGAGGCCAAGGGCTACAAGGAGGTGAAGAAGGACCTGATCATCAAGCTGC
CCAAGTACAGCCTGTTCGAGCTGGAGAACGGCCGGAAGCGGATGCTGGCCAGCGCCGGCGA
GCTGCAGAAGGGCAACGAGCTGGCCCTGCCCAGCAAGTACGTGAACTTCCTGTACCTGGCC
AGCCACTACGAGAAGCTGAAGGGCAGCCCCGAGGACAACGAGCAGAAGCAGCTGTTCGTGG
AGCAGCACAAGCACTACCTGGACGAGATCATCGAGCAGATCAGCGAGTTCAGCAAGCGGGT
GATCCTGGCCGACGCCAACCTGGACAAGGTGCTGAGCGCCTACAACAAGCACCGGGACAAG
CCCATCCGGGAGCAGGCCGAGAACATCATCCACCTGTTCACCCTGACCAACCTGGGCGCCCC
CGCCGCCTTCAAGTACTTCGACACCACCATCGACCGGAAGCGGTACACCAGCACCAAGGAG
GTGCTGGACGCCACCCTGATCCACCAGAGCATCACCGGCCTGTACGAGACCCGGATCGACCT
GAGCCAGCTGGGCGGCGACAGCGGCGGCAAGCGGCCCGCCGCCACCAAGAAGGCCGGCCA
GGCCAAGAAGAAGAAGGGCAGCTACCCCTACGACGTGCCCGACTACGCCTGAGCGGCCGCT
TAATTAAGCTGCCTTCTGCGGGGCTTGCCTTCTGGCCATGCCCTTCTTCTCTCCCTTGCACCTG
TACCTCTTGGTCTTTGAATAAAGCCTGAGTAGGAAGTCTAGAAAAAAAAAAAAAAAAAAAA
AAAAAAAAAAAAAAAAAAAAAAAAAAAAAAAAAAAAAAAAAAAAAAAAAAAAAAAAAA
AA

In some embodiments, an expression repressor comprises the amino acid sequence of SEQ ID NOs: 41 or 76. In some embodiments, an expression repressor described herein comprises an amino acid sequence of SEQ ID NO: 41 or 76 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9
(SEQ ID NO: 41)
MAPKKKRKVGGSGGSGQTGKKSEKGPVCWRKRVKSEYMRLRQLKRFRRAD
EVKSMFSSNRQKILERTEILNQEWKQRRIQPVHILTSVSSLRGTRECSVT
SDLDFPTQVIPLKTLNAVASVPIMYSWSPLQQNFMVEDETVLHNIPYMGD
EVLDQDGTFIEELIKNYDGKVHGDRECGFINDEIFVELVNALGQYNDDDD
DDDGDDPEEREEKQKDLEDHRDDKESRPPRKFPSDKIFEAISSMFPDKGT
AEELKEKYKELTEQQLPGALPPECTPNIDGPNAKSVQREQSLHSFHTLFC
RRCFKYDCFLHPFHATPNTYKRKNTETALDNKPCGPQCYQHLEGAKEFAA
ALTAERIKTPPKRPGGRRRGRLPNNSSRPSTPTINVLESKDTDSDREAGT
ETGGENNDKEEEEKKDETSSSSEANSRCQTPIKMKPNIEPPENVEWSGAE
ASMFRVLIGTYYDNFCAIARLIGTKTCRQVYEFRVKESSIIAPAPAEDVD
TPPRKKKRKHRLWAAHCRKIQLKKDGSSNHVYNYQPCDHPRQPCDSSCPC
VIAQNFCEKFCQCSSECQNRFPGCRCKAQCNTKQCPCYLAVRECDPDLCL
TCGAADHWDSKNVSCKNCSIQRGSKKHLLLAPSDVAGWGIFIKDPVQKNE
FISEYCGEIISQDEADRRGKVYDKYMCSFLFNLNNDFVVDATRKGNKIRF
ANHSVNPNCYAKVMMVNGDHRIGIFAKRAIQTGEELFFDYRYSQADALKY
VGIEREMEIPSTGGSGGSGGSGGSGGSGRPDKKYSIGLAIGTNSVGWAVI
TDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKK
IECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRD
MYVDQELDINRLSDYDVAAIVPQSFLKDDSIDNKVLTRSDKARGKSDNVP
SEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMER
SSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAP
AAFKYFDTTIDRKRYTSTKEVLDATLIHQSITGLYETRIDLSQLGGDSGG
KRPAATKKAGQAKKKKGSYPYDVPDYA
EZH2-dCas9 without HA tag
(SEQ ID NO: 76)
MAPKKKRKVGGSGGSGQTGKKSEKGPVCWRKRVKSEYMRLRQLKRFRRAD
EVKSMFSSNRQKILERTEILNQEWKQRRIQPVHILTSVSSLRGTRECSVT
SDLDFPTQVIPLKTLNAVASVPIMYSWSPLQQNFMVEDETVLHNIPYMGD
EVLDQDGTFIEELIKNYDGKVHGDRECGFINDEIFVELVNALGQYNDDDD
DDDGDDPEEREEKQKDLEDHRDDKESRPPRKFPSDKIFEAISSMFPDKGT
AEELKEKYKELTEQQLPGALPPECTPNIDGPNAKSVQREQSLHSFHTLFC
RRCFKYDCFLHPFHATPNTYKRKNTETALDNKPCGPQCYQHLEGAKEFAA
ALTAERIKTPPKRPGGRRRGRLPNNSSRPSTPTINVLESKDTDSDREAGT
ETGGENNDKEEEEKKDETSSSSEANSRCQTPIKMKPNIEPPENVEWSGAE
ASMFRVLIGTYYDNFCAIARLIGTKTCRQVYEFRVKESSIIAPAPAEDVD
TPPRKKKRKHRLWAAHCRKIQLKKDGSSNHVYNYQPCDHPRQPCDSSCPC
VIAQNFCEKFCQCSSECQNRFPGCRCKAQCNTKQCPCYLAVRECDPDLCL
TCGAADHWDSKNVSCKNCSIQRGSKKHLLLAPSDVAGWGIFIKDPVQKNE
FISEYCGEIISQDEADRRGKVYDKYMCSFLFNLNNDFVVDATRKGNKIRF
ANHSVNPNCYAKVMMVNGDHRIGIFAKRAIQTGEELFFDYRYSQADALKY
VGIEREMEIPSTGGSGGSGGSGGSGGSGRPDKKYSIGLAIGTNSVGWAVI
TDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIE
GDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHA
ILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKK
IECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRD
MYVDQELDINRLSDYDVAAIVPQSFLKDDSIDNKVLTRSDKARGKSDNVP
SEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMER
SSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAP
AAFKYFDTTIDRKRYTSTKEVLDATLIHQSITGLYETRIDLSQLGGDSGG
KRPAATKKAGQAKKKKGS

In some embodiments, an expression repressor comprises a DNA-targeting moiety comprising dCas9, e.g., an S. pyogenes dCas9, and a first repressor domain comprising EZH2 e.g., a EZH2 domain and a second repressor domain comprising KRAB, e.g., a KRAB domain. In some embodiments, the expression repressor is encoded by the nucleic acid sequence of SEQ ID NOs: 30 (e.g., a nucleic acid (e.g., cDNA) encoding the expression repressor). In some embodiments, a nucleic acid described herein comprises a nucleic acid sequence of SEQ ID NO: 30 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2- dCas9-KRAB (MR-29448)
(SEQ ID NO: 30)
AGGAAATAAGAGAGAAAAGAAGAGTAAGAAGAAATATAAGAGCCACCATGGCCCCCAAGA
AGAAGCGGAAGGTGGGCGGCAGCGGCGGCAGCGGCCAGACCGGCAAGAAGAGCGAGAAGG
GCCCCGTGTGCTGGCGGAAGCGGGTGAAGAGCGAGTACATGCGGCTGCGGCAGCTGAAGCG
GTTCCGGCGGGCCGACGAGGTGAAGAGCATGTTCAGCAGCAACCGGCAGAAGATCCTGGAG
CGGACCGAGATCCTGAACCAGGAGTGGAAGCAGCGGCGAATCCAGCCCGTGCACATCCTGA
CCAGCGTGAGCAGCCTGCGGGGCACCCGGGAGTGCAGCGTGACCAGCGACCTGGACTTCCC
CACCCAGGTGATCCCCCTAAAGACCCTGAACGCCGTGGCCAGCGTGCCCATCATGTACAGCT
GGAGCCCCCTGCAGCAGAACTTCATGGTGGAGGACGAGACCGTGCTGCACAACATCCCCTA
CATGGGCGACGAGGTGCTGGACCAGGACGGCACCTTCATCGAGGAGCTGATCAAGAACTAC
GACGGCAAGGTGCACGGCGACCGGGAGTGCGGCTTCATCAACGACGAGATCTTCGTGGAGC
TGGTGAACGCCCTGGGCCAGTACAACGACGACGACGACGACGACGACGGCGACGACCCCGA
GGAGCGGGAGGAGAAGCAGAAGGACCTGGAGGACCACCGGGACGACAAGGAGAGCCGGCC
CCCCCGGAAGTTCCCCAGCGACAAGATCTTCGAGGCCATCAGCAGCATGTTCCCCGACAAGG
GCACCGCCGAGGAGCTGAAGGAGAAGTACAAGGAGCTGACCGAGCAGCAGCTGCCCGGCG
CCCTGCCCCCCGAGTGCACCCCCAACATCGACGGCCCCAACGCCAAGAGCGTGCAGCGGGA
GCAGAGCCTGCACAGCTTCCACACCCTGTTCTGCCGGCGGTGCTTCAAGTACGACTGCTTCC
TGCACCCCTTCCACGCCACCCCCAACACCTACAAGCGGAAGAACACCGAGACCGCCCTGGA
CAACAAGCCCTGCGGCCCCCAGTGCTACCAGCACCTGGAGGGCGCCAAGGAGTTCGCCGCC
GCCCTGACCGCCGAGCGGATCAAGACCCCCCCCAAGCGGCCCGGCGGCCGGCGGCGGGGCC
GGCTGCCCAACAACAGCAGCCGGCCCAGCACCCCCACCATCAACGTGCTGGAGAGCAAGGA
CACCGACAGCGACCGGGAGGCCGGCACCGAGACCGGCGGCGAGAACAACGACAAGGAGGA
GGAGGAGAAGAAGGACGAGACCAGCAGCAGCAGCGAGGCCAACAGCCGGTGCCAGACCCC
CATCAAGATGAAGCCCAACATCGAGCCCCCCGAGAACGTGGAGTGGAGCGGCGCCGAGGCC
AGCATGTTCCGGGTGCTGATCGGCACCTACTACGACAACTTCTGCGCCATCGCCCGGCTGAT
CGGCACCAAGACCTGCCGGCAGGTGTACGAGTTCCGGGTGAAGGAGAGCAGCATCATCGCC
CCCGCCCCCGCCGAGGACGTGGACACCCCCCCCCGGAAGAAGAAGCGGAAGCACCGGCTGT
GGGCCGCCCACTGCCGGAAGATCCAGCTGAAGAAGGACGGCAGCAGCAACCACGTGTACAA
CTACCAGCCCTGCGACCACCCCCGGCAGCCCTGCGACAGCAGCTGCCCCTGCGTGATCGCCC
AGAACTTCTGCGAGAAGTTCTGCCAGTGCAGCAGCGAGTGCCAGAACCGGTTCCCCGGCTGC
CGGTGCAAGGCCCAGTGCAACACCAAGCAGTGCCCCTGCTACCTGGCCGTGCGGGAGTGCG
ACCCCGACCTGTGCCTGACCTGCGGCGCCGCCGACCACTGGGACAGCAAGAACGTGAGCTG
CAAGAACTGCAGCATCCAGCGGGGCAGCAAGAAGCACCTGCTGCTGGCCCCCAGCGACGTG
GCCGGCTGGGGCATCTTCATCAAGGACCCCGTGCAGAAGAACGAGTTCATCAGCGAGTACT
GCGGCGAGATCATCAGCCAGGACGAGGCCGACCGGCGGGGCAAGGTGTACGACAAGTACAT
GTGCAGCTTCCTGTTCAACCTGAACAACGACTTCGTGGTGGACGCCACCCGGAAGGGCAACA
AGATCCGGTTCGCCAACCACAGCGTGAACCCCAACTGCTACGCCAAGGTGATGATGGTGAA
CGGCGACCACCGGATCGGCATCTTCGCCAAGCGGGCCATCCAGACCGGCGAGGAGCTGTTC
TTCGACTACCGGTACAGCCAGGCCGACGCCCTGAAGTACGTGGGCATCGAGCGGGAGATGG
AGATCCCCAGCACCGGCGGCAGCGGCGGCAGCGGCGGCAGCGGCGGCAGCGGCGGCAGCG
GCCGACCCGACAAGAAGTACAGCATCGGCCTGGCCATCGGCACCAACAGCGTGGGCTGGGC
CGTGATCACCGACGAGTACAAGGTGCCCAGCAAGAAGTTCAAGGTGCTGGGCAACACCGAC
CGGCACAGCATCAAGAAGAACCTGATCGGCGCCCTGCTGTTCGACAGCGGCGAGACCGCCG
AGGCCACCCGGCTGAAGCGGACCGCCCGGCGGCGGTACACCCGGCGGAAGAACCGGATCTG
CTACCTGCAGGAGATCTTCAGCAACGAGATGGCCAAGGTGGACGACAGCTTCTTCCACCGGC
TGGAGGAGAGCTTCCTGGTGGAGGAGGACAAGAAGCACGAGCGGCACCCCATCTTCGGCAA
CATCGTGGACGAGGTGGCCTACCACGAGAAGTACCCCACCATCTACCACCTGCGGAAGAAG
CTGGTGGACAGCACCGACAAGGCCGACCTGCGGCTGATCTACCTGGCCCTGGCCCACATGAT
CAAGTTCCGGGGCCACTTCCTGATCGAGGGCGACCTGAACCCCGACAACAGCGACGTGGAC
AAGCTGTTCATCCAGCTGGTGCAGACCTACAACCAGCTGTTCGAGGAGAACCCCATCAACGC
CAGCGGCGTGGACGCCAAGGCCATCCTGAGCGCCCGGCTGAGCAAGAGCCGGCGGCTGGAG
AACCTGATCGCCCAGCTGCCCGGCGAGAAGAAGAACGGCCTGTTCGGCAACCTGATCGCCC
TGAGCCTGGGCCTGACCCCCAACTTCAAGAGCAACTTCGACCTGGCCGAGGACGCCAAGCT
GCAGCTGAGCAAGGACACCTACGACGACGACCTGGACAACCTGCTGGCCCAGATCGGCGAC
CAGTACGCCGACCTGTTCCTGGCCGCCAAGAACCTGAGCGACGCCATCCTGCTGAGCGACAT
CCTGCGGGTGAACACCGAGATCACCAAGGCCCCCCTGAGCGCCAGCATGATCAAGCGGTAC
GACGAGCACCACCAGGACCTGACCCTGCTGAAGGCCCTGGTGCGGCAGCAGCTGCCCGAGA
AGTACAAGGAGATCTTCTTCGACCAGAGCAAGAACGGCTACGCCGGCTACATCGACGGCGG
CGCCAGCCAGGAGGAGTTCTACAAGTTCATCAAGCCCATCCTGGAGAAGATGGACGGCACC
GAGGAGCTGCTGGTGAAGCTGAACCGGGAGGACCTGCTGCGGAAGCAGCGGACCTTCGACA
ACGGCAGCATCCCCCACCAGATCCACCTGGGCGAGCTGCACGCCATCCTGCGGCGGCAGGA
GGACTTCTACCCCTTCCTGAAGGACAACCGGGAGAAGATCGAGAAGATCCTGACCTTCCGG
ATCCCCTACTACGTGGGCCCCCTGGCCCGGGGCAACAGCCGGTTCGCCTGGATGACCCGGAA
ATCCGAGGAGACCATCACCCCCTGGAACTTCGAGGAGGTGGTGGACAAGGGCGCCAGCGCC
CAGAGCTTCATCGAGCGGATGACCAACTTCGACAAGAACCTGCCCAACGAGAAGGTGCTGC
CCAAGCACAGCCTGCTGTACGAGTACTTCACCGTGTACAACGAGCTGACCAAGGTGAAGTA
CGTGACCGAGGGCATGCGGAAGCCCGCCTTCCTGAGCGGCGAGCAGAAGAAGGCCATCGTG
GACCTGCTGTTCAAGACCAACCGGAAGGTGACCGTGAAGCAGCTGAAGGAGGACTACTTCA
AGAAGATCGAGTGCTTCGACAGCGTGGAGATCAGCGGCGTGGAGGACCGGTTCAACGCCAG
CCTGGGCACCTACCACGACCTGCTGAAGATCATCAAGGACAAGGACTTCCTGGACAACGAG
GAGAACGAGGACATCCTGGAGGACATCGTGCTGACCCTGACCCTGTTCGAGGACCGGGAGA
TGATCGAGGAGCGGCTGAAAACCTACGCCCACCTGTTCGACGACAAGGTGATGAAGCAGCT
GAAGCGGCGGCGGTACACCGGCTGGGGCCGGCTGAGCCGGAAGCTGATCAACGGCATCCGG
GACAAGCAGAGCGGCAAGACCATCCTGGACTTCCTGAAATCCGACGGCTTCGCCAACCGGA
ACTTCATGCAGCTGATCCACGACGACAGCCTGACCTTCAAGGAGGACATCCAGAAGGCCCA
GGTGAGCGGCCAGGGCGACAGCCTGCACGAGCACATCGCCAACCTGGCCGGCAGCCCCGCC
ATCAAGAAGGGCATCCTGCAGACCGTGAAGGTGGTGGACGAGCTGGTGAAGGTGATGGGCC
GGCACAAGCCCGAGAACATCGTGATCGAGATGGCCCGGGAGAACCAGACCACCCAGAAGG
GCCAGAAGAACAGCCGGGAGCGGATGAAGCGGATCGAGGAGGGCATCAAGGAGCTGGGCA
GCCAGATCCTGAAGGAGCACCCCGTGGAGAACACCCAGCTGCAGAACGAGAAGCTGTACCT
GTACTACCTGCAGAACGGCCGGGACATGTACGTGGACCAGGAGCTGGACATCAACCGGCTG
AGCGACTACGACGTGGCCGCCATCGTGCCCCAGAGCTTCCTGAAGGACGACAGCATCGACA
ACAAGGTGCTGACCCGGAGCGACAAGGCCCGGGGCAAGAGCGACAACGTGCCCAGCGAGG
AGGTGGTGAAGAAGATGAAGAACTACTGGCGGCAGCTGCTGAACGCCAAGCTGATCACCCA
GCGGAAGTTCGACAACCTGACCAAGGCCGAGCGGGGCGGCCTGAGCGAGCTGGACAAGGCC
GGCTTCATCAAGCGGCAGCTGGTGGAGACCCGGCAGATCACCAAGCACGTGGCCCAGATCC
TGGACAGCCGGATGAACACCAAGTACGACGAGAACGACAAGCTGATCCGGGAGGTGAAGG
TGATCACCCTGAAATCCAAGCTGGTGAGCGACTTCCGGAAGGACTTCCAGTTCTACAAGGTG
CGGGAGATCAACAACTACCACCACGCCCACGACGCCTACCTGAACGCCGTGGTGGGCACCG
CCCTGATCAAGAAGTACCCCAAGCTGGAGAGCGAGTTCGTGTACGGCGACTACAAGGTGTA
CGACGTGCGGAAGATGATCGCCAAGAGCGAGCAGGAGATCGGCAAGGCCACCGCCAAGTA
CTTCTTCTACAGCAACATCATGAACTTCTTCAAGACCGAGATCACCCTGGCCAACGGCGAGA
TCCGGAAGCGGCCCCTGATCGAGACCAACGGCGAGACCGGCGAGATCGTGTGGGACAAGGG
CCGGGACTTCGCCACCGTGCGGAAGGTGCTGAGCATGCCCCAGGTGAACATCGTGAAGAAA
ACCGAGGTGCAGACCGGCGGCTTCAGCAAGGAGAGCATCCTGCCCAAGCGGAACAGCGACA
AGCTGATCGCCCGGAAGAAGGACTGGGACCCCAAGAAGTACGGCGGCTTCGACAGCCCCAC
CGTGGCCTACAGCGTGCTGGTGGTGGCCAAGGTGGAGAAGGGCAAGAGCAAGAAGCTGAA
ATCCGTGAAGGAGCTGCTGGGCATCACCATCATGGAGCGGAGCAGCTTCGAGAAGAACCCC
ATCGACTTCCTGGAGGCCAAGGGCTACAAGGAGGTGAAGAAGGACCTGATCATCAAGCTGC
CCAAGTACAGCCTGTTCGAGCTGGAGAACGGCCGGAAGCGGATGCTGGCCAGCGCCGGCGA
GCTGCAGAAGGGCAACGAGCTGGCCCTGCCCAGCAAGTACGTGAACTTCCTGTACCTGGCC
AGCCACTACGAGAAGCTGAAGGGCAGCCCCGAGGACAACGAGCAGAAGCAGCTGTTCGTGG
AGCAGCACAAGCACTACCTGGACGAGATCATCGAGCAGATCAGCGAGTTCAGCAAGCGGGT
GATCCTGGCCGACGCCAACCTGGACAAGGTGCTGAGCGCCTACAACAAGCACCGGGACAAG
CCCATCCGGGAGCAGGCCGAGAACATCATCCACCTGTTCACCCTGACCAACCTGGGCGCCCC
CGCCGCCTTCAAGTACTTCGACACCACCATCGACCGGAAGCGGTACACCAGCACCAAGGAG
GTGCTGGACGCCACCCTGATCCACCAGAGCATCACCGGCCTGTACGAGACCCGGATCGACCT
GAGCCAGCTGGGCGGCGACAGCGGCGGCAAGCGGCCCGCCGCCACCAAGAAGGCCGGCCA
GGCCAAGAAGAAGAAGTCGGGCGGGGGTGGCTCAGACGCTAAGTCTCTGACCGCTTGGAGC
AGAACACTGGTCACCTTCAAGGACGTGTTCGTCGACTTCACAAGAGAGGAGTGGAAACTGC
TGGACACCGCCCAGCAGATCCTCTATAGAAACGTCATGCTGGAGAACTACAAGAATCTGGT
GTCTCTGGGCTACCAGCTGACCAAGCCCGACGTGATTCTGAGGCTGGAGAAGGGCGAGGAG
CCTTGGCTGGTGGAGAGAGAGATCCACCAAGAAACCCACCCCGACAGCGAAACCGCCTTCG
AGATCAAGAGCAGCGTGGGAGGTGGCGGATCGGGAAAGCGGCCCGCCGCCACCAAGAAGG
CCGGTCAGGCCAAGAAGAAGAAGGGCAGCTACCCCTACGACGTGCCCGACTACGCCTGAGC
GGCCGCTTAATTAAGCTGCCTTCTGCGGGGCTTGCCTTCTGGCCATGCCCTTCTTCTCTCCCTT
GCACCTGTACCTCTTGGTCTTTGAATAAAGCCTGAGTAGGAAGTCTAGAAAAAAAAAAAAA
AAAAAAAAAAAAAAAAAAAAAAAAAAAAAAAAAAAAAAAAAAAAAAAAAAAAAAAAAA
AAAAAAAAA

In some embodiments, an expression repressor comprises the amino acid sequence of SEQ ID NOs: 42 or 77. In some embodiments, an expression repressor described herein comprises an amino acid sequence of SEQ ID NO: 42 or 77 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9-KRAB
(SEQ ID NO: 42)
MAPKKKRKVGGSGGSGQTGKKSEKGPVCWRKRVKSEYMRL
RQLKRFRRADEVKSMFSSNRQKILERTEILNQEWKQRRIQ
PVHILTSVSSLRGTRECSVTSDLDFPTQVIPLKTLNAVAS
VPIMYSWSPLQQNFMVEDETVLHNIPYMGDEVLDQDGTFI
EELIKNYDGKVHGDRECGFINDEIFVELVNALGQYNDDDD
DDDGDDPEEREEKQKDLEDHRDDKESRPPRKFPSDKIFEA
ISSMFPDKGTAEELKEKYKELTEQQLPGALPPECTPNIDG
PNAKSVQREQSLHSFHTLFCRRCFKYDCFLHPFHATPNTY
KRKNTETALDNKPCGPQCYQHLEGAKEFAAALTAERIKTP
PKRPGGRRRGRLPNNSSRPSTPTINVLESKDTDSDREAGT
ETGGENNDKEEEEKKDETSSSSEANSRCQTPIKMKPNIEP
PENVEWSGAEASMFRVLIGTYYDNFCAIARLIGTKTCRQV
YEFRVKESSIIAPAPAEDVDTPPRKKKRKHRLWAAHCRKI
QLKKDGSSNHVYNYQPCDHPRQPCDSSCPCVIAQNFCEKF
CQCSSECQNRFPGCRCKAQCNTKQCPCYLAVRECDPDLCL
TCGAADHWDSKNVSCKNCSIQRGSKKHLLLAPSDVAGWGI
FIKDPVQKNEFISEYCGEIISQDEADRRGKVYDKYMCSFL
FNLNNDFVVDATRKGNKIRFANHSVNPNCYAKVMMVNGDH
RIGIFAKRAIQTGEELFFDYRYSQADALKYVGIEREMEIP
STGGSGGSGGSGGSGGSGRPDKKYSIGLAIGTNSVGWAVI
TDEYKVPSKKFKVLGNTDRHSIKKNLIGALLFDSGETAEA
TRLKRTARRRYTRRKNRICYLQEIFSNEMAKVDDSFFHRL
EESFLVEEDKKHERHPIFGNIVDEVAYHEKYPTIYHLRKK
LVDSTDKADLRLIYLALAHMIKFRGHFLIEGDLNPDNSDV
DKLFIQLVQTYNQLFEENPINASGVDAKAILSARLSKSRR
LENLIAQLPGEKKNGLFGNLIALSLGLTPNFKSNFDLAED
AKLQLSKDTYDDDLDNLLAQIGDQYADLFLAAKNLSDAIL
LSDILRVNTEITKAPLSASMIKRYDEHHQDLTLLKALVRQ
QLPEKYKEIFFDQSKNGYAGYIDGGASQEEFYKFIKPILE
KMDGTEELLVKLNREDLLRKQRTFDNGSIPHQIHLGELHA
ILRRQEDFYPFLKDNREKIEKILTFRIPYYVGPLARGNSR
FAWMTRKSEETITPWNFEEVVDKGASAQSFIERMTNFDKN
LPNEKVLPKHSLLYEYFTVYNELTKVKYVTEGMRKPAFLS
GEQKKAIVDLLFKTNRKVTVKQLKEDYFKKIECFDSVEIS
GVEDRFNASLGTYHDLLKIIKDKDFLDNEENEDILEDIVL
TLTLFEDREMIEERLKTYAHLFDDKVMKQLKRRRYTGWGR
LSRKLINGIRDKQSGKTILDFLKSDGFANRNFMQLIHDDS
LTFKEDIQKAQVSGQGDSLHEHIANLAGSPAIKKGILQTV
KVVDELVKVMGRHKPENIVIEMARENQTTQKGQKNSRERM
KRIEEGIKELGSQILKEHPVENTQLQNEKLYLYYLQNGRD
MYVDQELDINRLSDYDVAAIVPQSFLKDDSIDNKVLTRSD
KARGKSDNVPSEEVVKKMKNYWRQLLNAKLITQRKFDNLT
KAERGGLSELDKAGFIKRQLVETRQITKHVAQILDSRMNT
KYDENDKLIREVKVITLKSKLVSDFRKDFQFYKVREINNY
HHAHDAYLNAVVGTALIKKYPKLESEFVYGDYKVYDVRKM
IAKSEQEIGKATAKYFFYSNIMNFFKTEITLANGEIRKRP
LIETNGETGEIVWDKGRDFATVRKVLSMPQVNIVKKTEVQ
TGGFSKESILPKRNSDKLIARKKDWDPKKYGGFDSPTVAY
SVLVVAKVEKGKSKKLKSVKELLGITIMERSSFEKNPIDF
LEAKGYKEVKKDLIIKLPKYSLFELENGRKRMLASAGELQ
KGNELALPSKYVNFLYLASHYEKLKGSPEDNEQKQLFVEQ
HKHYLDEIIEQISEFSKRVILADANLDKVLSAYNKHRDKP
IREQAENIIHLFTLTNLGAPAAFKYFDTTIDRKRYTSTKE
VLDATLIHQSITGLYETRIDLSQLGGDSGGKRPAATKKAG
QAKKKKSGGGGSDAKSLTAWSRTLVTFKDVFVDFTREEWK
LLDTAQQILYRNVMLENYKNLVSLGYQLTKPDVILRLEKG
EEPWLVEREIHQETHPDSETAFEIKSSVGGGGSGKRPAAT
KKAGQAKKKKGSYPYDVPDYA
EZH2-dCas9-KRAB without HA tag
(SEQ ID NO: 77)
MAPKKKRKVGGSGGSGQTGKKSEKGPVCWRKRVKSEYMRL
RQLKRFRRADEVKSMFSSNRQKILERTEILNQEWKQRRIQ
PVHILTSVSSLRGTRECSVTSDLDFPTQVIPLKTLNAVAS
VPIMYSWSPLQQNFMVEDETVLHNIPYMGDEVLDQDGTFI
EELIKNYDGKVHGDRECGFINDEIFVELVNALGQYNDDDD
DDDGDDPEEREEKQKDLEDHRDDKESRPPRKFPSDKIFEA
ISSMFPDKGTAEELKEKYKELTEQQLPGALPPECTPNIDG
PNAKSVQREQSLHSFHTLFCRRCFKYDCFLHPFHATPNTY
KRKNTETALDNKPCGPQCYQHLEGAKEFAAALTAERIKTP
PKRPGGRRRGRLPNNSSRPSTPTINVLESKDTDSDREAGT
ETGGENNDKEEEEKKDETSSSSEANSRCQTPIKMKPNIEP
PENVEWSGAEASMFRVLIGTYYDNFCAIARLIGTKTCRQV
YEFRVKESSIIAPAPAEDVDTPPRKKKRKHRLWAAHCRKI
QLKKDGSSNHVYNYQPCDHPRQPCDSSCPCVIAQNFCEKF
CQCSSECQNRFPGCRCKAQCNTKQCPCYLAVRECDPDLCL
TCGAADHWDSKNVSCKNCSIQRGSKKHLLLAPSDVAGWGI
FIKDPVQKNEFISEYCGEIISQDEADRRGKVYDKYMCSFL
FNLNNDFVVDATRKGNKIRFANHSVNPNCYAKVMMVNGDH
RIGIFAKRAIQTGEELFFDYRYSQADALKYVGIEREMEIP
STGGSGGSGGSGGSGGSGRPDKKYSIGLAIGTNSVGWAVI
TDEYKVPSKKFKVLGNTDRHSIKKNLIGALLFDSGETAEA
TRLKRTARRRYTRRKNRICYLQEIFSNEMAKVDDSFFHRL
EESFLVEEDKKHERHPIFGNIVDEVAYHEKYPTIYHLRKK
LVDSTDKADLRLIYLALAHMIKFRGHFLIEGDLNPDNSDV
DKLFIQLVQTYNQLFEENPINASGVDAKAILSARLSKSRR
LENLIAQLPGEKKNGLFGNLIALSLGLTPNFKSNFDLAED
AKLQLSKDTYDDDLDNLLAQIGDQYADLFLAAKNLSDAIL
LSDILRVNTEITKAPLSASMIKRYDEHHQDLTLLKALVRQ
QLPEKYKEIFFDQSKNGYAGYIDGGASQEEFYKFIKPILE
KMDGTEELLVKLNREDLLRKQRTFDNGSIPHQIHLGELHA
ILRRQEDFYPFLKDNREKIEKILTFRIPYYVGPLARGNSR
FAWMTRKSEETITPWNFEEVVDKGASAQSFIERMTNFDKN
LPNEKVLPKHSLLYEYFTVYNELTKVKYVTEGMRKPAFLS
GEQKKAIVDLLFKTNRKVTVKQLKEDYFKKIECFDSVEIS
GVEDRFNASLGTYHDLLKIIKDKDFLDNEENEDILEDIVL
TLTLFEDREMIEERLKTYAHLFDDKVMKQLKRRRYTGWGR
LSRKLINGIRDKQSGKTILDFLKSDGFANRNFMQLIHDDS
LTFKEDIQKAQVSGQGDSLHEHIANLAGSPAIKKGILQTV
KVVDELVKVMGRHKPENIVIEMARENQTTQKGQKNSRERM
KRIEEGIKELGSQILKEHPVENTQLQNEKLYLYYLQNGRD
MYVDQELDINRLSDYDVAAIVPQSFLKDDSIDNKVLTRSD
KARGKSDNVPSEEVVKKMKNYWRQLLNAKLITQRKFDNLT
KAERGGLSELDKAGFIKRQLVETRQITKHVAQILDSRMNT
KYDENDKLIREVKVITLKSKLVSDFRKDFQFYKVREINNY
HHAHDAYLNAVVGTALIKKYPKLESEFVYGDYKVYDVRKM
IAKSEQEIGKATAKYFFYSNIMNFFKTEITLANGEIRKRP
LIETNGETGEIVWDKGRDFATVRKVLSMPQVNIVKKTEVQ
TGGFSKESILPKRNSDKLIARKKDWDPKKYGGFDSPTVAY
SVLVVAKVEKGKSKKLKSVKELLGITIMERSSFEKNPIDF
LEAKGYKEVKKDLIIKLPKYSLFELENGRKRMLASAGELQ
KGNELALPSKYVNFLYLASHYEKLKGSPEDNEQKQLFVEQ
HKHYLDEIIEQISEFSKRVILADANLDKVLSAYNKHRDKP
IREQAENIIHLFTLTNLGAPAAFKYFDTTIDRKRYTSTKE
VLDATLIHQSITGLYETRIDLSQLGGDSGGKRPAATKKAG
QAKKKKSGGGGSDAKSLTAWSRTLVTFKDVFVDFTREEWK
LLDTAQQILYRNVMLENYKNLVSLGYQLTKPDVILRLEKG
EEPWLVEREIHQETHPDSETAFEIKSSVGGGGSGKRPAAT
KKAGQAKKKKGS

In some embodiments, an expression repressor comprises a DNA-targeting moiety comprising dCas9, e.g., an S. pyogenes dCas9, and a first repressor domain comprising G9A e.g., a G9A domain and a second repressor domain comprising KRAB, e.g., a KRAB domain. In some embodiments, the expression repressor is encoded by the nucleic acid sequence of SEQ ID NOs: 31 (e.g., a nucleic acid (e.g., cDNA) encoding the expression repressor). In some embodiments, a nucleic acid described herein comprises a nucleic acid sequence of SEQ ID NO: 31 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-KRAB (MR-29442)
(SEQ ID NO: 31)
AGGAAATAAGAGAGAAAAGAAGAGTAAGAAGAAATATAAG
AGCCACCATGGCCCCCAAGAAGAAGCGGAAGGTGGGCATC
CACGGCGTGCCCGCCGCCTCGGGCGGGGGTGGCTCAGGAA
GCGCCGCTATCGCCGAAGTGCTGCTGAATGCCAGATGCGA
TCTGCATGCCGTGAACTACCACGGCGACACCCCTCTGCAT
ATCGCCGCTAGAGAGAGCTACCATGACTGTGTGCTGCTGT
TTCTGAGCAGAGGCGCCAATCCCGAGCTCAGAAACAAAGA
GGGCGACACCGCTTGGGATCTGACACCCGAGAGATCCGAC
GTGTGGTTCGCTCTGCAACTGAATAGAAAACTGAGACTGG
GCGTCGGCAATAGGGCCATTAGAACCGAGAAGATCATCTG
TAGGGACGTGGCTAGGGGCTACGAGAACGTGCCCATCCCT
TGTGTGAACGGAGTGGATGGAGAGCCTTGCCCCGAGGATT
ACAAATACATCAGCGAGAACTGCGAAACCTCCACCATGAA
TATCGATAGAAACATTACACACCTCCAGCACTGTACATGC
GTGGACGATTGCAGCAGCAGCAACTGTCTGTGCGGCCAAC
TGAGCATCAGATGCTGGTACGACAAGGATGGCAGACTGCT
GCAAGAGTTCAACAAGATCGAACCCCCTCTGATCTTCGAG
TGTAACCAAGCTTGCAGCTGTTGGAGGAACTGCAAGAATA
GGGTCGTGCAGTCCGGAATCAAGGTGAGACTGCAGCTGTA
TAGAACAGCTAAGATGGGATGGGGAGTCAGAGCTCTGCAG
ACCATCCCCCAAGGCACATTCATCTGTGAGTACGTCGGCG
AACTCATCAGCGACGCTGAGGCCGATGTGAGGGAGGACGA
CAGCTATCTCTTCGACCTCGACAACAAGGACGGCGAGGTG
TACTGCATCGACGCTAGATATTACGGCAACATCAGCAGAT
TCATCAACCACCTCTGCGACCCCAATATCATCCCCGTGAG
AGTGTTCATGCTCCATCAAGATCTGAGATTCCCTAGGATC
GCCTTCTTCAGCTCTAGAGACATTAGAACCGGCGAGGAGC
TGGGATTCGACTACGGCGACAGGTTCTGGGACATCAAGAG
CAAGTACTTCACATGCCAATGCGGCAGCGAGAAATGCAAG
CATAGCGCCGAGGCCATTGCTCTGGAGCAGTCTAGACTGG
CTAGGCTGGACCCTCACCCCGAGCTGCTGCCCGAACTGGG
ATCTCTGCCTCCCGTGAATACCGGAGGTGGCGGATCGGGA
GACAAGAAGTACAGCATCGGCCTGGCCATCGGCACCAACA
GCGTGGGCTGGGCCGTGATCACCGACGAGTACAAGGTGCC
CAGCAAGAAGTTCAAGGTGCTGGGCAACACCGACCGGCAC
AGCATCAAGAAGAACCTGATCGGCGCCCTGCTGTTCGACA
GCGGCGAGACCGCCGAGGCCACCCGGCTGAAGCGGACCGC
CCGGCGGCGGTACACCCGGCGGAAGAACCGGATCTGCTAC
CTGCAGGAGATCTTCAGCAACGAGATGGCCAAGGTGGACG
ACAGCTTCTTCCACCGGCTGGAGGAGAGCTTCCTGGTGGA
GGAGGACAAGAAGCACGAGCGGCACCCCATCTTCGGCAAC
ATCGTGGACGAGGTGGCCTACCACGAGAAGTACCCCACCA
TCTACCACCTGCGGAAGAAGCTGGTGGACAGCACCGACAA
GGCCGACCTGCGGCTGATCTACCTGGCCCTGGCCCACATG
ATCAAGTTCCGGGGCCACTTCCTGATCGAGGGCGACCTGA
ACCCCGACAACAGCGACGTGGACAAGCTGTTCATCCAGCT
GGTGCAGACCTACAACCAGCTGTTCGAGGAGAACCCCATC
AACGCCAGCGGCGTGGACGCCAAGGCCATCCTGAGCGCCC
GGCTGAGCAAGAGCCGGCGGCTGGAGAACCTGATCGCCCA
GCTGCCCGGCGAGAAGAAGAACGGCCTGTTCGGCAACCTG
ATCGCCCTGAGCCTGGGCCTGACCCCCAACTTCAAGAGCA
ACTTCGACCTGGCCGAGGACGCCAAGCTGCAGCTGAGCAA
GGACACCTACGACGACGACCTGGACAACCTGCTGGCCCAG
ATCGGCGACCAGTACGCCGACCTGTTCCTGGCCGCCAAGA
ACCTGAGCGACGCCATCCTGCTGAGCGACATCCTGCGGGT
GAACACCGAGATCACCAAGGCCCCCCTGAGCGCCAGCATG
ATCAAGCGGTACGACGAGCACCACCAGGACCTGACCCTGC
TGAAGGCCCTGGTGCGGCAGCAGCTGCCCGAGAAGTACAA
GGAGATCTTCTTCGACCAGAGCAAGAACGGCTACGCCGGC
TACATCGACGGCGGCGCCAGCCAGGAGGAGTTCTACAAGT
TCATCAAGCCCATCCTGGAGAAGATGGACGGCACCGAGGA
GCTGCTGGTGAAGCTGAACCGGGAGGACCTGCTGCGGAAG
CAGCGGACCTTCGACAACGGCAGCATCCCCCACCAGATCC
ACCTGGGCGAGCTGCACGCCATCCTGCGGCGGCAGGAGGA
CTTCTACCCCTTCCTGAAGGACAACCGGGAGAAGATCGAG
AAGATCCTGACCTTCCGGATCCCCTACTACGTGGGCCCCC
TGGCCCGGGGCAACAGCCGGTTCGCCTGGATGACCCGGAA
ATCCGAGGAGACCATCACCCCCTGGAACTTCGAGGAGGTG
GTGGACAAGGGCGCCAGCGCCCAGAGCTTCATCGAGCGGA
TGACCAACTTCGACAAGAACCTGCCCAACGAGAAGGTGCT
GCCCAAGCACAGCCTGCTGTACGAGTACTTCACCGTGTAC
AACGAGCTGACCAAGGTGAAGTACGTGACCGAGGGCATGC
GGAAGCCCGCCTTCCTGAGCGGCGAGCAGAAGAAGGCCAT
CGTGGACCTGCTGTTCAAGACCAACCGGAAGGTGACCGTG
AAGCAGCTGAAGGAGGACTACTTCAAGAAGATCGAGTGCT
TCGACAGCGTGGAGATCAGCGGCGTGGAGGACCGGTTCAA
CGCCAGCCTGGGCACCTACCACGACCTGCTGAAGATCATC
AAGGACAAGGACTTCCTGGACAACGAGGAGAACGAGGACA
TCCTGGAGGACATCGTGCTGACCCTGACCCTGTTCGAGGA
CCGGGAGATGATCGAGGAGCGGCTGAAAACCTACGCCCAC
CTGTTCGACGACAAGGTGATGAAGCAGCTGAAGCGGCGGC
GGTACACCGGCTGGGGCCGGCTGAGCCGGAAGCTGATCAA
CGGCATCCGGGACAAGCAGAGCGGCAAGACCATCCTGGAC
TTCCTGAAATCCGACGGCTTCGCCAACCGGAACTTCATGC
AGCTGATCCACGACGACAGCCTGACCTTCAAGGAGGACAT
CCAGAAGGCCCAGGTGAGCGGCCAGGGCGACAGCCTGCAC
GAGCACATCGCCAACCTGGCCGGCAGCCCCGCCATCAAGA
AGGGCATCCTGCAGACCGTGAAGGTGGTGGACGAGCTGGT
GAAGGTGATGGGCCGGCACAAGCCCGAGAACATCGTGATC
GAGATGGCCCGGGAGAACCAGACCACCCAGAAGGGCCAGA
AGAACAGCCGGGAGCGGATGAAGCGGATCGAGGAGGGCAT
CAAGGAGCTGGGCAGCCAGATCCTGAAGGAGCACCCCGTG
GAGAACACCCAGCTGCAGAACGAGAAGCTGTACCTGTACT
ACCTGCAGAACGGCCGGGACATGTACGTGGACCAGGAGCT
GGACATCAACCGGCTGAGCGACTACGACGTGGCCGCCATC
GTGCCCCAGAGCTTCCTGAAGGACGACAGCATCGACAACA
AGGTGCTGACCCGGAGCGACAAGGCCCGGGGCAAGAGCGA
CAACGTGCCCAGCGAGGAGGTGGTGAAGAAGATGAAGAAC
TACTGGCGGCAGCTGCTGAACGCCAAGCTGATCACCCAGC
GGAAGTTCGACAACCTGACCAAGGCCGAGCGGGGCGGCCT
GAGCGAGCTGGACAAGGCCGGCTTCATCAAGCGGCAGCTG
GTGGAGACCCGGCAGATCACCAAGCACGTGGCCCAGATCC
TGGACAGCCGGATGAACACCAAGTACGACGAGAACGACAA
GCTGATCCGGGAGGTGAAGGTGATCACCCTGAAATCCAAG
CTGGTGAGCGACTTCCGGAAGGACTTCCAGTTCTACAAGG
TGCGGGAGATCAACAACTACCACCACGCCCACGACGCCTA
CCTGAACGCCGTGGTGGGCACCGCCCTGATCAAGAAGTAC
CCCAAGCTGGAGAGCGAGTTCGTGTACGGCGACTACAAGG
TGTACGACGTGCGGAAGATGATCGCCAAGAGCGAGCAGGA
GATCGGCAAGGCCACCGCCAAGTACTTCTTCTACAGCAAC
ATCATGAACTTCTTCAAGACCGAGATCACCCTGGCCAACG
GCGAGATCCGGAAGCGGCCCCTGATCGAGACCAACGGCGA
GACCGGCGAGATCGTGTGGGACAAGGGCCGGGACTTCGCC
ACCGTGCGGAAGGTGCTGAGCATGCCCCAGGTGAACATCG
TGAAGAAAACCGAGGTGCAGACCGGCGGCTTCAGCAAGGA
GAGCATCCTGCCCAAGCGGAACAGCGACAAGCTGATCGCC
CGGAAGAAGGACTGGGACCCCAAGAAGTACGGCGGCTTCG
ACAGCCCCACCGTGGCCTACAGCGTGCTGGTGGTGGCCAA
GGTGGAGAAGGGCAAGAGCAAGAAGCTGAAATCCGTGAAG
GAGCTGCTGGGCATCACCATCATGGAGCGGAGCAGCTTCG
AGAAGAACCCCATCGACTTCCTGGAGGCCAAGGGCTACAA
GGAGGTGAAGAAGGACCTGATCATCAAGCTGCCCAAGTAC
AGCCTGTTCGAGCTGGAGAACGGCCGGAAGCGGATGCTGG
CCAGCGCCGGCGAGCTGCAGAAGGGCAACGAGCTGGCCCT
GCCCAGCAAGTACGTGAACTTCCTGTACCTGGCCAGCCAC
TACGAGAAGCTGAAGGGCAGCCCCGAGGACAACGAGCAGA
AGCAGCTGTTCGTGGAGCAGCACAAGCACTACCTGGACGA
GATCATCGAGCAGATCAGCGAGTTCAGCAAGCGGGTGATC
CTGGCCGACGCCAACCTGGACAAGGTGCTGAGCGCCTACA
ACAAGCACCGGGACAAGCCCATCCGGGAGCAGGCCGAGAA
CATCATCCACCTGTTCACCCTGACCAACCTGGGCGCCCCC
GCCGCCTTCAAGTACTTCGACACCACCATCGACCGGAAGC
GGTACACCAGCACCAAGGAGGTGCTGGACGCCACCCTGAT
CCACCAGAGCATCACCGGCCTGTACGAGACCCGGATCGAC
CTGAGCCAGCTGGGCGGCGACAGCGGCGGCAAGCGGCCCG
CCGCCACCAAGAAGGCCGGCCAGGCCAAGAAGAAGAAGTC
GGGCGGGGGTGGCTCAGACGCTAAGTCTCTGACCGCTTGG
AGCAGAACACTGGTCACCTTCAAGGACGTGTTCGTCGACT
TCACAAGAGAGGAGTGGAAACTGCTGGACACCGCCCAGCA
GATCCTCTATAGAAACGTCATGCTGGAGAACTACAAGAAT
CTGGTGTCTCTGGGCTACCAGCTGACCAAGCCCGACGTGA
TTCTGAGGCTGGAGAAGGGCGAGGAGCCTTGGCTGGTGGA
GAGAGAGATCCACCAAGAAACCCACCCCGACAGCGAAACC
GCCTTCGAGATCAAGAGCAGCGTGGGAGGTGGCGGATCGG
GAAAGCGGCCCGCCGCCACCAAGAAGGCCGGTCAGGCCAA
GAAGAAGAAGGGCAGCTACCCCTACGACGTGCCCGACTAC
GCCTGAGCGGCCGCTTAATTAAGCTGCCTTCTGCGGGGCT
TGCCTTCTGGCCATGCCCTTCTTCTCTCCCTTGCACCTGT
ACCTCTTGGTCTTTGAATAAAGCCTGAGTAGGAAGTCTAG
AAAAAAAAAAAAAAAAAAAAAAAAAAAAAAAAAAAAAAAA
AAAAAAAAAAAAAAAAAAAAAAAAAAAAAAAAAAAAAAAA

In some embodiments, an expression repressor comprises the amino acid sequence of SEQ ID NOs: 43 or 78. In some embodiments, an expression repressor described herein comprises an amino acid sequence of SEQ ID NO: 42 or 77 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-KRAB
(SEQ ID NO: 43)
MAPKKKRKVGIHGVPAASGGGGSGSAAIAEVLLNARCDLH
AVNYHGDTPLHIAARESYHDCVLLFLSRGANPELRNKEGD
TAWDLTPERSDVWFALQLNRKLRLGVGNRAIRTEKIICRD
VARGYENVPIPCVNGVDGEPCPEDYKYISENCETSTMNID
RNITHLQHCTCVDDCSSSNCLCGQLSIRCWYDKDGRLLQE
FNKIEPPLIFECNQACSCWRNCKNRVVQSGIKVRLQLYRT
AKMGWGVRALQTIPQGTFICEYVGELISDAEADVREDDSY
LFDLDNKDGEVYCIDARYYGNISRFINHLCDPNIIPVRVF
MLHQDLRFPRIAFFSSRDIRTGEELGFDYGDRFWDIKSKY
FTCQCGSEKCKHSAEAIALEQSRLARLDPHPELLPELGSL
PPVNTGGGGSGDKKYSIGLAIGTNSVGWAVITDEYKVPSK
KFKVLGNTDRHSIKKNLIGALLFDSGETAEATRLKRTARR
RYTRRKNRICYLQEIFSNEMAKVDDSFFHRLEESFLVEED
KKHERHPIFGNIVDEVAYHEKYPTIYHLRKKLVDSTDKAD
LRLIYLALAHMIKFRGHFLIEGDLNPDNSDVDKLFIQLVQ
TYNQLFEENPINASGVDAKAILSARLSKSRRLENLIAQLP
GEKKNGLFGNLIALSLGLTPNFKSNFDLAEDAKLQLSKDT
YDDDLDNLLAQIGDQYADLFLAAKNLSDAILLSDILRVNT
EITKAPLSASMIKRYDEHHQDLTLLKALVRQQLPEKYKEI
FFDQSKNGYAGYIDGGASQEEFYKFIKPILEKMDGTEELL
VKLNREDLLRKQRTFDNGSIPHQIHLGELHAILRRQEDFY
PFLKDNREKIEKILTFRIPYYVGPLARGNSRFAWMTRKSE
ETITPWNFEEVVDKGASAQSFIERMTNFDKNLPNEKVLPK
HSLLYEYFTVYNELTKVKYVTEGMRKPAFLSGEQKKAIVD
LLFKTNRKVTVKQLKEDYFKKIECFDSVEISGVEDRFNAS
LGTYHDLLKIIKDKDFLDNEENEDILEDIVLTLTLFEDRE
MIEERLKTYAHLFDDKVMKQLKRRRYTGWGRLSRKLINGI
RDKQSGKTILDFLKSDGFANRNFMQLIHDDSLTFKEDIQK
AQVSGQGDSLHEHIANLAGSPAIKKGILQTVKVVDELVKV
MGRHKPENIVIEMARENQTTQKGQKNSRERMKRIEEGIKE
LGSQILKEHPVENTQLQNEKLYLYYLQNGRDMYVDQELDI
NRLSDYDVAAIVPQSFLKDDSIDNKVLTRSDKARGKSDNV
PSEEVVKKMKNYWRQLLNAKLITQRKFDNLTKAERGGLSE
LDKAGFIKRQLVETRQITKHVAQILDSRMNTKYDENDKLI
REVKVITLKSKLVSDFRKDFQFYKVREINNYHHAHDAYLN
AVVGTALIKKYPKLESEFVYGDYKVYDVRKMIAKSEQEIG
KATAKYFFYSNIMNFFKTEITLANGEIRKRPLIETNGETG
EIVWDKGRDFATVRKVLSMPQVNIVKKTEVQTGGFSKESI
LPKRNSDKLIARKKDWDPKKYGGFDSPTVAYSVLVVAKVE
KGKSKKLKSVKELLGITIMERSSFEKNPIDFLEAKGYKEV
KKDLIIKLPKYSLFELENGRKRMLASAGELQKGNELALPS
KYVNFLYLASHYEKLKGSPEDNEQKQLFVEQHKHYLDEII
EQISEFSKRVILADANLDKVLSAYNKHRDKPIREQAENII
HLFTLTNLGAPAAFKYFDTTIDRKRYTSTKEVLDATLIHQ
SITGLYETRIDLSQLGGDSGGKRPAATKKAGQAKKKKSGG
GGSDAKSLTAWSRTLVTFKDVFVDFTREEWKLLDTAQQIL
YRNVMLENYKNLVSLGYQLTKPDVILRLEKGEEPWLVERE
IHQETHPDSETAFEIKSSVGGGGSGKRPAATKKAGQAKKK
KGSYPYDVPDYA
G9A-dCas9-KRAB without HA tag
(SEQ ID NO: 78)
MAPKKKRKVGIHGVPAASGGGGSGSAAIAEVLLNARCDLH
AVNYHGDTPLHIAARESYHDCVLLFLSRGANPELRNKEGD
TAWDLTPERSDVWFALQLNRKLRLGVGNRAIRTEKIICRD
VARGYENVPIPCVNGVDGEPCPEDYKYISENCETSTMNID
RNITHLQHCTCVDDCSSSNCLCGQLSIRCWYDKDGRLLQE
FNKIEPPLIFECNQACSCWRNCKNRVVQSGIKVRLQLYRT
AKMGWGVRALQTIPQGTFICEYVGELISDAEADVREDDSY
LFDLDNKDGEVYCIDARYYGNISRFINHLCDPNIIPVRVF
MLHQDLRFPRIAFFSSRDIRTGEELGFDYGDRFWDIKSKY
FTCQCGSEKCKHSAEAIALEQSRLARLDPHPELLPELGSL
PPVNTGGGGSGDKKYSIGLAIGTNSVGWAVITDEYKVPSK
KFKVLGNTDRHSIKKNLIGALLFDSGETAEATRLKRTARR
RYTRRKNRICYLQEIFSNEMAKVDDSFFHRLEESFLVEED
KKHERHPIFGNIVDEVAYHEKYPTIYHLRKKLVDSTDKAD
LRLIYLALAHMIKFRGHFLIEGDLNPDNSDVDKLFIQLVQ
TYNQLFEENPINASGVDAKAILSARLSKSRRLENLIAQLP
GEKKNGLFGNLIALSLGLTPNFKSNFDLAEDAKLQLSKDT
YDDDLDNLLAQIGDQYADLFLAAKNLSDAILLSDILRVNT
EITKAPLSASMIKRYDEHHQDLTLLKALVRQQLPEKYKEI
FFDQSKNGYAGYIDGGASQEEFYKFIKPILEKMDGTEELL
VKLNREDLLRKQRTFDNGSIPHQIHLGELHAILRRQEDFY
PFLKDNREKIEKILTFRIPYYVGPLARGNSRFAWMTRKSE
ETITPWNFEEVVDKGASAQSFIERMTNFDKNLPNEKVLPK
HSLLYEYFTVYNELTKVKYVTEGMRKPAFLSGEQKKAIVD
LLFKTNRKVTVKQLKEDYFKKIECFDSVEISGVEDRFNAS
LGTYHDLLKIIKDKDFLDNEENEDILEDIVLTLTLFEDRE
MIEERLKTYAHLFDDKVMKQLKRRRYTGWGRLSRKLINGI
RDKQSGKTILDFLKSDGFANRNFMQLIHDDSLTFKEDIQK
AQVSGQGDSLHEHIANLAGSPAIKKGILQTVKVVDELVKV
MGRHKPENIVIEMARENQTTQKGQKNSRERMKRIEEGIKE
LGSQILKEHPVENTQLQNEKLYLYYLQNGRDMYVDQELDI
NRLSDYDVAAIVPQSFLKDDSIDNKVLTRSDKARGKSDNV
PSEEVVKKMKNYWRQLLNAKLITQRKFDNLTKAERGGLSE
LDKAGFIKRQLVETRQITKHVAQILDSRMNTKYDENDKLI
REVKVITLKSKLVSDFRKDFQFYKVREINNYHHAHDAYLN
AVVGTALIKKYPKLESEFVYGDYKVYDVRKMIAKSEQEIG
KATAKYFFYSNIMNFFKTEITLANGEIRKRPLIETNGETG
EIVWDKGRDFATVRKVLSMPQVNIVKKTEVQTGGFSKESI
LPKRNSDKLIARKKDWDPKKYGGFDSPTVAYSVLVVAKVE
KGKSKKLKSVKELLGITIMERSSFEKNPIDFLEAKGYKEV
KKDLIIKLPKYSLFELENGRKRMLASAGELQKGNELALPS
KYVNFLYLASHYEKLKGSPEDNEQKQLFVEQHKHYLDEII
EQISEFSKRVILADANLDKVLSAYNKHRDKPIREQAENII
HLFTLTNLGAPAAFKYFDTTIDRKRYTSTKEVLDATLIHQ
SITGLYETRIDLSQLGGDSGGKRPAATKKAGQAKKKKSGG
GGSDAKSLTAWSRTLVTFKDVFVDFTREEWKLLDTAQQIL
YRNVMLENYKNLVSLGYQLTKPDVILRLEKGEEPWLVERE
IHQETHPDSETAFEIKSSVGGGGSGKRPAATKKAGQAKKK
KGS

In some embodiments, an expression repressor comprises a DNA-targeting moiety comprising dCas9, e.g., an S. pyogenes dCas9, and a first repressor domain comprising FOG1 e.g., a FOG1 domain and a second repressor domain comprising FOG1, e.g., a FOG1 domain. In some embodiments, the expression repressor is encoded by the nucleic acid sequence of SEQ ID NOs: 32 (e.g., a nucleic acid (e.g., cDNA) encoding the expression repressor). In some embodiments, a nucleic acid described herein comprises a nucleic acid sequence of SEQ ID NO: 32 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

FOG1-dCas9-FOG1 (MR-29434)
(SEQ ID NO: 32)
AGGAAATAAGAGAGAAAAGAAGAGTAAGAAGAAATATAAG
AGCCACCATGGCCCCCAAGAAGAAGCGGAAGGTGGGCATC
CACGGCGTGCCCGCCGCCTCGGGGGGGGTGGCTCAAGCAG
AAGGAAGCAGAGCAACCCCAGACAAATCAAGAGATCTCTG
GGCGACATGGAGGCCAGAGAGGAAGTGCAGCTGGTGGGCG
CCAGCCACATGGAGCAGAAGGCTACAGCCCCCGAGGCCCC
CAGCCCCGGAGGTGGCGGATCGGGAGACAAGAAGTACAGC
ATCGGCCTGGCCATCGGCACCAACAGCGTGGGCTGGGCCG
TGATCACCGACGAGTACAAGGTGCCCAGCAAGAAGTTCAA
GGTGCTGGGCAACACCGACCGGCACAGCATCAAGAAGAAC
CTGATCGGCGCCCTGCTGTTCGACAGCGGCGAGACCGCCG
AGGCCACCCGGCTGAAGCGGACCGCCCGGCGGCGGTACAC
CCGGCGGAAGAACCGGATCTGCTACCTGCAGGAGATCTTC
AGCAACGAGATGGCCAAGGTGGACGACAGCTTCTTCCACC
GGCTGGAGGAGAGCTTCCTGGTGGAGGAGGACAAGAAGCA
CGAGCGGCACCCCATCTTCGGCAACATCGTGGACGAGGTG
GCCTACCACGAGAAGTACCCCACCATCTACCACCTGCGGA
AGAAGCTGGTGGACAGCACCGACAAGGCCGACCTGCGGCT
GATCTACCTGGCCCTGGCCCACATGATCAAGTTCCGGGGC
CACTTCCTGATCGAGGGCGACCTGAACCCCGACAACAGCG
ACGTGGACAAGCTGTTCATCCAGCTGGTGCAGACCTACAA
CCAGCTGTTCGAGGAGAACCCCATCAACGCCAGCGGCGTG
GACGCCAAGGCCATCCTGAGCGCCCGGCTGAGCAAGAGCC
GGCGGCTGGAGAACCTGATCGCCCAGCTGCCCGGCGAGAA
GAAGAACGGCCTGTTCGGCAACCTGATCGCCCTGAGCCTG
GGCCTGACCCCCAACTTCAAGAGCAACTTCGACCTGGCCG
AGGACGCCAAGCTGCAGCTGAGCAAGGACACCTACGACGA
CGACCTGGACAACCTGCTGGCCCAGATCGGCGACCAGTAC
GCCGACCTGTTCCTGGCCGCCAAGAACCTGAGCGACGCCA
TCCTGCTGAGCGACATCCTGCGGGTGAACACCGAGATCAC
CAAGGCCCCCCTGAGCGCCAGCATGATCAAGCGGTACGAC
GAGCACCACCAGGACCTGACCCTGCTGAAGGCCCTGGTGC
GGCAGCAGCTGCCCGAGAAGTACAAGGAGATCTTCTTCGA
CCAGAGCAAGAACGGCTACGCCGGCTACATCGACGGCGGC
GCCAGCCAGGAGGAGTTCTACAAGTTCATCAAGCCCATCC
TGGAGAAGATGGACGGCACCGAGGAGCTGCTGGTGAAGCT
GAACCGGGAGGACCTGCTGCGGAAGCAGCGGACCTTCGAC
AACGGCAGCATCCCCCACCAGATCCACCTGGGCGAGCTGC
ACGCCATCCTGCGGCGGCAGGAGGACTTCTACCCCTTCCT
GAAGGACAACCGGGAGAAGATCGAGAAGATCCTGACCTTC
CGGATCCCCTACTACGTGGGCCCCCTGGCCCGGGGCAACA
GCCGGTTCGCCTGGATGACCCGGAAATCCGAGGAGACCAT
CACCCCCTGGAACTTCGAGGAGGTGGTGGACAAGGGCGCC
AGCGCCCAGAGCTTCATCGAGCGGATGACCAACTTCGACA
AGAACCTGCCCAACGAGAAGGTGCTGCCCAAGCACAGCCT
GCTGTACGAGTACTTCACCGTGTACAACGAGCTGACCAAG
GTGAAGTACGTGACCGAGGGCATGCGGAAGCCCGCCTTCC
TGAGCGGCGAGCAGAAGAAGGCCATCGTGGACCTGCTGTT
CAAGACCAACCGGAAGGTGACCGTGAAGCAGCTGAAGGAG
GACTACTTCAAGAAGATCGAGTGCTTCGACAGCGTGGAGA
TCAGCGGCGTGGAGGACCGGTTCAACGCCAGCCTGGGCAC
CTACCACGACCTGCTGAAGATCATCAAGGACAAGGACTTC
CTGGACAACGAGGAGAACGAGGACATCCTGGAGGACATCG
TGCTGACCCTGACCCTGTTCGAGGACCGGGAGATGATCGA
GGAGCGGCTGAAAACCTACGCCCACCTGTTCGACGACAAG
GTGATGAAGCAGCTGAAGCGGCGGCGGTACACCGGCTGGG
GCCGGCTGAGCCGGAAGCTGATCAACGGCATCCGGGACAA
GCAGAGCGGCAAGACCATCCTGGACTTCCTGAAATCCGAC
GGCTTCGCCAACCGGAACTTCATGCAGCTGATCCACGACG
ACAGCCTGACCTTCAAGGAGGACATCCAGAAGGCCCAGGT
GAGCGGCCAGGGCGACAGCCTGCACGAGCACATCGCCAAC
CTGGCCGGCAGCCCCGCCATCAAGAAGGGCATCCTGCAGA
CCGTGAAGGTGGTGGACGAGCTGGTGAAGGTGATGGGCCG
GCACAAGCCCGAGAACATCGTGATCGAGATGGCCCGGGAG
AACCAGACCACCCAGAAGGGCCAGAAGAACAGCCGGGAGC
GGATGAAGCGGATCGAGGAGGGCATCAAGGAGCTGGGCAG
CCAGATCCTGAAGGAGCACCCCGTGGAGAACACCCAGCTG
CAGAACGAGAAGCTGTACCTGTACTACCTGCAGAACGGCC
GGGACATGTACGTGGACCAGGAGCTGGACATCAACCGGCT
GAGCGACTACGACGTGGCCGCCATCGTGCCCCAGAGCTTC
CTGAAGGACGACAGCATCGACAACAAGGTGCTGACCCGGA
GCGACAAGGCCCGGGGCAAGAGCGACAACGTGCCCAGCGA
GGAGGTGGTGAAGAAGATGAAGAACTACTGGCGGCAGCTG
CTGAACGCCAAGCTGATCACCCAGCGGAAGTTCGACAACC
TGACCAAGGCCGAGCGGGGCGGCCTGAGCGAGCTGGACAA
GGCCGGCTTCATCAAGCGGCAGCTGGTGGAGACCCGGCAG
ATCACCAAGCACGTGGCCCAGATCCTGGACAGCCGGATGA
ACACCAAGTACGACGAGAACGACAAGCTGATCCGGGAGGT
GAAGGTGATCACCCTGAAATCCAAGCTGGTGAGCGACTTC
CGGAAGGACTTCCAGTTCTACAAGGTGCGGGAGATCAACA
ACTACCACCACGCCCACGACGCCTACCTGAACGCCGTGGT
GGGCACCGCCCTGATCAAGAAGTACCCCAAGCTGGAGAGC
GAGTTCGTGTACGGCGACTACAAGGTGTACGACGTGCGGA
AGATGATCGCCAAGAGCGAGCAGGAGATCGGCAAGGCCAC
CGCCAAGTACTTCTTCTACAGCAACATCATGAACTTCTTC
AAGACCGAGATCACCCTGGCCAACGGCGAGATCCGGAAGC
GGCCCCTGATCGAGACCAACGGCGAGACCGGCGAGATCGT
GTGGGACAAGGGCCGGGACTTCGCCACCGTGCGGAAGGTG
CTGAGCATGCCCCAGGTGAACATCGTGAAGAAAACCGAGG
TGCAGACCGGCGGCTTCAGCAAGGAGAGCATCCTGCCCAA
GCGGAACAGCGACAAGCTGATCGCCCGGAAGAAGGACTGG
GACCCCAAGAAGTACGGCGGCTTCGACAGCCCCACCGTGG
CCTACAGCGTGCTGGTGGTGGCCAAGGTGGAGAAGGGCAA
GAGCAAGAAGCTGAAATCCGTGAAGGAGCTGCTGGGCATC
ACCATCATGGAGCGGAGCAGCTTCGAGAAGAACCCCATCG
ACTTCCTGGAGGCCAAGGGCTACAAGGAGGTGAAGAAGGA
CCTGATCATCAAGCTGCCCAAGTACAGCCTGTTCGAGCTG
GAGAACGGCCGGAAGCGGATGCTGGCCAGCGCCGGCGAGC
TGCAGAAGGGCAACGAGCTGGCCCTGCCCAGCAAGTACGT
GAACTTCCTGTACCTGGCCAGCCACTACGAGAAGCTGAAG
GGCAGCCCCGAGGACAACGAGCAGAAGCAGCTGTTCGTGG
AGCAGCACAAGCACTACCTGGACGAGATCATCGAGCAGAT
CAGCGAGTTCAGCAAGCGGGTGATCCTGGCCGACGCCAAC
CTGGACAAGGTGCTGAGCGCCTACAACAAGCACCGGGACA
AGCCCATCCGGGAGCAGGCCGAGAACATCATCCACCTGTT
CACCCTGACCAACCTGGGCGCCCCCGCCGCCTTCAAGTAC
TTCGACACCACCATCGACCGGAAGCGGTACACCAGCACCA
AGGAGGTGCTGGACGCCACCCTGATCCACCAGAGCATCAC
CGGCCTGTACGAGACCCGGATCGACCTGAGCCAGCTGGGC
GGCGACAGCGGCGGCAAGCGGCCCGCCGCCACCAAGAAGG
CCGGCCAGGCCAAGAAGAAGAAGTCGGGCGGGGGTGGCTC
AAGCAGAAGGAAGCAGAGCAACCCCAGACAAATCAAGAGA
TCTCTGGGCGACATGGAGGCCAGAGAGGAAGTGCAGCTGG
TGGGCGCCAGCCACATGGAGCAGAAGGCTACAGCCCCCGA
GGCCCCCAGCCCCGGAGGTGGCGGATCGGGAAAGCGGCCC
GCCGCCACCAAGAAGGCCGGTCAGGCCAAGAAGAAGAAGG
GCAGCTACCCCTACGACGTGCCCGACTACGCCTGAGCGGC
CGCTTAATTAAGCTGCCTTCTGCGGGGCTTGCCTTCTGGC
CATGCCCTTCTTCTCTCCCTTGCACCTGTACCTCTTGGTC
TTTGAATAAAGCCTGAGTAGGAAGTCTAGAAAAAAAAAAA
AAAAAAAAAAAAAAAAAAAAAAAAAAAAAAAAAAAAAAAA
AAAAAAAAAAAAAAAAAAAAAAAAAAAAA

In some embodiments, an expression repressor comprises the amino acid sequence of SEQ ID NOs: 44 or 79. In some embodiments, an expression repressor described herein comprises an amino acid sequence of SEQ ID NO: 44 or 79 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

FOG1-dCas9-FOG1
(SEQ ID NO: 44)
MAPKKKRKVGIHGVPAASGGGGSSRRKQSNPRQIKRSLGD
MEAREEVQLVGASHMEQKATAPEAPSPGGGGSGDKKYSIG
LAIGTNSVGWAVITDEYKVPSKKFKVLGNTDRHSIKKNLI
GALLFDSGETAEATRLKRTARRRYTRRKNRICYLQEIFSN
EMAKVDDSFFHRLEESFLVEEDKKHERHPIFGNIVDEVAY
HEKYPTIYHLRKKLVDSTDKADLRLIYLALAHMIKFRGHF
LIEGDLNPDNSDVDKLFIQLVQTYNQLFEENPINASGVDA
KAILSARLSKSRRLENLIAQLPGEKKNGLFGNLIALSLGL
TPNFKSNFDLAEDAKLQLSKDTYDDDLDNLLAQIGDQYAD
LFLAAKNLSDAILLSDILRVNTEITKAPLSASMIKRYDEH
HQDLTLLKALVRQQLPEKYKEIFFDQSKNGYAGYIDGGAS
QEEFYKFIKPILEKMDGTEELLVKLNREDLLRKQRTFDNG
SIPHQIHLGELHAILRRQEDFYPFLKDNREKIEKILTFRI
PYYVGPLARGNSRFAWMTRKSEETITPWNFEEVVDKGASA
QSFIERMTNFDKNLPNEKVLPKHSLLYEYFTVYNELTKVK
YVTEGMRKPAFLSGEQKKAIVDLLFKTNRKVTVKQLKEDY
FKKIECFDSVEISGVEDRFNASLGTYHDLLKIIKDKDFLD
NEENEDILEDIVLTLTLFEDREMIEERLKTYAHLFDDKVM
KQLKRRRYTGWGRLSRKLINGIRDKQSGKTILDFLKSDGF
ANRNFMQLIHDDSLTFKEDIQKAQVSGQGDSLHEHIANLA
GSPAIKKGILQTVKVVDELVKVMGRHKPENIVIEMARENQ
TTQKGQKNSRERMKRIEEGIKELGSQILKEHPVENTQLQN
EKLYLYYLQNGRDMYVDQELDINRLSDYDVAAIVPQSFLK
DDSIDNKVLTRSDKARGKSDNVPSEEVVKKMKNYWRQLLN
AKLITQRKFDNLTKAERGGLSELDKAGFIKRQLVETRQIT
KHVAQILDSRMNTKYDENDKLIREVKVITLKSKLVSDFRK
DFQFYKVREINNYHHAHDAYLNAVVGTALIKKYPKLESEF
VYGDYKVYDVRKMIAKSEQEIGKATAKYFFYSNIMNFFKT
EITLANGEIRKRPLIETNGETGEIVWDKGRDFATVRKVLS
MPQVNIVKKTEVQTGGFSKESILPKRNSDKLIARKKDWDP
KKYGGFDSPTVAYSVLVVAKVEKGKSKKLKSVKELLGITI
MERSSFEKNPIDFLEAKGYKEVKKDLIIKLPKYSLFELEN
GRKRMLASAGELQKGNELALPSKYVNFLYLASHYEKLKGS
PEDNEQKQLFVEQHKHYLDEIIEQISEFSKRVILADANLD
KVLSAYNKHRDKPIREQAENIIHLFTLTNLGAPAAFKYFD
TTIDRKRYTSTKEVLDATLIHQSITGLYETRIDLSQLGGD
SGGKRPAATKKAGQAKKKKSGGGGSSRRKQSNPRQIKRSL
GDMEAREEVQLVGASHMEQKATAPEAPSPGGGGSGKRPAA
TKKAGQAKKKKGSYPYDVPDYA
FOG1-dCas9-FOG1 without HA tag
(SEQ ID NO: 79)
MAPKKKRKVGIHGVPAASGGGGSSRRKQSNPRQIKRSLGD
MEAREEVQLVGASHMEQKATAPEAPSPGGGGSGDKKYSIG
LAIGTNSVGWAVITDEYKVPSKKFKVLGNTDRHSIKKNLI
GALLFDSGETAEATRLKRTARRRYTRRKNRICYLQEIFSN
EMAKVDDSFFHRLEESFLVEEDKKHERHPIFGNIVDEVAY
HEKYPTIYHLRKKLVDSTDKADLRLIYLALAHMIKFRGHF
LIEGDLNPDNSDVDKLFIQLVQTYNQLFEENPINASGVDA
KAILSARLSKSRRLENLIAQLPGEKKNGLFGNLIALSLGL
TPNFKSNFDLAEDAKLQLSKDTYDDDLDNLLAQIGDQYAD
LFLAAKNLSDAILLSDILRVNTEITKAPLSASMIKRYDEH
HQDLTLLKALVRQQLPEKYKEIFFDQSKNGYAGYIDGGAS
QEEFYKFIKPILEKMDGTEELLVKLNREDLLRKQRTFDNG
SIPHQIHLGELHAILRRQEDFYPFLKDNREKIEKILTFRI
PYYVGPLARGNSRFAWMTRKSEETITPWNFEEVVDKGASA
QSFIERMTNFDKNLPNEKVLPKHSLLYEYFTVYNELTKVK
YVTEGMRKPAFLSGEQKKAIVDLLFKTNRKVTVKQLKEDY
FKKIECFDSVEISGVEDRFNASLGTYHDLLKIIKDKDFLD
NEENEDILEDIVLTLTLFEDREMIEERLKTYAHLFDDKVM
KQLKRRRYTGWGRLSRKLINGIRDKQSGKTILDFLKSDGF
ANRNFMQLIHDDSLTFKEDIQKAQVSGQGDSLHEHIANLA
GSPAIKKGILQTVKVVDELVKVMGRHKPENIVIEMARENQ
TTQKGQKNSRERMKRIEEGIKELGSQILKEHPVENTQLQN
EKLYLYYLQNGRDMYVDQELDINRLSDYDVAAIVPQSFLK
DDSIDNKVLTRSDKARGKSDNVPSEEVVKKMKNYWRQLLN
AKLITQRKFDNLTKAERGGLSELDKAGFIKRQLVETRQIT
KHVAQILDSRMNTKYDENDKLIREVKVITLKSKLVSDFRK
DFQFYKVREINNYHHAHDAYLNAVVGTALIKKYPKLESEF
VYGDYKVYDVRKMIAKSEQEIGKATAKYFFYSNIMNFFKT
EITLANGEIRKRPLIETNGETGEIVWDKGRDFATVRKVLS
MPQVNIVKKTEVQTGGFSKESILPKRNSDKLIARKKDWDP
KKYGGFDSPTVAYSVLVVAKVEKGKSKKLKSVKELLGITI
MERSSFEKNPIDFLEAKGYKEVKKDLIIKLPKYSLFELEN
GRKRMLASAGELQKGNELALPSKYVNFLYLASHYEKLKGS
PEDNEQKQLFVEQHKHYLDEIIEQISEFSKRVILADANLD
KVLSAYNKHRDKPIREQAENIIHLFTLTNLGAPAAFKYFD
TTIDRKRYTSTKEVLDATLIHQSITGLYETRIDLSQLGGD
SGGKRPAATKKAGQAKKKKSGGGGSSRRKQSNPRQIKRSL
GDMEAREEVQLVGASHMEQKATAPEAPSPGGGGSGKRPAA
TKKAGQAKKKKGS

In some embodiments, an expression repressor comprises a DNA-targeting moiety comprising dCas9, e.g., an S. pyogenes dCas9, and a repressor domain comprising DNMT, e.g., a DNMT3b domain. In some embodiments, the expression repressor is encoded by the nucleic acid sequence of SEQ ID NOs: 15 (e.g., a nucleic acid (e.g., cDNA) encoding the expression repressor). In some embodiments, a nucleic acid described herein comprises a nucleic acid sequence of SEQ ID NO: 15 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3b
(SEQ ID NO: 15)
AGGAAATAAGAGAGAAAAGAAGAGTAAGAAGAAATATAAG
AGCCACCATGGCCCCCAAGAAGAAGCGGAAGGTGGGCATC
CACGGCGTGCCCGCCGCCGACAAGAAGTACAGCATCGGCC
TGGCCATCGGCACCAACAGCGTGGGCTGGGCCGTGATCAC
CGACGAGTACAAGGTGCCCAGCAAGAAGTTCAAGGTGCTG
GGCAACACCGACCGGCACAGCATCAAGAAGAACCTGATCG
GCGCCCTGCTGTTCGACAGCGGCGAGACCGCCGAGGCCAC
CCGGCTGAAGCGGACCGCCCGGCGGCGGTACACCCGGCGG
AAGAACCGGATCTGCTACCTGCAGGAGATCTTCAGCAACG
AGATGGCCAAGGTGGACGACAGCTTCTTCCACCGGCTGGA
GGAGAGCTTCCTGGTGGAGGAGGACAAGAAGCACGAGCGG
CACCCCATCTTCGGCAACATCGTGGACGAGGTGGCCTACC
ACGAGAAGTACCCCACCATCTACCACCTGCGGAAGAAGCT
GGTGGACAGCACCGACAAGGCCGACCTGCGGCTGATCTAC
CTGGCCCTGGCCCACATGATCAAGTTCCGGGGCCACTTCC
TGATCGAGGGCGACCTGAACCCCGACAACAGCGACGTGGA
CAAGCTGTTCATCCAGCTGGTGCAGACCTACAACCAGCTG
TTCGAGGAGAACCCCATCAACGCCAGCGGCGTGGACGCCA
AGGCCATCCTGAGCGCCCGGCTGAGCAAGAGCCGGCGGCT
GGAGAACCTGATCGCCCAGCTGCCCGGCGAGAAGAAGAAC
GGCCTGTTCGGCAACCTGATCGCCCTGAGCCTGGGCCTGA
CCCCCAACTTCAAGAGCAACTTCGACCTGGCCGAGGACGC
CAAGCTGCAGCTGAGCAAGGACACCTACGACGACGACCTG
GACAACCTGCTGGCCCAGATCGGCGACCAGTACGCCGACC
TGTTCCTGGCCGCCAAGAACCTGAGCGACGCCATCCTGCT
GAGCGACATCCTGCGGGTGAACACCGAGATCACCAAGGCC
CCCCTGAGCGCCAGCATGATCAAGCGGTACGACGAGCACC
ACCAGGACCTGACCCTGCTGAAGGCCCTGGTGCGGCAGCA
GCTGCCCGAGAAGTACAAGGAGATCTTCTTCGACCAGAGC
AAGAACGGCTACGCCGGCTACATCGACGGCGGCGCCAGCC
AGGAGGAGTTCTACAAGTTCATCAAGCCCATCCTGGAGAA
GATGGACGGCACCGAGGAGCTGCTGGTGAAGCTGAACCGG
GAGGACCTGCTGCGGAAGCAGCGGACCTTCGACAACGGCA
GCATCCCCCACCAGATCCACCTGGGCGAGCTGCACGCCAT
CCTGCGGCGGCAGGAGGACTTCTACCCCTTCCTGAAGGAC
AACCGGGAGAAGATCGAGAAGATCCTGACCTTCCGGATCC
CCTACTACGTGGGCCCCCTGGCCCGGGGCAACAGCCGGTT
CGCCTGGATGACCCGGAAATCCGAGGAGACCATCACCCCC
TGGAACTTCGAGGAGGTGGTGGACAAGGGCGCCAGCGCCC
AGAGCTTCATCGAGCGGATGACCAACTTCGACAAGAACCT
GCCCAACGAGAAGGTGCTGCCCAAGCACAGCCTGCTGTAC
GAGTACTTCACCGTGTACAACGAGCTGACCAAGGTGAAGT
ACGTGACCGAGGGCATGCGGAAGCCCGCCTTCCTGAGCGG
CGAGCAGAAGAAGGCCATCGTGGACCTGCTGTTCAAGACC
AACCGGAAGGTGACCGTGAAGCAGCTGAAGGAGGACTACT
TCAAGAAGATCGAGTGCTTCGACAGCGTGGAGATCAGCGG
CGTGGAGGACCGGTTCAACGCCAGCCTGGGCACCTACCAC
GACCTGCTGAAGATCATCAAGGACAAGGACTTCCTGGACA
ACGAGGAGAACGAGGACATCCTGGAGGACATCGTGCTGAC
CCTGACCCTGTTCGAGGACCGGGAGATGATCGAGGAGCGG
CTGAAAACCTACGCCCACCTGTTCGACGACAAGGTGATGA
AGCAGCTGAAGCGGCGGCGGTACACCGGCTGGGGCCGGCT
GAGCCGGAAGCTGATCAACGGCATCCGGGACAAGCAGAGC
GGCAAGACCATCCTGGACTTCCTGAAATCCGACGGCTTCG
CCAACCGGAACTTCATGCAGCTGATCCACGACGACAGCCT
GACCTTCAAGGAGGACATCCAGAAGGCCCAGGTGAGCGGC
CAGGGCGACAGCCTGCACGAGCACATCGCCAACCTGGCCG
GCAGCCCCGCCATCAAGAAGGGCATCCTGCAGACCGTGAA
GGTGGTGGACGAGCTGGTGAAGGTGATGGGCCGGCACAAG
CCCGAGAACATCGTGATCGAGATGGCCCGGGAGAACCAGA
CCACCCAGAAGGGCCAGAAGAACAGCCGGGAGCGGATGAA
GCGGATCGAGGAGGGCATCAAGGAGCTGGGCAGCCAGATC
CTGAAGGAGCACCCCGTGGAGAACACCCAGCTGCAGAACG
AGAAGCTGTACCTGTACTACCTGCAGAACGGCCGGGACAT
GTACGTGGACCAGGAGCTGGACATCAACCGGCTGAGCGAC
TACGACGTGGCCGCCATCGTGCCCCAGAGCTTCCTGAAGG
ACGACAGCATCGACAACAAGGTGCTGACCCGGAGCGACAA
GGCCCGGGGCAAGAGCGACAACGTGCCCAGCGAGGAGGTG
GTGAAGAAGATGAAGAACTACTGGCGGCAGCTGCTGAACG
CCAAGCTGATCACCCAGCGGAAGTTCGACAACCTGACCAA
GGCCGAGCGGGGCGGCCTGAGCGAGCTGGACAAGGCCGGC
TTCATCAAGCGGCAGCTGGTGGAGACCCGGCAGATCACCA
AGCACGTGGCCCAGATCCTGGACAGCCGGATGAACACCAA
GTACGACGAGAACGACAAGCTGATCCGGGAGGTGAAGGTG
ATCACCCTGAAATCCAAGCTGGTGAGCGACTTCCGGAAGG
ACTTCCAGTTCTACAAGGTGCGGGAGATCAACAACTACCA
CCACGCCCACGACGCCTACCTGAACGCCGTGGTGGGCACC
GCCCTGATCAAGAAGTACCCCAAGCTGGAGAGCGAGTTCG
TGTACGGCGACTACAAGGTGTACGACGTGCGGAAGATGAT
CGCCAAGAGCGAGCAGGAGATCGGCAAGGCCACCGCCAAG
TACTTCTTCTACAGCAACATCATGAACTTCTTCAAGACCG
AGATCACCCTGGCCAACGGCGAGATCCGGAAGCGGCCCCT
GATCGAGACCAACGGCGAGACCGGCGAGATCGTGTGGGAC
AAGGGCCGGGACTTCGCCACCGTGCGGAAGGTGCTGAGCA
TGCCCCAGGTGAACATCGTGAAGAAAACCGAGGTGCAGAC
CGGCGGCTTCAGCAAGGAGAGCATCCTGCCCAAGCGGAAC
AGCGACAAGCTGATCGCCCGGAAGAAGGACTGGGACCCCA
AGAAGTACGGCGGCTTCGACAGCCCCACCGTGGCCTACAG
CGTGCTGGTGGTGGCCAAGGTGGAGAAGGGCAAGAGCAAG
AAGCTGAAATCCGTGAAGGAGCTGCTGGGCATCACCATCA
TGGAGCGGAGCAGCTTCGAGAAGAACCCCATCGACTTCCT
GGAGGCCAAGGGCTACAAGGAGGTGAAGAAGGACCTGATC
ATCAAGCTGCCCAAGTACAGCCTGTTCGAGCTGGAGAACG
GCCGGAAGCGGATGCTGGCCAGCGCCGGCGAGCTGCAGAA
GGGCAACGAGCTGGCCCTGCCCAGCAAGTACGTGAACTTC
CTGTACCTGGCCAGCCACTACGAGAAGCTGAAGGGCAGCC
CCGAGGACAACGAGCAGAAGCAGCTGTTCGTGGAGCAGCA
CAAGCACTACCTGGACGAGATCATCGAGCAGATCAGCGAG
TTCAGCAAGCGGGTGATCCTGGCCGACGCCAACCTGGACA
AGGTGCTGAGCGCCTACAACAAGCACCGGGACAAGCCCAT
CCGGGAGCAGGCCGAGAACATCATCCACCTGTTCACCCTG
ACCAACCTGGGCGCCCCCGCCGCCTTCAAGTACTTCGACA
CCACCATCGACCGGAAGCGGTACACCAGCACCAAGGAGGT
GCTGGACGCCACCCTGATCCACCAGAGCATCACCGGCCTG
TACGAGACCCGGATCGACCTGAGCCAGCTGGGCGGCGACA
GCGGCGGCAAGCGGCCCGCCGCCACCAAGAAGGCCGGCCA
GGCCAAGAAGAAGAAGTCGGGCGGGGGTGGCTCAGTGGAC
GTGCTGAGGAGAAGGAAGGACTGGAACGTGAGACTGCAAG
CCTTCTTCACCAGCGACACCGGACTGGAATACGAGGCCCC
TAAGCTGTACCCCGCCATTCCCGCCGCTAGACGCAGACCC
ATTAGAGTGCTCTCTCTGTTTGACGGAATCGCCACCGGCT
ATCTGGTGCTGAAAGAGCTGGGCATCAAGGTGGGCAAATA
CGTGGCCAGCGAGGTGTGCGAGGAGTCTATCGCCGTCGGA
ACCGTGAAACACGAGGGAAACATCAAATACGTGAACGACG
TGAGGAACATCACCAAGAAAAACATCGAGGAGTGGGGCCC
CTTCGATCTCGTGATCGGCGGCAGCCCTTGCAACGATCTC
TCCAATGTGAACCCCGCCAGAAAGGGACTGTACGAGGGCA
CCGGCAGACTGTTCTTCGAGTTCTACCATCTGCTGAACTA
CAGCAGACCCAAGGAGGGCGACGACAGACCCTTCTTCTGG
ATGTTCGAAAACGTGGTCGCCATGAAGGTGGGCGATAAGA
GGGACATCAGCAGATTTCTGGAGTGTAACCCCGTCATGAT
CGATGCCATTAAGGTCAGCGCCGCCCACAGAGCTAGGTAC
TTCTGGGGAAATCTGCCCGGCATGAATAGACCCGTGATCG
CTTCCAAGAACGACAAGCTGGAGCTGCAAGACTGTCTGGA
GTACAATAGAATCGCTAAGCTCAAGAAGGTCCAGACCATC
ACCACAAAATCCAACAGCATCAAGCAAGGCAAGAACCAGC
TGTTCCCCGTGGTCATGAACGGCAAGGAGGATGTGCTCTG
GTGCACCGAGCTGGAGAGGATCTTTGGCTTCCCCGTGCAC
TATACCGATGTGTCCAACATGGGAAGAGGCGCTAGACAGA
AACTGCTGGGAAGAAGCTGGAGCGTGCCCGTGATTAGACA
CCTCTTCGCCCCTCTGAAGGATTACTTCGCTTGCGAGGGA
GGTGGCGGATCGGGAAAGCGGCCCGCCGCCACCAAGAAGG
CCGGTCAGGCCAAGAAGAAGAAGGGCAGCTACCCCTACGA
CGTGCCCGACTACGCCTGAGCGGCCGCTTAATTAAGCTGC
CTTCTGCGGGGCTTGCCTTCTGGCCATGCCCTTCTTCTCT
CCCTTGCACCTGTACCTCTTGGTCTTTGAATAAAGCCTGA
GTAGGAAGTCTAGAAAAAAAAAAAAAAAAAAAAAAAAAAA
AAAAAAAAAAAAAAAAAAAAAAAAAAAAAAAAAAAAAAAA
AAAAAAAAAAAAA

In some embodiments, an expression repressor comprises the amino acid sequence of SEQ ID NOs: 16 or 37. In some embodiments, an expression repressor described herein comprises an amino acid sequence of SEQ ID NO: 16 or 37 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3b
(SEQ ID NO: 16)
MAPKKKRKVGIHGVPAADKKYSIGLAIGTNSVGWAVITDE
YKVPSKKFKVLGNTDRHSIKKNLI
GALLFDSGETAEATRLKRTARRRYTRRKNRICYLQEIFSN
EMAKVDDSFFHRLEESFLVEEDKKHERHPIFGNIVDEVAY
HEKYPTIYHLRKKLVDSTDKADLRLIYLALAHMIKFRGHF
LIEGDLNPDNSDVDKLFIQLVQTYNQLFEENPINASGVDA
KAILSARLSKSRRLENLIAQLPGEKKNGLFGNLIALSLGL
TPNFKSNFDLAEDAKLQLSKDTYDDDLDNLLAQIGDQYAD
LFLAAKNLSDAILLSDILRVNTEITKAPLSASMIKRYDEH
HQDLTLLKALVRQQLPEKYKEIFFDQSKNGYAGYIDGGAS
QEEFYKFIKPILEKMDGTEELLVKLNREDLLRKQRTFDNG
SIPHQIHLGELHAILRRQEDFYPFLKDNREKIEKILTFRI
PYYVGPLARGNSRFAWMTRKSEETITPWNFEEVVDKGASA
QSFIERMTNFDKNLPNEKVLPKHSLLYEYFTVYNELTKVK
YVTEGMRKPAFLSGEQKKAIVDLLFKTNRKVTVKQLKEDY
FKKIECFDSVEISGVEDRFNASLGTYHDLLKIIKDKDFLD
NEENEDILEDIVLTLTLFEDREMIEERLKTYAHLFDDKVM
KQLKRRRYTGWGRLSRKLINGIRDKQSGKTILDFLKSDGF
ANRNFMQLIHDDSLTFKEDIQKAQVSGQGDSLHEHIANLA
GSPAIKKGILQTVKVVDELVKVMGRHKPENIVIEMARENQ
TTQKGQKNSRERMKRIEEGIKELGSQILKEHPVENTQLQN
EKLYLYYLQNGRDMYVDQELDINRLSDYDVAAIVPQSFLK
DDSIDNKVLTRSDKARGKSDNVPSEEVVKKMKNYWRQLLN
AKLITQRKFDNLTKAERGGLSELDKAGFIKRQLVETRQIT
KHVAQILDSRMNTKYDENDKLIREVKVITLKSKLVSDFRK
DFQFYKVREINNYHHAHDAYLNAVVGTALIKKYPKLESEF
VYGDYKVYDVRKMIAKSEQEIGKATAKYFFYSNIMNFFKT
EITLANGEIRKRPLIETNGETGEIVWDKGRDFATVRKVLS
MPQVNIVKKTEVQTGGFSKESILPKRNSDKLIARKKDWDP
KKYGGFDSPTVAYSVLVVAKVEKGKSKKLKSVKELLGITI
MERSSFEKNPIDFLEAKGYKEVKKDLIIKLPKYSLFELEN
GRKRMLASAGELQKGNELALPSKYVNFLYLASHYEKLKGS
PEDNEQKQLFVEQHKHYLDEIIEQISEFSKRVILADANLD
KVLSAYNKHRDKPIREQAENIIHLFTLTNLGAPAAFKYFD
TTIDRKRYTSTKEVLDATLIHQSITGLYETRIDLSQLGGD
SGGKRPAATKKAGQAKKKKSGGGGSVDVLRRRKDWNVRLQ
AFFTSDTGLEYEAPKLYPAIPAARRRPIRVLSLFDGIATG
YLVLKELGIKVGKYVASEVCEESIAVGTVKHEGNIKYVND
VRNITKKNIEEWGPFDLVIGGSPCNDLSNVNPARKGLYEG
TGRLFFEFYHLLNYSRPKEGDDRPFFWMFENVVAMKVGDK
RDISRFLECNPVMIDAIKVSAAHRARYFWGNLPGMNRPVI
ASKNDKLELQDCLEYNRIAKLKKVQTITTKSNSIKQGKNQ
LFPVVMNGKEDVLWCTELERIFGFPVHYTDVSNMGRGARQ
KLLGRSWSVPVIRHLFAPLKDYFACEGGGGSGKRPAATKK
AGQAKKKKGSYPYDVPDYA
dCas9-DNMT3b without HA tag
(SEQ ID NO: 37)
MAPKKKRKVGIHGVPAADKKYSIGLAIGTNSVGWAVITDE
YKVPSKKFKVLGNTDRHSIKKNLIGALLFDSGETAEATRL
KRTARRRYTRRKNRICYLQEIFSNEMAKVDDSFFHRLEES
FLVEEDKKHERHPIFGNIVDEVAYHEKYPTIYHLRKKLVD
STDKADLRLIYLALAHMIKFRGHFLIEGDLNPDNSDVDKL
FIQLVQTYNQLFEENPINASGVDAKAILSARLSKSRRLEN
LIAQLPGEKKNGLFGNLIALSLGLTPNFKSNFDLAEDAKL
QLSKDTYDDDLDNLLAQIGDQYADLFLAAKNLSDAILLSD
ILRVNTEITKAPLSASMIKRYDEHHQDLTLLKALVRQQLP
EKYKEIFFDQSKNGYAGYIDGGASQEEFYKFIKPILEKMD
GTEELLVKLNREDLLRKQRTFDNGSIPHQIHLGELHAILR
RQEDFYPFLKDNREKIEKILTFRIPYYVGPLARGNSRFAW
MTRKSEETITPWNFEEVVDKGASAQSFIERMTNFDKNLPN
EKVLPKHSLLYEYFTVYNELTKVKYVTEGMRKPAFLSGEQ
KKAIVDLLFKTNRKVTVKQLKEDYFKKIECFDSVEISGVE
DRFNASLGTYHDLLKIIKDKDFLDNEENEDILEDIVLTLT
LFEDREMIEERLKTYAHLFDDKVMKQLKRRRYTGWGRLSR
KLINGIRDKQSGKTILDFLKSDGFANRNFMQLIHDDSLTF
KEDIQKAQVSGQGDSLHEHIANLAGSPAIKKGILQTVKVV
DELVKVMGRHKPENIVIEMARENQTTQKGQKNSRERMKRI
EEGIKELGSQILKEHPVENTQLQNEKLYLYYLQNGRDMYV
DQELDINRLSDYDVAAIVPQSFLKDDSIDNKVLTRSDKAR
GKSDNVPSEEVVKKMKNYWRQLLNAKLITQRKFDNLTKAE
RGGLSELDKAGFIKRQLVETRQITKHVAQILDSRMNTKYD
ENDKLIREVKVITLKSKLVSDFRKDFQFYKVREINNYHHA
HDAYLNAVVGTALIKKYPKLESEFVYGDYKVYDVRKMIAK
SEQEIGKATAKYFFYSNIMNFFKTEITLANGEIRKRPLIE
TNGETGEIVWDKGRDFATVRKVLSMPQVNIVKKTEVQTGG
FSKESILPKRNSDKLIARKKDWDPKKYGGFDSPTVAYSVL
VVAKVEKGKSKKLKSVKELLGITIMERSSFEKNPIDFLEA
KGYKEVKKDLIIKLPKYSLFELENGRKRMLASAGELQKGN
ELALPSKYVNFLYLASHYEKLKGSPEDNEQKQLFVEQHKH
YLDEIIEQISEFSKRVILADANLDKVLSAYNKHRDKPIRE
QAENIIHLFTLTNLGAPAAFKYFDTTIDRKRYTSTKEVLD
ATLIHQSITGLYETRIDLSQLGGDSGGKRPAATKKAGQAK
KKKSGGGGSVDVLRRRKDWNVRLQAFFTSDTGLEYEAPKL
YPAIPAARRRPIRVLSLFDGIATGYLVLKELGIKVGKYVA
SEVCEESIAVGTVKHEGNIKYVNDVRNITKKNIEEWGPFD
LVIGGSPCNDLSNVNPARKGLYEGTGRLFFEFYHLLNYSR
PKEGDDRPFFWMFENVVAMKVGDKRDISRFLECNPVMIDA
IKVSAAHRARYFWGNLPGMNRPVIASKNDKLELQDCLEYN
RIAKLKKVQTITTKSNSIKQGKNQLFPVVMNGKEDVLWCT
ELERIFGFPVHYTDVSNMGRGARQKLLGRSWSVPVIRHLF
APLKDYFACEGGGGSGKRPAATKKAGQAKKKKGS

Functional Characteristics

An expression repression system of the present disclosure can be used to decrease expression of a target gene in a cell. In some embodiments, decreasing expression comprises decreasing the level of RNA, e.g., mRNA, encoded by the target gene. In some embodiments, decreasing expression comprises decreasing the level of a protein encoded by the target gene. In some embodiments, decreasing expression comprises both decreasing the level of mRNA and protein encoded by the target gene. In some embodiments, the expression of a target gene in a cell contacted by or comprising the expression repression system is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of expression of the target gene in a cell not contacted by or comprising the expression repression system. Expression of a target gene may be assayed by methods known to those of skill in the art, including RT-PCR, ELISA, Western blot, and the methods of Examples 2-4.

An expression repression system of the present disclosure can be used to decrease expression of a target gene in a cell for a time period. In some embodiments, the expression of a target gene in a cell contacted by or comprising the expression repression system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the expression of a target gene in a cell contacted by or comprising the expression repression system is appreciably decreased for no more than 10, 9, 8, 7, 6, 5, 4, 3, 2, or 1 years.

In some embodiments, the expression of a target in a cell contacted by or comprising the expression repressor or the system is appreciably decreased for at least 1, 2, 3, 4, 5, 6, 7, 8, 9, or 10 cell divisions. An expression repressor or a system of the present disclosure can be used to methylate CpG nucleotides in a target region. In some embodiments, the methylation persists for at least 1 days, at least 2 days, at least 5 days, at least 7 days, at least 10 days, at least 15 days, or at least 22 days post-treatment with an expression repressor or an expression repression system disclosed herein.

An expression repression system may comprise a plurality of expression repressors, where each expression repressor comprises a repressor domain with a different functionality than the repressor domain of another expression repressor. For example, an expression repression system may comprise two expression repressors, where the first expression repressor comprises a first repressor domain comprising histone deacetylase functionality and the second expression repressor comprises a second repressor domain comprising DNA methyltransferase functionality. In some embodiments, an expression repression system comprises expression repressors comprising a combination of repressor domains whose functionalities are complementary to one another with regard to inhibiting expression of a target gene, e.g., where the functionalities together enable inhibition of expression and, optionally, do not inhibit or negligibly inhibit expression when present individually. In some embodiments, an expression repression system comprises a plurality of expression repressors, wherein each expression repressor comprises a repressor domain that complements the repressor domains of each other expression repressor, e.g., each repressor domain decreases expression of a target gene.

In some embodiments, an expression repression system comprises expression repressors comprising a combination of repressor domains whose functionalities synergize with one another with regard to inhibiting expression of a target gene. Without wishing to be bound by theory, it is thought that epigenetic modifications to a genomic locus are cumulative, in that multiple repressive epigenetic markers (e.g., multiple different types of epigenetic markers and/or more extensive marking of a given type) individually together reduce expression more effectively than individual modifications alone (e.g., producing a greater decrease in expression and/or a longer-lasting decrease in expression). In some embodiments, an expression repression system comprises a plurality of expression repressors, wherein each expression repressor comprises a repressor domain that synergizes with the repressor domains of each other expression repressor, e.g., each repressor domain decreases expression of a target gene. In some embodiments, an expression repression system (comprising a plurality of expression repressors comprising a plurality of different repressor domains which synergize with one another) is more effective at inhibiting expression of a target gene than an individual expression repressor, e.g., and its single repressor domain. In some embodiments, an expression repression system is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at inhibiting expression of a target gene than an individual expression repressor. The level of an epigenetic marker may be assayed by methods known to those of skill in the art, including whole genome bisulfite sequencing, reduced representation bisulfite sequencing, bisulfite amplicon sequencing, methylation arrays, pyrosequencing, ChIP-seq, or ChIP-qPCR.

Combinations of Repressors

In some embodiments, an expression repression system comprises a first expression repressor comprising a first repressor domain and a second expression repressor comprising a second repressor domain wherein the first repressor domain and second repressor domain are different from one another. In some embodiments, the first repressor domain is or comprises a first epigenetic modifying moiety (e.g., that increases or decreases a first epigenetic marker) or functional fragment thereof and the second repressor domain is or comprises a second epigenetic modifying moiety (e.g., that increases or decreases a second epigenetic marker) or functional fragment thereof. In some embodiments, the first repressor domain is or comprises a histone methyltransferase or functional fragment thereof and the second repressor domain is or comprises a DNA methyltransferase or functional fragment thereof. In some embodiments, the first repressor domain is or comprises a histone deacetylase or functional fragment thereof and the second repressor domain is or comprises a DNA methyltransferase or functional fragment thereof. In some embodiments, the first repressor domain is or comprises a histone deacetylase or functional fragment thereof and the second repressor domain is or comprises a histone methyltransferase or functional fragment thereof.

In some embodiments, the first repressor domain is or comprises KRAB (e.g., a KRAB domain), a SET domain (e.g., the SET domain of SETDB1 EZH2, G9A, or SUV39H1), histone demethylase LSD1, FOG1 (e.g., the N-terminal residues of FOG1), HDAC8, MQ1, DNMT1, DNMT3a/31, or KAP1, or a functional fragment of any thereof, and the second repressor domain is or comprises KRAB (e.g., a KRAB domain), a SET domain (e.g., the SET domain of SETDB1 EZH2, G9A, or SUV39H1), histone demethylase LSD1, FOG1 (e.g., the N-terminal residues of FOG1), DNMT3A (e.g., human DNMT3A), DNMT3B, DNMT3L, DNMT3A/3L complex, or bacterial MQ1, or a functional fragment of any thereof.

In some embodiments, the first repressor domain is or comprises KRAB or a functional variant or fragment thereof, and the second repressor domain is or comprises bacterial MQ1 or a functional variant or fragment thereof.

In some embodiments, the first repressor domain is or comprises KRAB or a functional variant or fragment thereof, and the second repressor domain is or comprises DNMT3A or a functional variant or fragment thereof.

In some embodiments, the first repressor domain is or comprises KRAB or a functional variant or fragment thereof, and the second repressor domain is or comprises DNMT3B or a functional variant or fragment thereof.

In some embodiments, the first repressor domain is or comprises KRAB or a functional variant or fragment thereof, and the second repressor domain is or comprises DNMT3L or a functional variant or fragment thereof.

In some embodiments, the first repressor domain is or comprises KRAB or a functional variant or fragment thereof, and the second repressor domain is or comprises DNMT3A/3L complex or a functional variant or fragment thereof.

In some embodiments, the first repressor domain is or comprises a SET domain or a functional variant or fragment thereof, and the second repressor domain is or comprises bacterial MQ1 or a functional variant or fragment thereof.

In some embodiments, the first repressor domain is or comprises LSD1 or a functional variant or fragment thereof, and the second repressor domain is or comprises bacterial MQ1 or a functional variant or fragment thereof.

In some embodiments, the first repressor domain is or comprises FOG1 or a functional variant or fragment thereof, and the second repressor domain is or comprises bacterial MQ1 or a functional variant or fragment thereof.

In some embodiments, the first repressor domain is or comprises KAP1 or a functional variant or fragment thereof, and the second repressor domain is or comprises bacterial MQ1 or a functional variant or fragment thereof.

In some embodiments, an expression repression system comprises a first expression repressor, wherein the first expression repressor is an expression repressor chosen from dCas9-MQ1, dCas9-DNMT1, dCas9-DNMT3a/31, G9A-dCas9, dCas9-HDAC8, dCas9-LSD1, EZH2-dCas9, EZH2-dCas9-KRAB, G9a-dCas9-KRAB, and Fog1-dCas9-Fog1, and a second expression repressor, wherein the second expression repressor is an expression repressor chosen from dCas9-MQ1, dCas9-DNMT1, dCas9-DNMT3a/31, G9A-dCas9, dCas9-HDAC8, dCas9-LSD1, EZH2-dCas9, EZH2-dCas9-KRAB, G9a-dCas9-KRAB, and Fog1-dCas9-Fog1.

In some embodiments, the expression repression system is encoded by a first nucleic acid encoding the first expression repressor, wherein expression is driven by a first promoter or IRES, and a second nucleic acid encoding the second expression repressor, wherein expression is driven by a second promoter or IRES.

### Target Sites

Expression repression systems disclosed herein are useful for modulating, e.g., decreasing, expression of a target gene in cell, e.g., in a subject or patient. A target gene may be any gene known to those of skill in the art. In some embodiments, a target gene is associated with a disease or condition in a subject, e.g., a mammal, e.g., a human, bovine, horse, sheep, chicken, rat, mouse, cat, or dog. A target gene may include coding sequences, e.g., exons, and/or non-coding sequences, e.g., introns, 3′UTR, or 5′UTR. In some embodiments, a target gene is operably linked to a transcription control element.

A DNA-targeting moiety suitable for use in an expression repressor of an expression repression system described herein may bind, e.g., specifically bind, to any site within a target gene, transcription control element operably linked to a target gene, or anchor sequence (e.g., an anchor sequence proximal to a target gene or associated with an anchor sequence-mediated conjunction operably linked to a target gene (e.g., an anchor sequence-mediated conjunction is operably linked to a target gene if disruption of the conjunction alters expression of the target gene)).

In some embodiments, a DNA-targeting moiety binds to a target gene. In some embodiments, a DNA-targeting moiety binds to a site within an exon of a target gene. In some embodiments, a DNA-targeting moiety binds to a site within an intron of a target gene. In some embodiments, a DNA-targeting moiety binds to a site at the boundary of an exon and an intron, e.g., a splice site, of a target gene. In some embodiments, a DNA-targeting moiety binds to a site within the 5′UTR of a target gene. In some embodiments, a DNA-targeting moiety binds to a site within the 3′UTR of a target gene. Target genes include, but are not limited to, the gene encoding β-2-microglobulin, the gene encoding MYC, the gene encoding HSPA1B, the gene encoding GATA1, the gene encoding APOB, the gene encoding FOXP3, the gene encoding CXCL1, the gene encoding CXCL2, the gene encoding CXCL3, the gene encoding CXCL4, the gene encoding CXCL5, the gene encoding CXCL6, the gene encoding CXCL7, and the gene encoding CXCL8.

In some embodiments, a DNA-targeting moiety binds to a transcription control element operably linked to a target gene, e.g., a promoter or enhancer. In some embodiments, a DNA-targeting moiety binds to a portion of or a site within a promoter operably linked to a target gene. In some embodiments, a DNA-targeting moiety binds to the transcription start site of a target gene. In some embodiments, a DNA-targeting moiety binds to a portion of or a site within an enhancer operably linked to a target gene. A promoter is, typically, a sequence element that initiates transcription of an associated gene. Promoters are typically near the 5′ end of a gene, not far from its transcription start site. As those of ordinary skill are aware, transcription of protein-coding genes in eukaryotic cells is typically initiated by binding of general transcription factors (e.g., TFIID, TFIIE, TFIIH, etc.) and Mediator to core promoter sequences as a preinitiation complex that directs RNA polymerase II to the transcription start site, and in many instances remains bound to the core promoter sequences even after RNA polymerase escapes and elongation of the primary transcript is initiated. In some embodiments, a promoter includes a sequence element such as TATA, Inr, DPE, or BRE, but those skilled in the art are well aware that such sequences are not necessarily required to define a promoter. Those skilled in the art are familiar with a variety of positive (e.g., enhancers) or negative (e.g., repressors or silencers) transcription control elements that are associated with genes. In some embodiments, a transcription control element is a transcription factor binding site. Typically, when a cognate regulatory protein is bound to such a transcription control element, transcription from the associated gene(s) is altered (e.g., increased or decreased).

In some embodiments, a DNA-targeting moiety binds to an anchor sequence, e.g., an anchor sequence proximal to a target gene or associated with an anchor sequence-mediated conjunction operably linked to a target gene (e.g., an anchor sequence-mediated conjunction is operably linked to a target gene if disruption of the conjunction alters expression of the target gene). In some embodiments, a DNA-targeting moiety binds to or proximal to an anchor sequence of an anchor sequence mediated conjunction (ASMC) comprising a target plurality of genes, e.g., human CXCL1-8. In general, an anchor sequence is a genomic sequence element to which a genomic complex component binds specifically. In some embodiments, binding of a genomic complex component to an anchor sequence nucleates complex formation, e.g., anchor sequence-mediated conjunction formation. Each anchor sequence-mediated conjunction comprises one or more anchor sequences, e.g., a plurality. In some embodiments, an anchor sequence-mediated conjunction can be disrupted to alter, e.g., inhibit, expression of a target gene. Such disruptions may modulate gene expression by, e.g., changing topological structure of DNA, e.g., by modulating the ability of a target gene to interact with a transcription control element (e.g., enhancing and silencing/repressive sequences).

A DNA-targeting moiety suitable for use in an expression repressor of an expression repression system described herein may bind, e.g., specifically bind, to a site that is proximal to a target gene (e.g., an exon, intron, or splice site within the target gene), proximal to a transcription control element operably linked to the target gene, or proximal to an anchor sequence, e.g., an anchor sequence proximal to a target gene or associated with an anchor sequence-mediated conjunction operably linked to a target gene (e.g., an anchor sequence-mediated conjunction is operably linked to a target gene if disruption of the conjunction alters expression of the target gene). As used herein, proximal refers to a closeness of two sites, e.g., nucleic acid sites, such that binding of an expression repressor at the first site and/or modification of the first site by an expression repressor will produce the same or substantially the same effect as binding and/or modification of the other site. For example, a DNA-targeting moiety may bind to a first site that is proximal to an enhancer (the second site), and the repressor domain associated with said DNA-targeting moiety may epigenetically modify the first site such that the enhancer's effect on expression of a target gene is modified, substantially the same as if the second site (the enhancer sequence) had been bound and/or modified. In some embodiments, a site proximal to a target gene (e.g., an exon, intron, or splice site within the target gene), proximal to a transcription control element operably linked to the target gene, or proximal to an anchor sequence is less than 5000, 4000, 3000, 2000, 1000, 900, 800, 700, 600, 500, 400, 300, 200, 100, 50, or 25 base pairs from the target gene (e.g., an exon, intron, or splice site within the target gene), transcription control element, or anchor sequence (and optionally at least 20, 25, 50, 100, 200, or 300 base pairs from the target gene (e.g., an exon, intron, or splice site within the target gene), transcription control element, or anchor sequence).

A DNA-targeting moiety suitable for use in an expression repressor of an expression repression system described herein may bind, e.g., specifically bind, to a site comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs (and optionally no more 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 nucleotides or base pairs). In some embodiments, a DNA-targeting moiety binds to a site comprising 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs.

Expression repression systems of the present disclosure may comprise two or more expression repressors. In some embodiments, the expression repressors of an expression repression system each comprise a different DNA-targeting moiety.

In some embodiments, an expression repression system comprises a first expression repressor comprising a DNA-targeting moiety that binds a target gene, e.g., an exon, intron, or exon intron boundary (e.g., splice site), and second expression repressor comprising a DNA-targeting moiety that binds the target gene, e.g., an exon, intron, or exon intron boundary (e.g., splice site). In some embodiments, an expression repression system comprises a first expression repressor comprising a DNA-targeting moiety that binds a target gene, e.g., an exon, intron, or exon intron boundary (e.g., splice site), and second expression repressor comprising a DNA-targeting moiety that binds to a transcription control element (e.g., promoter or enhancer) operably linked to the target gene. In some embodiments, an expression repression system comprises a first expression repressor comprising a DNA-targeting moiety that binds to a transcription control element (e.g., promoter or enhancer) operably linked to a target gene, and a second expression repressor comprising a DNA-targeting moiety that binds to a transcription control element (e.g., promoter or enhancer) operably linked to the target gene. In some embodiments, an expression repression system comprises a first expression repressor comprising a DNA-targeting moiety that binds to an anchor sequence proximal to a target gene or associated with an anchor sequence-mediated conjunction operably linked to a target gene, and a second expression repressor comprising a DNA-targeting moiety that binds to a transcription control element (e.g., promoter or enhancer) operably linked to the target gene. In some embodiments, an expression repression system comprises a first expression repressor comprising a DNA-targeting moiety that binds to an anchor sequence proximal to a target gene or associated with an anchor sequence-mediated conjunction operably linked to a target gene, and a second expression repressor comprising a DNA-targeting moiety that binds to the target gene, e.g., an exon, intron, or exon intron boundary (e.g., splice site). In some embodiments, an expression repression system comprises a first expression repressor comprising a DNA-targeting moiety that binds to an anchor sequence proximal to a target gene or associated with an anchor sequence-mediated conjunction operably linked to a target gene, and a second expression repressor comprising a DNA-targeting moiety that binds to an anchor sequence proximal to the target gene or associated with an anchor sequence-mediated conjunction operably linked to the target gene.

In some embodiments, an expression repression system comprises a first expression repressor comprising a DNA-targeting moiety that binds to a first site, e.g., in a promoter operably linked to a target gene, and a second expression repressor comprising a DNA-targeting moiety that binds to a second site, e.g., in the promoter operably linked to a target gene. The first site and second site may be different and non-overlapping sites, e.g., the first site and second site do not share any sequence in common. The first site and second site may be different but overlapping sites, e.g., the first site and second site comprise different sequences but share some sequence in common.

In some embodiments, a DNA-targeting moiety binds to a sequence selected from SEQ ID NOs: 1-21 or a sequence with no more than 10, 9, 8, 7, 6, 5, 4, 3, 2, or 1 alteration relative thereto.

(SEQ ID NO: 6)
ACGGCGGGCCACCAAGGAGA
(SEQ ID NO: 7)
CAGCGCCGCGCCTTTGGGAC
(SEQ ID NO: 8)
CCCTTTCGGCGGGGAGCAGGG
(SEQ ID NO: 9)
GCTGCGCTGGGGGAGCCAGAG
(SEQ ID NO: 10)
AGCGCCCCTCCACGCGTTCAC

In some embodiments, the first DNA-targeting moiety binds to a sequence comprising any one of SEQ ID NOS: 1-21 and the second DNA-targeting moiety binds to a sequence comprising any one of SEQ ID NOS: 1-21, wherein the first and the second DNA-targeting moiety binds to the same sequence. In some embodiments, the first DNA-targeting moiety binds to a sequence comprising any one of SEQ ID NOS: 1-21 and the second DNA-targeting moiety binds to a sequence comprising any one of SEQ ID NOS: 1-21 wherein the first and the second targeting moiety binds to different sequences.

In some embodiments, an expression repressor binds a genomic locus having a sequence set forth herein, e.g., any one of SEQ ID NOS: 1-21. It is understood that, in many cases, the genomic locus being bound comprises double stranded DNA, and this locus can be described by giving the sequence of its sense strand or its antisense strand. Thus, a gRNA having a given spacer sequence may cause expression repressor to bind to a particular genomic locus, wherein one strand of the genomic locus has a sequence similar or identical to the spacer sequence, and the other strand of the genomic locus has the complementary sequence. Typically, gRNA binding to the genomic locus will involve some unwinding of the genomic locus and pairing of the gRNA spacer with the strand to which the spacer is complementary.

In some embodiments, an anchor sequence comprises a nucleating polypeptide binding motif, e.g., a CTCF-binding motif:

N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO: 81), where N is any nucleotide.

A CTCF-binding motif may also be in an opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO: 82). where N is any nucleotide

In some embodiments, an anchor sequence comprises SEQ ID NO: 81 or SEQ ID NO: 82 or a sequence at least 75%, at least 80%, at least 90%, at least 95%, at least 96%, at least 97%, at least 98%, at least 99% identical to either SEQ ID NO: 81 or SEQ ID NO: 82.

In some embodiments, an anchor sequence comprises a nucleating polypeptide binding motif, e.g., a YY1-binding motif: CCGCCATNTT (SEQ ID NO: 83), where N is any nucleotide.

A YY1-binding motif may also be in an opposite orientation, e.g., AANATGGCGG (SEQ ID NO: 84), where N is any nucleotide.

A targeting moiety suitable for use in an expression repressor of an expression repression system described herein may bind, e.g., specifically bind, to a site comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs (and optionally no more 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 nucleotides or base pairs). In some embodiments, a DNA-targeting moiety binds to a site comprising 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs.

Expression repression systems of the present disclosure may comprise two or more expression repressors. In some embodiments, the expression repressors of an expression repression system each comprise a different targeting moiety.

In some embodiments, an expression repression system comprises a first expression repressor comprising a DNA-targeting moiety that binds a target gene, e.g., an exon, intron, or exon intron boundary (e.g., splice site), and second expression repressor comprising a DNA-targeting moiety that binds the target gene, e.g., an exon, intron, or exon intron boundary (e.g., splice site). In some embodiments, an expression repression system comprises a first expression repressor comprising a DNA-targeting moiety that binds a target gene, e.g., an exon, intron, or exon intron boundary (e.g., splice site), and second expression repressor comprising a DNA-targeting moiety that binds to a transcription control element (e.g., promoter or enhancer) operably linked to the target gene. In some embodiments, an expression repression system comprises a first expression repressor comprising a DNA-targeting moiety that binds to a transcription control element (e.g., promoter or enhancer) operably linked to a target gene, and a second expression repressor comprising a DNA-targeting moiety that binds to a transcription control element (e.g., promoter or enhancer) operably linked to the target gene. In some embodiments, an expression repression system comprises a first expression repressor comprising a DNA-targeting moiety that binds to an anchor sequence proximal to a target gene, e or associated with an anchor sequence-mediated conjunction operably linked to a target gene, and a second expression repressor comprising a DNA-targeting moiety that binds to a transcription control element (e.g., promoter or enhancer) operably linked to the target gene. In some embodiments, an expression repression system comprises a first expression repressor comprising a DNA-targeting moiety that binds to an anchor sequence proximal to a target gene, or associated with an anchor sequence-mediated conjunction operably linked to a target gene, and a second expression repressor comprising a DNA-targeting moiety that binds to the target gene, e.g., an exon, intron, or exon intron boundary (e.g., splice site). In some embodiments, an expression repression system comprises a first expression repressor comprising a DNA-targeting moiety that binds to an anchor sequence proximal to a target gene, or associated with an anchor sequence-mediated conjunction operably linked to a target gene, and a second expression repressor comprising a DNA-targeting moiety that binds to an anchor sequence proximal to the target gene, or associated with an anchor sequence-mediated conjunction operably linked to the target gene.

### Other Compositions

Nucleic Acids and Vectors

The present disclosure is further directed, in part, to nucleic acids encoding expression repressors or expression repression systems described herein. In some embodiments, an expression repression system may be provided via a composition comprising a nucleic acid encoding the expression repression system, e.g., expression repressor(s) of the expression repression system, wherein the nucleic acid is associated with sufficient other sequences to achieve expression of the expression repression system, e.g., expression repressor(s) of the expression repression system, in a system of interest (e.g., in a particular cell, tissue, organism, etc.).

In some particular embodiments, the present disclosure provides compositions of nucleic acids that encode an expression repression system, one or more expression repressors, or polypeptide portion thereof. In some such embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc.). In some embodiments, provided nucleic acids that encode an expression repression system, one or more expression repressors, or polypeptide portion thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.). In some embodiments, a composition for delivering an expression repression system described herein is or comprises a vector, e.g., a viral vector, comprising one or more nucleic acids encoding one or more components of an expression repression system, e.g., expression repressor(s) of the expression repression system as described herein.

In some embodiments, a composition for delivering an expression repressor or an expression repression system described herein is or comprises RNA, e.g., mRNA, comprising one or more nucleic acids encoding one or more components of an expression repressor or an expression repression system, e.g., expression repressor(s) of the expression repression system as described herein.

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter.

The present disclosure should not interpreted to be limited to use of any particular promoter or category of promoters (e.g. constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

Cells

The present disclosure is further directed, in part, to cells comprising an expression repressor or an expression repression system described herein. Any cell, e.g., cell line, e.g., a cell line suitable for expression of a recombinant polypeptide, known to one of skill in the art is suitable to comprise an expression repressor or an expression repression system described herein. In some embodiments, a cell, e.g., cell line, may be used to express an expression repressor or an expression repression system, e.g., expression repressor(s), described herein. In some embodiments, a cell, e.g., cell line, may be used to express or amplify a nucleic acid, e.g., a vector, encoding an expression repressor or an expression repression system, e.g., expression repressor(s), described herein. In some embodiments, a cell comprises a nucleic acid encoding an expression repressor or an expression repression system, e.g., expression repressor(s), described herein.

In some embodiments, a cell comprises a first nucleic acid encoding a first component of an expression repressor or an expression repression system, e.g., a first expression repressor, and a second nucleic acid encoding a second component of the expression repression system, e.g., a second expression repressor. In some embodiments, wherein a cell comprises nucleic acid encoding an expression repression system comprising two or more expression repressors, the sequences encoding each expression repressor are disposed on separate nucleic acid molecules, e.g., on different vectors, e.g., a first vector encoding a first expression repressor and a second vector encoding a second expression repressor. In some embodiments, the sequences encoding each expression repressor are disposed on the same nucleic acid molecule, e.g., on the same vector. In some embodiments, some or all of the nucleic acid encoding the expression repression system is integrated into the genomic DNA of the cell. In some embodiments, the nucleic acid encoding a first expression repressor of an expression repression system is integrated into the genomic DNA of a cell, and the nucleic acid encoding a second expression repressor of an expression repression system is not integrated into the genomic DNA of a cell (e.g., is situated on a vector). In some embodiments, the nucleic acid(s) encoding a first and a second expression repressor of an expression repression system are integrated into the genomic DNA of a cell, e.g., at the same (e.g., adjacent or colocalized) or different sites in the genomic DNA.

Examples of cells that may comprise and/or express an expression repression system or expression repressor described herein include, but are not limited to, hepatocytes, neuronal cells, endothelial cells, myocytes, and lymphocytes.

The present disclosure is further directed, in part, to a cell made by a method or process described herein. In some embodiments, the disclosure provides a cell produced by: providing an expression repression system described herein, providing the cell, and contacting the cell with the expression repression system (or a nucleic acid encoding the expression repression system, or a composition comprising said expression repression system or nucleic acid). Without wishing to be bound by theory, a cell contacted with an expression repression system described herein may exhibit: a decrease in expression of a target gene and/or a modification of epigenetic markers associated with the target gene, a transcription control element operably linked to the target gene, or an anchor sequence proximal to the target gene or associated with an anchor sequence-mediated conjunction operably linked to the target gene compared to a similar cell that has not been contacted by the expression repression system. In some embodiments, a cell exhibiting said decrease in expression of a target gene and/or modification of epigenetic markers does not comprise the expression repression system. The decrease in expression and/or modification of epigenetic markers may persist, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after contact with the expression repression system. In some embodiments, a cell previously contacted by an expression repression system retains the decrease in expression and/or modification of epigenetic markers after the expression repression system is no longer present in the cell, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after the expression repression system is no longer present in the cell.

Methods of Making Expression Repression Systems and/or Expression Repressors

In some embodiments, an expression repressor comprises or is a protein and may thus be produced by methods of making proteins. In some embodiments, an expression repression system, e.g., the expression repressor(s) of an expression repression system, comprise one or more proteins and may thus be produced by methods of making proteins. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise non transcribed elements such as an origin of replication, a suitable promoter, and other 5′ or 3′ flanking non transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein. Compositions described herein may include a lipid nanoparticle encapsulating a vector, such as a viral vector, e.g., a lentiviral vector, encoding a recombinant protein. In some embodiments, a lipid nanoparticle encapsulating a vector, e.g., a viral vector, may comprise a nucleic acid encoding a recombinant protein.

Proteins comprise one or more amino acids. Amino acids include any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

### Pharmaceutical Compositions, Formulation, Delivery, and Administration

The present disclosure is further directed, in part, to pharmaceutical compositions comprising an expression repressor or an expression repression system, e.g., expression repressor(s), described herein, to pharmaceutical compositions comprising nucleic acids encoding the expression repressor or the expression repression system, e.g., expression repressor(s), described herein, and/or to and/or compositions that deliver an expression repressor or an expression repression system, e.g., expression repressor(s), described herein to a cell, tissue, organ, and/or subject.

As used herein, the term “pharmaceutical composition” refers to an active agent (e.g., an expression repressor or nucleic acids of the expression receptor, e.g., an expression repression system, e.g., expression repressor(s) of an expression repression system, or nucleic acid encoding the same), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, a pharmaceutical composition comprising an expression repressor of the present disclosure comprises an expression repressor or nucleic acid(s) encoding the same. In some embodiments, a pharmaceutical composition comprising an expression repression system of the present disclosure comprises or each of the expression repressors of the expression repression system or nucleic acid(s) encoding the same (e.g., if an expression repression system comprises a first expression repressor and a second expression repressor, the pharmaceutical composition comprises the first and second expression repressor). In some embodiments, a pharmaceutical composition comprises less than all of the expression repressors of an expression repression system comprising a plurality of expression repressors. For example, an expression repression system may comprise a first expression repressor and a second expression repressor, and a first pharmaceutical composition may comprise the first expression repressor or nucleic acid encoding the same and a second pharmaceutical composition may comprise the second expression repressor or nucleic acid encoding the same.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid, or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate, and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing, and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, pharmaceutical compositions may be formulated for delivery to a cell and/or to a subject via any route of administration. Modes of administration to a subject may include injection, infusion, inhalation, intranasal, intraocular, topical delivery, intercannular delivery, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein a genomic complex modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to epithelial, connective, muscular, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof.

In some embodiments, a composition of the present disclosure crosses a blood-brain-barrier, a placental membrane, or a blood-testis barrier.

In some embodiments, a pharmaceutical composition as provided herein is administered systemically.

In some embodiments, administration is non-parenteral and a therapeutic is a parenteral therapeutic.

In some embodiments, a pharmaceutical composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to an active agent alone. In some embodiments, a pharmaceutical composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to an active agent alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to an active agent alone. Pharmaceutical compositions described herein may be formulated for example including a carrier, such as a pharmaceutical carrier and/or a polymeric carrier, e.g., a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

### Uses

The present disclosure is further directed to uses of the expression repression systems disclosed herein. Among other things, in some embodiments such provided technologies may be used to achieve modulation, e.g., repression, of target gene expression and, for example, enable control of target gene activity, delivery, and penetrance, e.g., in a cell. In some embodiments, a cell is a mammalian, e.g., human, cell. In some embodiments, a cell is a somatic cell. In some embodiments, a cell is a primary cell. For example, in some embodiments, a cell is a mammalian somatic cell. In some embodiments, a mammalian somatic cell is a primary cell. In some embodiments, a mammalian somatic cell is a non-embryonic cell.

### Dosage

The dosage of the administered agent or composition can vary based on, e.g., the condition being treated, the severity of the disease, the subject's individual parameters, including age, physiological condition, size and weight, duration of treatment, the type of treatment to be performed (if any), the particular route of administration and similar factors. Thus, the dose administered of the agents described herein can depend on such various parameters. The dosage of an administered composition may also vary depending upon other factors as the subject's sex, general medical condition, and severity of the disorder to be treated. It may be desirable to provide the subject with a dosage of an expression repressor or an expression repression system or combination of expression repressors disclosed herein as circumstances dictate. In some embodiments, the subject is provided with a dosage of an expression repressor, or an expression repression system or combination of expression repressors disclosed herein as circumstances dictate. Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

### Lipid Nanoparticles

Expression repressors or expression repression systems as described herein can be delivered using any biological delivery system/formulation including a particle, for example, a nanoparticle delivery system. Nanoparticles include particles with a dimension (e.g. diameter) between about 1 and about 1000 nanometers, between about 1 and about 500 nanometers in size, between about 1 and about 100 nm, between about 30 nm and about 200 nm, between about 50 nm and about 300 nm, between about 75 nm and about 200 nm, between about 100 nm and about 200 nm, and any range therebetween. A nanoparticle has a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In some embodiments, nanoparticles have a greatest dimension ranging between 25 nm and 200 nm. Nanoparticles as described herein comprise delivery systems that may be provided in any form, including but not limited to solid, semi-solid, emulsion, or colloidal nanoparticles. A nanoparticle delivery system may include but not limited to lipid-based systems, liposomes, micelles, micro-vesicles, exosomes, or gene gun. In one embodiment, the nanoparticle is a lipid nanoparticle (LNP). In some embodiments, the LNP is a particle that comprises a plurality of lipid molecules physically associated with each other by intermolecular forces.

In some embodiments, an LNP may comprise multiple components, e.g., 3-4 components. In one embodiment, the expression repressor or a pharmaceutical composition comprising said expression repressor (or a nucleic acid encoding the same, or pharmaceutical composition comprising said expression repressor nucleic acid) is encapsulated in an LNP. In one embodiment, the expression repression system or a pharmaceutical composition comprising said expression repression system (or a nucleic acid encoding the same, or pharmaceutical composition comprising said expression repression system nucleic acid) is encapsulated in an LNP. In some embodiments, the nucleic acid encoding the first expression repressor and the nucleic acid encoding the second expression repressor are present in same LNP. In some embodiments, the nucleic acid encoding the first expression repressor and the nucleic acid encoding the second expression repressor are present in different LNPs. Preparation of LNPs and the modulating agent encapsulation may be used/and or adapted from Rosin et al, Molecular Therapy, vol. 19, no. 12, pages 1286-2200, December 2011). In some embodiments, lipid nanoparticle compositions disclosed herein are useful for expression of protein encoded by mRNA. In some embodiments, nucleic acids, when present in the lipid nanoparticles, are resistant in aqueous solution to degradation with a nuclease.

In some embodiments, the LNP formulations may include a CCD lipid, a neutral lipid, and/or a helper lipid. In some embodiments, the LNP formulation comprises an ionizable lipid. In some embodiments, an ionizable lipid may be a cationic lipid, an ionizable cationic lipid, or an amine-containing lipid that can be readily protonated. In some embodiments, the lipid is a cationic lipid that can exist in a positively charged or neutral form depending on pH. In some embodiments, the cationic lipid is a lipid capable of being positively charged, e.g., under physiological conditions. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol, and polymer conjugated lipids. In some embodiments, LNP formulation (e.g., MC3 and/or SSOP) includes cholesterol, PEG, and/or a helper lipid. The LNPs may be, e.g., microspheres (including unilamellar and multilamellar vesicles, lamellar phase lipid bilayers that, in some embodiments, are substantially spherical.

In some embodiments, the LNP can comprise an aqueous core, e.g., comprising a nucleic acid encoding an expression repressor or a system as disclosed herein. In some embodiments of the present disclosure, the cargo for the LNP formulation includes at least one guide RNA. In some embodiments, the cargo, e.g., a nucleic acid encoding an expression repressor, or a system as disclosed herein, may be adsorbed to the surface of an LNP, e.g., an LNP comprising a cationic lipid. In some embodiments, the cargo, e.g., a nucleic acid encoding an expression repressor, or a system as disclosed herein may be associated with the LNP. In some embodiments, the cargo, e.g., a nucleic acid encoding an expression repressor, or a system as disclosed herein, may be encapsulated, e.g., fully encapsulated and/or partially encapsulated in an LNP.

In some embodiments, an LNP comprising a cargo may be administered for systemic delivery, e.g., delivery of a therapeutically effective dose of cargo that can result in a broad exposure of an active agent within an organism. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery. In some embodiments, an LNP comprising a cargo may be administered for local delivery, e.g., delivery of an active agent directly to a target site within an organism.

The LNPs may be formulated as a dispersed phase in an emulsion, micelles, or an internal phase in a suspension. In some embodiments, the LNPs are biodegradable. In some embodiments, the LNPs do not accumulate to cytotoxic levels or cause toxicity in vivo at a therapeutically effective dose. In some embodiments, the LNPs do not accumulate to cytotoxic levels or cause toxicity in vivo after repeat administrations at a therapeutically effective dose. In some embodiments, the LNPs do not cause an innate immune response that leads to a substantially adverse effect at a therapeutically effective dose.

In some embodiments, the LNP used, comprises the formula (6Z,9Z,28Z,31Z)-heptatriacont-6,9,28,31-tetraene-19-yl 4-(dimethylamino) butanoate or ssPalmO-phenyl-P4C2 (ssPalmO-Phe, SS-OP). In some embodiments, the LNP formulation comprises the formula, (6Z,9Z,28Z,31Z)-heptatriacont-6,9,28,31-tetraene-19-yl 4-(dimethylamino)butanoate (MC3), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., MC3 LNP or ssPalmO-phenyl-P4C2 (ssPalmO-Phe, SS-OP), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., SSOP-LNP.

Liposomes are spherical vesicle structures composed of a uni- or multilamellar lipid bilayer surrounding internal aqueous compartments and a relatively impermeable outer lipophilic phospholipid bilayer.

Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

### Modulating Gene Expression

The present disclosure is further directed, in part, to a method of modulating, e.g., decreasing, expression of a target gene, comprising providing an expression repressor (or a nucleic acid encoding the same, or pharmaceutical composition comprising said expression repressor nucleic acid) or an expression repression system described herein (or a nucleic acid encoding the same, or pharmaceutical composition comprising said expression repression system or nucleic acid), and contacting the target gene, and/or operably linked transcription control element(s) with the expression repressor or the expression repression system. In some embodiments, modulating, e.g., decreasing expression of a target gene, comprises modulation of transcription of a target gene, as compared with a reference value, e.g., transcription of a target gene, in absence of the expression repressor or the expression repression system. In some embodiments, the method of modulating, e.g., decreasing, expression of a target gene, are used ex vivo, e.g., on a cell from a subject, e.g., a mammalian subject, e.g., a human subject. In some embodiments, the method of modulating, e.g., decreasing, expression of a target gene, are used in vivo, e.g., on a mammalian subject, e.g., a human subject. In some embodiments, the method of modulating, e.g., decreasing, expression of a target gene, are used in vitro, e.g., on a cell or cell line described herein.

The present disclosure is further directed, in part to a method of treating a condition associated with over-expression of a target gene in a subject, comprising administering to the subject an expression repression system described herein (or a nucleic acid encoding the same, or pharmaceutical composition comprising said expression repression system or nucleic acid). Conditions associated with over-expression of particular genes are known to those of skill in the art. Such conditions include, but are not limited to, metabolic disorders, neuromuscular disorders, cancer (e.g., solid tumors), fibrosis, diabetes, urea disorders, immune disorders, inflammation, and arthritis.

The present disclosure is further directed, in part to a method of treating a condition associated with mis-regulation of the expression of a target gene in a subject, comprising administering to the subject an expression repression system described herein (or a nucleic acid encoding the same, or pharmaceutical composition comprising said expression repression system or nucleic acid). Without wishing to be bound by theory, it is thought that an expression repression system may be used to target (e.g., decrease expression of) a gene which modulates the expression of a target gene, thus altering expression of the target gene by altering expression of the modulating gene. Conditions associated with mis-regulation of the expression of particular genes are known to those of skill in the art. Such conditions include, but are not limited to metabolic disorders, neuromuscular disorders, cancer (e.g., solid tumors), fibrosis, diabetes, urea disorders, immune disorders, inflammation, and arthritis.

Methods and compositions as provided herein may treat a condition associated with over-expression or mis-regulation of a target gene by stably or transiently altering (e.g., decreasing) transcription of a target gene. In some embodiments, such a modulation persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, a method or composition provided herein may decrease expression of a target gene in a cell by at least 10, 20, 30, 40, 50, 60, 70, 80, 90, or 100% (and optionally up to 100%) relative to expression of the target gene in a cell not contacted by the composition or treated with the method.

In some embodiments, a method provided herein may modulate, e.g., decrease, expression of a target gene by disrupting a genomic complex, e.g., an anchor sequence-mediated conjunction, associated with said target gene. A gene that is associated with an anchor sequence-mediated conjunction may be at least partially within a conjunction (that is, situated sequence-wise between a first and second anchor sequences), or it may be external to a conjunction in that it is not situated sequence-wise between a first and second anchor sequences, but is located on the same chromosome and in sufficient proximity to at least a first or a second anchor sequence such that its expression can be modulated by controlling the topology of the anchor sequence-mediated conjunction. Those of ordinary skill in the art will understand that distance in three-dimensional space between two elements (e.g., between the gene and the anchor sequence-mediated conjunction) may, in some embodiments, be more relevant than distance in terms of base pairs. In some embodiments, an external but associated gene is located within 2 Mb, within 1.9 Mb, within 1.8 Mb, within 1.7 Mb, within 1.6 Mb, within 1.5 Mb, within 1.4 Mb, with 1.3 Mb, within 1.3 Mb, within 1.2 Mb, within 1.1 Mb, within 1 Mb, within 900 kb, within 800 kb, within 700 kb, within 500 kb, within 400 kb, within 300 kb, within 200 kb, within 100 kb, within 50 kb, within 20 kb, within 10 kb, or within 5 kb of the first or second anchor sequence.

In some embodiments, modulating expression of a gene comprises altering accessibility of a transcriptional control sequence to a gene. A transcriptional control sequence, whether internal or external to an anchor sequence-mediated conjunction, can be an enhancing sequence or a silencing (or repressive) sequence.

### Epigenetic Modification

The present disclosure is further directed, in part, to a method of epigenetically modifying a target gene, a transcription control element operably linked to a target gene, or an anchor sequence (e.g., an anchor sequence proximal to a target gene or associated with an anchor sequence-mediated conjunction operably linked to a target gene), the method comprising providing an expression repression system (e.g., expression repressor(s)), or nucleic acid encoding the same or pharmaceutical composition comprising said expression repression system or nucleic acid; and contacting the target gene or a transcription control element operably linked to the target gene with the expression repression system, thereby epigenetically modifying the target gene or a transcription control element operably linked to the target gene.

In some embodiments, a method of epigenetically modifying a target gene or a transcription control element operably linked to a target gene comprises increasing or decreasing DNA methylation of the target gene or a transcription control element operably linked to a target gene. In some embodiments, a method of epigenetically modifying a target gene or a transcription control element operably linked to a target gene comprises increasing or decreasing histone methylation of a histone associated with the target gene or a transcription control element operably linked to a target gene. In some embodiments, a method of epigenetically modifying a target gene or a transcription control element operably linked to a target gene comprises decreasing histone acetylation of a histone associated with the target gene, or a transcription control element operably linked to a target gene. In some embodiments, a method of epigenetically modifying a target gene or a transcription control element operably linked to a target gene comprises increasing or decreasing histone sumoylation of a histone associated with the target gene or a transcription control element operably linked to a target gene. In some embodiments, a method of epigenetically modifying a target gene or a transcription control element operably linked to a target gene comprises increasing or decreasing histone phosphorylation of a histone associated with the target gene or a transcription control element operably linked to a target gene.

In some embodiments, a method of epigenetically modifying a target gene or a transcription control element operably linked to a target gene may decrease the level of the epigenetic modification by at least 10, 20, 30, 40, 50, 60, 70, 80, 90, or 100% (and optionally up to 100%) relative to the level of the epigenetic modification at that site in a cell not contacted by the composition or treated with the method. In some embodiments, a method of epigenetically modifying a target gene or a transcription control element operably linked to a target gene may increase the level of the epigenetic modification by at least 10, 20, 30, 40, 50, 60, 70, 80, 90, 100, 120, 140, 160, 180, 200, 300, 400, 500, 600, 700, 800, 900, or 1000% (and optionally up to 200, 300, 400, 500, 600, 700, 800, 900, 1000, or 2000%) relative to the level of the epigenetic modification at that site in a cell not contacted by the composition or treated with the method. In some embodiments epigenetic modification of a target gene or a transcription control element operably linked to a target gene may modify the level of expression of the target gene, e.g., as described herein.

In some embodiments, an epigenetic modification produced by a method described herein persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, an expression repression system for use in a method of epigenetically modifying a target gene or a transcription control element operably linked to a target gene comprises an expression repressor comprising a repressor domain that is or comprises an epigenetic modifying moiety. For example, a repressor domain may be or comprise an epigenetic modifying moiety with DNA methyltransferase activity, and an endogenous or naturally occurring target sequence (e.g. a target gene or transcription control element) may be altered to increase its methylation (e.g., decreasing interaction of a transcription factor with a portion of target gene or transcription control element, decreasing binding of a nucleating protein to an anchor sequence, and/or disrupting or preventing an anchor sequence-mediated conjunction), or may be altered to decrease its methylation (e.g., increasing interaction of a transcription factor with a portion of a target gene or transcription control element, increasing binding of a nucleating protein to an anchor sequence, and/or promoting or increasing strength of an anchor sequence-mediated conjunction).

### Kits

The present disclosure further directed, in part, to a kit comprising an expression repressor or an expression repression system, e.g., expression repressor(s), described herein. In some embodiments, a kit comprises an expression repressor or an expression repression system (e.g., the expression repressor(s) of the expression repression system) and instructions for the use of said expression repressor or expression repression system. In some embodiments, a kit comprises a nucleic acid encoding the expression repressor or the expression repression system or a component thereof (e.g., the expression repressor(s) of the expression repression system) and instructions for the use of said nucleic acid and/or said expression repressor or the expression repression system. In some embodiments, a kit comprises a cell comprising a nucleic acid encoding the expression repressor or the expression repression system or a component thereof (e.g., the expression repressor(s) of the expression repression system) and instructions for the use of said cell, nucleic acid, and/or said expression repression system.

In some embodiments, a kit comprises a unit dosage of an expression repressor or an expression repression system, e.g., expression repressor(s), described herein, or a unit dosage of a nucleic acid, e.g., a vector, encoding an expression repressor or an expression repression system, e.g., expression repressor(s), described herein.

In some embodiments the kit further comprises b) a set of instructions comprising at least one method for treating a disease or modulating, e.g., decreasing the expression of target gene within a cell with said composition. In some embodiments, the kits can optionally include a delivery vehicle for said composition (e.g., a lipid nanoparticle). The reagents may be provided suspended in the excipient and/or delivery vehicle or may be provided as a separate component which can be later combined with the excipient and/or delivery vehicle. In some embodiments, the kits may optionally contain additional therapeutics to be co-administered with the compositions to affect the desired target gene expression. While the instructional materials typically comprise written or printed materials, they are not limited to such. Any medium capable of storing such instructions and communicating them to an end user is contemplated. Such media include but are not limited to electronic storage media (e.g., magnetic discs, tapes, cartridges, chips), optical media (e.g., CD ROM), and the like. Such media may include addresses to internet sites that provide such instructional materials.

In some embodiments, a kit comprises a unit dosage of an expression repressor an expression repression system, e.g., expression repressor(s), described herein, or a unit dosage of a nucleic acid, e.g., a vector, encoding an expression repression system, e.g., expression repressor(s), described herein.

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### EXAMPLES

### Example 1: Target Selection and Guide Design

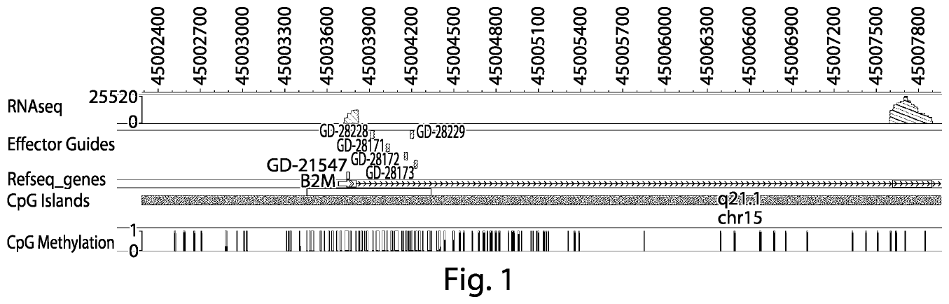

The β-2-microglobulin (β2M) gene was targeted for down-regulation using an expression repression system of the disclosure. A first expression repressor comprising an S. pyogenes dCas9 and a KRAB domain (Sp-dCas9-KRAB, corresponding to SEQ ID NOs: 91-93) and a second expression repressor comprising S. aureus dCas9 and MQ1 (Sa-dCas9-MQ1, corresponding to SEQ ID Nos: 94-96) were used. Guide RNAs (comprising target-binding sequences corresponding to SEQ ID NOs: 1-5) targeted each expression repressor to an area of CpG hypomethylation in the promoter region of the β2M gene (FIG. 1), where methylation runs from 0 to 1 (0 to 100%) and where higher methylation is depicted by red bars. Each bar represents a CpG site. Blue guides (GD-28228 and GD-28229) were used to target Sp-dCas9-KRAB and orange guides (GD-28171, GD-28172 and GD-28173) were used to target Sa-dCas9-MQ1 to the CpG Island in the β2M promoter region.

### Example 2: β2M Expression Repression Using One or Two Expression Repressors

The guide RNAs of Example 1 were used to target the Sp-dCas9-KRAB expression repressor, the Sa-dCas9-MQ1 expression repressor, or both to the promoter region of β2M and the effects on β2M expression monitored by qPCR. mRNA encoding the expression repressor(s) and guide RNA(s) were delivered to cells in lipid nanoparticles (LNPs).

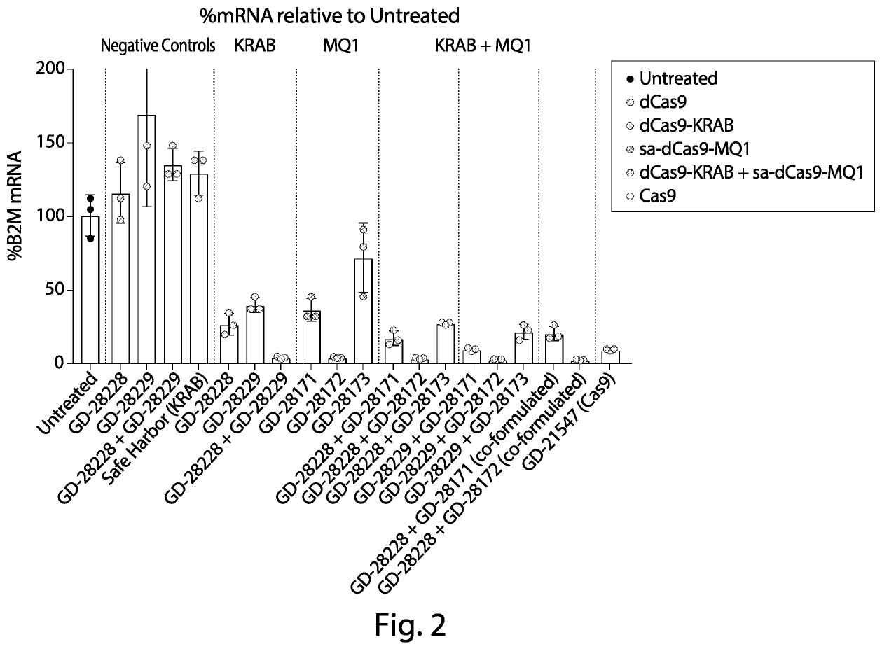

HepG2 cells were seeded in a 96 well plate at a density of 30,000 cells/well in 100 μL RPMI (+10% FBS, +Penn/Strep). Cells were allowed to attach for 24 hours prior to treatment with 0.125 ug RNA (1:1 mRNA: sgRNA by weight) per guide. RNA was delivered by LNP formulated with COATSOME SS-OP ionizable lipid (NOF America). LNPs were formulated with one mRNA and one guide, with the exception of the samples noted as “co-formulated” which were prepared with two guides, maintaining the same mRNA: guide weight ratio. Cells were incubated with the LNPs for 72 hours, following which the cells were harvested for β2M mRNA analysis by qPCR (FIG. 2), with β2M mRNA levels given relative to untreated control cells.

dCas9 alone targeted to the β2M promoter did not decrease β2M mRNA levels. The data shows that Sp-dCas9-KRAB or Sa-dCas9-MQ1 alone decreased β2M mRNA levels, with some variation in the decrease depending on the guide RNA(s) used. Sp-dCas9-KRAB and Sa-dCas9-MQ1 together decreased β2M mRNA levels on average more than either expression repressor alone, again with some variation in the decrease depending on the guide RNA(s) used. Use of an expression repression system comprising one or two expression repressors decreased expression of a target gene The expression repression system comprising two expression repressors appeared to decrease expression of the target gene more than a single expression repressor.

### Example 3: β2M Expression Repression Across Doses and Using Flow Cytometry

Guide RNA of Example 1 (GD-28228 for Sp-dCas9 and Sp-dCas9-KRAB; GD-28172 for Sa-dCas9-MQ1; or GD-28228 and GD-28172 for Sp-dCas9-KRAB and Sa-dCas9-MQ1) was used to target the Sp-dCas9-KRAB expression repressor, the Sa-dCas9-MQ1 expression repressor, or both to the promoter region of β2M and the effects on β2M expression monitored by qPCR and also by flow cytometry (using a fluorescent β2M to monitor β2M protein levels). mRNA encoding the expression repressor(s) and guide RNA(s) were delivered to cells in LNPs at different dosages and formulations.

HepG2 cells were seeded in a 12-well plate at a density of 330,000 cells/well 1.5 mL RPMI (+10% FBS, +Penn/Strep). Cells were allowed to attach for 24 hours prior to treatment with 1.25 ug or 2.5 ug RNA (1:1 mRNA: sgRNA by weight) per guide in 1 mL media. RNA was delivered by LNP formulated with COATSOME SS-OP ionizable lipid (NOF America). LNPs were formulated with one mRNA and one guide, with the exception of the samples noted as “co-formulated” which were prepared with two guides, maintaining the same mRNA: guide weight ratio. Cells were incubated with the LNPs for 72 hours, following which the cells were harvested for FACS and mRNA analysis by qPCR.

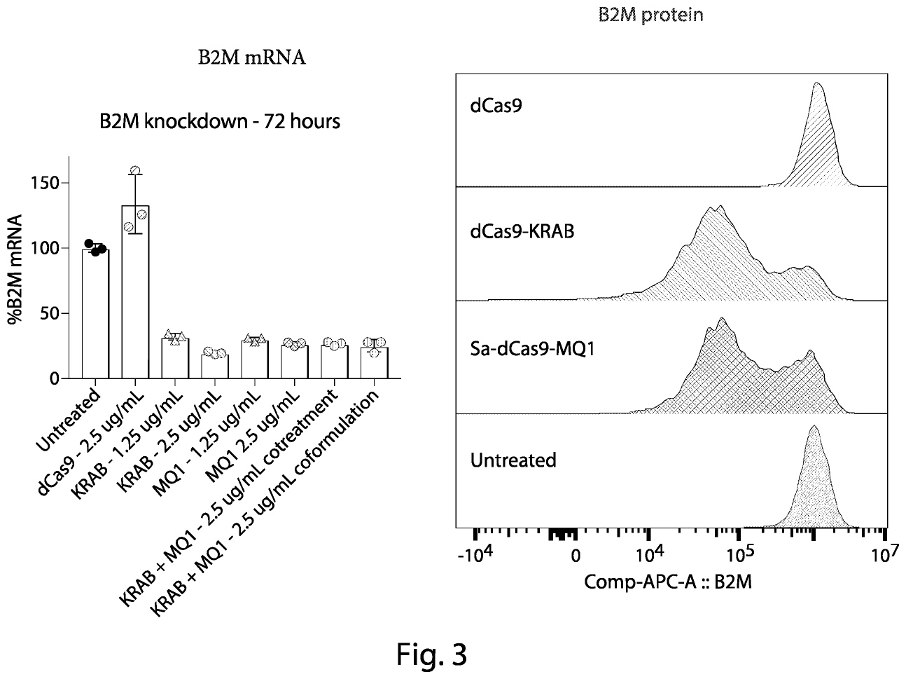

As seen in Example 2, dCas9 alone targeted to the β2M promoter did not decrease β2M mRNA levels. Sp-dCas9-KRAB or Sa-dCas9-MQ1 alone, and Sp-dCas9-KRAB and Sa-dCas9-MQ1 together decreased β2M mRNA levels (FIG. 3). Both 1.25 ug/ml and 2.5 ug/ml doses of the expression repressors (alone or together) decreased β2M mRNA levels, with the higher dose of Sp-dCas9-KRAB appearing to give a greater decrease than the lower dose. FACS monitoring of β2M protein levels showed that Sp-dCas9-KRAB or Sa-dCas9-MQ1 treated cells showed a decrease in the population of cells having the untreated level of β2M protein and the appearance of a new population of cells with a decreased level of β2M protein. These data are consistent with those of Example 2, showing that use of an expression repression system comprising one or two expression repressors decreases expression of a target gene, and that individual expression repressors decrease the levels of a protein encoded by the target gene too.

### Example 4: β2M Expression Repression Over Time

Guide RNA (GD-28228 for Sp-dCas9 and Sp-dCas9-KRAB; GD-28172 for Sa-dCas9-MQ1; or GD-28228 and GD-28172 for Sp-dCas9-KRAB and Sa-dCas9-MQ1) of Example 1 was used to target the Sp-dCas9-KRAB expression repressor, the Sa-dCas9-MQ1 expression repressor, or both to the promoter region of β2M and the effects on β2M expression monitored by qPCR. Cells were collected at different days after transfection to extract total RNA and probe for expression changes.

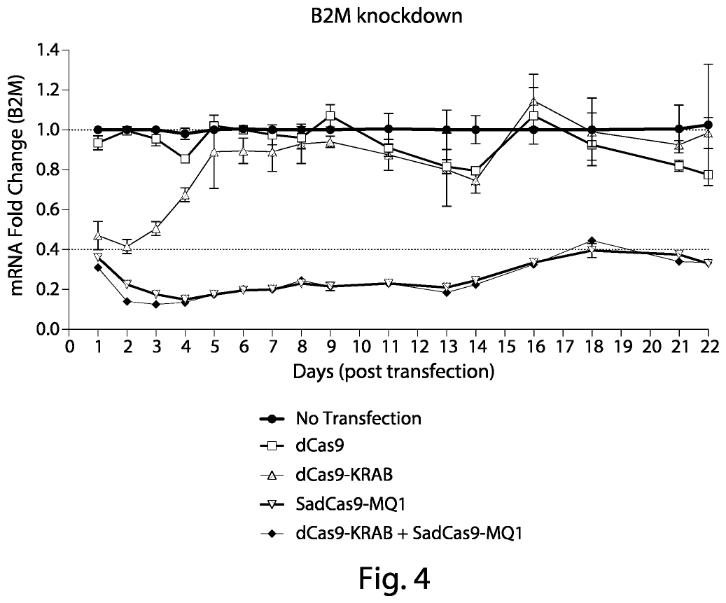

K562 cells were plated in a 24-well plate and transfected with MC3-based LNP formulation enclosing: dCas9 mRNA with β2M targeting guide RNA, Sp-dCas9-KRAB mRNA with β2M targeting guide RNA, Sa-dCas9-MQ1 mRNA with β2M targeting guide RNA, or a mixture of Sp-dCas9-KRAB and Sa-dCas9-MQ1 mRNAs with β2M targeting guide RNAs. Cells were collected at different days after transfection to extract total RNA and probe for expression changes in β2M mRNA (FIG. 4). Dotted lines indicate average untreated β2M mRNA levels over time (top) and average knockdown levels of β2M mRNA 24 hours post transfection (bottom).

Over a 22-day period, non-transfected (blue line) and dCas9 transfected (red line) cells don't change β2M expression, whereas Sp-dCas9-KRAB (green line) transfected cells have lower β2M mRNA levels in the initial days post-transfection, with maximum effect on day 2, but the repression is lost after day 5. Sa-dCas9-MQ1 (purple line) transfected cells show significant repression (up to 85% by day 4) of $2M mRNA levels and the repression is retained and then slowly the repression decreases somewhat as seen by the line remaining just under the dotted line in the figure at the end of the test period. Sp-dCas9-KRAB and Sa-dCas9-MQ1 combined transfection (orange line) did not give any added repression than what is already observed for Sa-dCas9-MQ1 alone (purple line).

These data show that use of an expression repression system comprising one or two expression repressors decreases expression of a target gene. Individual expression repressors such as those comprising MQ1 can repress expression of a target gene for an extended duration, and expression repression systems comprising more than one expression repressor, e.g., one of which is an expression repressor comprising MQ1, can repress expression of a target gene for an extended duration as well.

### Example 5: Durable Repression of B2M Expression after Promoter Methylation by DNA Methyltransferases

This example demonstrates methylation of B2M promoter region by treatment with LNPs containing various DNMTs and LNPs containing a guide targeting the dCas9 fusions to the B2M promoter region cause durable repression of B2M expression.

K-562 cells were seeded in 24 well plates at a density of 200,000 cells per well in 750 μL of growth medium (RPMI+10% FBS+pen/strep). mRNAs encoding effectors, dCas9-MQ1 (SEQ ID NO: 33), dCas9-DNMT3a/3L (m) (“m” denoting that the 3a region has the human sequence but the 3L region has the mouse sequence) (SEQ ID NO:36), dCas9-DNMT3a/3L (h (“h” denoting that both the 3a region and the 3L region have the human sequences) (SEQ ID NO:35), dCas9-DNMT1 (SEQ ID NO: 35), and dCas9-DNMT3B were formulated in MC3 lipids using a Precision NanoSystems NanoAssemblr Spark instrument. Single guide RNAs (sgRNAs) of SEQ ID NO: 21 were formulated in the same lipids in the same instrument in separate formulations. Stock LNP concentrations were 100 μg/mL. Cells were treated with LNPs encapsulating mRNAs and LNPs encapsulating sgRNAs in a 1:1 ratio at a final lipid concentration of 1.25 μg/mL and incubated thereafter. Each effector and guide combination were transfected in quadruplicate. At different timepoints after transfection, 200 μL of cells were removed and processed to extract total RNA and probe for expression changes and 200 μL of cells were moved into 550 μL of fresh media. This procedure continued until 18 days after the initial transfection. RNA was extracted using the RNeasy 96 Plus kits (Qiagen), converted to cDNA using LunaScript RT SuperMix (New England Biolabs) and used for RT-qPCR using a B2M specific TaqMan primer/probe set Assay with and the TaqMan™ Fast Advanced Master Mix (Thermo Fisher Scientific). Data is presented as fold change compared to un-transfected control using the ddCT method.

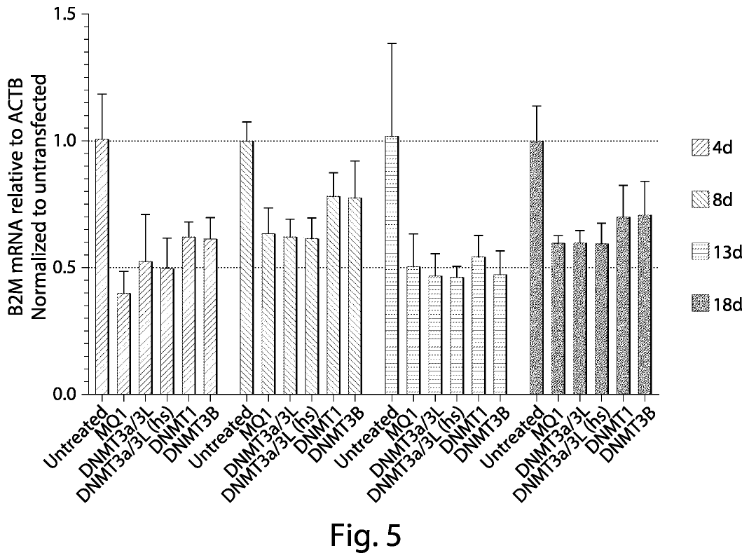

Results show, all the constructs described in this Example were able to repress B2M expression ranging from about 35% to 60%, 4 days after treatment relative to an untreated sample (FIG. 5). All the constructs were able to repress B2M expression ranging from about 15% to 30% at the end of 8 days relative to an untreated sample. At the end of 13 days, all effectors were able to repress B2M expression by about 45-55%, and at the end of 18 days, all effectors were able to repress B2M expression by about 30-40% relative to an untreated sample (FIG. 5).

### Example 6: Identification of Durable Repressors or Repressor Systems that Downregulate MYC Expression by Targeting a CTCF Site Located Upstream of MYC Gene in K-562 Cells

This example demonstrates identification of individual expression repressors or expression repression systems that downregulate MYC expression by targeting the CTCF site located upstream of MYC gene.

K-562 cells were grown in 96 well plates at a density of 1×10⁶ cell/mL in 100 μL of growth medium (RPMI+10% FBS+pen/strep). A sgRNA pool was prepared by mixing GD-29639 (SEQ ID NO: 11) and GD-29640 (SEQ ID NO: 12) in 1:1 ratio. A repression master plate was prepared by adding 5 μl of mRNAs encoding effectors or effector combinations from Table 4 in each well. After addition, 17.5 ng of each mRNA or combination of mRNAs were present in each well. After plating the master plates were stored frozen until the time of transfection.

TABLE 4
List of Expression repressors or expression
repression systems tested

| | Expression repressors or expression repression systems |
|---|---|
| 1 | No effector (Untreated) |
| 2 | dCas9 |
| 3 | dCas9-DNMT1 |
| 4 | dCas9-DNMT3B |
| 5 | G9A-dCas9 |
| 6 | dCas9-MQ1 |
| 7 | dCas9-HDAC8 |
| 8 | dCas9-DNMT3a/3L (h) |
| 9 | dCas9-DNMT3a/3L (m) |
| 10 | EZH2-dCas9 |
| 11 | dCas9-LSD1 |
| 12 | dCas9-KRAB |
| 13 | EZH2-dCas9-KRAB |
| 14 | G9A-dCas9-KRAB |
| 15 | FOG1-dCas9- FOG1 |
| 16 | dCas9-KRAB + FOG1-dCas9- FOG1 |
| 17 | EZH2-dCas9-KRAB + dCas9-HDAC8 |
| 18 | dCas9-LSD1 + dCas9-HDAC8 |
| 19 | G9A-dCas9-KRAB + EZH2-dCas9-KRAB |
| 20 | G9A-dCas9 + dCas9-HDAC8 |
| 21 | EZH2-dCas9 + dCas9-HDAC8 |
| 22 | dCas9-LSD1 + G9A-dCas9-KRAB |
| 23 | dCas9-LSD1 + EZH2-dCas9-KRAB |
| 24 | dCas9-LSD1 + EZH2-dCas9 |
| 25 | G9A-dCas9-KRAB + dCas9-HDAC8 |
| 26 | G9A-dCas9 + EZH2-dCas9 |
| 27 | dCas9-MQ1 + EZH2-dCas9-KRAB |
| 28 | dCas9-LSD1 + G9A-dCas9 |
| 29 | dCas9-MQ1 + dCas9-HDAC8 |
| 30 | dCas9-MQ1 + G9A-dCas9-KRAB |

Prior to transfection, 5 μl of sgRNA pool were added in each well so that 8.75 ng of each sgRNA were present in each well. The effector mRNA-sgRNA mix were diluted with Opti-MEM media (Thermo Fisher) to a total volume of 40 μl in each well. A transfection master mix of Lipofectamine™ MessengarMAX™ (Thermo Fisher) and Opti-MEM were prepared using a ratio of 0.3 μL of Lipofectamine™ MessengarMAX™ per 100 ng of total RNA as described in Table 5. Immediately before transfection, 35 μL of transfection master mix were added to each well containing RNA to bring the final volume to 75 μl and were incubated for 5 minutes at room temperature and was used to transfect K-562 cells. After transfection the cells were incubated at 37° C. with 5% CO₂ in standard incubator.

TABLE 5

| | | Per 96-well | Per 2, 96-well | Per 4, 96-well |
|---|---|---|---|---|
| Reagent | Per well | plate | plates | plates |
| MessengerMax | 4.2 | 189 | 378 | 756 |
| (μL) | | | | |
| Optimem (μL) | 30.8 | 1386 | 2772 | 5544 |

24 hours after transfection, 100 μL of fresh media was added to each well. Sample collection started 48 hours post transfection and continued until 6 days. Cell suspension samples were collected at 48 hours, 72 hours, and 144-hour time point. Each time samples were removed for analysis equivalent volumes of fresh media was added and the incubations were continued. RNA was extracted using the Rneasy® Plus 96 kits (Qiagen), converted to cDNA using LunaScript® RT SuperMix (New England Biolabs) and used for RT-qPCR using a MYC specific Taqman primer/probe set assay with the Tagman™ Fast Advanced Master Mix (Thermo Scientific). Data was presented as fold change compared to dCas9 treated control using the ddCT method. The untreated and dCas9 samples were used as calibrators.

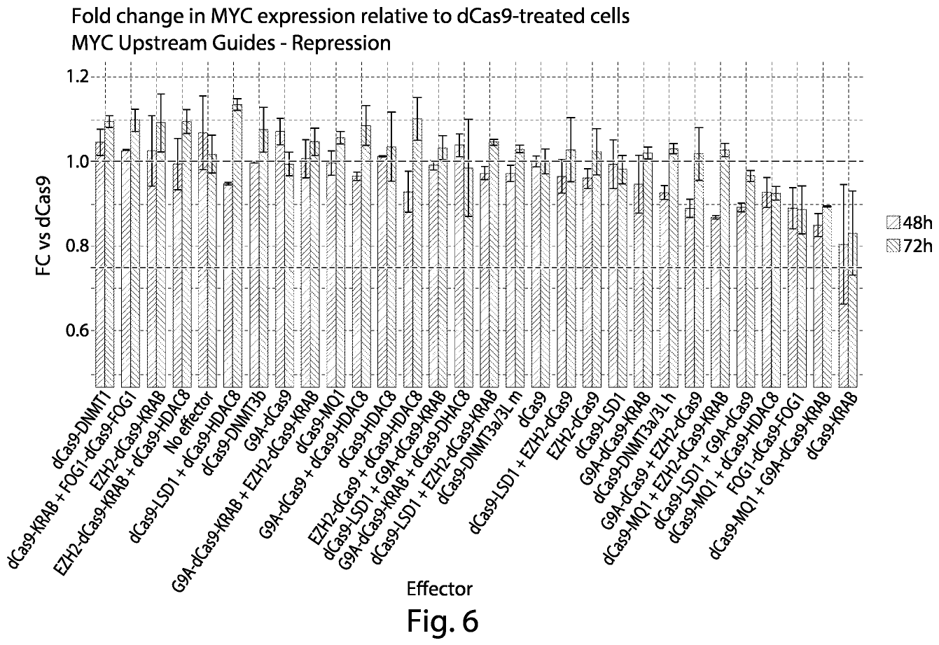

The experiment showed that, at least, cells treated with dCas9-DNMT3a/3L (h), dCas9-KRAB, FOG1-dCas9-FOG1, G9A-dCas9+EZH2-dCas9, dCas9-MQ1+EZH2-dCas9-KRAB, dCas9-LSD1+G9A-dCas9, dCas9-MQ1+dCas9-HDAC8, and dCas9-MQ1+G9A-dCas9-KRAB showed repression of MYC expression at 48 hour and/or 72-hour time point (FIG. 6).

### Example 7: Identification of Durable Repressors or Repression Systems that Downregulate β2M Expression by Targeting a Region Upstream of the β2M Transcription Start Site in K-562 Cells

This example demonstrates identification of individual expression repressors or expression repression systems that downregulate B2M expression by targeting the upstream region of β2M promoter.

K-562 cells were grown in 96 well plates at a density of 1×10⁶ cell/mL in 100 μL of growth medium (RPMI+10% FBS+pen/strep). A sgRNA pool was prepared by mixing GD-27634 (SEQ ID NO: 13) and GD-29500 (SEQ ID NO: 14) in 1:1 ratio. The effectors as described in Table X and sgRNA pool mix was used to transfect the cells following the protocol described in Example 6. Cell suspension samples were collected at 48 hours, 72 hours, 144-hour, 192 hours, and 240 hour time point and were processed using the protocol described in Example 6. Data was presented as fold change compared to dCas9 treated control using the ddCT method. The untreated and dCas9 samples were used as calibrators.

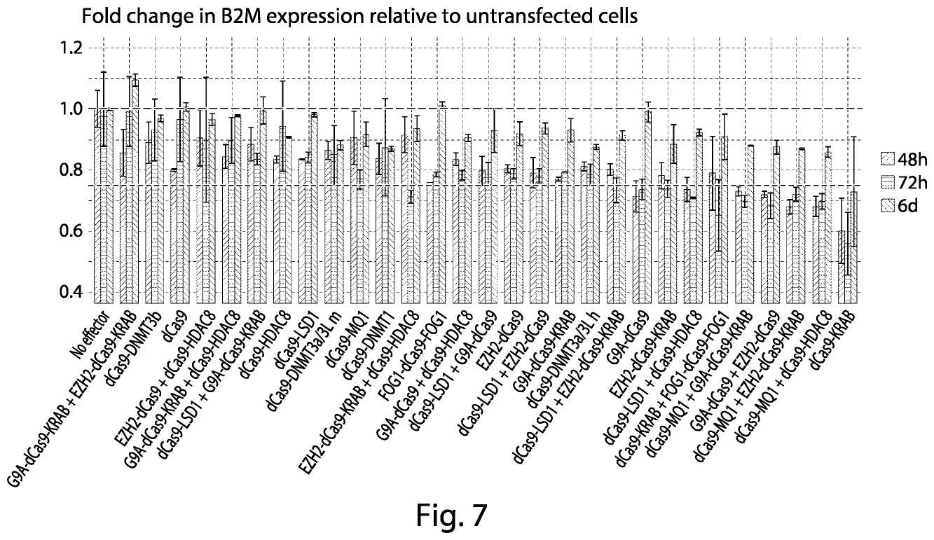

The data showed that, at least, cells treated with dCas9-HDAC8, dCas9-LSD1, dCas9-MQ1, FOG1-dCas9-FOG1, G9A-dCas9-KRAB, dCas9-DNMT3a/3L (h), G9A-dCas9, EZH2-dCas9-KRAB, dCas9-KRAB, EZH2-dCas9-KRAB+dCas9-HDAC8, dCas9-LSD1+EZH2-dCas9-KRAB, dCas9-LSD1+dCas9-HDAC8, dCas9-KRAB+FOG1-dCas9-FOG1, dCas9-MQ1+G9A-dCas9-KRAB, G9A-dCas9+EZH2-dCas9, dCas9-MQ1+EZH2-dCas9-KRAB, and dCas9-MQ1+dCas9-HDAC8 showed repression of β2M expression at 48 hour, 72 hour and/or 144 hour time point (FIG. 7).

### Example 8: Identification of Durable Repressors or Repression Systems that Downregulate HSPA1B Expression by Targeting a Region Downstream of the HSPA1B Transcription Start Site in K-562 Cells

This example demonstrates identification of individual expression repressors or expression repression systems that downregulate HSPA1B expression by targeting the downstream region of HSPA1B promoter.

K-562 cells were grown in 96 well plates at a density of 1×10⁶ cell/mL in 100 μL of growth medium (RPMI+10% FBS+pen/strep). A sgRNA pool was prepared by mixing GD-29542 (SEQ ID NO: 17) and GD-29544 (SEQ ID NO: 18) in 1:1 ratio. The effectors as described in Table 4 and sgRNA pool mix was used to transfect the cells following the protocol described in Example 6. Cell suspension samples were collected at 48 hours, 72 hours, and 144-hour time point and were processed using the protocol described in Example 6. Data was presented as fold change compared to dCas9 treated control using the ddCT method. The untreated and dCas9 samples were used as calibrators.

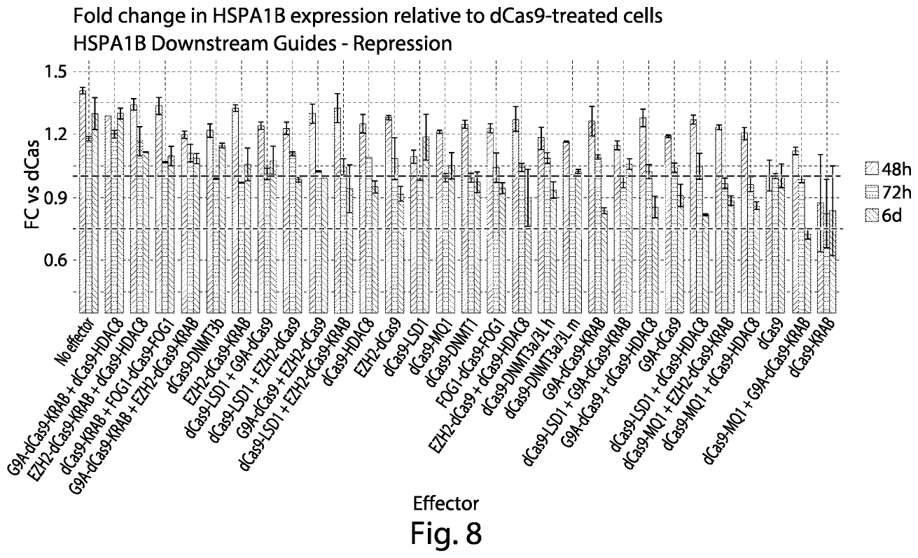

The data showed that, at least, cells treated with dCas9-HDAC8, EZH2-dCas9, dCas9-MQ1, dCas9-DNMT1, dCas9-DNMT3a/3L (h), dCas9-DNMT3a/3L (m), G9A-dCas9-KRAB, FOG1-dCas9-FOG1, G9A-dCas9, dCas9-KRAB, G9A-dCas9+dCas9-HDAC8, dCas9-LSD1+G9A-dCas9-KRAB, dCas9-LSD1+EZH2-dCas9, G9A-dCas9+EZH2-dCas9, dCas9-LSD1+EZH2-dCas9-KRAB, EZH2-dCas9+dCas9-HDAC8, dCas9-LSD1+dCas9-HDAC8, dCas9-MQ1+EZH2-dCas9-KRAB, dCas9-MQ1+dCas9-HDAC8, G9A-dCas9-KRAB+EZH2-dCas9-KRAB, dCas9-LSD1+G9A-dCas9, and dCas9-MQ1+G9A-dCas9-KRAB showed repression of HSPA1B expression at 48 hour, 72 hour and/or 144 hour time point (FIG. 8).

### Example 9: Identification of Durable Repressors or Repression Systems that Downregulate GATA1 Expression in K-562 Cells

This example demonstrates identification of individual expression repressors or expression repression systems that downregulate GATA1 expression in K-562 cells.

K-562 cells were grown in 96 well plates at a density of 1×10⁶ cell/mL in 100 μL of growth medium (RPMI+10% FBS+pen/strep). A sgRNA pool was prepared by mixing GD-29536 (SEQ ID NO: 19) and GD-29693 (SEQ ID NO: 20) in 1:1 ratio. The effectors as described in Table 4 and sgRNA pool mix was used to transfect the cells following the protocol described in Example 6. Cell suspension samples were collected at 48 hours, 72 hours, and 144-hour time point and were processed using the protocol described in Example 6. Data was presented as fold change compared to dCas9 treated control using the ddCT method. The untreated and dCas9 samples were used as calibrators.

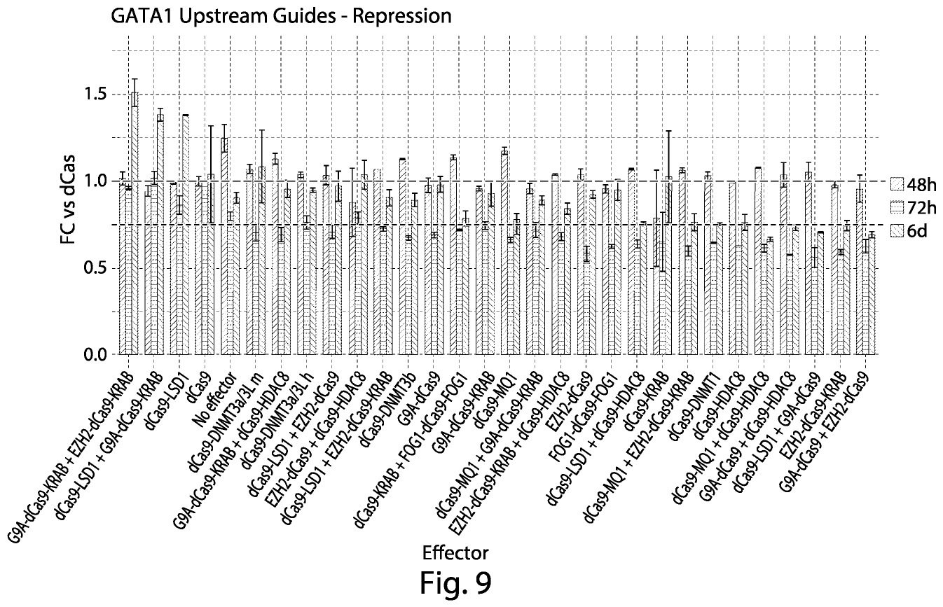

The data showed that, at least, cells treated with dCas9-HDAC8, dCas9-LSD1+dCas9-HDAC8, dCas9-MQ1+dCas9-HDAC8, and G9A-dCas9+dCas9-HDAC8 showed repression of GATA1 expression at 48-hour, 72 hour and/or 144 hour time point (FIG. 9).

### EQUIVALENTS

It is to be understood that while the invention has been described in conjunction with the detailed description thereof, the foregoing description is intended to illustrate and not limit the scope of the invention, which is defined by the scope of the appended claims. Some aspects, advantages, and modifications are within the scope of the following claims.

We claim:

1. An expression repression system comprising:
a first expression repressor comprising a first DNA-targeting moiety and a first repressor domain, and
a second expression repressor comprising a second DNA-targeting moiety and a second repressor domain,
wherein the first DNA-targeting moiety specifically binds a first DNA sequence, and the second DNA-targeting moiety specifically binds a second DNA sequence different from the first DNA sequence, and
wherein the first repressor domain is different from the second repressor domain.

2. An expression repression system comprising:
a first expression repressor comprising a first DNA-targeting moiety and a first repressor domain, and
a second expression repressor comprising a second DNA-targeting moiety and a second repressor domain,
wherein the first or second repressor domain comprises an MQ1 domain or functional variant or fragment thereof,
wherein the first repressor domain is different from the second repressor domain.

3. The expression repression system of claim 2, wherein:
the first DNA-targeting moiety specifically binds a first DNA sequence, and the second DNA-targeting moiety specifically binds a second DNA sequence different from the first DNA sequence; or
the first DNA-targeting moiety and the second DNA-targeting moiety specifically bind the same DNA sequence.

4. The expression repression system of any of claims 1-3,
wherein the first DNA-targeting moiety comprises a first CRISPR/Cas molecule comprising a first CRISPR/Cas protein and first guide RNA, and the second DNA-targeting moiety comprises a second CRISPR/Cas molecule comprising a second CRISPR/Cas protein and a second guide RNA.

5. The expression repression system of any of claims 1-4:
wherein the first CRISPR/Cas protein comprises a different amino acid sequence than the second CRISPR/Cas protein.

6. The expression repression system of any of claims 1-5, wherein the first or second DNA-targeting moiety comprises a CRISPR/Cas molecule comprising a Cas protein or Cpf1 protein chosen from Table 1 or a variant (e.g., mutant) of any thereof.

7. The expression repression system of any of claims 1-6, wherein the first DNA-targeting moiety comprises a first CRISPR/Cas molecule comprising a Cas protein or Cpf1 protein chosen from Table 1 or a variant (e.g., mutant) of any thereof, and the second DNA-targeting moiety comprises a second CRISPR/Cas molecule comprising a different Cas protein or Cpf1 protein chosen from Table 1 or a variant (e.g., mutant) of any thereof.

8. The expression repression system of any of claims 1-7, wherein the first or second repressor domain comprises a histone methyltransferase activity (e.g., a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof), a histone demethylase activity (e.g., a protein chosen from KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof), a histone deacetylase activity (e.g., a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof), a DNA methyltransferase activity (e.g., a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof), or a transcription repressor activity (e.g., a protein chosen from KRAB, MeCP2, HP1, RBBP4, REST, FOG1, SUZ12, or a functional variant or fragment of any thereof).

9. The expression repression system of any of claims 1-8, wherein:
the first repressor domain comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof, and
the second repressor domain comprises a different protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, N066, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, MQ1, DNMT1, DNMT3A1, DNMT3A2, DNMT3B1, DNMT3B2, DNMT3B3, DNMT3B4, DNMT3B5, DNMT3B6, DNMT3L, KRAB, MeCP2, HP1, RBBP4, REST, FOG1, SUZ12, or a functional variant or fragment thereof.

10. The expression repression system of any of claims 1-9, wherein the first repressor domain comprises KRAB, and the second repressor domain comprises DNMT3A (e.g., human DNMT3A).

11. The expression repression system of any of claims 1-9, wherein the first repressor domain comprises KRAB, and the second repressor domain comprises bacterial MQ1.

12. The expression repression system of any of claims 1-11, wherein the first or second DNA-targeting moiety is selected from a TAL effector molecule, a CRISPR/Cas molecule, a zinc finger domain, a tetR domain, a meganuclease, or an oligonucleotide.

13. The expression repression system of any of claims 1-12, wherein the first or second DNA-targeting moiety is or comprises a CRISPR/Cas molecule comprising a CRISPR/Cas protein (e.g., that is or comprises a Cas protein or Cpf1 protein chosen from Table 1 or a variant (e.g., mutant) of any thereof) and a guide RNA.

14. The expression repression system of any of claims 1-13, wherein:
the first DNA-targeting moiety is or comprises a CRISPR/Cas molecule comprising a CRISPR/Cas protein that is or comprises a Cas protein or Cpf1 protein chosen from Table 1 or a variant (e.g., mutant) of any thereof), and
the second DNA-targeting moiety is or comprises a CRISPR/Cas molecule comprising a different CRISPR/Cas protein that is or comprises a Cas protein or Cpf1 protein chosen from Table 1 or a variant (e.g., mutant) of any thereof).

15. The expression repression system of any of claims 1-14, wherein the first or the second DNA-targeting moiety specifically binds to:
a promoter sequence or to DNA proximal to a promoter sequence, e.g., a promoter sequence operably linked to a target gene;
an enhancer sequence or to DNA proximal to an enhancer sequence, e.g., an enhancer sequence that affects expression of and/or is operably linked to a target gene;
a target gene, e.g., sequence comprised between the transcription start site and stop codon of a target gene;
the transcription start site of a target gene;
an intron, e.g., a splice site associated with an intron, of a target gene; or
an exon of a target gene.

16. The expression repression system of any of claims 1-15, wherein the first DNA sequence is a first site within or proximal to a promoter operably linked to a target gene and the second DNA sequence is a second site within or proximal to the promoter operably linked to the target gene.

17. The expression repression system of any of claims 1-16, wherein the distance between the first DNA sequence and the second DNA sequence is at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 base pairs or any size therebetween, e.g., 20-500 base pairs.

18. The expression repression system of any of claims 1-17, wherein the distance between the first DNA sequence and the second DNA sequence is no more than 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 base pairs or any size therebetween, e.g., 20-500 base pairs.

19. The expression repression system of any of claims 1-18, wherein the target gene is 3-2-microglobulin, MYC, HSPA1B, GATA1, APOB, FOXP3, CXCL1, CXCL2, CXCL3, CXCL4, CXCL5, CXCL6, CXCL6, CXCL7, and/or CXCL8.

20. The expression repression system of any of claims 1-19, wherein the first DNA-targeting moiety does not appreciably bind to the second DNA sequence and the second DNA-targeting moiety does not appreciably bind to the first DNA sequence, e.g., wherein the first DNA-targeting moiety binds the second DNA sequence with a K_{D} of at least 500, 600, 700, 800, 900, 1000, 2000, 5000, 10,000, or 100,000 nM, and the second DNA-targeting moiety binds the first DNA sequence with binds with a K_{D} of at least 500, 600, 700, 800, 900, 1000, 2000, 5000, 10,000, or 100,000 nM.

21. The expression repression system of any of claims 1-20, wherein binding of the first expression repressor to the first DNA sequence and/or binding of the second expression repressor to the second DNA sequence decreases expression of a target gene, e.g., a target gene operably linked to the first and/or second DNA sequence, in a cell, e.g., by 10, 20, 30, 40, 50, 60, 70, 80, 90, or 100% compared to expression in the absence of the first and/or second expression repressor.

22. The expression repression system of any of claims 1-21, wherein binding of the first and/or second expression repressor to the first and/or second DNA sequence appreciably decreases expression of a target gene, e.g., a target gene operably linked to the first and/or second DNA sequence, for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in Examples 2-4.

23. The expression repression system of either of claims 21 or 22, wherein expression is decreased by 10, 20, 30, 40, 50, 60, 70, 80, 90, or 100% compared to expression in the absence of the first and second expression repressors, e.g., as measured by ELISA or as described in Examples 2-4.

24. The expression repression system of any of claims 21-23, wherein binding of the first expression repressor to the first DNA sequence and the second expression repressor to the second DNA sequence appreciably decreases expression of a target gene, e.g., a target gene operably linked to the first DNA sequence, for a time period of at least 1, 2, 3, 4, 5, 6, 7, 8, 9, 10, 11, or 12 hours, or at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in Examples 2-4.

25. The expression repression system of any of claims 21-24, wherein the decrease in expression resulting from the binding of the first expression repressor to the first DNA sequence and the second expression repressor to the second DNA sequence is greater than the decrease in expression resulting from the binding of the first expression repressor to the first DNA sequence or the binding of the second expression repressor to the second DNA sequence individually.

26. The expression repression system of claim 25, wherein the binding of the first expression repressor to the first DNA sequence and the second expression repressor to the second DNA sequence decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× more than either the binding of the first expression repressor to the first DNA sequence or the binding of the second expression repressor to the second DNA sequence individually, e.g., as measured by ELISA or as described in Examples 2-4.

27. The expression repression system of any of claim 21-26, wherein the decrease in expression resulting from the binding of the first expression repressor to the first DNA sequence and the second expression repressor to the second DNA sequence persists for a longer time (e.g., more hours, days, or cell divisions) than the decrease in expression resulting from the binding of the first expression repressor to the first DNA sequence or the binding of the second expression repressor to the second DNA sequence individually.

28. The expression repression system of claim 27, wherein the binding of the first expression repressor to the first DNA sequence and the second expression repressor to the second DNA sequence decreases expression 1.05× (i.e., 1.05 times), 1.1×, 1.15×, 1.2×, 1.25×, 1.3×, 1.35×, 1.4×, 1.45×, 1.5×, 1.6×, 1.7×, 1.8×, 1.9×, 2×, 3×, 4×, 5×, 6×, 7×, 8×, 9×, 10×, 20×, 50×, or 100× longer (e.g., as measured in hours, days, or cell divisions) than either the binding of the first expression repressor to the first DNA sequence or the binding of the second expression repressor to the second DNA sequence individually, e.g., as measured by ELISA or as described in Examples 2-4.

29. The expression repression system of any of claims 1-28, wherein
the first expression repressor comprises a first DNA-targeting moiety comprising a CRISPR/Cas molecule comprising an S. pyogenes CRISPR/Cas protein selected from Table 1 or variant (e.g., mutant) thereof, e.g., an S. pyogenes dCas9, and a first repressor domain comprising KRAB or a functional variant or fragment thereof; and
the second expression repressor comprises a second DNA-targeting moiety comprising a CRISPR/Cas molecule comprising a S. aureus CRISPR/Cas protein selected from Table 1 or variant (e.g., mutant) thereof, e.g., an S. aureus dCas9, and a second repressor domain comprising bacterial MQ1 or a functional variant or fragment thereof.

30. A polypeptide comprising:
a DNA-targeting moiety; and
a repressor domain comprising bacterial MQ1 or a functional variant or fragment thereof.

31. A nucleic acid encoding:
the first expression repressor,
the second expression repressor,
the expression repression system (e.g., the first expression repressor and the second expression repressor), or the polypeptide of any of claims 1-30.

32. A vector comprising the nucleic acid of claim 31.

33. A cell comprising the expression repression system, nucleic acid, vector, or polypeptide of any of claims 1-32.

34. A pharmaceutical composition comprising the expression repression system, nucleic acid, polypeptide, or vector of any of claims 1-32, and at least one pharmaceutically acceptable excipient or carrier.

35. A method of decreasing expression of a target genomic sequence in a cell, comprising:
providing the expression repression system, nucleic acid, polypeptide, pharmaceutical composition, or vector of any of claims 1-32 or 34; and
contacting the cell with the expression repression system, nucleic acid, polypeptide, pharmaceutical composition, or vector,
thereby decreasing expression of the target genomic sequence.

36. A method of epigenetically modifying a target genomic sequence in a cell, comprising:
providing the expression repression system, nucleic acid, polypeptide, pharmaceutical composition, or vector of any of claims 1-32 or 34; and
contacting the cell with the expression repression system, nucleic acid, vector, polypeptide, or pharmaceutical composition,
thereby epigenetically modifying the target genomic sequence.

37. The method of either of claims 35 or 36, wherein the target genomic sequence is:
a target gene (e.g., a site within a target gene, e.g., an exon, intron, or splice site), e.g., the gene encoding β-2-microglobulin (β2M),
a transcription control element operably linked to a target gene, or
an anchor sequence proximal to a target gene or associated with an anchor sequence-mediated conjunction operably linked to the target gene.

38. The method of any of claims 35-37, wherein expression is:
decreased by 10, 20, 30, 40, 50, 60, 70, 80, 90, or 100% compared to expression in an otherwise similar cell in the absence of the expression repression system; and/or
decreased for a time period of at least 1, 2, 3, 4, 5, 6, 7, 8, 9, 10, 11, 12, 13, 14, 15, 16, 17, 18, 19, 20, 21, 22, 23, 24, or 25 days, or at least 1, 2, 3, 4, 5, 6, 7, 8, 9, or 10 cell divisions, e.g., as measured by ELISA or as described in Examples 2-4.

39. The method of any of claims 35-38, wherein the decrease in expression comprises a decrease in:
the level of RNA, e.g., mRNA, encoded by the target gene; and/or
the level of a protein encoded by the target gene.

40. The method of any of claims 35-39, wherein providing comprises:
contacting the cell with a nucleic acid, e.g., vector, encoding the expression repression system to the cell comprising the target gene; or
contacting the cell with a lipid nanoparticle (LNP) comprising the expression repression system or a nucleic acid, e.g., vector, encoding the expression repression system.

41. A method of treating a condition associated with over-expression or mis-regulation of a target gene in a subject, comprising:
administering the expression repression system, nucleic acid, polypeptide, pharmaceutical composition, or vector of any of claims 1-32 or 34 to the subject,
thereby treating a condition associated with over-expression of a target gene in a subject.

42. A method of making a cell comprising an expression repression system, the method comprising:
providing the expression repression system, nucleic acid, pharmaceutical composition, or vector of any of claims 1-29, 31, 32 or 34; and
contacting the cell with the expression repression system, nucleic acid, vector, or pharmaceutical composition,
thereby making a cell comprising an expression repression system.
